(19) United States
(12) Patent Application Publication
(10) Pub. No.: US 2022/0089609 A1
(43) Pub. Date: Mar. 24, 2022

(54) SUBSTITUTED QUINAZOLINONE DERIVATIVES AND THEIR USE AS POSITIVE ALLOSTERIC MODULATORS OF MGLUR4

(71) Applicant: DOMAIN THERAPEUTICS, IIIkirch-Graffenstaden (FR)

(72) Inventors: Camille AMALRIC, Strasbourg (FR); Anaïs BARRE, IIIkirch-Graffenstaden (FR); Stephan SCHANN, IIIkirch-Graffenstaden (FR); Stanislas MAYER, Eschau (FR); Ismet DORANGE, Stockholm (SE); Baptiste MANTEAU, Ways (BE); Anne-Laure BLAYO, Souffelweyersheim (FR)

(73) Assignee: DOMAIN THERAPEUTICS, IIIkirch-Graffenstaden (FR)

(21) Appl. No.: 17/263,071

(22) PCT Filed: Jul. 26, 2019
(86) PCT No.: PCT/EP2019/070178

§ 371 Date: Jan. 25, 2021

Related U.S. Application Data
(60) Provisional application No. 62/703,582, filed on Jul. 26, 2018.

(30) Foreign Application Priority Data
Jul. 26, 2018 (EP) 18315019.2

(51) Int. Cl. C07D 498/10; C07D 487/04; C07D 401/14; C07D 495/04; C07D 413/14; C07D 405/14; C07D 519/00; A61P 25/16

(52) U.S. Cl. CPC C07D 498/10; C07D 487/04; C07D 401/14; A61P 25/16; C07D 413/14; C07D 405/14; C07D 519/00; C07D 495/04

(57) ABSTRACT

The present invention relates to novel quinazolinone derivatives of formula (I) as well as pharmaceutical compositions containing these compounds. The compounds of formula (I) as provided herein can act as positive allosteric modulators of metabotropic glutamate receptor subtype 4 (mGluR4), and can thus be used as therapeutic agents, particularly in the treatment or prevention of conditions associated with altered glutamatergic signalling and/or functions or conditions which can be affected by alteration of glutamate level or signalling.

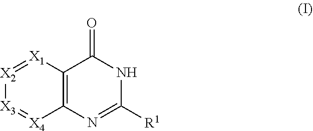

The present invention relates to novel quinazolinone derivatives of formula (I) as well as pharmaceutical compositions containing these compounds. The compounds of formula (I) as provided herein can act as positive allosteric modulators of metabotropic glutamate receptor subtype 4 (mGluR4), and can thus be used as therapeutic agents, particularly in the treatment or prevention of conditions associated with altered glutamatergic signalling and/or functions or conditions which can be affected by alteration of glutamate level or signalling.

Glutamate is the major amino-acid transmitter in the mammalian central nervous system (CNS). Glutamate plays a major role in numerous physiological functions, such as learning and memory but also sensory perception, development of synaptic plasticity, motor control, respiration and regulation of cardiovascular function. Furthermore, glutamate is at the center of several different neurological and psychiatric diseases, where there is an imbalance in glutamatergic neurotransmission.

Glutamate mediates synaptic neurotransmission through the activation of ionotropic glutamate receptor channels (iGluRs), namely the NMDA, AMPA and kainate receptors which are responsible for fast excitatory transmission (Nakanishi S et al., (1998) Brain Res. Rev., 26:230-235).

In addition, glutamate activates metabotropic glutamate receptors (mGluRs) which have a more modulatory role that contributes to the fine-tuning of synaptic efficacy. The mGluRs are G protein-coupled receptors (GPCRs) with seven-transmembrane spanning domains and belong to GPCR family 3 along with the calcium-sensing, GABAb and pheromone receptors. The mGluR family is composed of eight members. They are classified into three groups (group I comprising mGluR1 and mGluR5; group II comprising mGluR2 and mGluR3; group III comprising mGluR4, mGluR6, mGluR7 and mGluR8) according to sequence homology, pharmacological profile and nature of intracellular signalling cascades activated (Schoepp D et al., (1999) Neuropharmacology, 38: 1431-1476).

Glutamate activates the mGluRs through binding to the large extracellular amino-terminal domain of the receptor, herein called the orthosteric binding site. This activation induces a conformational change of the receptor which results in the activation of the G-protein and intracellular signalling pathways.

In the central nervous system, mGluR4 receptors are expressed most intensely in the cerebellar cortex, basal ganglia, sensory relay nuclei of the thalamus and hippocampus (Bradley S R et al., (1999) Journal of Comparative Neurology, 407:33-46; Corti C et al., (2002) Neuroscience, 1 10:403-420). The mGluR4 subtype is negatively coupled to adenylate cyclase via activation of the Gi/o protein, is expressed primarily on presynaptic terminals, functioning as an autoreceptor or heteroreceptor and activation of mGluR4 leads to decreases in transmitter release from presynaptic terminals (Corti C et al., (2002) Neuroscience, 1 10:403-420; Millan C et al., (2002) Journal of Biological Chemistry, 277:47796-47803; Valenti O et al., (2003) Journal of Neuroscience, 23:7218-7226). In certain brain tissues such as rodent cerebellar cortex, mGluR4 receptors can also couple to Gq protein and PLC effector system, again to reduce glutamate synaptic transmission (Chardonnet S et al., (2017) Neuropharmacology, 121:247-260).

Until the recent discovery of a variable pocket, responsible for intra group III mGluR subtype selectivity, and neighboring the glutamate binding site (Goudet C et al., (2012) FASEB J, 26(4): 1682-93; Selvam C et al., (2018) J Med Chem, 61(5): 1969-89), orthosteric agonists of mGluR4 were mostly not selective and could therefore activate the other group III mGluRs (Schoepp D et al., (1999) Neuropharmacology, 38: 1431-1476). The group III orthosteric agonist L-AP4 (L-2-amino-4-phosphonobutyrate) was able to reduce motor deficits in animal models of Parkinson's disease (Valenti O et al., (2003) J. Neurosci., 23:7218-7226) and decrease excitotoxicity (Bruno V et al., (2000) J. Neurosci., 20; 6413-6420) and these effects appear to be mediated through mGluR4 (Marino M J et al., (2005) Curr. Topics Med. Chem., 5: 885-895). In addition to L-AP4, ACPT-1, another selective group III mGluR agonist has been shown to cause a dose and structure-dependent decrease in haloperidol-induced catalepsy and attenuated haloperidol-increased Proenkephalin mRNA expression in the striatum (Konieczny J et al., (2007) Neuroscience, 145:611-620). Furthermore, Lopez et al. (2007, J. Neuroscience, 27:6701-6711) have shown that bilateral infusions of ACPT-I or L-AP4 into the globus pallidus fully reversed the severe akinetic deficits produced by 6-hydroxydopamine lesions of nigrostriatal dopamine neurons in a reaction-time task without affecting the performance of controls. In addition, the reversal of haloperidol-induced catalepsy by intrapallidal ACPT-1 was prevented by concomitant administration of a selective group III receptor antagonist (R5)-alpha-cyclopropyl-4-phosphonophenylglycine. These results suggest that, among mGluR subtypes, group III mGluR and especially mGluR4 are very interesting novel drug targets for the treatment of Parkinson's disease (for review see Conn P J et al., (2005) Nature Review Neuroscience, 6:787-798 and more recently, Charvin D, (2018) Neuropharmacology, 135:308-315).

The common endpoint of Parkinson's disease (PD) pathology is a progressive degeneration of the dopaminergic neurons located in the pars compacta of the substantia nigra (SNpc) that project and release dopamine into the striatum. PD symptoms usually appear when more than 60% of SNpc neurons have already disappeared. This results in profound movement disturbances including rest tremor, rigidity and stiffness, gait and balance control dysfunctions and dementia that dramatically deteriorate patients and family quality of life.

Current treatments aim at substituting the missing dopamine or mimicking its effects by chronically providing patients with the dopamine precursor L-DOPA, inhibitors of dopamine catabolic enzymes (MAO inhibitors) or direct dopamine receptors agonists. Although these treatments proved relatively efficient in controlling the main symptoms of PD, their chronic administration is associated with serious side effects. Classical treatment of Parkinsonism typically involves the use of levodopa combined with carbidopa (SINEMET™) or benserazide (MADOPAR™). Dopamine agonists such as bromocriptine (PARLODEL™), lisuride and pergolide (CELANCE™) act directly on dopamine receptors and are also used for the treatment of Parkinsonism. These molecules have the same side-effect profile as levodopa. For example, the efficacy of L-DOPA following few years of treatment invariably tends to diminish in intensity and stability leading to uneven on/off periods that require an increase in dosing. In addition, chronic administration of high doses of L-DOPA is associated with the occurrence of involuntary movements (dyskinesia). Levodopa-induced dyskinesia affects almost all PD patients treated with levodopa at some point during the disease course, although various attempts have been made to manage this disorder (Rascol O et al., (2015), Mov Disord, 30(11):1451-1460). Therefore, and since 86% of the PD patients are currently under levodopa treatment, there is an urgent clinical need to improve levodopa-induced dyskinesia (Hechtner M C et al., (2014) Park Relat Disord, 20: 969-74). Massive supply of dopamine in the brain has also been associated with psychiatric disturbances including depression, psychotic symptoms, obsessive behaviours sleep disturbances etc. Finally, none of the compounds of the current pharmacopeia for PD have demonstrated neuroprotective activity that would delay disease progression. Therefore, to address these important unmet medical needs, efforts are required to develop new treatments for PD that target the neurochemical systems downstream dopamine itself.

The control of movements by dopamine in healthy subjects follows a complex pattern of neurochemical systems and brain structures interactions for which a model has been described in the last decades (Wichmann T and Delong M R, (2003) Adv Neurol 91:9-18). This model is now evolving toward a more elaborated understanding of basal ganglia functioning (for reviews see Kravitz A V et al., (2010) Nature, 466: 622-26; Cui G et al., (2013) Nature, 494: 238-42; Cazorla M et al., (2015) Mov Disord, 30: 895-903). The basal ganglia that is composed mainly of the substantia nigra (SN), and the striatal and thalamic complex constitutes the cornerstone of these interactions. The internal capsule of the globus pallidus (GPi) and SN pars reticulata (SNpr) fulfil the roles of relays between cortical areas that directly control movements and the basal ganglia itself. GPi and SNpr receive both an inhibitory direct connection (direct pathway) and an excitatory indirect input (indirect pathway) from the basal ganglia. Both pathways are modulated by dopamine with opposite valence so that the direct pathway is stimulated while the indirect pathway is inhibited by dopamine. Consequently in the diseased brain, the lack of dopamine leads to a dysregulation of the output activity of both the direct and indirect pathways. In particular, the indirect pathway gets overactivated, which is reflected by increased GABA release into the globus pallidus external segment (GPe). Consequently, glutamate release is increased in the SN pars compacta (SNpc), GPi and SNpr. These distortions of the balance of neurotransmission in the direct and indirect pathways are believed to result in movement control abnormalities and the precipitation of neurodegeneration of dopaminergic neurons. Fine analysis of these pathways provided insights on the possibility to target neurochemical pathways downstream dopamine to restore its function in the PD brain without interfering directly with it. In particular, metabotropic glutamate receptors (mGluRs) have been shown to modulate neurotransmitter release at the presynaptic level. Specifically, mGluR4 predominantly expressed in the brain in discrete areas was demonstrated to dampen glutamate and GABA neurotransmissions at the subthalamic nucleus (STN)—SNpc (Valenti O et al., (2005) J Pharmacol Exp Ther 313:1296-1304) and striatum—GPe (Valenti O et al., (2003) J Neurosci 23:7218-7226; Cuomo D et al., (2009) J Neurochem, 109: 1096-1105)) synapses, respectively. mGluR4 is more abundant in striato-pallidal synapses than in striato-nigral synapses, and its localization suggests function as a presynaptic heteroreceptor on GABAergic neurons (Bradley S R et al., (1999) Journal of Comparative Neurology, 407:33-46) suggesting that selective activation or positive modulation of mGluR4 would decrease GABA release in this synapse thereby decreasing output of the indirect pathway and reducing or eliminating the Parkinson's disease symptoms. Moreover, mGluR4 is also expressed presynaptically in the corticostriatal glutamatergic terminals that target the indirect pathway neurons (Bradley S R et al., (1999) J Comp Neurol, 407: 33-46). Activation of mGluR4 at this site is expected to preferentially inhibit stimulation of the already hyperactive indirect pathway, while preserving the excitation of the direct pathway, thereby normalizing basal ganglia output (Bennouar K E et al., (2013) Neuropharmacology, 66: 158-69; Gubellini P et al., (2014) Neuropharmacology, 85: 166-77; Iskhakova L et al., (2016) Brain Struct Funct, 221(9): 4589-99).

Furthermore, behavioural analyses confirmed the beneficial effects of stimulation of mGluR4 in both chronic and acute rat models of motor symptoms of PD. For example, the cataleptic behaviour observed following haloperidol administration and reserpine-induced immobility were both reversed by positive allosteric modulator (PAM) such as VU0155041 (Niswender C M et al., (2008) Mol Pharmacol 74:1345-1358; Niswender C M et al., (2016) ACS Chem Neurosci, 7: 1201-11; Le Poul E et al., (2012) J Pharmacol Exp Therapeut, 343: 167-77; Charvin D et al., (2017) J Med Chem, 60(20): 8515-37). These basic models mimic key features of the human disease that are rigidity and akinesia, respectively. More advanced models of PD motor symptoms (such rat unilateral or bilateral 6-OHDA, or MitoPark mouse transgenic model) were also used to demonstrate efficacy of PAMs activating group III mGluR and in particular mGluR4 (Dube A et al., (2014) J Neurol Sci, 510(14): 452-53; Niswender C M et al., (2016) ACS Chem Neurosci, 7: 1201-11; Le Poul E et al., (2012) J Pharmacol Exp Therapeut, 343: 167-77; Charvin D et al., (2017) J Med Chem, 60(20): 8515-37).

Finally, the increased release of glutamate is believed to participate, at least in part, in the degeneration of the remaining dopaminergic neurons thereby worsening the condition and reducing treatment efficacy. Hence, the mGluR4 positive allosteric modulator (PAM) PHCCC, which reduces glutamate release, also protects neurons from further degenerating in rats treated with the neurotoxin 6-hydroxydopamine (6-OHDA) that selectively destroys dopaminergic neurons (Vernon A C, (2009) J Neurosci 29: 12842-12844; Betts M J et al., (2012) Br J Pharmacol, 166: 2317-30). Similar results were obtained with PHCCC in 1-methyl-4-phenyl-1,2,3,6-tetrahydropyridine) (MPTP) (Battaglia G et al., (2006) J Neurosci, 26: 7222-29) or in NMDA-lesioned mice using group III mGluR agonist (+)-4-phosphonophenylglycine PPG (Bruno V et al., (2000) J Neurosci, 20: 6413-20).

More recently, mGluR4 PAM compounds from invention WO 2017/032874 were found to be highly effective in the prevention and/or treatment of levodopa-induced dyskinesia (LID), as demonstrated in an MPTP monkey model. These results confirm the therapeutic potential of group III mGluR activators to decrease incidence of dyskinesia, already published using other mGluR4 PAM compound, namely LuAF21934, in a 6-OHDA rat model of LID a few years ago (Bennouar K E et al., (2013) Neuropharmacology, 66: 158-69).

Altogether these results suggest that stimulation of mGluR4 and more generally group III mGluRs has great potential to alleviate PD symptoms in patients, including levodopa-induced dyskinesia, and provide neuroprotection to the remaining neurons.

A new avenue for developing selective compounds acting at mGluRs is to identify molecules that act through allosteric mechanisms, modulating the receptor by binding to a site different from the highly conserved orthosteric binding site.

Positive allosteric modulators of mGluRs have emerged recently as novel pharmacological entities offering this attractive alternative. This type of molecule has been discovered for mGluR1, mGluR2, mGluR3, mGluR4, mGluR5, mGluR7 and mGluR8 (Knoflach F et al. (2001) Proc. Natl. Acad. Sci. USA, 98: 13402-13407; Johnson M P et al., (2002) Neuropharmacology, 43:799-808; O'Brien J A et al., (2003) Mol. Pharmacol., 64:731-740; Johnson M P et al, (2003) J. Med. Chem., 46:3189-3192; Marino M J et al., (2003) Proc. Natl. Acad. Sci. USA, 100: 13668-13673; Mitsukawa K et al., (2005) Proc. Natl. Acad. Sci. USA, 102(51): 18712-18717; Wilson J et al., (2005) Neuropharmacology, 49:278; Mutel V, (2002) Expert Opin. Ther. Patents, 12: 1-8; Kew J N, (2004) Pharmacol. Ther., 104(3):233-244; Johnson M. et al., (2004) Biochem. Soc. Trans., 32:881-887; Ritzen A, Mathiesen, J M and Thomsen C, (2005) Basic Clin. Pharmacol. Toxicol., 97:202-213; Schann S et al., (2010) J Med Chem, 53(24): 8775-79).

Looking closer at groups III mGluRs, examples of allosteric ligands were so far mostly described for the mGluR subtype 4 (mGluR4). PHCCC, MPEP and SIB1893 (Maj M et al., (2003) Neuropharmacology, 45(7), 895-903; Mathiesen J M et al., (2003) Br. J, Pharmacol. 138(6), 1026-30) were the first ones described in 2003. More recently, more potent positive allosteric modulators were reported in the literature by different universities and private companies (Niswender C M et al., (2008) Mol. Pharmacol. 74(5), 1345-58; Niswender C M et al., (2008) Bioorg. Med. Chem. Lett 18(20), 5626-30; Williams R et al., (2009) Bioorg. Med. Chem. Lett. 19(3), 962-6; Engers D W et al., (2009) J. Med. Chem., 52(14): 4115-18; Le Poul E et al., (2012) J Pharmacol Exp Therapeut, 343: 167-77; Bennouar K E et al., (2013) Neuropharmacology, 66: 158-69; Dube A et al., (2014) J Neurol Sci, 510(14): 452-53) and in two patent publications describing families of amido and heteroaromatic compounds (WO 2009/010454 and WO 2009/010455), also published in Charvin D et al. ((2017) J Med Chem, 60: 8515-37).

Regarding other group III mGluR subtypes, fewer allosteric ligands were identified so far. AMN082 is a mGluR7 specific allosteric agonist binding in the seven transmembrane domain of the receptor, while XAP044 is an antagonist binding in the large amino terminal extracellular domain, but at a different site than glutamate itself (Mitsukawa K et al., (2005) PNAS, 102(51): 18712-17; Gee C E et al., (2014) J Biol Chem, 289(16): 10975-87). Other mGluR7 Negative Allosteric Modulator (NAM) chemical series with undisclosed structures are currently being developed by Pragma Therapeutics for hearing and stress disorders. AZ12216052 is a mGluR8 PAM discovered by Astra Zeneca and which was shown to reduce measures of anxiety in several rodent models (Duvoisin et al., (2010) Behav Brain Res, 212(2): 168-73).

PHCCC (N-phenyl-7-(hydroxyimino)cyclopropa[6]chromen-la-carboxamide), a positive allosteric modulator of mGluR4 not active on other mGluRs (Maj et al., (2003) Neuropharmacology, 45:895-906), has been shown to be efficacious in animal models of Parkinson's disease thus representing a potential novel therapeutic approach for Parkinson's disease as well as for other motor disorders and disturbances (Marino et al., (2003) Proc. Nat. Acad. Sci. USA, 100: 13668-13673), and neurodegeneration in Parkinson's disease (Marino et al., (2005) Curr. Topics Med. Chem., 5:885-895; Valenti et al., (2005) J. Pharmacol. Exp. Ther., 313: 1296-1304; Vernon et al., (2005) Eur. J. Neurosci., 22: 1799-1806, Battaglia et al., (2006) J. Neurosci., 26:7222-7229)). Since these seminal publications, other mGluR4 positive modulators, and more generally group III mGluR positive modulators, have shown promising results in animal models of Parkinson's disease and neurodegeneration (Conn J et al., (2005) Nat Rev. Neuroscience, 6(10), 787-98; Vernon A C et al., (2007) J. Pharmacol. Exp. Then, 320(1), 397-409; Lopez S et al., (2008) Neuropharmacology, 55(4), 483-90; Vernon A C et al., (2008) Neuroreport, 19(4), 475-8; Niswender C M et al., (2008) Mol. Pharmacol. 74(5), 1345-58). The other subtypes of group III mGlu receptors, namely mGluR7 and mGluR8, have also been demonstrated as having potential neuroprotective (Wang W Y et al., (2012) Neuroscience, 205: 167-77) and anti-parkinsonian activities (for review see Amalric M et al., (2013) Neuropharm, 66: 53-64; Amalric M, (2015) CurrOpin Pharmacol, 20: 29-34; Gubellini P et al., (2017) The Receptors, Humana Press, 33-57; Litim N et al., (2017) Neuropharm, 115: 166-179). Indeed, mGluR7 specific allosteric agonist AMN082 has been shown to reverse haloperidol-induced catalepsy and akinesia in the reserpine-treated rat (Greco B et al., (2010) J Pharmacol Exp Ther, 332(3): 1064-71; Broadstock M et al., (2012) British J of Pharmacol, 165(4b): 1034-45; Konieczny J and Lenda T, (2013) Pharmacol Rep, 65(5): 1194-203). Implication of subtype mGluR8 was also tested using specific agonist (S)-3,4-dicarboxyphenylglycine (DCPG) which was shown to reverse prolonged (3 doses of reserpine or haloperidol administered spaced over 18-20 h period prior measurement) but no acute (single dose of reserpine or haloperidol administered 2 h prior measurement) catalepsy and akinesia, indicating that activating this subtype may be of particular interest to restore pro-motor effects in case of prolonged dopamine depletion (Johnson K A et al., (2013) Neuropharmacol, 66: 187-95).

PHCCC showed neuroprotection against beta Amyloid Protein- and NMDA-toxicity in mixed cultures of mouse cortical neurons, thereby demonstrating the capacity of mGluR4 positive modulators to protect against neurodegeneration in Alzheimer's disease or due to ischemic or traumatic insult (Maj et al., (2003) Neuropharmacology, 45:895-906). Other studies validate the potential use of group III mGluR modulators for treatment of Alzheimer's disease. Interesting data going in this direction come from in vivo data using mGluR7 knock-out mice, which showed that group III mGluR7 promotes short term memory (Holscher C et al., (2004) Behav Brain Res, 154(2): 473-81).

Neuroprotective potential of the group III mGlu receptor agonist ACPT-I was recently demonstrated in animal models of ischemic stroke using both in vitro and in vivo studies, which revealed that group III mGluR activation may be not only neuroprotective against ischemic neuronal damage, but may also diminish the post-ischemic functional deficits (Domin H et al., (2016) Neuropharmacology, 102: 276-94).

mGluR4 positive allosteric modulators such as PHCCC or ADX88178 have also been shown to be active in animal models of anxiety (Stachowicz et al., (2004) Eur. J. Pharmacol., 498: 153-156; Kalinichev M et al., (2014) J Pharmacol Exp Ther, 350(3): 495-505) and depression (Palucha A et al., (2004) Neuropharmacology 46(2), 151-9). Previously, group III mGluR agonist ACPT-1 had been shown to produce a dose-dependent anti-conflict effect after intrahippocampal administration and anti-depressant-like effects in rats after intracerebroventricular administration (Tatarczynska et al., (2002) Pol. J. Pharmacol., 54(6):707-710). Anti-depressant effects were potentiated when using combination of both PHCCC and ACPT-1 compounds (Klak K et al., (2006) Amino Acids 32(2), 169-72). More recently, ACPT-1 has also been shown to have anxiolytic-like effects in the stress-induced hyperthermia, in the elevated-plus maze in mice and in the Vogel conflict test in rats when injected intraperitoneally (Stachowicz et al., (2009) Neuropharmacology, 57(3): 227-234). Activation of group III mGluR8 also reduces anxiety-like behavior in rodent models, as demonstrated by animal treatment with mGluR 8 specific agonist DCPG or PAM AZ12216052 (Duvoisin et al., (2010) Behav Brain Res, 212(2): 168-73; for review see Raber J and Duvoisin R M, (2015) Expert Opin Investig Drugs, 24(4): 519-28).

Group III mGluR modulators showed positive results in several animal models of schizophrenia (Paiucha-Poniewiera A et al., (2008) Neuropharmacology, 55(4), 517-24). Similarly, ADX88178, a brain-penetrant positive allosteric modulator of the mGlu4 receptor was shown to be active in rodent models of obsessive compulsive disorder (OCD), fear and psychosis (Kalinichev M et al., (2014) J Pharmacol Exp Ther, 350(3): 495-505).

In addition, mGluR4 positive modulators were shown to relieve autistic-like syndrome in rodent models of autism spectrum disorder (Becker J A et al., (2014) Neuropsychopharmacology, 39(9): 2049-2060), while activators of mGluR7, another subtype of group III mGlu receptors, are under investigation by Vanderbilt University for treatment of Rett syndrome (Gogliotti R G et al., (2017) Sci Transl Med, 9(403)).

Association between epilepsy and mGluR4 transcriptional level and/or genetic variants has recently been published (Parihar R et al., (2014) J Genet, 93(1): 193-197; Dammann F et al., (2018) Epilepsy Res, 139: 157-163), which indicates that mGluR4 modulators may be useful as treatments for epilepsy.

The [beta]-chemokine RANTES is importantly involved in neuronal inflammation and has been implicated in the pathophysiology of multiple sclerosis. Activation of Group III mGluRs with L-AP4 reduced the synthesis and release of RANTES in wild-type cultured astrocytes, whereas the ability of L-AP4 to inhibit RANTES was greatly decreased in astrocyte cultures from mGluR4 knockout mice (Besong et al., (2002) Journal of Neuroscience, 22:5403-5411). Expression of mGluR4 on dendritic cells can influence the TH17/Treg balance (Hansen A M and Caspi R R, (2010) Nat Med, 16(8): 856-8; Zhao G et al., (2017) Int Immunopharmacol, 46: 80-86). Activation of mGluR4 via endogenous agonist cinnabarinic acid or highly selective and potent PAM ADX88178 is protective in mouse model of multiple sclerosis, namely experimental autoimmune encephalomyelitis (EAE) (Fazio F et al., (2014) Neuropharmacology, 81: 237-43; Volpi C et al., (2016) Neuropharmacology, 102: 59-71). The underlying mechanism was recently deciphered: activation of mGluR4 with ADX88178 attenuates LPS-induced inflammation in primary microglia, leading to a decrease in the expression of TNFα, MHCII, and iNOS, markers of pro-inflammatory responses (Ponnazhagan R et al., (2016) J Neuroimmune Pharmacol, 11(2): 231-7). Altogether, these data suggest that positive allosteric modulators of mGluR4 may be an effective treatment for neuroinflammatory disorders of the central nervous system, including multiple sclerosis and related disorders (for review see Levite M, (2017) J Neural Transm, 124(7): 775-98).

Two different variants of the mGluR4 receptor are expressed in taste tissues and may function as receptors for the umami taste sensation (Monastyrskaia et al., (1999) Br. J Pharmacol., 128: 1027-1034; Toyono et al., (2002) Arch. Histol. Cytol., 65:91-96; Eschle B K., (2008) Neuroscience, 155(2), 522-9). Thus positive allosteric modulators of mGluR4 may be useful as taste agents, flavour agents, flavour enhancing agents or food additives.

There is anatomical evidence that the majority of vagal afferents innervating gastric muscle express group III mGluRs (mGluR4, mGluR6, mGluR7 and mGluR8) and actively transport receptors to their peripheral endings (Page et al., (2005) Gastroenterology, 128:402-10). Recently, it was shown that the activation of peripheral group III mGluRs inhibited vagal afferents mechanosensitivity in vitro which translates into reduced triggering of transient lower esophageal sphincter relaxations and gastroesophageal reflux in vivo (Young et al., (2008) Neuropharmacol, 54:965-975). Labelling for mGluR4 and mGluR8 was abundant in gastric vagal afferents in the nodose ganglion, at their termination sites in the nucleus tractus solitarius and in gastric vagal motoneurons. These data suggest that positive allosteric modulators of group III mGluR may be an effective treatment for gastroesophageal reflux disease (GERD) and lower esophageal disorders and gastro-intestinal disorders.

Activation of mGluR4 receptors which are expressed in a- and F-cells in the islets of Langerhans inhibits glucagon secretion. Molecules which activate or potentiate the agonist activity of these receptors may be an effective treatment for hyperglycemia, one of the symptoms of type 2 diabetes (Uehara et al., (2004) Diabetes, 53:998-1006).

Moreover, mGluR4 signaling is also a mechanism involved in modulation of chronic pain (Goudet C et al., (2008) Pain, 137(1), 112-24; Zhang H M et al., (2009) Neuroscience, 158(2), 875-84; Zussy C et al., (2018) Mol Psychiatry, 23(3): 509-520; for review see Palazzo E et al., (2017) J Neurochem, 141(4): 507-519).

Finally, mGluR4 was shown to be expressed in prostate cancer cell-line (Pessimissis N et al., (2009) Anticancer Res. 29(1), 371-377), colorectal carcinoma (Chang H J et al., (2005) CIL Cancer Res. 1 1 (9), 3288-95) or more recently in osteosarcoma (Yang et al., (2014), J Cancer Res Clin Oncol, 140(3):419-426; Wang et al., (2016), Mol Cli Oncol, 4(1):65-69), and its activation with PHCCC was shown to inhibit growth of medulloblastomas (Iacoveili L et al., (2006) J. Neurosci. 26(32) 8388-97). Similarly, very recent data in neuroblastoma and glioma cell lines have demonstrated that mGluR8 overexpression induced a decreased cell proliferation, increased apoptosis and elevated vulnerability to some cytotoxic agents (Jantas D et al., (2018) Cancer Lett, 3835(18): 30400-2). Group III mGluR modulators may therefore also be used for the treatment of cancers.

Further prior art documents in relation with structurally-related compounds are as follows:

WO 01/083456 deals with condensed heteroaryl derivatives.

WO 02/028841 relates to reagents for labelling biomolecules having an aldehyde or keto function.

WO 03/048152 is directed to inflammation modulators.

WO 2004/024162 discloses 2-amino-4-quinazolinones as LXR nuclear receptor binding compounds.

WO 2004/041755 describes quinazolinone compounds as calcilytics.

WO 2004/065392 discloses certain substituted quinoline and quinazoline compounds as inhibitors of ALK5 kinase.

WO 2004/078733 deals with condensed pyrimidines and pyridines and their use as ALK-5 receptor ligands.

WO 2004/078733 relates to quinazolinones useful as modulators of ion channels.

WO 2005/035526 relates to bicyclic compounds and their therapeutic use.

WO 2006/051290 is directed to pharmaceutical compositions.

WO 2006/071095 discloses quinazoline derivatives for the treatment and prevention of obesity.

WO 2008/020302 describes heteroaromatic quinoline-based compounds.

WO 2009/064388 deals with inhibitors of human methionine aminopeptidase 1 and methods of treating disorders.

WO 2009/111943 relates to compounds as estrogen related receptor modulators and uses thereof.

WO 2010/018458 is directed to phenol derivatives and methods of use thereof.

WO 2010/056758 discloses quinazoline derivatives as kinase inhibitors.

WO 2010/106436 describes certain anti-inflammatory agents.

WO 2010/136475 deals with substituted quinazolines as fungicides.

WO 2011/011522 relates to potent small molecule inhibitors of authophagy and methods of use thereof.

WO 2011/045258 is directed to condenzed azine derivatives for the treatment of diseases related to the aceytlcholine receptor.

WO 2011/082337 discloses therapeutic compounds and related methods of use.

WO 2011/104183 relates to microbiocidal, particularly fungicidal, 2-(pyridin-2-yl)pyrimidines for use in agriculture or horticulture.

WO 2012/028578 discloses substituted fused pyrimidinones and dihydropyrimidinones for raising the tolerance of plants towards abiotic stress, and also for strengthening plant growth and/or for increasing plant yield.

WO 2013/003586 describes certain quinazoline derivatives as striatal-enriched tyrosine phosphatase (STEP) inhibitors.

WO 2015/015318 deals with certain quinazolinones as bromodomain inhibitors.

WO 2016/199943 is directed to heterocyclic compounds as BET family protein inhibitors.

CN 103319408 describes compounds for preventing and treating cardiovascular diseases.

Further prior art documents, including documents in relation with mGluR4 PAM compounds, are as follows:

EP 2953532; WO 2011/050305; WO 2011/029104; WO 2011/100607; WO 2011/051478; WO 2012/009001; WO 2013/107862; WO 2014/117920; WO 2014/121883; WO 2014/121885; WO 2015/044075; WO 2015/104271; WO 2016/146600; WO 2016/030444; WO 2017/009275; US 2018/057490; US 2018/057491; US 2018/021312; US 2018/022744; US 2018/022745; and US 2018/022746.

The citation of any reference in this application is not an admission that the reference is relevant prior art to this application.

The present invention provides novel compounds that exhibit highly potent positive allosteric modulator activity on mGluR4, which renders them particularly suitable as therapeutic agents. The invention also provides compounds that are positive allosteric modulators of mGluR4 and show advantageous pharmacokinetic properties.

The present invention thus solves the problem of providing novel and/or improved therapeutic agents for the medical intervention in conditions associated with altered glutamatergic signalling and/or functions and conditions which can be affected by alteration of glutamate level or signalling.

Accordingly, the present invention provides a compound of formula (I)

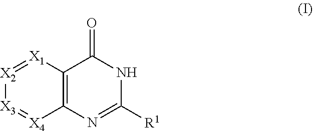

as described and defined herein below, or a pharmaceutically acceptable salt thereof.

The compounds of the present invention have been found to be potent positive allosteric modulators of metabotropic glutamate receptor subtype 4 (mGluR4), and can thus advantageously be used as therapeutic agents, particularly in the treatment or prevention of conditions associated with altered glutamatergic signalling and/or functions or conditions which can be affected by alteration of glutamate level or signalling.

In the context of the present invention, it has surprisingly been found that the lactam nitrogen ring atom comprised in the quinazolinone ring of the compounds of formula (I) needs to be unsubstituted, as also shown in formula (I), for the compounds to exhibit mGluR4 positive allosteric modulator (PAM) activity. This is demonstrated by comparison of the mGluR4 PAM activities of the compounds depicted in the following scheme:

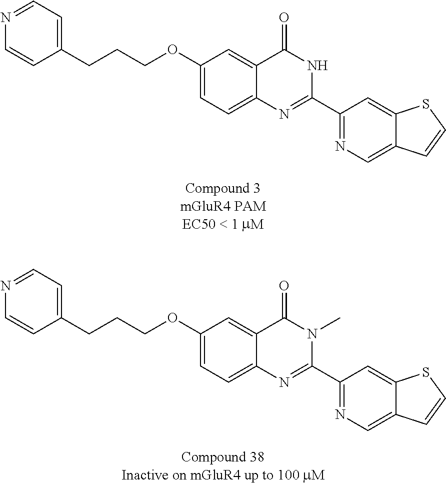 [SMILES: O=c1[nH]c(-c2cc3sccc3cn2)nc2ccc(OCCCc3ccncc3)cc12 ; Cn1c(-c2cc3sccc3cn2)nc2ccc(OCCCc3ccncc3)cc2c1=O]

Compound 3 according to the invention, in which the lactam nitrogen ring atom of the quinazolinone ring is unsubstituted, is a positive allosteric modulator (PAM) of mGluR4 having an EC50 lower than 1 μM.

In contrast thereto, the reference compound 38, which is an N-substituted analogue of compound 3 bearing a methyl substituent at the lactam nitrogen ring atom of the quinazolinone ring, has no PAM activity on mGluR4 up to 100 μM.

It has further surprisingly been found that the aromatic ring group R¹ contained in the compounds of formula (I) needs to be linked to the remainder of the compound [i.e., to the quinazolinone ring comprised in formula (I)] through a ring carbon atom and needs to contain a nitrogen ring atom in ortho-position, i.e. in the position adjacent to the ring carbon atom that is linked to the remainder of the compound of formula (I) [i.e., to the quinazolinone ring comprised in formula (I)], in order to exhibit mGluR4 PAM activity. This is demonstrated by comparison of the mGluR4 PAM activities of the compounds depicted in the following scheme:

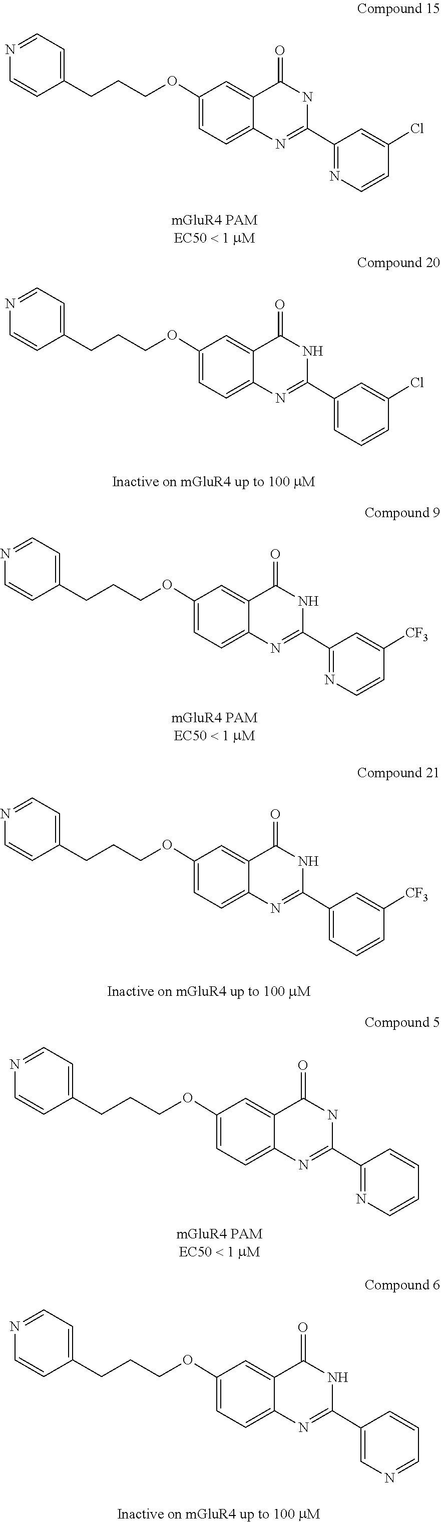

The compounds 15, 9 and 5 according to the invention, which contain an aromatic ring group R¹ having a nitrogen ring atom in the position adjacent to the carbon ring atom that connects the aromatic ring (R¹) to the remainder of the compound (i.e., to the quinazolinone ring comprised in the respective compound), are positive allosteric modulators (PAMs) of mGluR4 having an EC50 lower than 1 μM.

In contrast thereto, the reference compounds 20, 21 and 6, which contain an aromatic ring group R¹ that does not have a nitrogen ring atom in the specific position adjacent to the carbon ring atom which connects the aromatic ring to the remainder of the respective compound, have no PAM activity on mGluR4 up to 100 μM.

The groups/variables in the compound of formula (I) or the pharmaceutically acceptable salt thereof, which is provided in accordance with the present invention, have the following meanings:

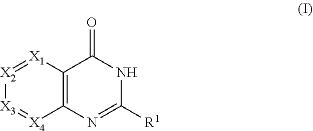

R¹ is selected from any one of the following groups:

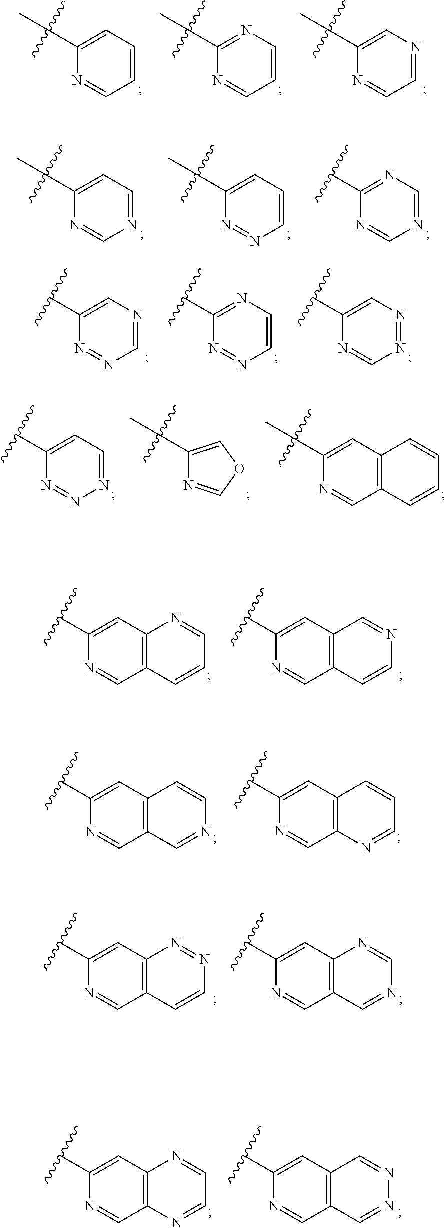

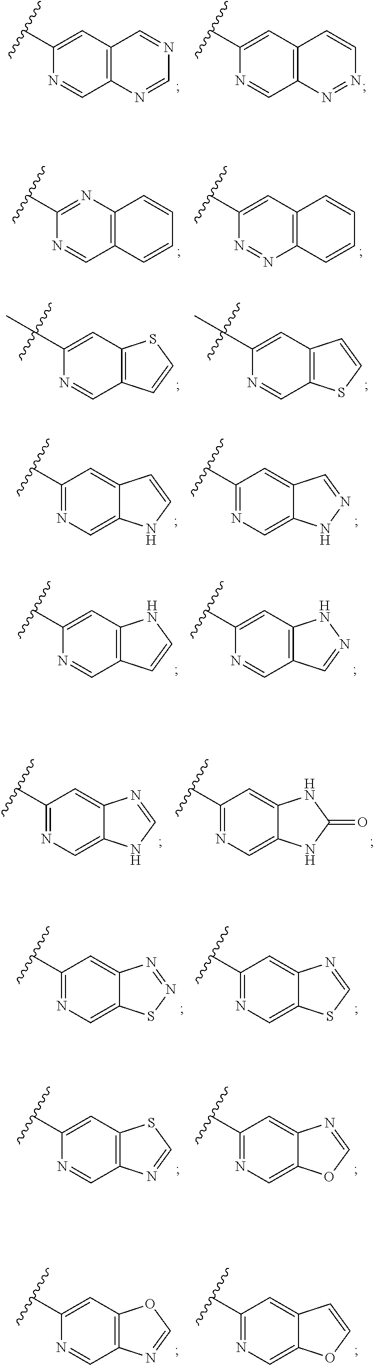

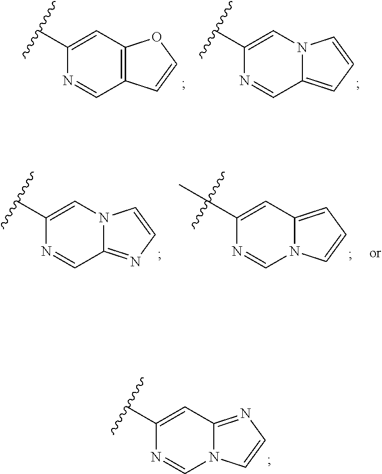 [SMILES: *c1cc2occc2cn1 ; *c1cn2cccc2cn1 ; *c1cn2ccnc2cn1 ; *c1cc2cccn2cn1 ; *c1cc2nccn2cn1]

wherein each one of the above-depicted groups is optionally substituted with one or more groups R¹¹.

Each R¹¹ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R¹².

Each R¹² is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl.

The ring atoms X₁, X₂, X₃ and X₄ in formula (I) have the following meanings: X₁ is C(R^{X1}) or N; X₂ is C(-L-R^{X2}) or N; X₃ is C(R^{X3}) or N; and X₄ is C(R^{X4}) or N; wherein at least one of the ring atoms X₁, X₂, X₃ and X₄ is not N.

R^{X1} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R^{X11}.

Each R^{X11} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl.

L is selected from a covalent bond, C₁₋₁₀ alkylene, C₂₋₁₀ alkenylene, and C₂₋₁₀ alkynylene, wherein one or more —CH₂— units comprised in said C₁₋₁₀ alkylene, said C₂₋₁₀ alkenylene, or said C₂₋₁₀ alkynylene are each optionally replaced by a group independently selected from —O—, —CO—, —C(═O)O—, —O—C(═O)—, —NH—, —N(C₁₋₅ alkyl)-, —NH—CO—, —N(C₁₋₅ alkyl)-CO—, —CO—NH—, —CO—N(C₁₋₅ alkyl)-, —S—, —SO—, —SO₂—, —SO₂—NH—, —SO₂—N(C₁₋₅ alkyl)-, —NH—SO₂—, —N(C₁₋₅ alkyl)-SO₂—, carbocyclylene, and heterocyclylene, wherein said carbocyclylene and said heterocyclylene are each optionally substituted with one or more groups independently selected from C₁₋₄ alkyl, —OH, —O(C₁₋₄ alkyl), —SH, —S(C₁₋₅ alkyl), —NH₂, —NH(C₁₋₄ alkyl), —N(C₁₋₄ alkyl)(C₁₋₄ alkyl), halogen, C₁₋₅ haloalkyl, —O—(C₁₋₅ haloalkyl), and —CN, and further wherein said C₁₋₁₀ alkylene, said C₂₋₁₀ alkenylene, and said C₂₋₁₀ alkynylene are each optionally substituted with one or more groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

R^{X2} is selected from C₂₋₁₀ alkyl, carbocyclyl, heterocyclyl, and -L¹-R^{X21}, wherein said C₂₋₁₀ alkyl, said carbocyclyl and said heterocyclyl are each optionally substituted with one or more groups R^{X22}.

L¹ is selected from a covalent bond, C₁₋₁₀ alkylene, C₂₋₁₀ alkenylene, and C₂₋₁₀ alkynylene, wherein one or more —CH₂— units comprised in said C₁₋₁₀ alkylene, said C₂₋₁₀ alkenylene, or said C₂₋₁₀ alkynylene are each optionally replaced by a group independently selected from —O—, —CO—, —C(═O)O—, —O—C(═O)—, —NH—, —N(C₁₋₅ alkyl)-, —NH—CO—, —N(C₁₋₅ alkyl)-CO—, —CO—NH—, —CO—N(C₁₋₅ alkyl)-, —S—, —SO—, —SO₂—, —SO₂—NH—, —SO₂—N(C₁₋₅ alkyl)-, —NH—SO₂—, and —N(C₁₋₅ alkyl)-SO₂—, and further wherein said C₁₋₁₀ alkylene, said C₂₋₁₀ alkenylene, and said C₂₋₁₀ alkynylene are each optionally substituted with one or more groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

R^{X21} is selected from C₂₋₅ alkyl, carbocyclyl, and heterocyclyl, wherein said carbocyclyl and said heterocyclyl are each optionally substituted with one or more groups R^{X22}.

Each R^{X22} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R^{X23}.

Each R^{X23} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), cycloalkyl, and heterocycloalkyl.

R^{X3} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R^{X31}.

Each R^{X31} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl.

R^{X4} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R^{X41}.

Each R^{X41} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl.

In accordance with the present invention, the following compounds are excluded from formula (I):

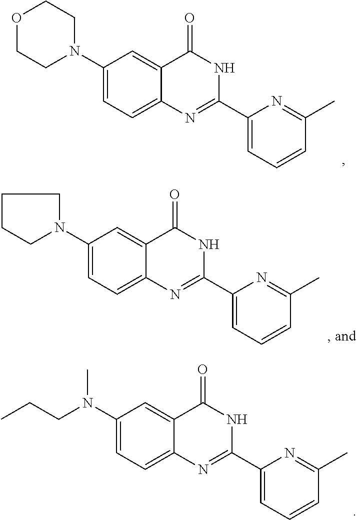

The present invention also relates to a pharmaceutical composition comprising a compound of formula (I) or a pharmaceutically acceptable salt thereof, in combination with a pharmaceutically acceptable excipient. Accordingly, the invention relates to a compound of formula (I) or a pharmaceutically acceptable salt thereof, or a pharmaceutical composition comprising any of the aforementioned entities and a pharmaceutically acceptable excipient, for use as a medicament.

The invention further relates to a compound of formula (I) or a pharmaceutically acceptable salt thereof, or a pharmaceutical composition comprising any of the aforementioned entities and a pharmaceutically acceptable excipient, for use in the treatment or prevention of a condition associated with altered glutamatergic signalling and/or functions or a condition which can be affected by alteration of glutamate level or signalling.

Moreover, the present invention relates to the use of a compound of formula (I) or a pharmaceutically acceptable salt thereof in the preparation of a medicament for the treatment or prevention of a condition associated with altered glutamatergic signalling and/or functions or a condition which can be affected by alteration of glutamate level or signalling.

The invention likewise relates to a method of treating or preventing a condition associated with altered glutamatergic signalling and/or functions or a condition which can be affected by alteration of glutamate level or signalling, the method comprising administering a compound of formula (I) or a pharmaceutically acceptable salt thereof, or a pharmaceutical composition comprising any of the aforementioned entities in combination with a pharmaceutically acceptable excipient, to a subject (preferably a human) in need thereof. It will be understood that a therapeutically effective amount of the compound of formula (I) or the pharmaceutically acceptable salt thereof, or of the pharmaceutical composition, is to be administered in accordance with this method.

The conditions to be treated or prevented in accordance with the present invention, i.e. the conditions associated with altered glutamatergic signalling and/or functions or the conditions which can be affected by alteration of glutamate level or signalling, include in particular: epilepsy, including newborn, infantile, childhood and adult syndromes, partial (localization-related) and generalized epilepsies, with partial and generalized, convulsive and non-convulsive seizures, with and without impairment of consciousness, and status epilepticus; Dementias and related diseases, including dementias of the Alzheimer's type (DAT), Alzheimer's disease, Pick's disease, vascular dementias, Lewy-body disease, dementias due to metabolic, toxic and deficiency diseases (including alcoholism, hypothyroidism, and vitamin B12 deficiency), AIDS-dementia complex, Creutzfeld-Jacob disease and atypical subacute spongiform encephalopathy; Parkinsonism and movement disorders, including Parkinson's disease, multiple system atrophy, progressive supranuclear palsy, corticobasal degeneration, hepatolenticular degeneration, chorea (including Huntington's disease and hemiballismus), athetosis, dystonias (including spasmodic torticollis, occupational movement disorder, Gilles de la Tourette syndrome), tardive or drug induced dyskinesias (including levodopa-induced dyskinesia), tremor and myoclonus; Motor neuron disease or amyotrophic lateral sclerosis (ALS); Other neurodegenerative and/or hereditary disorders of the nervous system, including spinocerebrellar degenerations such as Friedrich's ataxia and other hereditary cerebellar ataxias, predominantly spinal muscular atrophies, hereditary neuropathies, and phakomatoses; Disorders of the peripheral nervous system, including trigeminal neuralgia, facial nerve disorders, disorders of the other cranial nerves, nerve root and plexus disorders, mononeuritis such as carpal tunnel syndrome and sciatica, hereditary and idiopathic peripheral neuropathies, inflammatory and toxic neuropathies; Multiple sclerosis and other autoimmune diseases, including lupus (i.e., systemic lupus erythematosus) and psoriasis; Infantile cerebral palsy (spastic), monoplegic, paraplegic or tetraplegic; Hemiplegia and hemiparesis, flaccid or spastic, and other paralytic syndromes; Cerebrovascular disorders, including subarachnoid hemorrhage, intracerebral hemorrhage, occlusion and stenosis of precerebral arteries, occlusion of cerebral arteries including thrombosis and embolism, brain ischemia, stroke, transient ischemic attacks, atherosclerosis, cerebrovascular dementias, aneurysms, cerebral deficits due to cardiac bypass surgery and grafting; Migraine, including classical migraine and variants such as cluster headache; Headache; Myoneural disorders including myasthenia gravis, acute muscle spasms, myopathies including muscular dystrophies, mytotonias and familial periodic paralysis; Disorders of the eye and visual pathways, including retinal disorders, and visual disturbances; Intracranial trauma/injury and their sequels; Trauma/injury to nerves and spinal cord and their sequels; Poisoning and toxic effects of nonmedicinal substances; Accidental poisoning by drugs, medicinal substances and biologicals acting on the central, peripheral and autonomic system; Neurological and psychiatric adverse effects of drugs, medicinal and biological substances; Disturbance of sphincter control and sexual function; Social skill disorders such as autism or autism spectrum disorders, or fragile X syndrome; Mental disorders usually diagnosed in infancy, childhood or adolescence, including: mental retardation, learning disorders, motor skill disorders, communication disorders, pervasive developmental disorders, attention deficit and disruptive behaviour disorders, feeding and eating disorders, TIC disorders, elimination disorders; Delirium and other cognitive disorders; Substance related disorders including: alcohol-related disorders, nicotine-related disorders, disorders related to cocaine, opioids, cannabis, hallucinogens and other drugs; Schizophrenia and other psychotic disorders; Mood disorders, including depressive disorders and bipolar disorders; Anxiety disorders, including panic disorders, phobias, obsessive-compulsive disorders, stress disorders, generalized anxiety disorders; Eating disorders, including anorexia and bulimia; Sleep disorders, including dyssomnias (insomnia, hypersomnia, narcolepsy, breathing related sleep disorder) and parasomnias; Medication-induced movement disorders (including neuroleptic-induced parkinsonism and tardive dyskinesia); Endocrine and metabolic diseases including diabetes, disorders of the endocrine glands, hypoglycaemia; Acute and chronic pain; Nausea and vomiting; Irritable bowel syndrome; or cancers.

Preferably, the condition to be treated or prevented in accordance with the present invention is selected from: Dementias and related diseases, including dementias of the Alzheimer's type (DAT), Alzheimer's disease, Pick's disease, vascular dementias, Lewy-body disease, dementias due to metabolic, toxic and deficiency diseases (including alcoholism, hypothyroidism, and vitamin B12 deficiency), AIDS-dementia complex, Creutzfeld-Jacob disease and atypical subacute spongiform encephalopathy; Parkinsonism and movement disorders, including Parkinson's disease, multiple system atrophy, progressive supranuclear palsy, corticobasal degeneration, hepatolenticular degeneration, chorea (including Huntington's disease and hemiballismus), athetosis, dystonias (including spasmodic torticollis, occupational movement disorder, Gilles de la Tourette syndrome), tardive or drug induced dyskinesias (including levodopa-induced dyskinesia), tremor and myoclonus; Social skill disorders such as autism or autism spectrum disorders, or fragile X syndrome; Acute and chronic pain; Anxiety disorders, including panic disorders, phobias, obsessive-compulsive disorders, stress disorders and generalized anxiety disorders; Schizophrenia and other psychotic disorders; Mood disorders, including depressive disorders and bipolar disorders; Endocrine and metabolic diseases including diabetes, disorders of the endocrine glands and hypoglycaemia; or cancers. More preferably, the condition to be treated or prevented in accordance with the present invention is Parkinson's disease.

The present invention furthermore provides a method of identifying a test agent that binds to metabotropic glutamate receptor 4 (mGluR4), or in other words for determining the capability of one or more test agent(s) to bind to the receptor, comprising the following steps: (a) contacting mGluR4 with a compound of the present invention (i.e., a compound of formula (I) or a pharmaceutically acceptable salt thereof) which is labeled, preferably radio-labeled or fluorescence-labeled, under conditions that permit binding of the compound to mGluR4, thereby generating a bound, labeled compound; (b) detecting a signal that corresponds to the amount of the bound, labeled compound in the absence of test agent; (c) contacting the bound, labeled compound with a test agent; (d) detecting a signal that corresponds to the amount of the bound labeled compound in the presence of test agent; and (e) comparing the signal detected in step (d) to the signal detected in step (b) to determine whether the test agent binds to mGluR4. As will be understood, a substantially unchanged signal detected in step (d) in comparison with the signal detected in step (b) indicates that the test agent does not bind to the receptor, or binds to the receptor less strongly than the compounds according to the invention. A decreased or increased signal detected in step (d) in comparison with the signal detected in step (b) indicates that the test agent binds to the receptor. Thus, agents that bind to mGluR4 can be identified among the test agents employed in the above method. It will further be understood that it is preferred to remove unbound labeled compounds, e.g. in a washing step, before carrying out steps (b) and (d).

The mGluR4 which is used in the above method may be a human form (see, e.g., Flor P J et al., Neuropharmacology. 1995. 34:149-155; Makoff A et al., Brain Res. Mol. Brain Res. 1996. 37:239-248; or Wu S et al., Brain Res. Mol. Brain Res. 1998. 53:88-97), e.g. a protein of the accession number NP_000832 or a protein having at least 80% (preferably, at least 90%; more preferably, at least 95%; even more preferably, at least 99%) amino acid identity to said protein of the accession number NP_000832, or a non-human form, including e.g. a mouse form or rat form (see, e.g., Tanabe Y et al., Neuron. 1992. 8:169-179), or a homolog thereof found in a different species (e.g. in a different mammalian species), or a mutein of any of the aforementioned entitites which mutein retains the mGluR4 activity. Said mutain can preferably be obtained by substitution, insertion, addition and/or deletion of one or more (such as, e.g., 1 to 20, including 1 to 10 or 1 to 3) amino acid residues of said aforementioned entitites. The mGluR4 used in the above method may also be a functional fragment of any of the aforementioned entitites (including said muteins), i.e. a fragment which retains the mGluR4 activity of the respective aforementioned entity or, in other words, a fragment having essentially the same biological activity (i.e., at least about 60% activitiy, preferably at least about 70% activity, more preferably at least about 80% activity, even more preferably at least about 90% activity) as the respective aforementioned entity. A person skilled in the art is readily in a position to determine whether mGluR4 activity is retained using techniques known in the art, e.g. knock-out and rescue experiments. Furthermore, the mGluR4 used in the above method may also be a compound comprising any one or more of the aforementioned entitites (including, without limitation, a protein of the accession number NP_000832, a protein having at least 80% amino acid identity to said protein of the accession number NP_000832, or a functional fragment thereof), wherein the mGluR4 activity is retained. Preferably, the mGluR4 used in the above method is a human form.

The present invention also relates to the use of a compound of formula (I) or a pharmaceutically acceptable salt thereof as a positive allosteric modulator of mGluR4 (i.e., as an mGluR4 PAM) in research, particularly as a research tool compound. Accordingly, the invention refers to the in vitro use of a compound of formula (I) or a pharmaceutically acceptable salt thereof as an mGluR4 PAM and, in particular, to the in vitro use of a compound of formula (I) or a pharmaceutically acceptable salt thereof as a research tool compound acting as an mGluR4 PAM. The invention likewise relates to a method, particularly an in vitro method, of effecting positive allosteric modulation of mGluR4, the method comprising the application of a compound of formula (I) or a pharmaceutically acceptable salt thereof. The invention further relates to a method of effecting positive allosteric modulation of mGluR4, the method comprising applying a compound of formula (I) or a pharmaceutically acceptable salt thereof to a test sample (e.g., a biological sample) or a test animal (i.e., a non-human test animal). The invention also refers to a method, particularly an in vitro method, of effecting positive allosteric modulation of mGluR4 in a sample (e.g., a biological sample), the method comprising applying a compound of formula (I) or a pharmaceutically acceptable salt thereof to said sample. The present invention further provides a method of effecting positive allosteric modulation of mGluR4, the method comprising contacting a test sample (e.g., a biological sample) or a test animal (i.e., a non-human test animal) with a compound of formula (I) or a pharmaceutically acceptable salt thereof. The mGluR4 is preferably human mGluR4. The terms “sample”, “test sample” and “biological sample” include, without being limited thereto: a cell, a cell culture or a cellular or subcellular extract; biopsied material obtained from an animal (e.g., a human), or an extract thereof; or blood, serum, plasma, saliva, urine, feces, or any other body fluid, or an extract thereof. It is to be understood that the term “in vitro” is used in this specific context in the sense of “outside a living human or animal body”, which includes, in particular, experiments performed with cells, cellular or subcellular extracts, and/or biological molecules in an artificial environment such as an aqueous solution or a culture medium which may be provided, e.g., in a flask, a test tube, a Petri dish, a microtiter plate, etc.

The compound of formula (I) or the pharmaceutically acceptable salt thereof, which is provided in accordance with the present invention, will be described in more detail in the following:

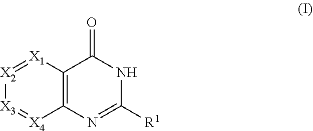

R¹ is selected from any one of the following groups:

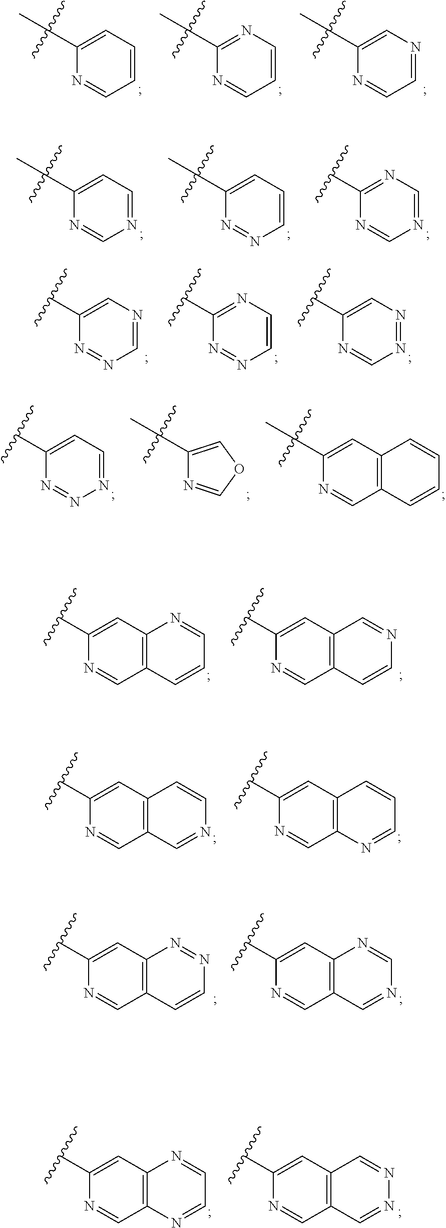

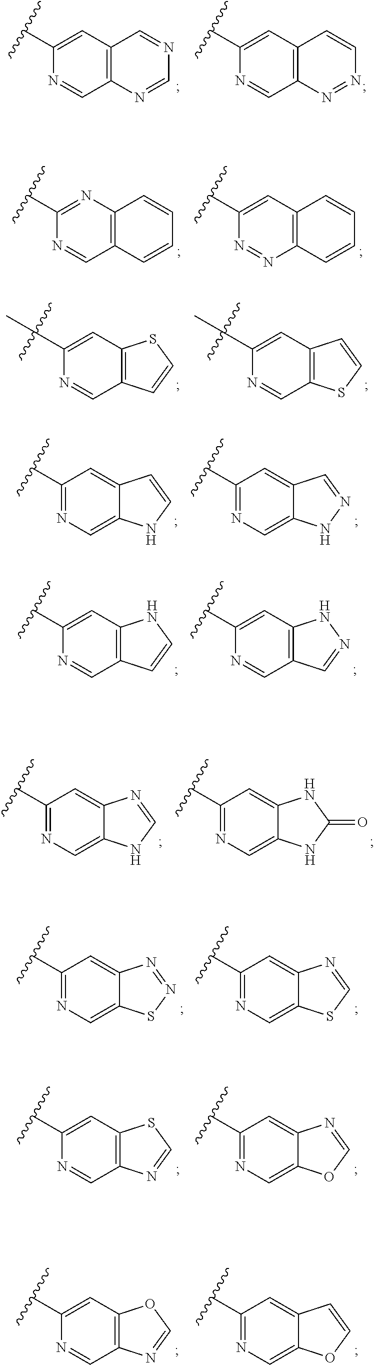

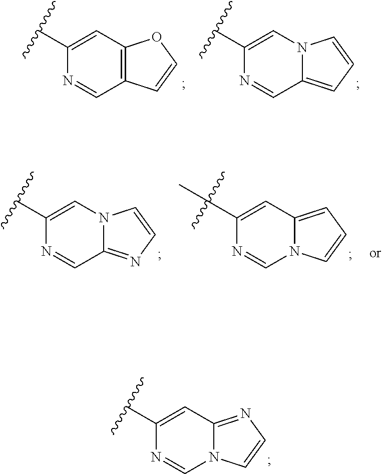 [SMILES: *c1cc2occc2cn1 ; *c1cn2cccc2cn1 ; *c1cn2ccnc2cn1 ; *c1cc2cccn2cn1 ; *c1cc2nccn2cn1]

wherein each one of the above-depicted groups is optionally substituted with one or more (e.g., one, two, or three) groups R¹¹.

Examples of R¹ include any of the respective groups R¹ comprised in any of the specific compounds of the invention disclosed in the examples section.

Preferably, R¹ is either selected from any one of the following groups:

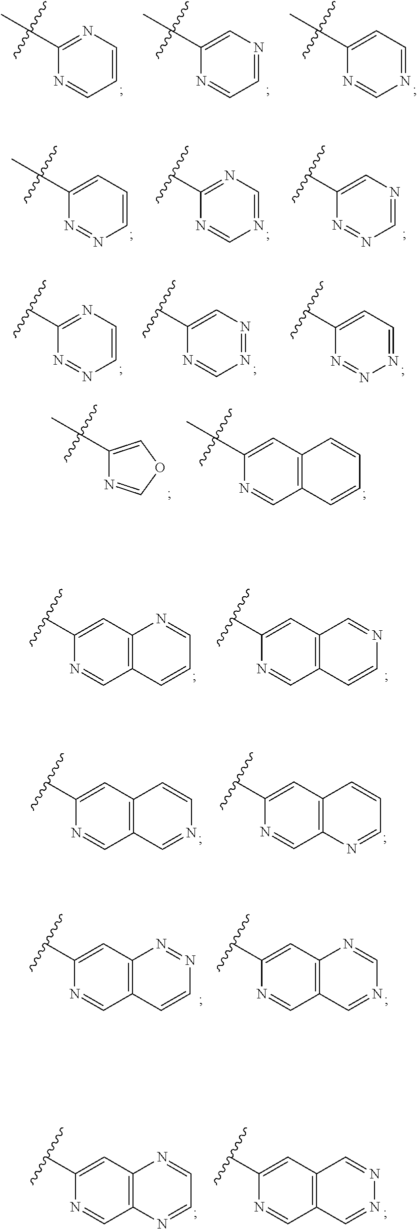

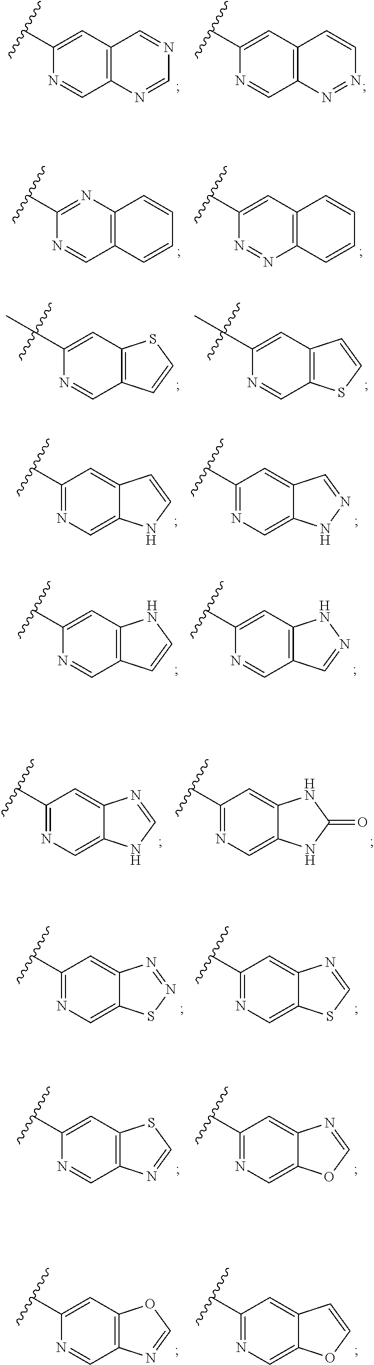

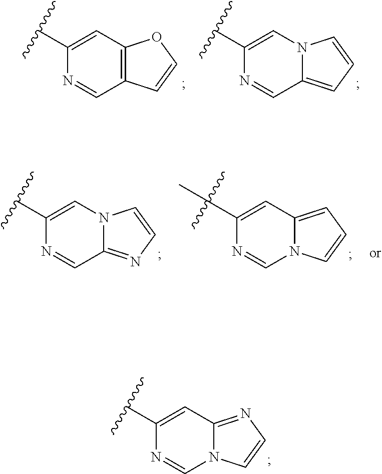 [SMILES: *c1cc2occc2cn1 ; *c1cn2cccc2cn1 ; *c1cn2ccnc2cn1 ; *c1cc2cccn2cn1 ; *c1cc2nccn2cn1]

wherein each one of the above-depicted groups is optionally substituted with one or more groups R¹¹;

or R is a group

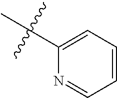 [SMILES: *c1ccccn1]

which is optionally substituted with one or more groups R.

More preferably, R¹ is selected from one of the following groups:

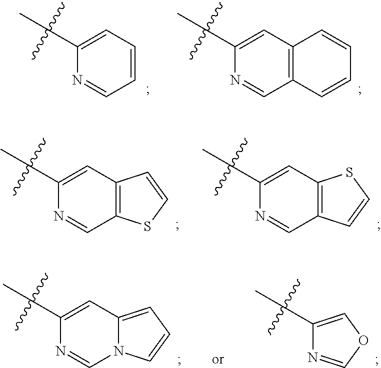 [SMILES: *c1ccccn1 ; *c1cc2ccccc2cn1 ; *c1cc2ccsc2cn1 ; *c1cc2sccc2cn1 ; *c1cc2cccn2cn1 ; *c1cocn1]

wherein each one of the above-depicted groups is optionally substituted with one or more groups R¹¹.

Even more preferably, R¹ is selected from one of the following groups:

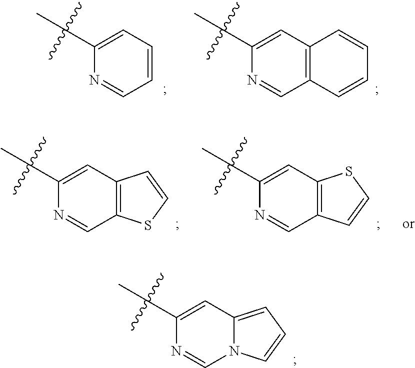 [SMILES: *c1ccccn1 ; *c1cc2ccccc2cn1 ; *c1cc2ccsc2cn1 ; *c1cc2sccc2cn1 ; *c1cc2cccn2cn1]

wherein each one of the above-depicted groups is optionally substituted with one or more groups R¹¹.

Even more preferably, R¹ is selected from one of the following groups:

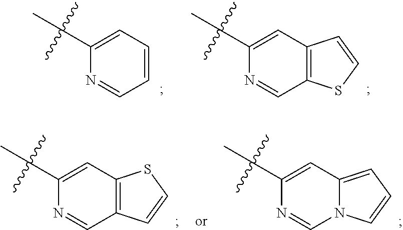 [SMILES: *c1ccccn1 ; *c1cc2ccsc2cn1 ; *c1cc2sccc2cn1 ; *c1cc2cccn2cn1]

wherein each one of the above-depicted groups is optionally substituted with one or more groups R¹¹.

Yet even more preferably, R¹ is selected from one of the following groups:

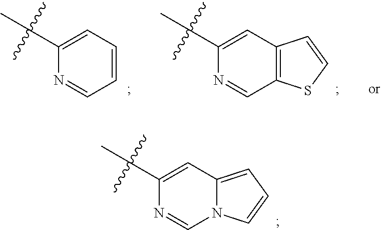 [SMILES: *c1ccccn1 ; *c1cc2ccsc2cn1 ; *c1cc2cccn2cn1]

wherein each one of the above-depicted groups is optionally substituted with one or more groups R¹¹.

Still more preferably, R¹ is a group:

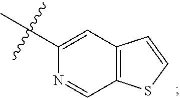 [SMILES: *c1cc2ccsc2cn1]

wherein the above-depicted group is optionally substituted with one or more groups R¹¹.

Each R¹¹ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl,

wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more (e.g., one, two, or three) groups R¹²,

and further wherein each R¹² is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl.

Preferably, each R¹¹ is independently selected from C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl).

More preferably, each R¹¹ is independently selected from C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), and —(C₀₋₃ alkylene)-CN.

Even more preferably, each R¹¹ is independently selected from C₁₋₅ alkyl (e.g., methyl or ethyl), —OH, —O(C₁₋₅ alkyl) (e.g., methoxy or ethoxy), halogen (e.g., —F or —Cl), C₁₋₅ haloalkyl (e.g., —CF₃), —O—(C₁₋₅ haloalkyl) (e.g., —OCF₃), and —CN.

Each R^{11A} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-heteroaryl, —(C₀₋₃ alkylene)-cycloalkyl, and —(C₀₋₃ alkylene)-heterocycloalkyl,

wherein the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more (e.g., one, two, or three) groups R¹²,

and further wherein each R¹² is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl.

Preferably, each R^{11A} is independently selected from C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl).

More preferably, each R^{11A} is independently selected from C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), and —(C₀₋₃ alkylene)-CN.

Even more preferably, each R^{11A} is independently selected from C₁₋₅ alkyl (e.g., methyl or ethyl), —OH, —O(C₁₋₅ alkyl) (e.g., methoxy or ethoxy), halogen (e.g., —F or —Cl), C₁₋₅ haloalkyl (e.g., —CF₃), —O—(C₁₋₅ haloalkyl) (e.g., —OCF₃), and —CN.

As explained above, R may be a pyridin-2-yl group having the structure

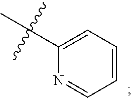 [SMILES: *c1ccccn1]

wherein said pyridin-2-yl group is optionally substituted with one or more groups R¹¹.

In that case, it is preferred that said pyridin-2-yl group is a substituted pyridin-2-yl group selected from any one of the following groups:

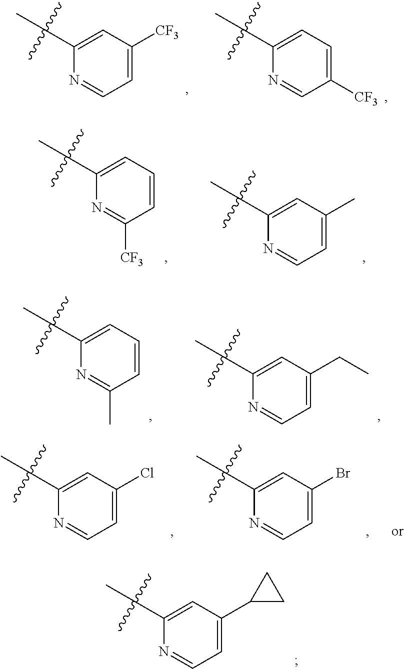

wherein each one of the above-depicted groups is optionally further substituted with one or more (e.g., one or two) groups R¹¹.

More preferably, said pyridin-2-yl group is a substituted pyridin-2-yl group selected from any one of the following groups:

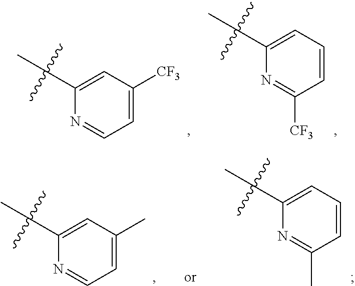

wherein each one of the above-depicted groups is optionally further substituted with one or more groups (e.g., one group) R¹¹.

Even more preferably, said pyridin-2-yl group is a trifluoromethyl- or methyl-substituted pyridin-2-yl group selected from any one of the following groups:

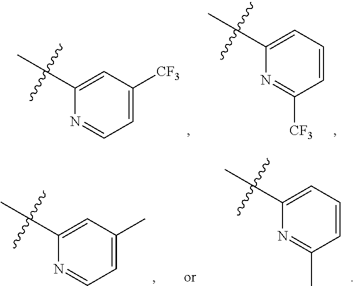

Accordingly, it is particularly preferred that R¹ is selected from any one of the following groups:

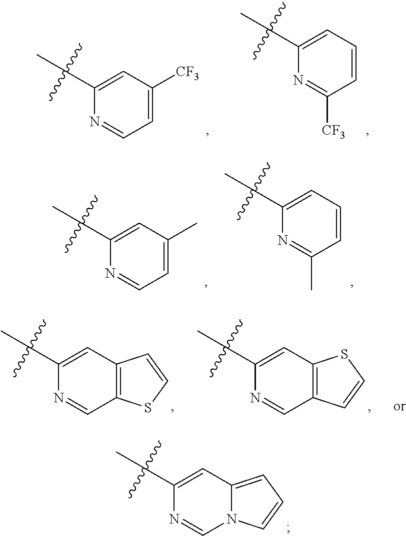

wherein each one of the above-depicted groups is optionally further substituted with one or more groups R¹¹ (and wherein the above-depicted groups are preferably not further substituted with any groups R¹¹).

Still more preferably, R¹ is:

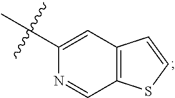 [SMILES: *c1cc2ccsc2cn1]

wherein the above-depicted group is optionally substituted with one or more groups R¹¹.

Most preferably, R¹ is:

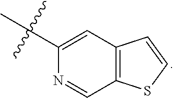 [SMILES: *c1cc2ccsc2cn1]

The ring atoms X₁, X₂, X₃ and X₄ in formula (I) have the following meanings: X₁ is C(R^{X1}) or N; X₂ is C(-L-R^{X2}) or N; X₃ is C(R^{X3}) or N; and X₄ is C(R^{X4}) or N; wherein at least one of the ring atoms X₁, X₂, X₃ and X₄ is not N.

Preferably, X₄ is C(R^{X1}) or N; X₂ is C(-L-R^{X2}); X₃ is C(R^{X3}) or N; and X₄ is C(R^{X4}) or N.

More preferably, X₄ is C(R^{X1}) or N; X₂ is C(-L-R^{X2}); X₃ is C(R^{X3}) or N; and X₄ is C(R^{X4}).

Even more preferably, X₁ is C(R^{X1}) or N; X₂ is C(-L-R^{X2}); X₃ is C(R^{X3}) or N; and X₄ is C(R^{X4}); wherein one or none of X₁ and X₃ is N (i.e., at least one of X₁ and X₃ is not N).

Yet even more preferably, X₁ is C(R^{X1}) or N; X₂ is C(-L-R^{X2}); X₃ is C(R^{X3}); and X₄ is C(R^{X4}).

Still more preferably, X₄ is C(R^{X1}), X₂ is C(-L-R^{X2}), X₃ is C(R^{X3}), and X₄ is C(R^{X4}).

Thus, in accordance with the above definitions of X₁, X₂, X₃ and X₄, it is particularly preferred that the compound of formula (I) has the following structure:

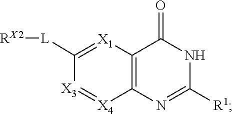

and it is even more preferred that the compound of formula (I) has the following structure:

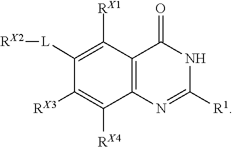

R^{X1} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl,

wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more (e.g., one, two, or three) groups R^{X11},

and further wherein each R^{X11} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl.

Preferably, R^{X1} is selected from hydrogen, C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl).

More preferably, R^{X1} is selected from hydrogen, C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), and —(C₀₋₃ alkylene)-CN.

Even more preferably, R^{X1} is selected from hydrogen, C₁₋₅ alkyl, —OH, —O(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN.

Yet even more preferably, R^{X1} is hydrogen.

L is selected from a covalent bond, C₁₋₁₀ alkylene, C₂₋₁₀ alkenylene, and C₂₋₁₀ alkynylene,

wherein one or more (e.g., one or two) —CH₂— units comprised in said C₁₋₁₀ alkylene, said C₂₋₁₀ alkenylene, or said C₂₋₁₀ alkynylene are each optionally replaced by a group independently selected from —O—, —CO—, —C(═O)O—, —O—C(═O)—, —NH—, —N(C₁₋₅ alkyl)-, —NH—CO—, —N(C₁₋₅ alkyl)-CO—, —CO—NH—, —CO—N(C₁₋₅ alkyl)-, —S—, —SO—, —SO₂—, —SO₂—NH—, —SO₂—N(C₁₋₅ alkyl)-, —NH—SO₂—, —N(C₁₋₅ alkyl)-SO₂—, carbocyclylene (e.g., cycloalkylene or arylene), and heterocyclylene (e.g., heterocycloalkylene or heteroarylene), wherein said carbocyclylene (or said cycloalkylene or arylene) and said heterocyclylene (or said heterocycloalkylene or heteroarylene) are each optionally substituted with one or more groups independently selected from C₁₋₄ alkyl, —OH, —O(C₁₋₄ alkyl), —SH, —S(C₁₋₅ alkyl), —NH₂, —NH(C₁₋₄ alkyl), —N(C₁₋₄ alkyl)(C₁₋₄ alkyl), halogen, C₁₋₅ haloalkyl, —O—(C₁₋₅ haloalkyl), and —CN, and further wherein said C₁₋₁₀ alkylene, said C₂₋₁₀ alkenylene, and said C₂₋₁₀ alkynylene are

each optionally substituted with one or more (e.g., one, two, or three) groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

It will be understood that if X₂ is C(-L-R^{X2}) and L is a covalent bond, then the group R^{X2} is directly attached to the corresponding ring carbon atom of the quinazolinone ring of the compounds of formula (I), as illustrated in the following:

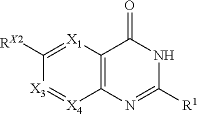

Preferably, L is a covalent bond or C₁₋₁₀ alkylene,

wherein one or two —CH₂— units comprised in said C₁₋₁₀ alkylene are each optionally replaced by a group independently selected from —O—, —CO—, —C(═O)O—, —O—C(═O)—, —NH—, —N(C₁₋₅ alkyl)-, —NH—CO—, —N(C₁₋₅ alkyl)-CO—, —CO—NH—, —CO—N(C₁₋₅ alkyl)-, —S—, —SO—, —SO₂—, —SO₂—NH—, —SO₂—N(C₁₋₅ alkyl)-, —NH—SO₂—, —N(C₁₋₅ alkyl)-SO₂—, cycloalkylene, arylene, heterocycloalkylene, and heteroarylene, wherein said cycloalkylene, said arylene, said heterocycloalkylene and said heteroarylene are each optionally substituted with one or more (e.g., one, two, or three) groups independently selected from C₁₋₄ alkyl, —OH, —O(C₁₋₄ alkyl), —SH, —S(C₁₋₅ alkyl), —NH₂, —NH(C₁₋₄ alkyl), —N(C₁₋₄ alkyl)(C₁₋₄ alkyl), halogen, C₁₋₅ haloalkyl, —O—(C₁₋₅ haloalkyl), and —CN,

and further wherein said C₁₋₁₀ alkylene is optionally substituted with one or more (e.g., one, two, or three) groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

More preferably, L is a covalent bond or C₁₋₈ alkylene,

wherein one —CH₂— unit comprised in said C₁₋₈ alkylene is optionally replaced by a group selected from —O—, —CO—, —NH—, and —N(C₁₋₅ alkyl)-,

and further wherein said C₁₋₈ alkylene is optionally substituted with one or more (e.g., one, two, or three) groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Even more preferably, L is selected from a covalent bond, C₁₋₅ alkylene (e.g., —CH₂—, —CH₂CH₂—, or —CH₂CH₂CH₂—), —O—, —O—(C₁₋₅ alkylene)- (e.g., —O—CH₂—, —O—CH₂CH₂—, —O—CH₂CH₂CH₂— or —O—CH₂CH₂CH₂CH₂—), —CO—, —(C₁₋₅ alkylene)-CO— (e.g., —CH₂—CO—), —NH—, —NH—(C₁₋₅ alkylene)-, —N(C₁₋₅ alkyl)-, and —N(C₁₋₅ alkyl)-(C₁₋₅ alkylene)-,

wherein said C₁₋₅ alkylene or the C₁₋₅ alkylene moiety comprised in any of said —O—(C₁₋₅ alkylene)-, said —NH—(C₁₋₅ alkylene)-, and said —N(C₁₋₅ alkyl)-(C₁₋₅ alkylene)- is optionally substituted with one or more groups independently selected from halogen, —CF₃, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Yet even more preferably, L is selected from a covalent bond, C₁₋₅ alkylene (e.g., —CH₂—, —CH₂CH₂—, or —CH₂CH₂CH₂—), —O—, —O—(C₁₋₅ alkylene)- (e.g., —O—CH₂—, —O—CH₂CH₂—, —O—CH₂CH₂CH₂— or —O—CH₂CH₂CH₂CH₂—), —NH—, —NH—(C₁₋₅ alkylene)-, —N(C₁₋₅ alkyl)-, and —N(C₁₋₅ alkyl)-(C₁₋₅ alkylene)-,

wherein said C₁₋₅ alkylene or the C₁₋₅ alkylene moiety comprised in any of said —O—(C₁₋₅ alkylene)-, said —NH—(C₁₋₅ alkylene)-, and said —N(C₁₋₅ alkyl)-(C₁₋₅ alkylene)- is optionally substituted with one or more groups independently selected from halogen, —CF₃, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Still more preferably, L is selected from a covalent bond, C₁₋₅ alkylene (e.g., —CH₂—, —CH₂CH₂—, or —CH₂CH₂CH₂—), —O—, and —O—(C₁₋₅ alkylene)- (e.g., —O—CH₂—, —O—CH₂CH₂—, —O—CH₂CH₂CH₂— or —O—CH₂CH₂CH₂CH₂—).

R^{X2} is selected from C₂₋₁₀ alkyl, carbocyclyl (e.g., cycloalkyl or aryl), heterocyclyl (e.g., heterocycloalkyl or heteroaryl), and -L¹-R^{X21}, wherein said C₂₋₁₀ alkyl, said carbocyclyl and said heterocyclyl are each optionally substituted with one or more (e.g., one, two, or three) groups R^{X22}.

For example, R^{X2} may be selected from C₂₋₁₀ alkyl, carbocyclyl (e.g., cycloalkyl or aryl), heterocycloalkyl, and heteroaryl, wherein said heterocycloalkyl is a monocyclic heterocycloalkyl or a spiro-ring heterocycloalkyl, and further wherein said C₂₋₁₀ alkyl, said carbocyclyl, said heterocycloalkyl and said heteroaryl are each optionally substituted with one or more (e.g., one, two, or three) groups R^{X22}.

Preferably, R^{X2} is selected from C₂₋₁₀ alkyl, cycloalkyl, aryl, heterocycloalkyl, and heteroaryl, wherein said C₂₋₁₀ alkyl, said cycloalkyl, said aryl, said heterocycloalkyl, and said heteroaryl are each optionally substituted with one or more (e.g., one, two, or three) groups R^{X22}; said heterocycloalkyl may be, e.g., a monocyclic heterocycloalkyl or a spiro-ring heterocycloalkyl.

More preferably, R^{X2} is selected from cycloalkyl, aryl, heterocycloalkyl, and heteroaryl, wherein said cycloalkyl, said aryl, said heterocycloalkyl, and said heteroaryl are each optionally substituted with one or more (e.g., one, two, or three) groups R^{X22}.

Even more preferably, R^{X2} is selected from azetidinyl (e.g., azetidin-3-yl), oxetanyl (e.g., oxetan-3-yl), pyrrolidinyl (e.g., pyrrolidin-1-yl or pyrrolidin-3-yl), oxopyrrolidinyl (e.g., 2-oxo-pyrrolidin-1-yl or 5-oxo-pyrrolidin-3-yl), tetrahydrofuranyl (e.g., tetrahydrofuran-3-yl), piperidinyl (e.g., piperidin-1-yl, piperidin-3-yl or piperidin-4-yl), oxopiperidinyl (e.g., 2-oxo-piperidin-4-yl or 6-oxo-piperidin-3-yl), piperazinyl (e.g., piperazin-1-yl), oxopiperazinyl (e.g., 3-oxo-piperazin-1-yl), morpholinyl (e.g., morpholin-4-yl), thiomorpholinyl (e.g., thiomorpholin-4-yl), dioxidothiomorpholinyl (e.g., 1,1-dioxidothiomorpholin-4-yl), tetrahydropyranyl (e.g., tetrahydropyran-4-yl), oxazepanyl (e.g., [1,4]oxazepan-4-yl), 2-oxa-6-aza-spiro[3.3]heptanyl (e.g., 2-oxa-6-aza-spiro[3.3]heptan-6-yl), 2-oxa-7-aza-spiro[3.5]nonyl (e.g., 2-oxa-7-aza-spiro[3.5]non-7-yl), 6-oxa-2-aza-spiro[3.4]octyl (e.g., 6-oxa-2-aza-spiro[3.4]oct-2-yl), 3-oxa-9-aza-spiro[5.5]undecyl (e.g., 3-oxa-9-aza-spiro[5.5]undec-9-yl), 7-oxa-2-aza-spiro[4.5]decyl (e.g., 7-oxa-2-aza-spiro[4.5]dec-2-yl), 8-oxa-2-aza-spiro[4.5]decyl (e.g., 8-oxa-2-aza-spiro[4.5]dec-2-yl), 3-oxa-8-aza-bicyclo[3.2.1]octyl (e.g., 3-oxa-8-aza-bicyclo[3.2.1]oct-8-yl), 8-oxa-3-aza-bicyclo[3.2.1]octyl (e.g., 8-oxa-3-aza-bicyclo[3.2.1]oct-3-yl), phenyl, oxazolyl (e.g., oxazol-4-yl), pyridinyl (e.g., pyridin-3-yl or pyridin-4-yl), pyrazinyl (e.g., pyrazin-2-yl), and pyrimidinyl (e.g., pyrimidin-5-yl), wherein each one of the aforementioned cyclic groups is optionally substituted with one or more groups R^{X22}.

Yet even more preferably, R^{X2} is selected from azetidinyl (e.g., azetidin-3-yl), oxetanyl (e.g., oxetan-3-yl), pyrrolidinyl (e.g., pyrrolidin-3-yl), oxopyrrolidinyl (e.g., 2-oxo-pyrrolidin-1-yl), tetrahydrofuranyl (e.g., tetrahydrofuran-3-yl), piperidinyl (e.g., piperidin-3-yl or piperidin-4-yl), oxopiperidinyl (e.g., 6-oxo-piperidin-3-yl), piperazinyl (e.g., piperazin-1-yl), morpholinyl (e.g., morpholin-4-yl), tetrahydropyranyl (e.g., tetrahydropyran-4-yl), 2-oxa-7-aza-spiro[3.5]nonyl (e.g., 2-oxa-7-aza-spiro[3.5]non-7-yl), 6-oxa-2-aza-spiro[3.4]octyl (e.g., 6-oxa-2-aza-spiro[3.4]oct-2-yl), 3-oxa-9-aza-spiro[5.5]undecyl (e.g., 3-oxa-9-aza-spiro[5.5]undec-9-yl), 7-oxa-2-aza-spiro[4.5]decyl (e.g., 7-oxa-2-aza-spiro[4.5]dec-2-yl), 8-oxa-2-aza-spiro[4.5]decyl (e.g., 8-oxa-2-aza-spiro[4.5]dec-2-yl), phenyl, oxazolyl (e.g., oxazol-4-yl), pyridinyl (e.g., pyridin-3-yl or pyridin-4-yl), pyrazinyl (e.g., pyrazin-2-yl), and pyrimidinyl (e.g., pyrimidin-5-yl), wherein each one of the aforementioned cyclic groups is optionally substituted with one or more groups R^{X22}.

L¹ is selected from a covalent bond, C₁₋₁₀ alkylene, C₂₋₁₀ alkenylene, and C₂₋₁₀ alkynylene, wherein one or more (e.g., one or two) —CH₂— units comprised in said C₁₋₁₀ alkylene, said C₂₋₁₀ alkenylene, or said C₂₋₁₀ alkynylene are each optionally replaced by a group independently selected from —O—, —CO—, —C(═O)O—, —O—C(═O)—, —NH—, —N(C₁₋₅ alkyl)-, —NH—CO—, —N(C₁₋₅ alkyl)-CO—, —CO—NH—, —CO—N(C₁₋₅ alkyl)-, —S—, —SO—, —SO₂—, —SO₂—NH—, —SO₂—N(C₁₋₅ alkyl)-, —NH—SO₂—, and —N(C₁₋₅ alkyl)-SO₂—, and further wherein said C₁₋₁₀ alkylene, said C₂₋₁₀ alkenylene, and said C₂₋₁₀ alkynylene are each optionally substituted with one or more (e.g., one, two, or three) groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Preferably, L¹ is a covalent bond or C₁₋₁₀ alkylene, wherein one or two —CH₂— units comprised in said C₁₋₁₀ alkylene are each optionally replaced by a group independently selected from —O—, —CO—, —C(═O)O—, —O—C(═O)—, —NH—, —N(C₁₋₅ alkyl)-, —NH—CO—, —N(C₁₋₅ alkyl)-CO—, —CO—NH—, —CO—N(C₁₋₅ alkyl)-, —S—, —SO—, —SO₂—, —SO₂—NH—, —SO₂—N(C₁₋₅ alkyl)-, —NH—SO₂—, and —N(C₁₋₅ alkyl)-SO₂—, and further wherein said C₁₋₁₀ alkylene is optionally substituted with one or more (e.g., one, two, or three) groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

More preferably, L¹ is a covalent bond or C₁₋₈ alkylene, wherein one —CH₂— unit comprised in said C₁₋₈ alkylene is optionally replaced by a group selected from —O—, —CO—, —NH—, and —N(C₁₋₅ alkyl)-, and further wherein said C₁₋₈ alkylene is optionally substituted with one or more (e.g., one, two, or three) groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Even more preferably, L¹ is selected from a covalent bond, C₁₋₅ alkylene (e.g., —CH₂—, —CH₂CH₂—, or —CH₂CH₂CH₂—), —O—, —O—(C₁₋₅ alkylene)- (e.g., —O—CH₂—, —O—CH₂CH₂—, —O—CH₂CH₂CH₂— or —O—CH₂CH₂CH₂CH₂—), —NH—, —NH—(C₁₋₅ alkylene)-, —N(C₁₋₅ alkyl)-, and —N(C₁₋₅ alkyl)-(C₁₋₅ alkylene)-, wherein said C₁₋₅ alkylene or the C₁₋₅ alkylene moiety comprised in any of said —O—(C₁₋₅ alkylene)-, said —NH—(C₁₋₅ alkylene)-, and said —N(C₁₋₅ alkyl)-(C₁₋₅ alkylene)- is optionally substituted with one or more groups independently selected from halogen, —CF₃, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Yet even more preferably, L¹ is selected from a covalent bond, C₁₋₅ alkylene (e.g., —CH₂—, —CH₂CH₂—, or —CH₂CH₂CH₂—), —O—, and —O—(C₁₋₅ alkylene)- (e.g., —O—CH₂—, —O—CH₂CH₂—, —O—CH₂CH₂CH₂— or —O—CH₂CH₂CH₂CH₂—).

R^{X21} is selected from C₂₋₅ alkyl, carbocyclyl (e.g., cycloalkyl or aryl), and heterocyclyl (e.g., heterocycloalkyl or heteroaryl), wherein said carbocyclyl and said heterocyclyl are each optionally substituted with one or more (e.g., one, two, or three) groups R^{X22}.

Preferably, R^{X21} is selected from C₂₋₅ alkyl, cycloalkyl, aryl, heterocycloalkyl, and heteroaryl, wherein said cycloalkyl, said aryl, said heterocycloalkyl, and said heteroaryl are each optionally substituted with one or more (e.g., one, two, or three) groups R^{X22}.

Each R^{X22} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl,

wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more (e.g., one, two, or three) groups R^{X23},

and further wherein each R^{X23} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), cycloalkyl, and heterocycloalkyl.

Preferably, each R^{X22} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl.

More preferably, each R^{X22} is independently selected from C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), and —(C₀₋₃ alkylene)-CN.

Even more preferably, each R^{X22} is independently selected from C₁₋₅ alkyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN.

Examples of -L-R^{X2} include any of the respective groups -L-R^{X2} comprised in the specific compounds of the invention disclosed in the examples section.

In accordance with the above definitions of L and R^{X2}, it is particularly preferred that the group -L-R^{X2} in the compound of formula (I) is —R^{X2} or —(C₁₋₈ alkylene)-R^{X2}, wherein one —CH₂-unit comprised in said C₁₋₈ alkylene is optionally replaced by a group selected from —O—, —CO—, —NH—, and —N(C₁₋₅ alkyl)-, wherein said C₁₋₈ alkylene is optionally substituted with one or more (e.g., one, two, or three) groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein R^{X2} is selected from cycloalkyl, aryl, heterocycloalkyl, and heteroaryl, wherein said cycloalkyl, said aryl, said heterocycloalkyl, and said heteroaryl are each optionally substituted with one or more (e.g., one, two, or three) groups R^{X22}.

More preferably, the group -L-R^{X2} is selected from —R^{X2}, —(C₁₋₅ alkylene)-R^{X2}, —O—R^{X2}, and —O—(C₁₋₅ alkylene)-R^{X2}, wherein R^{X2} is selected from cycloalkyl, aryl, heterocycloalkyl, and heteroaryl, wherein said cycloalkyl, said aryl, said heterocycloalkyl, and said heteroaryl are each optionally substituted with one or more (e.g., one, two, or three) groups R^{X22}.

In the above definitions of the group -L-R^{X2}, it is even more preferred that R^{X2} is selected from azetidinyl (e.g., azetidin-3-yl), oxetanyl (e.g., oxetan-3-yl), pyrrolidinyl (e.g., pyrrolidin-3-yl), oxopyrrolidinyl (e.g., 2-oxo-pyrrolidin-1-yl), tetrahydrofuranyl (e.g., tetrahydrofuran-3-yl), piperidinyl (e.g., piperidin-3-yl or piperidin-4-yl), oxopiperidinyl (e.g., 6-oxo-piperidin-3-yl), piperazinyl (e.g., piperazin-1-yl), morpholinyl (e.g., morpholin-4-yl), tetrahydropyranyl (e.g., tetrahydropyran-4-yl), 2-oxa-7-aza-spiro[3.5]nonyl (e.g., 2-oxa-7-aza-spiro[3.5]non-7-yl), 6-oxa-2-aza-spiro[3.4]octyl (e.g., 6-oxa-2-aza-spiro[3.4]oct-2-yl), 3-oxa-9-aza-spiro[5.5]undecyl (e.g., 3-oxa-9-aza-spiro[5.5]undec-9-yl), 7-oxa-2-aza-spiro[4.5]decyl (e.g., 7-oxa-2-aza-spiro[4.5]dec-2-yl), 8-oxa-2-aza-spiro[4.5]decyl (e.g., 8-oxa-2-aza-spiro[4.5]dec-2-yl), phenyl, oxazolyl (e.g., oxazol-4-yl), pyridinyl (e.g., pyridin-3-yl or pyridin-4-yl), pyrazinyl (e.g., pyrazin-2-yl), and pyrimidinyl (e.g., pyrimidin-5-yl), wherein each one of the aforementioned cyclic groups is optionally substituted with one or more groups R^{X22}.

It is yet even more preferred that -L-R^{X2} is selected from any of the following groups:

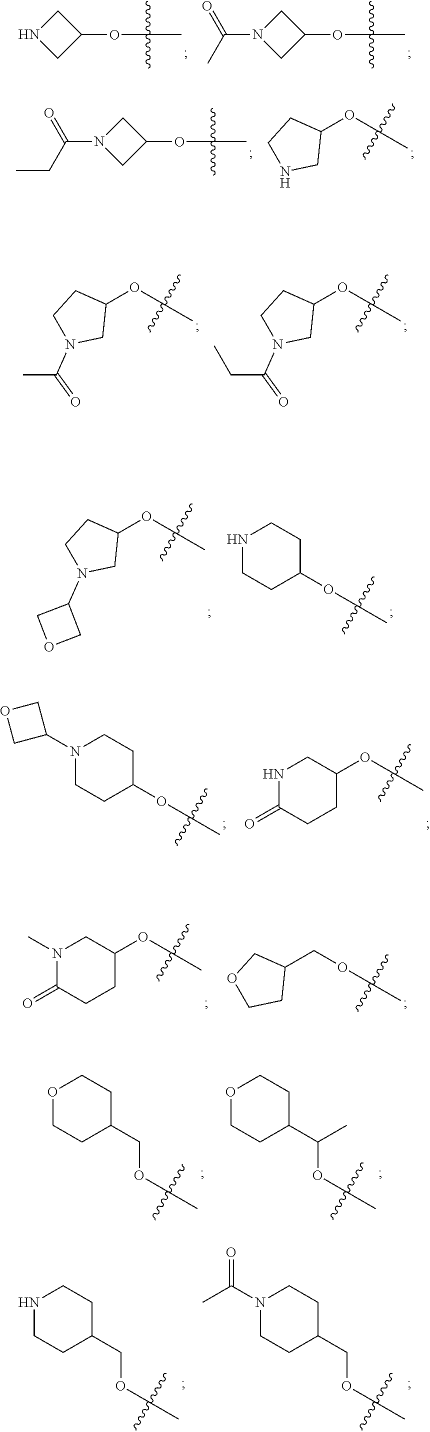

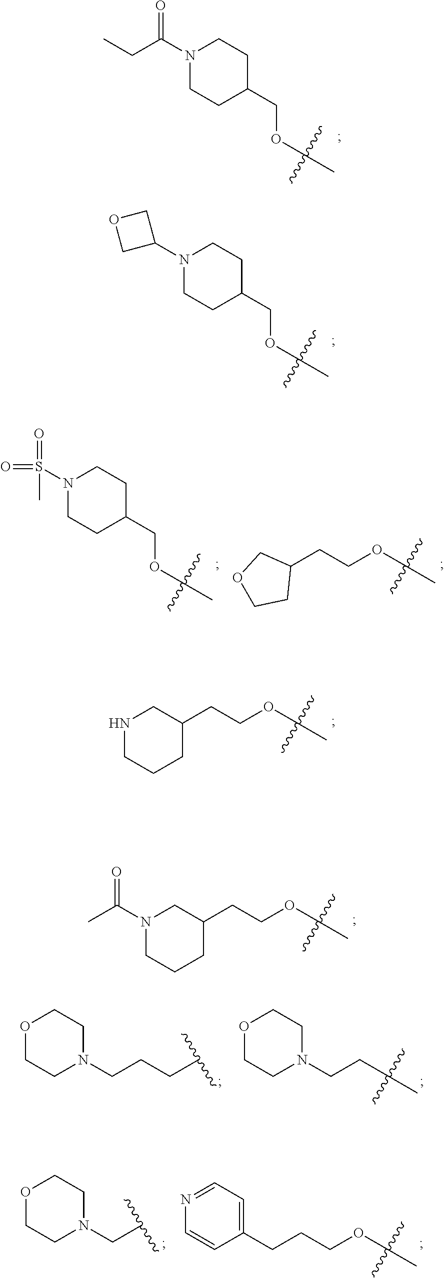

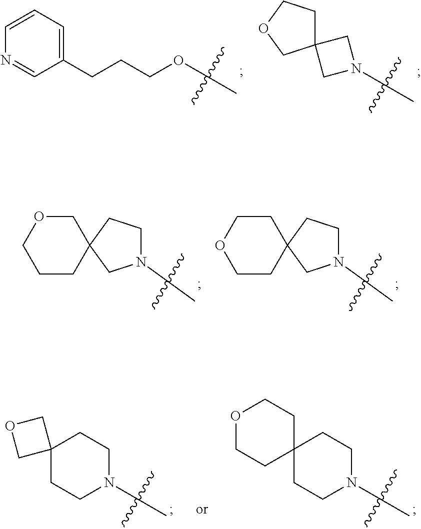 [SMILES: *OCCCc1cccnc1 ; *N1CC2(CCOC2)C1 ; *N1CCC2(CCCOC2)C1 ; *N1CCC2(CCOCC2)C1 ; *N1CCC2(CC1)COC2 ; *N1CCC2(CCOCC2)CC1]

wherein the cyclic moiety in each of the above-depicted groups is optionally further substituted with one or more (e.g., one or two) groups R^{X22}.

Yet even more preferably, -L-R^{X2} is selected from any of the following groups:

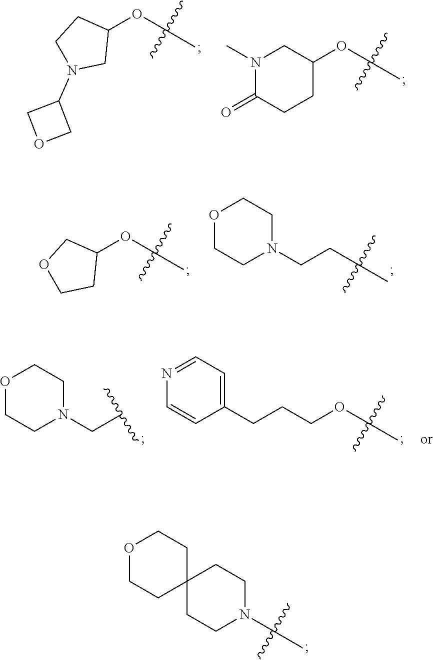

wherein the cyclic moiety in each of the above-depicted groups is optionally further substituted with one or more (e.g., one or two) groups R^{X22}.

Still more preferably, -L-R^{X2} is selected from any of the following groups:

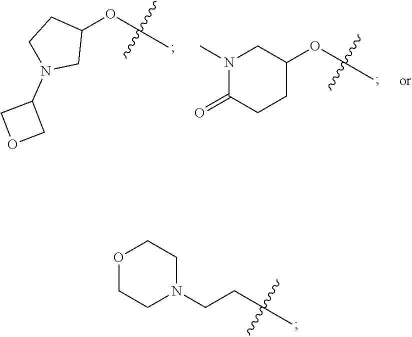

wherein the cyclic moiety in each of the above-depicted groups is optionally further substituted with one or more (e.g., one or two) groups R^{X22}.

Most preferably, -L-R^{X2} is:

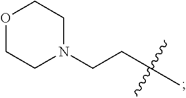 [SMILES: *CCN1CCOCC1]

wherein the cyclic moiety in the above-depicted group is optionally further substituted with one or more (e.g., one or two) groups R^{X22}.

R^{X3} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl,

wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more (e.g., one, two, or three) groups R^{X31},

and further wherein each R^{X31} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl.

The heterocycloalkyl moiety in the aforementioned group —(C₀₋₃ alkylene)-heterocycloalkyl may be, e.g., a monocyclic heterocycloalkyl or a spiro-ring heterocycloalkyl.

Preferably, R^{X3} is selected from hydrogen, C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl).

More preferably, R^{X3} is selected from hydrogen, C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), and —(C₀₋₃ alkylene)-CN.

Even more preferably, R^{X3} is selected from hydrogen, C₁₋₅ alkyl (e.g., methyl or ethyl), —OH, —O(C₁₋₅ alkyl) (e.g., methoxy or ethoxy), halogen (e.g., —F or —Cl), and C₁₋₅ haloalkyl (e.g., —CF₃).

Yet even more preferably, R^{X3} is selected from hydrogen, —OH, and —OCH₃. It is particularly preferred that R^{X3} is hydrogen.

R^{X4} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl,

wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more (e.g., one, two, or three) groups R^{X41},

and further wherein each R^{X41} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl.

Preferably, R^{X4} is selected from hydrogen, C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), aryl, heteroaryl, cycloalkyl, and heterocycloalkyl,

wherein said aryl, said heteroaryl, said cycloalkyl, and said heterocycloalkyl are each optionally substituted with one or more (e.g., one, two, or three) groups independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl.

More preferably, R^{X4} is selected from hydrogen, C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, cycloalkyl, and heterocycloalkyl.

Even more preferably, R^{X4} is selected from hydrogen, C₁₋₅ alkyl (e.g., methyl or ethyl), —O—C₁₋₅ alkyl (e.g., methoxy or ethoxy), halogen (e.g., —F or —Cl), C₁₋₅ haloalkyl (e.g., —CF₃), and C₃₋₇ cycloalkyl (e.g., cyclopropyl).

Even more preferably, R^{X4} is selected from hydrogen, methyl, —OCH₃, halogen (e.g., —F or —Cl), and cyclopropyl. For example, R^{X4} may be methyl, —OCH₃, halogen, or cyclopropyl.

Yet even more preferably, R^{X4} is selected from hydrogen, methyl, halogen (e.g., —F or —Cl), and cyclopropyl. It is particularly preferred that R^{X4} is selected from methyl, —F and —Cl.

Still more preferably, R^{X4} is methyl.

In accordance with the present invention, the following compounds are excluded from formula (I):

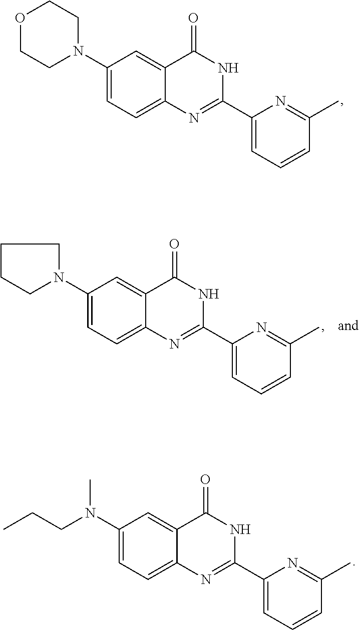

Thus, the above-depicted compounds as well as pharmaceutically acceptable salts thereof are excluded from the present invention.

It is furthermore preferred that the following compound is also excluded from the present invention:

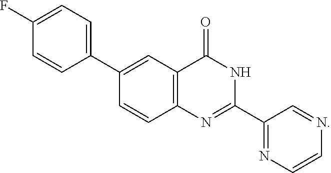 [SMILES: O=c1[nH]c(-c2cnccn2)nc2ccc(-c3ccc(F)cc3)cc12]

It is particularly preferred that the compound of formula (I) according to the invention is one of the specific compounds of formula (I) described further below in the examples section of this specification, either in non-salt form (e.g., free base/acid form) or as a pharmaceutically acceptable salt of the respective compound.

Accordingly, it is particularly preferred that the compound of formula (I) is selected from:
6-(3-Pyridin-4-yl-propoxy)-2-pyrrolo[1,2-c]pyrimidin-3-yl-3H-quinazolin-4-one;
2-Isoquinolin-3-yl-6-(3-pyridin-4-yl-propoxy)-3H-quinazolin-4-one;
6-(3-Pyridin-4-yl-propoxy)-2-thieno[3,2-c]pyridin-6-yl-3H-quinazolin-4-one;
6-(3-Pyridin-4-yl-propoxy)-2-thieno[2,3-c]pyridin-5-yl-3H-quinazolin-4-one;
2-Pyridin-2-yl-6-(3-pyridin-4-yl-propoxy)-3H-quinazolin-4-one;
2-(4-Methoxy-pyridin-2-yl)-6-(3-pyridin-4-yl-propoxy)-3H-quinazolin-4-one;
2-(5-Fluoro-pyridin-2-yl)-6-(3-pyridin-4-yl-propoxy)-3H-quinazolin-4-one;
6-(3-Pyridin-4-yl-propoxy)-2-(5-trifluoromethyl-pyridin-3-yl)-3H-quinazolin-4-one;
6-[3-(4-Pyridyl)propoxy]-2-[5-(trifluoromethyl)-2-pyridyl]-3H-quinazolin-4-one;
2-(4-Methyl-2-pyridyl)-6-[3-(4-pyridyl)propoxy]-3H-quinazolin-4-one;
2-(6-Methyl-2-pyridyl)-6-[3-(4-pyridyl)propoxy]-3H-quinazolin-4-one;
2-(5-Methylpyrazin-2-yl)-6-[3-(4-pyridyl)propoxy]-3H-quinazolin-4-one;
2-[5-Chloro-4-(trifluoromethyl)-2-pyridyl]-6-[3-(4-pyridyl)propoxy]3H-quinazolin-4-one;
2-(4-Chloro-2-pyridyl)-6-[3-(4-pyridyl)propoxy]-3H-quinazolin-4-one;
2-(4-Ethyl-2-pyridyl)-6-[3-(4-pyridyl)propoxy]-3H-quinazolin-4-one;
6-[3-(4-Pyridyl)propoxy]-2-[6-(trifluoromethyl)-2-pyridyl]-3H-quinazolin-4-one;
2-(4-Bromo-2-pyridyl)-6-[3-(4-pyridyl)propoxy]-3H-quinazolin-4-one;
2-(4-Cyclopropyl-2-pyridyl)-6-[3-(4-pyridyl)propoxy]-3H-quinazolin-4-one;
2-(2-Methyl-oxazol-4-yl)-6-(3-pyridin-4-yl-propoxy)-3H-quinazolin-4-one;
6-(2-Pyridin-3-yl-ethoxy)-2-thieno[3,2-c]pyridin-6-yl-3H-quinazolin-4-one;
6-(4-Bromo-benzyloxy)-2-thieno[3,2-c]pyridin-6-yl-3H-quinazolin-4-one;
Tert-butyl 3-(4-hydroxy-2-pyrrolo[1,2-c]pyrimidin-3-yl-quinazolin-6-yl)oxyazetidine-1-carboxylate;
6-(Azetidin-3-yloxy)-2-pyrrolo[1,2-c]pyrimidin-3-yl-3H-quinazolin-4-one;
6-(1-Pyrimidin-4-yl-azetidin-3-yloxy)-2-pyrrolo[1,2-c]pyrimidin-3-yl-3H-quinazolin-4-one;
3-(4-Hydroxy-2-thieno[2,3-c]pyridin-5-yl-quinazolin-6-yloxy)-azetidine-1-carboxylic acid tert-butyl ester;
6-(Azetidin-3-yloxy)-2-thieno[2,3-c]pyridin-5-yl-3H-quinazolin-4-one;
6-(1-Propionyl-azetidin-3-yloxy)-2-thieno[2,3-c]pyridin-5-yl-3H-quinazolin-4-one;
6-(Piperidin-4-yloxy)-2-thieno[3,2-c]pyridin-6-yl-3H-quinazolin-4-one;
6-(1-Propionyl-piperidin-4-yloxy)-2-thieno[3,2-c]pyridin-6-yl-3H-quinazolin-4-one;
6-(2-Morpholin-4-yl-ethoxy)-2-pyrrolo[1,2-c]pyrimidin-3-yl-3H-quinazolin-4-one;
6-(2-Methoxy-ethoxy)-2-pyrrolo[1,2-c]pyrimidin-3-yl-3H-quinazolin-4-one;
6-(2-Morpholin-4-yl-ethoxy)-2-thieno[3,2-c]pyridin-6-yl-3H-quinazolin-4-one;
6-(2-Methoxy-ethoxy)-2-thieno[3,2-c]pyridin-6-yl-3H-quinazolin-4-one;
6-(3-Pyridin-3-yl-propoxy)-2-thieno[3,2-c]pyridin-6-yl-3H-quinazolin-4-one;
4-(4-Oxo-2-pyridin-2-yl-3,4-dihydro-quinazolin-6-yloxy)-piperidine-1-carboxylic acid tert-butyl ester;
6-(Piperidin-4-yloxy)-2-pyridin-2-yl-3H-quinazolin-4-one;
6-(1-Acetyl-piperidin-4-yloxy)-2-pyridin-2-yl-3H-quinazolin-4-one;
4-[4-Oxo-2-(4-trifluoromethyl-pyridin-2-yl)-3,4-dihydro-quinazolin-6-yloxymethyl]-piperidine-1-carboxylic acid tert-butyl ester;
6-(Piperidin-4-ylmethoxy)-2-(4-trifluoromethyl-pyridin-2-yl)-3H-quinazolin-4-one;
6-(1-Acetyl-piperidin-4-ylmethoxy)-2-(4-trifluoromethyl-pyridin-2-yl)-3H-quinazolin-4-one;
tert-butyl 4-[(4-oxo-2-thieno[2,3-c]pyridin-5-yl-3H-quinazolin-6-yl)oxymethyl]piperidine-1-carboxylate;
6-(4-piperidylmethoxy)-2-thieno[2,3-c]pyridin-5-yl-3H-quinazolin-4-one;
6-(1-Acetyl-piperidin-4-ylmethoxy)-2-thieno[2,3-c]pyridin-5-yl-3H-quinazolin-4-one;
6-(1-Propionyl-piperidin-4-ylmethoxy)-2-thieno[2,3-c]pyridin-5-yl-3H-quinazolin-4-one;
3-(4-Oxo-2-thieno[2,3-c]pyridin-5-yl-3,4-dihydro-quinazolin-6-yloxy)-pyrrolidine-1-carboxylic acid tert-butyl ester;
6-(Pyrrolidin-3-yloxy)-2-thieno[2,3-c]pyridin-5-yl-3H-quinazolin-4-one;
6-(1-Acetyl-pyrrolidin-3-yloxy)-2-thieno[2,3-c]pyridin-5-yl-3H-quinazolin-4-one;
4-[4-Oxo-2-(4-trifluoromethyl-pyridin-2-yl)-3,4-dihydro-quinazolin-6-yl]-piperazine-1-carboxylic acid tert-butyl ester;
6-Piperazin-1-yl-2-(4-trifluoromethyl-pyridin-2-yl)-3H-quinazolin-4-one;
6-(4-Propionyl-piperazin-1-yl)-2-(4-trifluoromethyl-pyridin-2-yl)-3H-quinazolin-4-one;
4-(4-Oxo-2-thieno[2,3-c]pyridin-5-yl-3,4-dihydro-quinazolin-6-yl)-piperidine-1-carboxylic acid tert-butyl ester;
6-Piperidin-4-yl-2-thieno[2,3-c]pyridin-5-yl-3H-quinazolin-4-one;
6-(1-Acetyl-piperidin-4-yl)-2-thieno[2,3-c]pyridin-5-yl-3H-quinazolin-4-one;
6-[2-(Tetrahydro-pyran-4-yl)-ethoxy]-2-(4-trifluoromethyl-pyridin-2-yl)-3H-quinazolin-4-one;
6-[3-(3-Fluoro-pyridin-4-yl)-propoxy]-2-thieno[2,3-c]pyridin-5-yl-3H-quinazolin-4-one;
6-[3-(4-Methanesulfonyl-phenyl)-propoxy]-2-thieno[2,3-c]pyridin-5-yl-3H-quinazolin-4-one;
6-(3-Pyrazin-2-yl-propoxy)-2-thieno[2,3-c]pyridin-5-yl-3H-quinazolin-4-one;
6-[3-(3-Methoxy-pyridin-4-yl)-propoxy]-2-thieno[2,3-c]pyridin-5-yl-3H-quinazolin-4-one;
6-[3-(2-Methyl-pyridin-4-yl)-propoxy]-2-thieno[2,3-c]pyridin-5-yl-3H-quinazolin-4-one;
6-(3-Oxazol-4-yl-propoxy)-2-thieno[2,3-c]pyridin-5-yl-3H-quinazolin-4-one;
8-Methyl-6-(3-pyridin-4-yl-propoxy)-2-thieno[2,3-c]pyridin-5-yl-3H-quinazolin-4-one;
6-(3-Pyridin-4-yl-propoxy)-2-thieno[2,3-c]pyridin-5-yl-3H-pyrido[3,2-d]pyrimidin-4-one;
6-(3-Pyridin-4-yl-propoxy)-2-(4-trifluoromethyl-pyridin-2-yl)-3H-pyrido[3,2-d]pyrimidin-4-one;
6-(3-Pyridin-4-yl-propoxy)-2-(4-trifluoromethyl-pyridin-2-yl)-3H-pyrido[2,3-d]pyrimidin-4-one;
6-(3-Pyridin-4-yl-propoxy)-2-(4-trifluoromethyl-pyridin-2-yl)-3H-pyrido[3,4-d]pyrimidin-4-one;
6-(3-Pyridin-4-yl-propoxy)-2-thieno[2,3-c]pyridin-5-yl-7-trifluoromethyl-3H-quinazolin-4-one;
5-Chloro-6-(3-pyridin-4-yl-propoxy)-2-thieno[2,3-c]pyridin-5-yl-3H-quinazolin-4-one;
8-Chloro-6-(3-pyridin-4-yl-propoxy)-2-thieno[2,3-c]pyridin-5-yl-3H-quinazolin-4-one;
8-Cyclopropyl-6-(3-pyridin-4-yl-propoxy)-2-thieno[2,3-c]pyridin-5-yl-3H-quinazolin-4-one;
8-Ethyl-6-(3-pyridin-4-yl-propoxy)-2-thieno[2,3-c]pyridin-5-yl-3H-quinazolin-4-one;
8-Fluoro-6-(3-pyridin-4-yl-propoxy)-2-thieno[2,3-c]pyridin-5-yl-3H-quinazolin-4-one;
8-Methyl-6-(tetrahydro-pyran-4-ylmethoxy)-2-thieno[2,3-c]pyridin-5-yl-3H-quinazolin-4-one;
8-Methyl-6-(2-oxetan-3-yl-ethoxy)-2-thieno[2,3-c]pyridin-5-yl-3H-quinazolin-4-one;
8-Methyl-6-[2-(tetrahydro-furan-3-yl)-ethoxy]-2-thieno[2,3-c]pyridin-5-yl-3H-quinazolin-4-one;
8-Methyl-6-[2-(tetrahydro-pyran-4-yl)-ethoxy]-2-thieno[2,3-c]pyridin-5-yl-3H-quinazolin-4-one;
8-Methyl-6-(tetrahydro-furan-3-ylmethoxy)-2-thieno[2,3-c]pyridin-5-yl-3H-quinazolin-4-one;
R-8-Methyl-6-(tetrahydro-furan-3-ylmethoxy)-2-thieno[2,3-c]pyridin-5-yl-3H-quinazolin-4-one;
S-8-Methyl-6-(tetrahydro-furan-3-ylmethoxy)-2-thieno[2,3-c]pyridin-5-yl-3H-quinazolin-4-one;
8-Methyl-6-(1-methyl-6-oxo-piperidin-3-yloxy)-2-thieno[2,3-c]pyridin-5-yl-3H-quinazolin-4-one;
R-8-Methyl-6-((1-methyl-6-oxopiperidin-3-yl)oxy)-2-(thieno[2,3-c]pyridin-5-yl)quinazolin-4(3H)-one;
S-8-Methyl-6-((1-methyl-6-oxopiperidin-3-yl)oxy)-2-(thieno[2,3-c]pyridin-5-yl)quinazolin-4(3H)-one;
8-Methyl-6-(1-propionyl-pyrrolidin-3-yloxy)-2-thieno[2,3-c]pyridin-5-yl-3H-quinazolin-4-one;
R-8-Methyl-6-(1-propionyl-pyrrolidin-3-yloxy)-2-thieno[2,3-c]pyridin-5-yl-3H-quinazolin-4-one;
S-8-Methyl-6-(1-propionyl-pyrrolidin-3-yloxy)-2-thieno[2,3-c]pyridin-5-yl-3H-quinazolin-4-one;
8-Methyl-6-(1-oxetan-3-yl-pyrrolidin-3-yloxy)-2-thieno[2,3-c]pyridin-5-yl-3H-quinazolin-4-one;
R-8-Methyl-6-(1-oxetan-3-yl-pyrrolidin-3-yloxy)-2-thieno[2,3-c]pyridin-5-yl-3H-quinazolin-4-one;
S-8-Methyl-6-(1-oxetan-3-yl-pyrrolidin-3-yloxy)-2-thieno[2,3-c]pyridin-5-yl-3H-quinazolin-4-one;
8-Methyl-6-[2-(2-oxa-7-aza-spiro[3.5]non-7-yl)-ethoxy]-2-thieno[2,3-c]pyridin-5-yl-3H-quinazolin-4-one;
8-methyl-6-(piperidin-4-ylmethoxy)-2-thieno[2,3-c]pyridin-5-yl-3H-quinazolin-4-one;
8-Methyl-6-(1-oxetan-3-yl-piperidin-4-ylmethoxy)-2-thieno[2,3-c]pyridin-5-yl-3H-quinazolin-4-one;
8-Methyl-6-(1-propionyl-piperidin-4-ylmethoxy)-2-thieno[2,3-c]pyridin-5-yl-3H-quinazolin-4-one;
6-(1-Methanesulfonyl-piperidin-4-ylmethoxy)-8-methyl-2-thieno[2,3-c]pyridin-5-yl-3H-quinazolin-4-one;
8-Methyl-6-(2-oxa-7-aza-spiro[3.5]non-7-yl)-2-thieno[2,3-c]pyridin-5-yl-3H-quinazolin-4-one;
8-Methyl-6-(6-oxa-2-aza-spiro[3.4]oct-2-yl)-2-thieno[2,3-c]pyridin-5-yl-3H-quinazolin-4-one;
8-Methyl-6-(3-oxa-9-aza-spiro[5.5]undec-9-yl)-2-thieno[2,3-c]pyridin-5-yl-3H-quinazolin-4-one;
8-Methyl-6-(7-oxa-2-aza-spiro[4.5]dec-2-yl)-2-thieno[2,3-c]pyridin-5-yl-3H-quinazolin-4-one;
8-Methyl-6-(8-oxa-2-aza-spiro[4.5]dec-2-yl)-2-thieno[2,3-c]pyridin-5-yl-3H-quinazolin-4-one;
6-(2-Hydroxy-2-methyl-propylamino)-8-methyl-2-thieno[2,3-c]pyridin-5-yl-3H-quinazolin-4-one;
8-Methyl-6-(2-piperidin-3-yl-ethoxy)-2-thieno[2,3-c]pyridin-5-yl-3H-quinazolin-4-one;
6-[2-(1-Acetyl-piperidin-3-yl)-ethoxy]-8-methyl-2-thieno[2,3-c]pyridin-5-yl-3H-quinazolin-4-one;
6-[2-(4-Acetyl-piperazin-1-yl)-ethoxy]-8-methyl-2-thieno[2,3-c]pyridin-5-yl-3H-quinazolin-4-one;
3-(8-Methyl-4-oxo-2-thieno[2,3-c]pyridin-5-yl-3,4-dihydro-quinazolin-6-yl)-propionaldehyde;
8-Methyl-6-(3-morpholin-4-yl-propyl)-2-thieno[2,3-c]pyridin-5-yl-3H-quinazolin-4-one;
8-Methyl-6-(2-morpholin-4-yl-ethyl)-2-thieno[2,3-c]pyridin-5-yl-3H-quinazolin-4-one;
8-Methyl-6-(3-pyridin-4-yl-propoxy)-2-(4-trifluoromethyl-pyridin-2-yl)-3H-quinazolin-4-one;
8-Methyl-6-(tetrahydro-furan-3-ylmethoxy)-2-(4-trifluoromethyl-pyridin-2-yl)-3H-quinazolin-4-one;
8-Methyl-6-(1-propionyl-azetidin-3-yloxy)-2-(4-trifluoromethyl-pyridin-2-yl)-3H-quinazolin-4-one;
8-Methyl-6-(1-oxetan-3-yl-piperidin-4-yloxy)-2-(4-trifluoromethyl-pyridin-2-yl)-3H-quinazolin-4-one;
8-Methyl-6-(3-oxa-9-aza-spiro[5.5]undec-9-yl)-2-(4-trifluoromethyl-pyridin-2-yl)-3H-quinazolin-4-one;
8-Methyl-6-(3-pyridin-4-yl-propoxy)-2-pyrrolo[1,2-c]pyrimidin-3-yl-3H-quinazolin-4-one;
8-Methyl-2-pyrrolo[1,2-c]pyrimidin-3-yl-6-(tetrahydro-furan-3-ylmethoxy)-3H-quinazolin-4-one;
8-Methyl-6-(3-oxa-9-aza-spiro[5.5]undec-9-yl)-2-pyrrolo[1,2-c]pyrimidin-3-yl-3H-quinazolin-4-one;
8-Methyl-6-(1-oxetan-3-yl-piperidin-4-yloxy)-2-pyrrolo[1,2-c]pyrimidin-3-yl-3H-quinazolin-4-one;
8-Methyl-6-[1-(tetrahydro-pyran-4-yl)-ethoxy]-2-thieno[2,3-c]pyridin-5-yl-3H-quinazolin-4-one;
R-8-Methyl-6-[1-(tetrahydro-pyran-4-yl)-ethoxy]-2-thieno[2,3-c]pyridin-5-yl-3H-quinazolin-4-one;
S-8-Methyl-6-[1-(tetrahydro-pyran-4-yl)-ethoxy]-2-thieno[2,3-c]pyridin-5-yl-3H-quinazolin-4-one;
8-Methyl-6-[1-(tetrahydro-pyran-4-yl)-ethoxy]-2-(4-trifluoromethyl-pyridin-2-yl)-3H-quinazolin-4-one;
R-8-Methyl-6-[1-(tetrahydro-pyran-4-yl)-ethoxy]-2-(4-trifluoromethyl-pyridin-2-yl)-3H-quinazolin-4-one;
S-8-Methyl-6-[1-(tetrahydro-pyran-4-yl)-ethoxy]-2-(4-trifluoromethyl-pyridin-2-yl)-3H-quinazolin-4-one;
6-[(3-fluorotetrahydrofuran-3-yl)methoxy]-8-methyl-2-[4-(trifluoromethyl)-2-pyridyl]-3H-quinazolin-4-one;
8-methyl-6-(3-oxa-9-azaspiro[5.5]undecan-9-yl)-2-thieno[2,3-c]pyridin-5-yl-3-(2-trimethylsilylethoxymethyl)pyrido[3,2-d]pyrimidin-4-one;
8-methyl-6-(morpholinomethyl)-2-thieno[2,3-c]pyridin-5-yl-3H-quinazolin-4-one;
8-methyl-6-(morpholinomethyl)-2-[4-(trifluoromethyl)-2-pyridyl]-3H-quinazolin-4-one;
8-methyl-6-(1-propanoylazetidin-3-yl)oxy-2-thieno[2,3-c]pyridin-5-yl-3H-quinazolin-4-one;
8-methyl-6-(2-morpholinoethyl)-2-[4-(trifluoromethyl)-2-pyridyl]-3H-quinazolin-4-one;
8-Methyl-6-[(1-methyl-6-oxo-3-piperidyl)oxy]-2-pyrrolo[1,2-c]pyrimidin-3-yl-3H-quinazolin-4-one;
8-Methyl-6-(morpholinomethyl)-2-(thieno[3,2-c]pyridin-6-yl)quinazolin-4(3H)-one;
8-Methyl-6-(3-oxa-9-azaspiro[5.5]undecan-9-yl)-2-(thieno[3,2-c]pyridin-6-yl)quinazolin-4(3H)-one;
8-Methyl-6-(2-[1,4]oxazepan-4-yl-ethyl)-2-thieno[2,3-b]pyridin-5-yl-3H-quinazolin-4-one;
8-Methyl-6-(2-[1,4]oxazepan-4-yl-ethyl)-2-thieno[3,2-b]pyridin-6-yl-3H-quinazolin-4-one;
8-Methyl-6-(2-morpholin-4-yl-ethyl)-2-thieno[3,2-c]pyridin-6-yl-3H-quinazolin-4-one;
8-Methyl-6-(2-morpholin-4-yl-ethyl)-2-pyrrolo[1,2-c]pyrimidin-3-yl-3H-quinazolin-4-one;
8-Methyl-6-(morpholinomethyl)-2-pyrrolo[1,2-c]pyrimidin-3-yl-3H-quinazolin-4-one;
8-Methyl-6-(2-morpholino-2-oxoethyl)-2-(thieno[3,2-c]pyridin-6-yl)quinazolin-4(3H)-one;
8-Methyl-6-(2-morpholino-2-oxo-ethyl)-2-thieno[2,3-c]pyridin-5-yl-3H-quinazolin-4-one;
8-Methyl-6-(2-piperidin-1-yl-ethyl)-2-thieno[2,3-b]pyridin-5-yl-3H-quinazolin-4-one;
8-Methyl-6-(1-methyl-6-oxo-piperidin-3-yloxy)-2-thieno[3,2-c]pyridin-6-yl-3H-quinazolin-4-one;
8-Methyl-6-(1-methyl-2-oxo-piperidin-4-ylmethoxy)-2-thieno[2,3-c]pyridin-5-yl-3H-quinazolin-4-one
8-Methyl-6-(1-piperidylmethyl)-2-thieno[2,3-c]pyridin-5-yl-3H-quinazolin-4-one;
8-Methyl-6-(2-morpholino-2-oxo-ethyl)-2-thieno[3,2-c]pyridin-6-yl-3H-quinazolin-4-one;
8-Methyl-6-[(4-methylpiperazin-1-yl)methyl]-2-thieno[2,3-c]pyridin-5-yl-3H-quinazolin-4-one;
8-Methyl-6-(pyrrolidin-1-ylmethyl)-2-thieno[2,3-c]pyridin-5-yl-3H-quinazolin-4-one;
8-Methyl-6-(2-morpholino-2-oxo-ethyl)-2-pyrrolo[1,2-c]pyrimidin-3-yl-3H-quinazolin-4-one;
8-Methyl-6-(morpholine-4-carbonyl)-2-pyrrolo[1,2-c]pyrimidin-3-yl-3H-quinazolin-4-one;
8-Methyl-6-(1-methyl-6-oxo-piperidin-3-yloxy)-2-(4-trifluoromethyl-pyridin-2-yl)-3H-quinazolin-4-one;
8-Methyl-2-thieno[2,3-c]pyridin-5-yl-6-(thiomorpholinomethyl)-3H-quinazolin-4-one;
8-Methyl-6-[2-(1,4-oxazepan-4-yl)-2-oxo-ethyl]-2-thieno[2,3-c]pyridin-5-yl-3H-quinazolin-4-one;
8-Methyl-6-(pyrrolidin-1-ylmethyl)-2-thieno[3,2-c]pyridin-6-yl-3H-quinazolin-4-one;
8-Methyl-6-(1-methyl-5-oxo-pyrrolidin-3-yl)oxy-2-thieno[3,2-c]pyridin-6-yl-3H-quinazolin-4-one;
8-Methyl-6-[(3R)-1-methyl-5-oxo-pyrrolidin-3-yl]oxy-2-thieno[3,2-c]pyridin-6-yl-3H-quinazolin-4-one;
8-Methyl-6-[(3S)-1-methyl-5-oxo-pyrrolidin-3-yl]oxy-2-thieno[3,2-c]pyridin-6-yl-3H-quinazolin-4-one;
Benzyl 3-[(8-methyl-4-oxo-2-thieno[3,2-c]pyridin-6-yl-3H-quinazolin-6-yl)oxy]pyrrolidine-1-carboxylate;
Benzyl (3S)-3-[(8-methyl-4-oxo-2-thieno[3,2-c]pyridin-6-yl-3H-quinazolin-6-yl)oxy]pyrrolidine-1-carboxylate;
Benzyl (3R)-3-[(8-methyl-4-oxo-2-thieno[3,2-c]pyridin-6-yl-3H-quinazolin-6-yl)oxy]pyrrolidine-1-carboxylate;
8-Methyl-6-[2-(4-methyl-3-oxo-piperazin-1-yl)ethyl]-2-thieno[2,3-c]pyridin-5-yl-3H-quinazolin-4-one;
8-Methyl-6-[2-(4-methyl-3-oxo-piperazin-1-yl)ethyl]-2-[4-(trifluoromethyl)-2-pyridyl]-3H-quinazolin-4-one;
8-Methyl-6-[2-(2-oxa-6-azaspiro[3.3]heptan-6-yl)ethyl]-2-thieno[2,3-c]pyridin-5-yl-3H-quinazolin-4-one;
8-Methyl-6-[2-(2-oxa-6-azaspiro[3.3]heptan-6-yl)ethyl]-2-thieno[3,2-c]pyridin-6-yl-3H-quinazolin-4-one;
8-Methyl-6-[(4-methyl-3-oxo-piperazin-1-yl)methyl]-2-thieno[3,2-c]pyridin-6-yl-3H-quinazolin-4-one;
6-(2-((2-Methoxyethyl)(methyl)amino)ethyl)-8-methyl-2-(thieno[3,2-c]pyridin-6-yl)quinazolin-4(3H)-one;
6-(2-(1,1-Dioxidothiomorpholino)ethyl)-8-methyl-2-(thieno[3,2-c]pyridin-6-yl)quinazolin-4(3H)-one;
6-[(1,1-Dioxo-1,4-thiazinan-4-yl)methyl]-8-methyl-2-thieno[3,2-c]pyridin-6-yl-3H-quinazolin-4-one;
6-(((2-Methoxyethyl)(methyl)amino)methyl)-8-methyl-2-(thieno[3,2-c]pyridin-6-yl)quinazolin-4(3H)-one;
6-[(4-Methoxy-1-piperidyl)methyl]-8-methyl-2-thieno[3,2-c]pyridin-6-yl-3H-quinazolin-4-one;
6-[(2,2-Dimethylmorpholin-4-yl)methyl]-8-methyl-2-thieno[3,2-c]pyridin-6-yl-3H-quinazolin-4-one;
8-Chloro-6-(2-morpholinoethyl)-2-(4-(trifluoromethyl)pyridin-2-yl)quinazolin-4(3H)-one;
8-Methyl-6-(2-oxa-7-azaspiro[3.5]nonan-7-ylmethyl)-2-thieno[3,2-c]pyridin-6-yl-3H-quinazolin-4-one;
N,N-Dimethyl-1-((8-methyl-4-oxo-2-(thieno[3,2-c]pyridin-6-yl)-3,4-dihydroquinazolin-6-yl)methyl)piperidine-4-carboxamide;
6-((4-(Methoxymethyl)piperidin-1-yl)methyl)-8-methyl-2-(thieno[3,2-c]pyridin-6-yl)quinazolin-4(3H)-one;
8-Methoxy-6-(2-morpholinoethyl)-2-(4-(trifluoromethyl)pyridin-2-yl)quinazolin-4(3H)-one;
8-Bromo-6-(2-morpholinoethyl)-2-(4-(trifluoromethyl)pyridin-2-yl)quinazolin-4(3H)-one;
6-(2-(2,2-Dimethylmorpholino)ethyl)-8-methyl-2-(4-(trifluoromethyl)pyridin-2-yl)quinazolin-4(3H)-one;
8-Methyl-6-((4-methyl-3-oxopiperazin-1-yl)methyl)-2-(4-(trifluoromethyl)pyridin-2-yl)quinazolin-4(3H)-one;
6-(2-(8-Oxa-3-azabicyclo[3.2.1]octan-3-yl)ethyl)-8-methyl-2-(4-(trifluoromethyl)pyridin-2-yl)quinazolin-4(3H)-one;
6-(2-(3-Oxa-8-azabicyclo[3.2.1]octan-8-yl)ethyl)-8-methyl-2-(4-(trifluoromethyl)pyridin-2-yl)quinazolin-4(3H)-one;
6-(2-(4-Hydroxypiperidin-1-yl)ethyl)-8-methyl-2-(4-(trifluoromethyl)pyridin-2-yl)quinazolin-4(3H)-one;
6-(2-(4,4-Difluoropiperidin-1-yl)ethyl)-8-methyl-2-(4-(trifluoromethyl)pyridin-2-yl)quinazolin-4(3H)-one;
6-(2-(4-Methoxypiperidin-1-yl)ethyl)-8-methyl-2-(4-(trifluoromethyl)pyridin-2-yl)quinazolin-4(3H)-one;
8-Methyl-6-(2-morpholinoethyl)-2-(4-(trifluoromethyl)pyridin-2-yl)pyrido[3,2-d]pyrimidin-4(3H)-one;

and pharmaceutically acceptable salts of any one of the aforementioned compounds.

The present invention also relates to each of the intermediates described further below in the examples section of this specification, including any one of these intermediates in non-salt form or in the form of a salt (e.g., a pharmaceutically acceptable salt) of the respective compound. Such intermediates can be used, in particular, in the synthesis of the compounds of formula (I).

For a person skilled in the field of synthetic chemistry, various ways for the preparation of the compounds of formula (I) will be readily apparent. For example, the compounds of formula (I) can be prepared as described in the following and, in particular, they can be prepared in accordance with or in analogy to the synthetic routes described in the examples section.

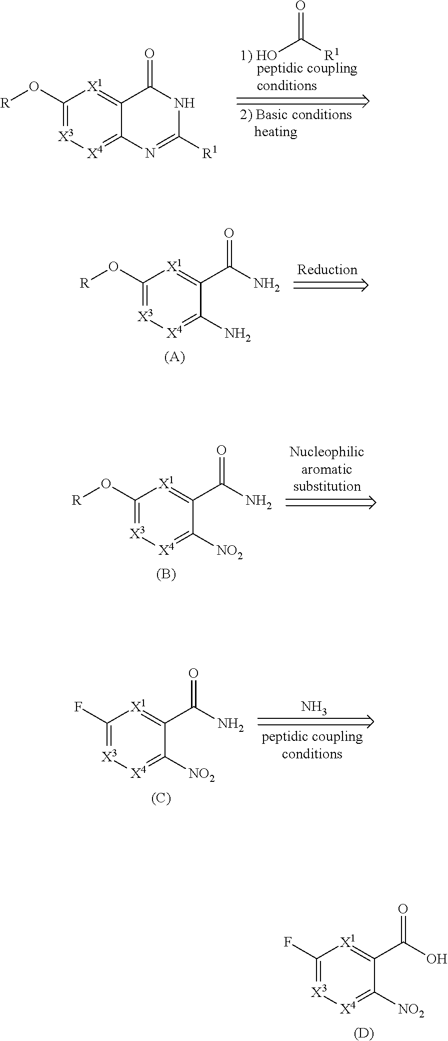

When -L-R^{X2}═—O—R, the compounds of formula (I) can be prepared from the corresponding anthranilic amides of formula (A) and a carboxylic acid under peptidic coupling conditions (Valeur et al., (2009) Chem. Soc. Rev., 38: 606-631), typically using BOP (benzotriazol-1-yloxy-tris(dimethylamino)-phosphonium hexafluorophosphate) as a coupling reagent, followed by a cyclisation step under basic conditions at high temperature. The anthranilic acids of formula (A) can be prepared by reduction of the corresponding nitro derivatives of formula (B). Typical conditions are the use of hydrogen with palladium on carbon, or the use of metals such as iron (Orlandi et al., (2018) Org. Process Res. Dev., 22: 430-445). The —O—R chain can be introduced from the fluorinated derivatives of formula (C) and an alcohol by nucleophilic aromatic substitution. Typical conditions are the use of a base at high temperature (Bunnett et al., (1951) Chem. Rev., 49: 273-412). The group -L-R^{X2} can be introduced directly from the corresponding alcohol, or the —O—R chain can be modified afterward to form -L-R^{X2}. For example, deprotection steps and/or further functionalizations can be carried out. Finally, the anthranilic amides of formula (C) can be obtained from the corresponding anthranilic acids of formula (D) in the presence of ammonia under peptidic coupling conditions, typically using BOP as a coupling reagent.

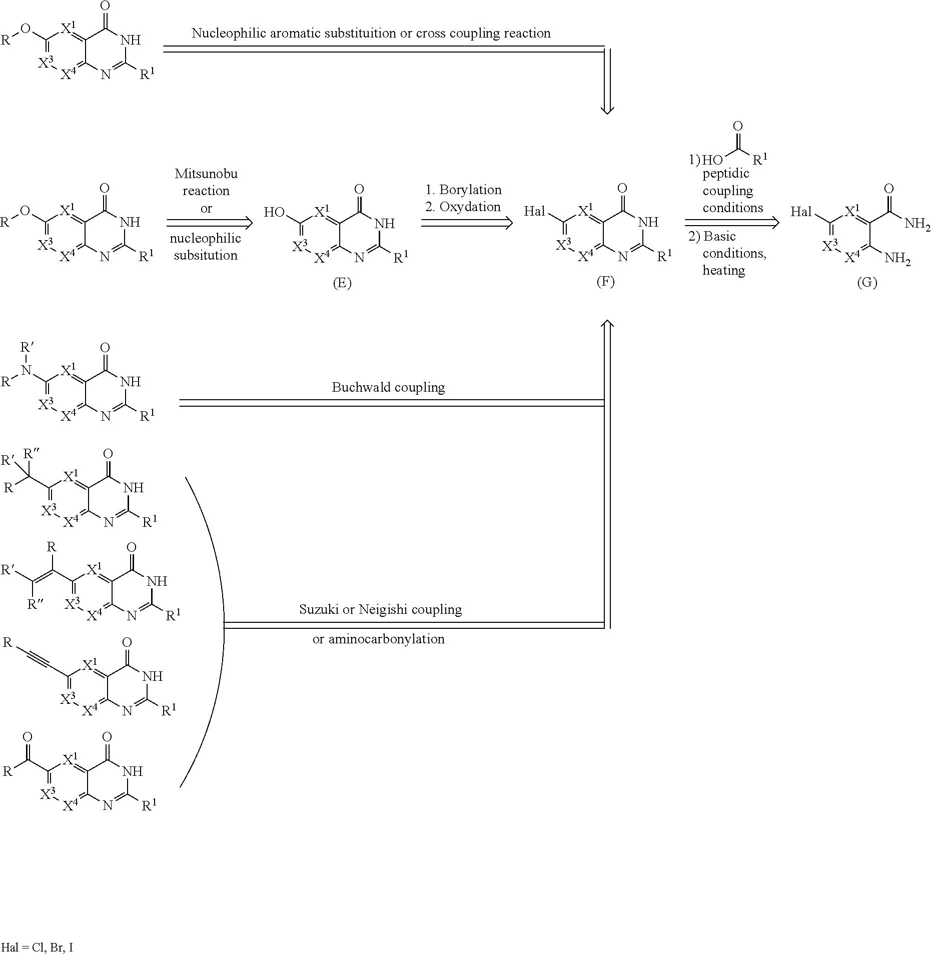

Alternatively, when -L-R^{X2}═—O—R, the compounds of formula (I) can be prepared from the compounds of formula (F) and an alcohol by nucleophilic substitution reaction or cross coupling reaction such as Ullmann-type reactions (Altman et al., (2008) J. Org. Chem., 73: 284-286) or a pallado-catalyzed coupling reaction (Bruno et al., (2013) Org. Lett., 15: 2876-2879) or by photoredox-nickel catalyzed C—O coupling reaction (Terrett et al, (2015) Nature, 524: 330). The group -L-R^{X2} can be introduced directly from the corresponding alcohol, or the —O—R chain can be modified afterward to form -L-R^{X2}. For example, deprotection steps and/or further functionalization can be carried out.

Alternatively, when -L-R^{X2}═—O—R, the compounds of formula (I) can be prepared from the compounds of formula (E) by Mitsunobu reaction with an alcohol (Swamy et al., (2009) Chem. Rev., 109: 2551-265), or by nucleophilic substitution from a halide or pseudo-halide derivative. The group -L-R^{X2} can be introduced directly from the corresponding alcohol or halide or pseudo-halide, or the —O—R chain can be modified afterward to form -L-R^{X2}. For example, deprotection steps and/or further functionalization can be carried out.

When -L-R^{X2}═—N—RR′, the compounds of formula (I) can be prepared from the compounds of formula (F) from an amine via Buchwald-Hartwig reaction (Heravi et al., (2018) J. Organomet. Chem., 861: 17). The group -L-R^{X2} can be introduced directly from the corresponding amine, or the —N—RR′ chain can be modified afterward to form -L-R^{X2}. For example, deprotection steps and/or further functionalization can be carried out.

When -L-R^{X2}═—CRR′R″, or —CR═R′R″, or C═R the compounds of formula (I) can be prepared from the compounds of formula (F) from an organometallic reagent for example by Suzuki (Maluenda et al., (2015) Molecules, 20: 7528) or Neigishi (Haas et al., (2016) ACS Catal., 6: 1540) coupling or by palladium catalyzed aminocarbonylation reaction (Wannberg et al., (2003) J. Org. Chem., 14: 5750). The group -L-R^{X2} can be introduced directly from the corresponding organometallic reagent, or the —CRR′R″, or —CR═R′R″, or C═R chain can be modified afterward to form -L-R^{X2}. For example, hydroboration and/or deprotection and/or further functionalization can be carried out.

The compound of formula (F) can be prepared from the corresponding anthranilic amides of formula (G) and a carboxylic acid under peptidic coupling conditions, typically using BOP as a coupling reagent, followed by cyclization under basic conditions and heating.

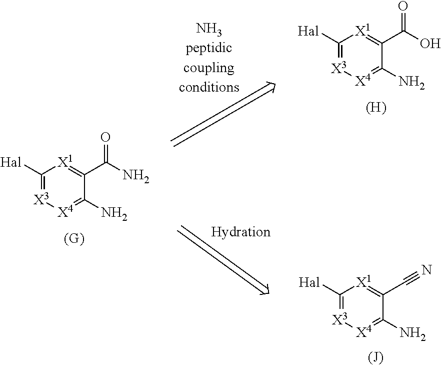

The anthranilic amides of formula (G) can be prepared from the corresponding acids of formula (H) in the presence of ammonia under peptidic coupling conditions, typically using BOP as a coupling reagent.

Alternatively, the anthranilic amides of formula (G) can be prepared from the corresponding nitrile derivative of formula (J) by hydration reaction.

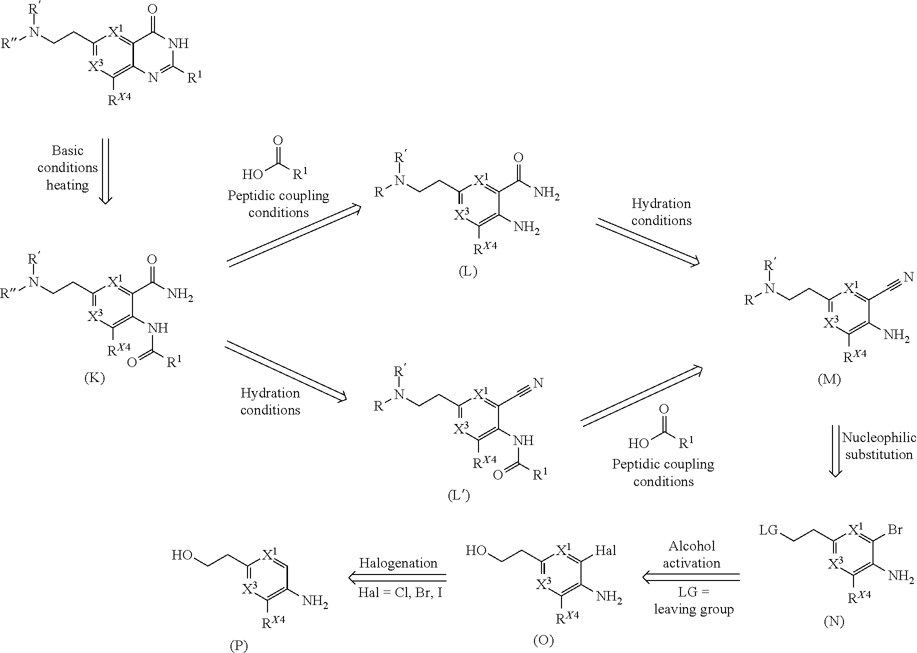

When -L-R^{X2}═—CH₂—CH₂—NRR′, the compounds of formula (I) can be prepared from the compounds of formula (K) by cyclisation under basic heating conditions. The compounds of formula (K) can be prepared from the nitrile derivatives of formula (M) by hydration and peptide coupling reactions with an acid regardless of the step order. Typical hydration conditions are the use of a strong acid such as hydrochloric acid or sulfuric acid, or a strong base such a potassium carbonate or milder conditions such as aqueous hydrogen peroxide and dimethyl sulfoxide in presence of base, such as potassium carbonate or sodium hydroxide. The peptidic coupling reaction can be performed with various ways of activating the carboxylic acid for example using BOP, T3P (propylphosphonic anhydride), oxalyl chloride or phosphorus oxychloride (Valeur et al., (2009) Chem. Soc. Rev., 38: 606-631). The amine HNRR′ can be introduced to give compounds of formulas (M) by activation of alcohol derivatives of formulas (O) to form a leaving group such as a mesylate, a tosylate, a triflate, or a halide, followed by nucleophilic substitution. Typical conditions are the use of a base such as triethylamine or potassium carbonate. Compounds of formulas (O) can be prepared by halogenation of compounds of formula (P) typically using N-bromosucinimide or iodine as a halogenation reagent.

The group —R^{X4} can be modified in the course of the synthesis, for example by electrophilic halogenation of the compounds of formula (O) or (M), or by pallado-catalysed coupling such as Suzuki coupling on the compounds of formula (O) or (M) to introduce alkyl groups from halogens (Maluenda et al., (2015) Molecules, 20: 7528).

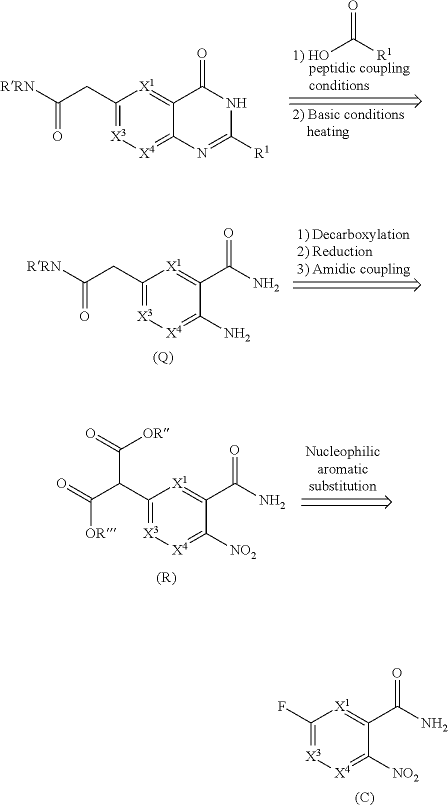

When -L-R^{X2}═—CH₂—C(O)—NRR′ the compounds of formula (I) can be prepared from the anthranilic amides of formula (Q) and a carboxylic acid under peptidic coupling conditions (Valeur et al., (2009) Chem. Soc. Rev., 38: 606-631), typically using BOP as a coupling reagent, followed by a cyclisation step under basic conditions at high temperature. One possibility to synthezise compounds of formula (Q) is by decarboxylation of compounds of formula (R) followed by reduction of the resulting nitro derivatives and amidic coupling with an amine NRR′ typically using BOP as a coupling reagent. Compounds of formula (R) can be prepared by nucleophilic aromatic substitution on fluorinated derivatives or formula (C) with a dialkyl malonate in the presence of a base at high temperature.

Additionally, compounds of formula (I) can be further functionalized to provide other compounds of formula (I), for example by cross-coupling reactions when X₁, X₃ and/or X₄═C-Hal.

While performing all the above described syntheses, the lactam NH bond can be temporarily protected, for example by a SEM protecting group.

The diverse reactants used to introduce -L-R^{X2} and the starting materials are either commercially available or can be prepared by classical organic chemistry reactions as described in the examples.

The following definitions apply throughout the present specification and the claims, unless specifically indicated otherwise.

The term “hydrocarbon group” refers to a group consisting of carbon atoms and hydrogen atoms.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms, and the term “C₀₋₃ alkylene” indicates that a covalent bond (corresponding to the option “C₀ alkylene”) or a C₁₋₃ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene.

As used herein, the term “alkenylene” refers to an alkenediyl group, i.e. a divalent unsaturated acyclic hydrocarbon group which may be linear or branched and comprises one or more (e.g., one or two) carbon-to-carbon double bonds while it does not comprise any carbon-to-carbon triple bond. A “C₂₋₈ alkenylene” denotes an alkenylene group having 2 to 8 carbon atoms. Unless defined otherwise, the term “alkenylene” preferably refers to C₂₋₄ alkenylene (including, in particular, linear C₂₋₄ alkenylene).

As used herein, the term “alkynylene” refers to an alkynediyl group, i.e. a divalent unsaturated acyclic hydrocarbon group which may be linear or branched and comprises one or more (e.g., one or two) carbon-to-carbon triple bonds and optionally one or more (e.g., one or two) carbon-to-carbon double bonds. A “C₂₋₈ alkynylene” denotes an alkynylene group having 2 to 8 carbon atoms. Unless defined otherwise, the term “alkynylene” preferably refers to C₂₋₄ alkynylene (including, in particular, linear C₂₋₄ alkynylene).

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “carbocyclylene” refers to a carbocyclyl group, as defined herein above, but having two points of attachment, i.e. a divalent hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclylene” preferably refers to arylene, cycloalkylene or cycloalkenylene.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

As used herein, the term “heterocyclylene” refers to a heterocyclyl group, as defined herein above, but having two points of attachment, i.e. a divalent ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclylene” preferably refers to heteroarylene, heterocycloalkylene or heterocycloalkenylene.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “arylene” refers to an aryl group, as defined herein above, but having two points of attachment, i.e. a divalent aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Arylene” may, e.g., refer to phenylene (e.g., phen-1,2-diyl, phen-1,3-diyl, or phen-1,4-diyl), naphthylene (e.g., naphthalen-1,2-diyl, naphthalen-1,3-diyl, naphthalen-1,4-diyl, naphthalen-1,5-diyl, naphthalen-1,6-diyl, naphthalen-1,7-diyl, naphthalen-2,3-diyl, naphthalen-2,5-diyl, naphthalen-2,6-diyl, naphthalen-2,7-diyl, or naphthalen-2,8-diyl), 1,2-dihydronaphthylene, 1,2,3,4-tetrahydronaphthylene, indanylene, indenylene, anthracenylene, phenanthrenylene, 9H-fluorenylene, or azulenylene. Unless defined otherwise, an “arylene” preferably has 6 to 14 ring atoms, more preferably 6 to 10 ring atoms, even more preferably refers to phenylene or naphthylene, and most preferably refers to phenylene (particularly phen-1,4-diyl).

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, 3-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “heteroarylene” refers to a heteroaryl group, as defined herein above, but having two points of attachment, i.e. a divalent aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three, or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroarylene” may, e.g., refer to thienylene (i.e., thiophenylene; e.g., thien-2,3-diyl, thien-2,4-diyl, or thien-2,5-diyl), benzo[b]thienylene, naphtho[2,3-b]thienylene, thianthrenylene, furylene (i.e., furanylene; e.g., furan-2,3-diyl, furan-2,4-diyl, or furan-2,5-diyl), benzofuranylene, isobenzofuranylene, chromanylene, chromenylene, isochromenylene, chromonylene, xanthenylene, phenoxathiinylene, pyrrolylene, imidazolylene, pyrazolylene, pyridylene (i.e., pyridinylene), pyrazinylene, pyrimidinylene, pyridazinylene, indolylene, isoindolylene, indazolylene, indolizinylene, purinylene, quinolylene, isoquinolylene, phthalazinylene, naphthyridinylene, quinoxalinylene, cinnolinylene, pteridinylene, carbazolylene, 3-carbolinylene, phenanthridinylene, acridinylene, perimidinylene, phenanthrolinylene, phenazinylene, thiazolylene (e.g., thiazol-2,4-diyl, thiazol-2,5-diyl, or thiazol-4,5-diyl), isothiazolylene (e.g., isothiazol-3,4-diyl, isothiazol-3,5-diyl, or isothiazol-4,5-diyl), phenothiazinylene, oxazolylene (e.g., oxazol-2,4-diyl, oxazol-2,5-diyl, or oxazol-4,5-diyl), isoxazolylene (e.g., isoxazol-3,4-diyl, isoxazol-3,5-diyl, or isoxazol-4,5-diyl), oxadiazolylene (e.g., 1,2,4-oxadiazol-3,5-diyl, 1,2,5-oxadiazol-3,4-diyl, or 1,3,4-oxadiazol-2,5-diyl), thiadiazolylene (e.g., 1,2,4-thiadiazol-3,5-diyl, 1,2,5-thiadiazol-3,4-diyl, or 1,3,4-thiadiazol-2,5-diyl), phenoxazinylene, pyrazolo[1,5-a]pyrimidinylene, 1,2-benzoisoxazolylene, benzothiazolylene, benzothiadiazolylene, benzoxazolylene, benzisoxazolylene, benzimidazolylene, benzo[b]thiophenylene (i.e., benzothienylene), triazolylene (e.g., 1H-1,2,3-triazolylene, 2H-1,2,3-triazolylene, 1H-1,2,4-triazolylene, or 4H-1,2,4-triazolylene), benzotriazolylene, 1H-tetrazolylene, 2H-tetrazolylene, triazinylene (e.g., 1,2,3-triazinylene, 1,2,4-triazinylene, or 1,3,5-triazinylene), furo[2,3-c]pyridinylene, dihydrofuropyridinylene (e.g., 2,3-dihydrofuro[2,3-c]pyridinylene or 1,3-dihydrofuro[3,4-c]pyridinylene), imidazopyridinylene (e.g., imidazo[1,2-a]pyridinylene or imidazo[3,2-a]pyridinylene), quinazolinylene, thienopyridinylene, tetrahydrothienopyridinylene (e.g., 4,5,6,7-tetrahydrothieno[3,2-c]pyridinylene), dibenzofuranylene, 1,3-benzodioxolylene, benzodioxanylene (e.g., 1,3-benzodioxanylene or 1,4-benzodioxanylene), or coumarinylene. Unless defined otherwise, the term “heteroarylene” preferably refers to a divalent 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroarylene” refers to a divalent 5 or 6 membered monocyclic ring comprising one or more (e.g., one, two or three) ring heteroatoms independently selected from O, S, and N, wherein one or more S ring atoms (if present) and/or one or more N ring atoms (if present) are optionally oxidized, and wherein one or more carbon ring atoms are optionally oxidized. A “heteroarylene”, including any of the specific heteroarylene groups described herein, may be attached through two carbon ring atoms, particularly through those two carbon ring atoms that have the greatest distance from one another (in terms of the number of ring atoms separating them by the shortest possible connection) within one single ring or within the entire ring system of the corresponding heteroarylene.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members.

As used herein, the term “cycloalkylene” refers to a cycloalkyl group, as defined herein above, but having two points of attachment, i.e. a divalent saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkylene” may, e.g., refer to cyclopropylene (e.g., cyclopropan-1,1-diyl or cyclopropan-1,2-diyl), cyclobutylene (e.g., cyclobutan-1,1-diyl, cyclobutan-1,2-diyl, or cyclobutan-1,3-diyl), cyclopentylene (e.g., cyclopentan-1,1-diyl, cyclopentan-1,2-diyl, or cyclopentan-1,3-diyl), cyclohexylene (e.g., cyclohexan-1,1-diyl, cyclohexan-1,2-diyl, cyclohexan-1,3-diyl, or cyclohexan-1,4-diyl), cycloheptylene, decalinylene (i.e., decahydronaphthylene), or adamantylene. Unless defined otherwise, “cycloalkylene” preferably refers to a C₃₋₁₁ cycloalkylene, and more preferably refers to a C₃₋₇ cycloalkylene. A particularly preferred “cycloalkylene” is a divalent monocyclic saturated hydrocarbon ring having 3 to 7 ring members.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “heterocycloalkylene” refers to a heterocycloalkyl group, as defined herein above, but having two points of attachment, i.e. a divalent saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkylene” may, e.g., refer to aziridinylene, azetidinylene, pyrrolidinylene, imidazolidinylene, pyrazolidinylene, piperidinylene, piperazinylene, azepanylene, diazepanylene (e.g., 1,4-diazepanylene), oxazolidinylene, isoxazolidinylene, thiazolidinylene, isothiazolidinylene, morpholinylene, thiomorpholinylene, oxazepanylene, oxiranylene, oxetanylene, tetrahydrofuranylene, 1,3-dioxolanylene, tetrahydropyranylene, 1,4-dioxanylene, oxepanylene, thiiranylene, thietanylene, tetrahydrothiophenylene (i.e., thiolanylene), 1,3-dithiolanylene, thianylene, thiepanylene, decahydroquinolinylene, decahydroisoquinolinylene, or 2-oxa-5-aza-bicyclo[2.2.1]hept-5-ylene. Unless defined otherwise, “heterocycloalkylene” preferably refers to a divalent 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkylene” refers to a divalent 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized.

As used herein, the term “cycloalkenyl” refers to an unsaturated alicyclic (i.e., non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₁ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “cycloalkenylene” refers to a cycloalkenyl group, as defined herein above, but having two points of attachment, i.e. a divalent unsaturated alicyclic (i.e., non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenylene” may, e.g., refer to cyclopropenylene, cyclobutenylene, cyclopentenylene, cyclohexenylene, cyclohexadienylene, cycloheptenylene, or cycloheptadienylene. Unless defined otherwise, “cycloalkenylene” preferably refers to a C₃₋₁₁ cycloalkenylene, and more preferably refers to a C₃₋₇ cycloalkenylene. A particularly preferred “cycloalkenylene” is a divalent monocyclic unsaturated alicyclic hydrocarbon ring having 3 to 7 ring members and containing one or more (e.g., one or two; preferably one) carbon-to-carbon double bonds.

As used herein, the term “heterocycloalkenyl” refers to an unsaturated alicyclic (i.e., non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “heterocycloalkenylene” refers to a heterocycloalkenyl group, as defined herein above, but having two points of attachment, i.e. a divalent unsaturated alicyclic (i.e., non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenylene” may, e.g., refer to imidazolinylene, tetrahydropyridinylene, dihydropyridinylene, pyranylene, thiopyranylene, dihydropyranylene, dihydrofuranylene, dihydropyrazolylene, dihydropyrazinylene, dihydroisoindolylene, octahydroquinolinylene, or octahydroisoquinolinylene. Unless defined otherwise, “heterocycloalkenylene” preferably refers to a divalent 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenylene” refers to a divalent 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “halogen” refers to fluoro (—F), chloro (—Cl), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A particularly preferred “haloalkyl” group is —CF₃.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, the preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

As used herein, the term “about” preferably refers to ±10% of the indicated numerical value, more preferably to ±5% of the indicated numerical value, and in particular to the exact numerical value indicated.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The scope of the present invention embraces all pharmaceutically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation.

Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts.

Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts.

Preferred pharmaceutically acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically acceptable salt of the compound of formula (I) is a hydrochloride salt. Accordingly, it is preferred that the compound of formula (I), including any one of the specific compounds of formula (I) described herein, is in the form of a hydrochloride salt, a hydrobromide salt, a mesylate salt, a sulfate salt, a tartrate salt, a fumarate salt, an acetate salt, a citrate salt, or a phosphate salt, and it is particularly preferred that the compound of formula (I) is in the form of a hydrochloride salt.

It will be understood that the present invention also relates to the compounds of formula (I), including any one of the specific compounds described herein, in non-salt form.

Moreover, the scope of the invention embraces the compounds of formula (I) in any solvated form, including, e.g., solvates with water (i.e., as a hydrate) or solvates with organic solvents such as, e.g., methanol, ethanol or acetonitrile (i.e., as a methanolate, ethanolate or acetonitrilate). All physical forms, including any amorphous or crystalline forms (i.e., polymorphs), of the compounds of formula (I) are also encompassed within the scope of the invention. It is to be understood that such solvates and physical forms of pharmaceutically acceptable salts of the compounds of the formula (I) are likewise embraced by the invention.

Furthermore, the compounds of formula (I) may exist in the form of different isomers, in particular stereoisomers (including, e.g., geometric isomers (or cis/trans isomers), enantiomers, atropisomers, and diastereomers) or tautomers (including, in particular, prototropic tautomers). All such isomers of the compounds of formula (I) are contemplated as being part of the present invention, either in admixture or in pure or substantially pure form.

As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization.

The present invention further encompasses any tautomers of the compounds provided herein (e.g., keto/enol tautomers or lactam/lactim tautomers), including in particular the following tautomers of the compounds of formula (I):

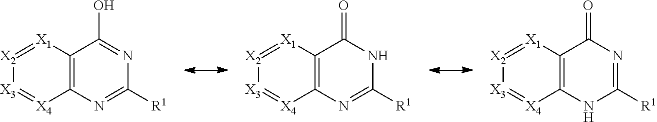

The scope of the invention also embraces compounds of formula (I), in which one or more atoms are replaced by a specific isotope of the corresponding atom.

For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers, trackers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by ¹⁵O atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms.

In general, it is preferred that none of the atoms in the compounds of formula (I) are replaced by specific isotopes.

The compounds of formula (I) can also be employed in the form of a pharmaceutically acceptable prodrug, i.e., as derivatives of the compounds of formula (I) which have chemically or metabolically cleavable groups and become, by solvolysis or under physiological conditions, the compounds of formula (I) which are pharmaceutically active in vivo. Prodrugs of the compounds according to the the present invention may be formed in a conventional manner with a functional group of the compounds such as, e.g., with an amino, hydroxy or carboxy group. The prodrug form often offers advantages in terms of solubility, tissue compatibility or delayed release in a mammalian organism (see, Bundgaard, H., Design of Prodrugs, pp. 7-9, 21-24, Elsevier, Amsterdam 1985). Prodrugs include acid derivatives, such as, e.g., esters prepared by reaction of the parent acidic compound with a suitable alcohol, or amides prepared by reaction of the parent acid compound with a suitable amine. If a compound of the present invention has a carboxyl group, an ester derivative prepared by reacting the carboxyl group with a suitable alcohol or an amide derivative prepared by reacting the carboxyl group with a suitable amine is exemplified as a prodrug. An especially preferred ester derivative as a prodrug is methylester, ethylester, n-propylester, isopropylester, n-butylester, isobutylester, tert-butylester, morpholinoethylester, N,N-diethylglycolamidoester or α-acetoxyethylester. If a compound of the present invention has a hydroxy group, an acyloxy derivative prepared by reacting the hydroxyl group with a suitable acylhalide or a suitable acid anhydride is exemplified as a prodrug. An especially preferred acyloxy derivative as a prodrug is —OC(═O)—CH₃, —OC(═O)—C₂H₅, —OC(═O)-(tert-Bu), —OC(═O)—C₁₅H₃₁, —OC(═O)-(m-COONa-Ph), —OC(═O)—CH₂CH₂COONa, —O(C═O)—CH(NH₂)CH₃ or —OC(═O)—CH₂—N(CH₃)₂. If a compound of the present invention has an amino group, an amide derivative prepared by reacting the amino group with a suitable acid halide or a suitable mixed anhydride is exemplified as a prodrug. An especially preferred amide derivative as a prodrug is —NHC(═O)—(CH₂)₂OCH₃ or —NHC(═O)—CH(NH₂)CH₃.

The compounds provided in accordance with the present invention, i.e. the compounds of formula (I) and/or pharmaceutically acceptable salts thereof, may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of the invention or the above described pharmaceutical compositions comprising a compound of the invention may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate, or poly-D-(−)-3-hydroxybutyric acid. Sustained-release pharmaceutical compositions also include liposomally entrapped compounds. The present invention thus also relates to liposomes containing a compound of the invention.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to an emulsification/spray drying process.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Preferred routes of administration are oral administration or parenteral administration, with oral administration being particularly preferred. Accordingly, it is particularly preferred that the compounds or the pharmaceutical compositions of the present invention are to be administered orally (particularly by oral ingestion or swallowing).

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds according to the invention for oral administration to a human (of approximately 70 kg body weight) may be 0.05 to 2000 mg, preferably 0.1 mg to 1000 mg, of the active ingredient per unit dose. The unit dose may be administered, e.g., 1 to 3 times per day. The unit dose may also be administered 1 to 7 times per week, e.g., with not more than one administration per day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The compound of formula (I) or the pharmaceutically acceptable salt thereof, or the pharmaceutical composition comprising the compound of formula (I) or a pharmaceutically acceptable salt thereof, can be administered in monotherapy (e.g., without concomitantly administering any further therapeutic agents, or without concomitantly administering any further therapeutic agents against the same disease that is to be treated or prevented with the compound of formula (I)). However, the compound of formula (I) or the pharmaceutically acceptable salt thereof, or the pharmaceutical composition comprising the compound of formula (I) or a pharmaceutically acceptable salt thereof, can also be administered in combination with one or more further therapeutic agents. If the compound of formula (I) or the pharmaceutically acceptable salt thereof is used in combination with a second therapeutic agent active against the same disease or condition, the dose of each compound may differ from that when the corresponding compound is used alone, in particular, a lower dose of each compound may be used. The combination of the compound of formula (I) or the pharmaceutically acceptable salt thereof with one or more further therapeutic agents may comprise the simultaneous/concomitant administration of the compound of formula (I) or the pharmaceutically acceptable salt thereof and the further therapeutic agent(s) (either in a single pharmaceutical formulation or in separate pharmaceutical formulations), or the sequential/separate administration of the compound of formula (I) or the pharmaceutically acceptable salt thereof and the further therapeutic agent(s). If administration is sequential, either the compound of formula (I) or the pharmaceutically acceptable salt thereof according to the present invention, or the one or more further therapeutic agents may be administered first. If administration is simultaneous, the one or more further therapeutic agents may be included in the same pharmaceutical formulation as the compound of formula (I) or the pharmaceutically acceptable salt thereof, or they may be administered in two or more different (separate) pharmaceutical formulations.

Preferably, the one or more further therapeutic agents to be administered in combination with a compound of the present invention are selected from levodopa, levodopa with selective extracerebral decarboxylase inhibitors, carbidopa, entacapone, COMT inhibitors, dopamine agonists, dopamine receptor agonists, apomorphine, anticholinergics, cholinergic agonists, butyrophenone neuroleptic agents, diphenylbutylpiperidine neuroleptic agents, heterocyclic dibenzazepine neuroleptic agents, indolone neuroleptic agents, phenothiazine neuroleptic agents, thioxanthene neuroleptic agents, NMDA receptor antagonists, MAO-B inhibitors, mGluR3 PAMs or agonists, mGluR4 PAMs or agonists, mGluR5 antagonists, and A2A antagonists.

In particular, for the treatment or prevention of Parkinson's disease, the compound of formula (I) or the pharmaceutically acceptable salt thereof, or the pharmaceutical composition comprising the compound of formula (I) or a pharmaceutically acceptable salt thereof, can also be administered in combination with one or more further antiparkinson agents. Such further antiparkinson agents may, for example, be selected from levodopa, melevodopa, etilevodopa, droxidopa, aplindore, apomorphine, bromocriptine, cabergoline, ciladopa, dihydroergocryptine, lisuride, pardoprunox, pergolide, piribedil, pramipexole, ropinirole, rotigotine, ladostigil, lazabemide, mofegiline, pargyline, rasagiline, selegiline, entacapone, nitecapone, tolcapone, benserazide, carbidopa, methyldopa, benzatropine, biperiden, bornaprine, chlorphenoxamine, cycrimine, dexetimide, dimenhydrinate, diphenhydramine, etanautine, etybenzatropine, mazaticol, metixene, orphenadrine, phenglutarimide, piroheptine, procyclidine, profenamine, trihexyphenidyl, tropatepine, amantadine, budipine, memantine, methylxanthines, rimantadine, UWA-101, and pharmaceutically acceptable salts of any of these agents. Preferred antiparkinson agents include levodopa, carbidopa, and/or biperiden, particularly levodopa.

For the treatment or prevention of Parkinson's disease, the combined administration of the compound or the pharmaceutical composition of the present invention with one or more further antiparkinson agents may be effected, e.g., by simultaneous/concomitant administration (either in a single pharmaceutical formulation or in separate pharmaceutical formulations) or by sequential/separate administration.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The term “treatment” of a condition, disorder or disease, as used herein, is well known in the art. “Treatment” of a condition, disorder or disease implies that a condition, disorder or disease is suspected or has been diagnosed in a patient/subject. A patient/subject suspected of suffering from a condition, disorder or disease typically shows specific clinical and/or pathological symptoms which a skilled person can easily attribute to a specific pathological condition (i.e., diagnose a condition, disorder or disease).

The “treatment” of a condition, disorder or disease may, for example, lead to a halt in the progression of the condition, disorder or disease (e.g., no deterioration of symptoms) or a delay in the progression of the condition, disorder or disease (in case the halt in progression is of a transient nature only). The “treatment” of a condition, disorder or disease may also lead to a partial response (e.g., amelioration of symptoms) or complete response (e.g., disappearance of symptoms) of the subject/patient suffering from the condition, disorder or disease. Accordingly, the “treatment” of a condition, disorder or disease may also refer to an amelioration of the condition, disorder or disease, which may, e.g., lead to a halt in the progression of the condition, disorder or disease or a delay in the progression of the condition, disorder or disease. Such a partial or complete response may be followed by a relapse. It is to be understood that a subject/patient may experience a broad range of responses to a treatment (such as the exemplary responses as described herein above). The treatment of a condition, disorder or disease may, inter alia, comprise curative treatment (preferably leading to a complete response and eventually to healing of the condition, disorder or disease) and palliative treatment (including symptomatic relief).

The term “prevention” of a condition, disorder or disease, as used herein, is also well known in the art. For example, a patient/subject suspected of being prone to suffer from a condition, disorder or disease may particularly benefit from a prevention of the condition, disorder or disease. The subject/patient may have a susceptibility or predisposition for a condition, disorder or disease, including but not limited to hereditary predisposition. Such a predisposition can be determined by standard methods or assays, using, e.g., genetic markers or phenotypic indicators. It is to be understood that a condition, disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

It is to be understood that the present invention specifically relates to each and every combination of features and embodiments described herein, including any combination of general and/or preferred features/embodiments. In particular, the invention specifically relates to each combination of meanings (including general and/or preferred meanings) for the various groups and variables comprised in formula (I).

In this specification, a number of documents including patents, patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The present invention particularly relates to the following items:
1. A compound of formula (I)

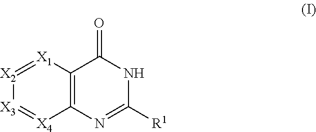

wherein:
   R¹ is selected from any one of the following groups:

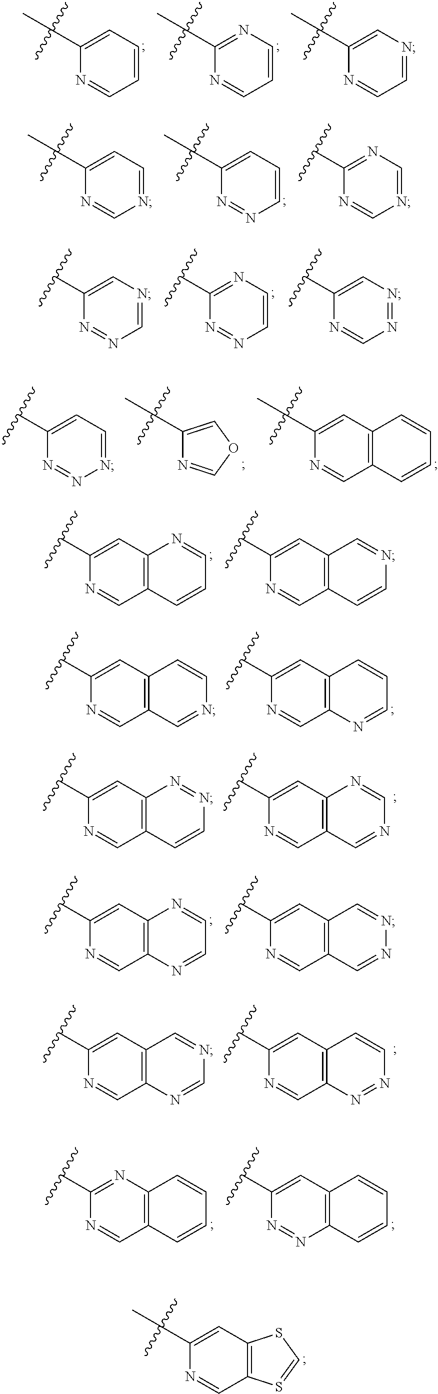

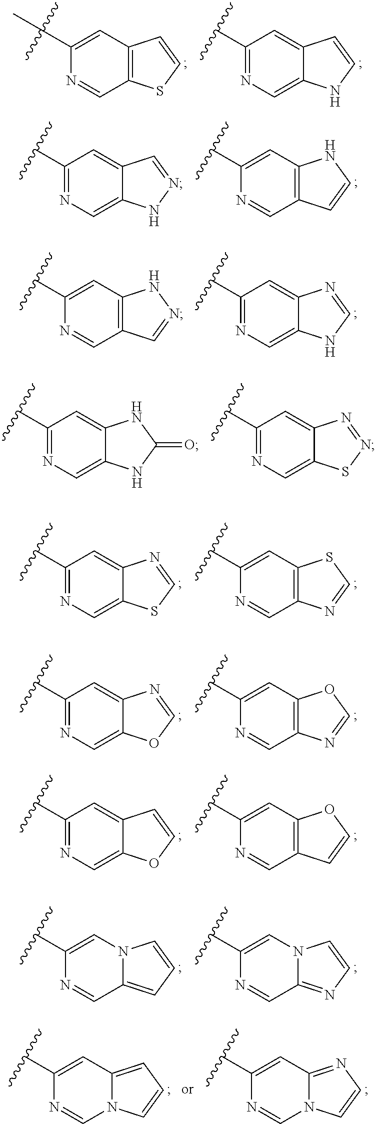

wherein each one of the above-depicted groups is optionally substituted with one or more groups R¹¹;
each R¹¹ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R¹²;
each R¹² is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl;
X₁ is C(R^{X1}) or N;
X₂ is C(-L-R^{X2}) or N;
X₃ is C(R^{X3}) or N;
X₄ is C(R^{X4}) or N;
wherein at least one of the ring atoms X₁, X₂, X₃ and X₄ is not N;
R^{X1} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R^{X11};
each R^{X11} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl;
L is selected from a covalent bond, C₁₋₁₀ alkylene, C₂₋₁₀ alkenylene, and C₂₋₁₀ alkynylene, wherein one or more —CH₂— units comprised in said C₁₋₁₀ alkylene, said C₂₋₁₀ alkenylene, or said C₂₋₁₀ alkynylene are each optionally replaced by a group independently selected from —O—, —CO—, —C(═O)O—, —O—C(═O)—, —NH—, —N(C₁₋₅ alkyl)-, —NH—CO—, —N(C₁₋₅ alkyl)-CO—, —CO—NH—, —CO—N(C₁₋₅ alkyl)-, —S—, —SO—, —SO₂—, —SO₂—NH—, —SO₂—N(C₁₋₅ alkyl)-, —NH—SO₂—, —N(C₁₋₅ alkyl)-SO₂—, carbocyclylene, and heterocyclylene, wherein said carbocyclylene and said heterocyclylene are each optionally substituted with one or more groups independently selected from C₁₋₄ alkyl, —OH, —O(C₁₋₄ alkyl), —SH, —S(C₁₋₅ alkyl), —NH₂, —NH(C₁₋₄ alkyl), —N(C₁₋₄ alkyl)(C₁₋₄ alkyl), halogen, C₁₋₅ haloalkyl, —O—(C₁₋₅ haloalkyl), and —CN, and further wherein said C₁₋₁₀ alkylene, said C₂₋₁₀ alkenylene, and said C₂₋₁₀ alkynylene are each optionally substituted with one or more groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
R^{X2} is selected from C₂₋₁₀ alkyl, carbocyclyl, heterocyclyl, and -L¹-R^{X21}, wherein said C₂₋₁₀ alkyl, said carbocyclyl and said heterocyclyl are each optionally substituted with one or more groups R^{X22};
L¹ is selected from a covalent bond, C₁₋₁₀ alkylene, C₂₋₁₀ alkenylene, and C₂₋₁₀ alkynylene, wherein one or more —CH₂— units comprised in said C₁₋₁₀ alkylene, said C₂₋₁₀ alkenylene, or said C₂₋₁₀ alkynylene are each optionally replaced by a group independently selected from —O—, —CO—, —C(═O)O—, —O—C(═O)—, —NH—, —N(C₁₋₅ alkyl)-, —NH—CO—, —N(C₁₋₅ alkyl)-CO—, —CO—NH—, —CO—N(C₁₋₅ alkyl)-, —S—, —SO—, —SO₂—, —SO₂—NH—, —SO₂—N(C₁₋₅ alkyl)-, —NH—SO₂—, and —N(C₁₋₅ alkyl)-SO₂—, and further wherein said C₁₋₁₀ alkylene, said C₂₋₁₀ alkenylene, and said C₂₋₁₀ alkynylene are each optionally substituted with one or more groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
R^{X21} is selected from C₂₋₅ alkyl, carbocyclyl, and heterocyclyl, wherein said carbocyclyl and said heterocyclyl are each optionally substituted with one or more groups R^{X22};
each R^{X22} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R^{X23};
each R^{X23} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), cycloalkyl, and heterocycloalkyl;
R^{X3} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R^{X31};
each R^{X31} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl;
R^{X4} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R^{X41};
each R^{X41} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl;
or a pharmaceutically acceptable salt thereof;
with the proviso that the following compounds are excluded from formula (I):

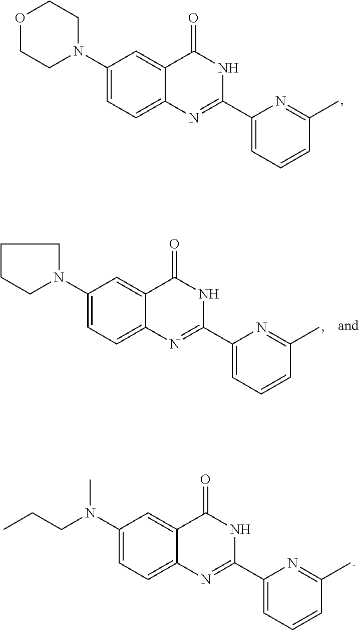

2. The compound of item 1, wherein R¹ is either selected from one of the following groups:

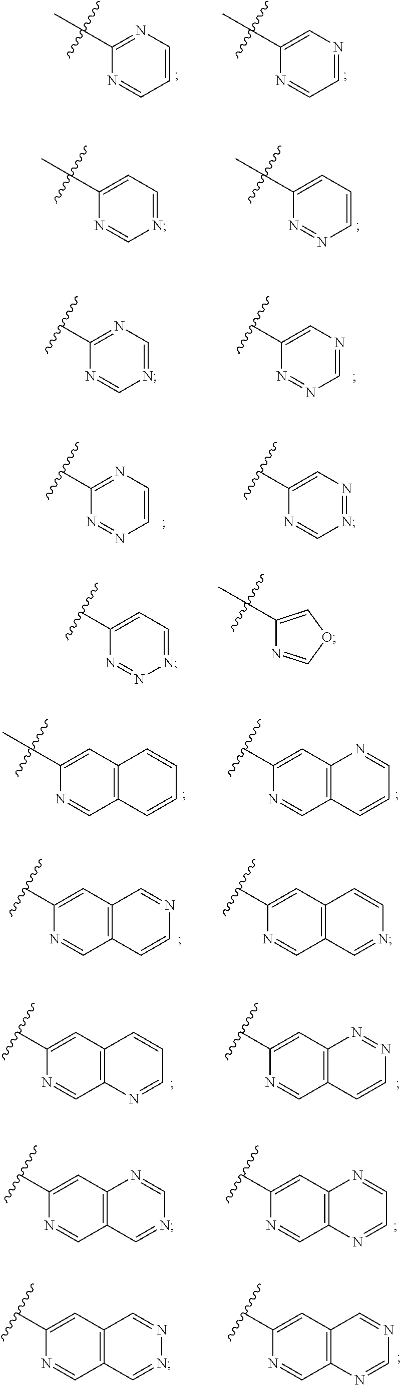

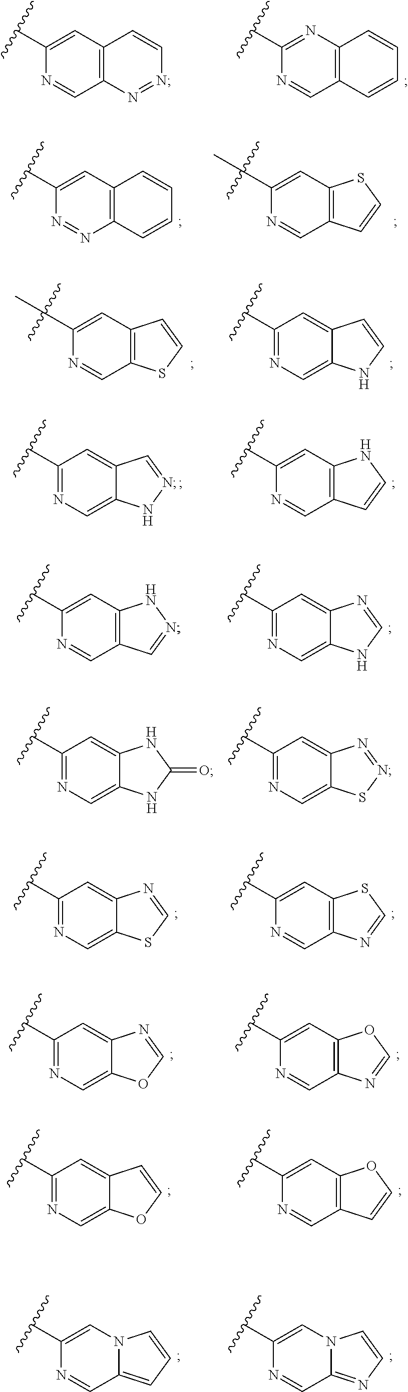

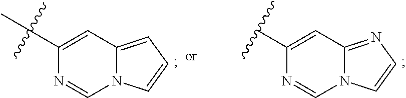 [SMILES: *c1cc2cccn2cn1 ; *c1cc2nccn2cn1]

wherein each one of the above-depicted groups is optionally substituted with one or more groups R¹¹;
or wherein R is selected from any one of the following groups:

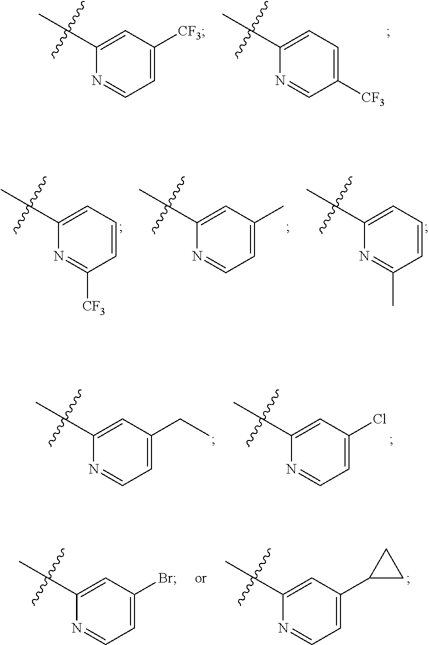

wherein each one of the above-depicted groups is optionally further substituted with one or more groups R¹¹.
3. The compound of item 1, wherein R¹ is selected from one of the following groups:

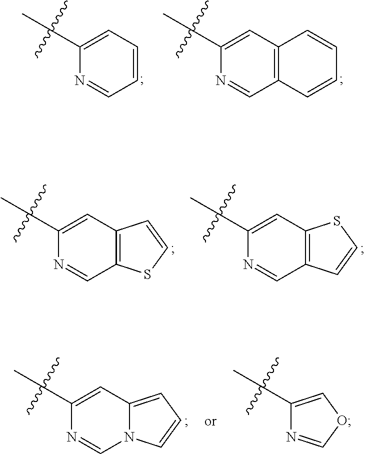 [SMILES: *c1ccccn1 ; *c1cc2ccccc2cn1 ; *c1cc2ccsc2cn1 ; *c1cc2sccc2cn1 ; *c1cc2cccn2cn1 ; *c1cocn1]

wherein each one of the above-depicted groups is optionally substituted with one or more groups R¹¹.
4. The compound of item 1, wherein R¹ is selected from one of the following groups:

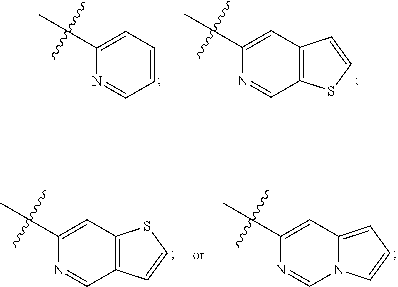 [SMILES: *c1ccccn1 ; *c1cc2ccsc2cn1 ; *c1cc2sccc2cn1 ; *c1cc2cccn2cn1]

wherein each one of the above-depicted groups is optionally substituted with one or more groups R¹¹;
and wherein it is preferred that R¹ is a group:

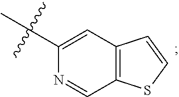 [SMILES: *c1cc2ccsc2cn1]

wherein the above-depicted group is optionally substituted with one or more groups R¹¹.
5. The compound of any one of items 1 to 4, wherein each R¹¹ is independently selected from C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), and —(C₀₋₃ alkylene)-CN.
6. The compound of any one of items 1 to 5, wherein X₂ is C(-L-R^{X2}).
7. The compound of any one of items 1 to 5, wherein X₁ is C(R^{X1}), X₂ is C(-L-R^{X2}), X₃ is C(R^{X3}), and X₄ is C(R^{X4}).
8. The compound of any one of items 1 to 7, wherein R^{X1} is selected from hydrogen, C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), and —(C₀₋₃ alkylene)-CN.
9. The compound of any one of items 1 to 8, wherein L is selected from a covalent bond, C₁₋₅ alkylene, —O—, —O—(C₁₋₅ alkylene)-, —NH—, —NH—(C₁₋₅ alkylene)-, —N(C₁₋₅ alkyl)-, and —N(C₁₋₅ alkyl)-(C₁₋₅ alkylene)-, wherein said C₁₋₅ alkylene or the C₁₋₅ alkylene moiety comprised in any of said —O—(C₁₋₅ alkylene)-, said —NH—(C₁₋₅ alkylene)-, and said —N(C₁₋₅ alkyl)-(C₁₋₅ alkylene)- is optionally substituted with one or more groups independently selected from halogen, —CF₃, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).
10. The compound of any one of items 1 to 8, wherein L is selected from a covalent bond, C₁₋₅ alkylene, —O—, and —O—(C₁₋₅ alkylene)-.
11. The compound of any one of items 1 to 10, wherein R^{X2} is selected from cycloalkyl, aryl, heterocycloalkyl, and heteroaryl, wherein said cycloalkyl, said aryl, said heterocycloalkyl, and said heteroaryl are each optionally substituted with one or more groups R^{X22}.
12. The compound of any one of items 1 to 8, wherein the group -L-R^{X2} is selected from —R^{X2}, —(C₁₋₅ alkylene)-R^{X2}, —O—R^{X2}, and —O—(C₁₋₅ alkylene)-R^{X2}, wherein R^{X2} is selected from cycloalkyl, aryl, heterocycloalkyl, and heteroaryl, wherein said cycloalkyl, said aryl, said heterocycloalkyl, and said heteroaryl are each optionally substituted with one or more groups R^{X22}.
13. The compound of any one of items 1 to 12, wherein R^{X2} is selected from azetidinyl, oxetanyl, pyrrolidinyl, oxopyrrolidinyl, tetrahydrofuranyl, piperidinyl, oxopiperidinyl, piperazinyl, morpholinyl, tetrahydropyranyl, 2-oxa-7-aza-spiro[3.5]nonyl, 6-oxa-2-aza-spiro[3.4]octyl, 3-oxa-9-aza-spiro[5.5]undecyl, 7-oxa-2-aza-spiro[4.5]decyl, 8-oxa-2-aza-spiro[4.5]decyl, phenyl, oxazolyl, pyridinyl, pyrazinyl, and pyrimidinyl, wherein each one of the aforementioned cyclic groups is optionally substituted with one or more groups R^{X22}.
14. The compound of any one of items 1 to 13, wherein R^{X3} is selected from hydrogen, C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), and —(C₀₋₃ alkylene)-CN.
15. The compound of any one of items 1 to 14, wherein R^{X4} is selected from hydrogen, C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, cycloalkyl, and heterocycloalkyl.
16. The compound of any one of items 1 to 14, wherein R^{X4} is selected from hydrogen, methyl, —OCH₃, halogen, and cyclopropyl.
17. The compound of any one of items 1 to 14, wherein R^{X4} is selected from methyl, —OCH₃, halogen, and cyclopropyl.
18. The compound of item 1, wherein said compound is selected from:
6-(3-Pyridin-4-yl-propoxy)-2-pyrrolo[1,2-c]pyrimidin-3-yl-3H-quinazolin-4-one;
2-Isoquinolin-3-yl-6-(3-pyridin-4-yl-propoxy)-3H-quinazolin-4-one;
6-(3-Pyridin-4-yl-propoxy)-2-thieno[3,2-c]pyridin-6-yl-3H-quinazolin-4-one;
6-(3-Pyridin-4-yl-propoxy)-2-thieno[2,3-c]pyridin-5-yl-3H-quinazolin-4-one;
2-Pyridin-2-yl-6-(3-pyridin-4-yl-propoxy)-3H-quinazolin-4-one;
2-(4-Methoxy-pyridin-2-yl)-6-(3-pyridin-4-yl-propoxy)-3H-quinazolin-4-one;
2-(5-Fluoro-pyridin-2-yl)-6-(3-pyridin-4-yl-propoxy)-3H-quinazolin-4-one;
6-(3-Pyridin-4-yl-propoxy)-2-(5-trifluoromethyl-pyridin-3-yl)-3H-quinazolin-4-one;
6-[3-(4-Pyridyl)propoxy]-2-[5-(trifluoromethyl)-2-pyridyl]-3H-quinazolin-4-one;
2-(4-Methyl-2-pyridyl)-6-[3-(4-pyridyl)propoxy]-3H-quinazolin-4-one;
2-(6-Methyl-2-pyridyl)-6-[3-(4-pyridyl)propoxy]-3H-quinazolin-4-one;
2-(5-Methylpyrazin-2-yl)-6-[3-(4-pyridyl)propoxy]-3H-quinazolin-4-one;
2-[5-Chloro-4-(trifluoromethyl)-2-pyridyl]-6-[3-(4-pyridyl)propoxy]3H-quinazolin-4-one;
2-(4-Chloro-2-pyridyl)-6-[3-(4-pyridyl)propoxy]-3H-quinazolin-4-one;
2-(4-Ethyl-2-pyridyl)-6-[3-(4-pyridyl)propoxy]-3H-quinazolin-4-one;
6-[3-(4-Pyridyl)propoxy]-2-[6-(trifluoromethyl)-2-pyridyl]-3H-quinazolin-4-one;
2-(4-Bromo-2-pyridyl)-6-[3-(4-pyridyl)propoxy]-3H-quinazolin-4-one;
2-(4-Cyclopropyl-2-pyridyl)-6-[3-(4-pyridyl)propoxy]-3H-quinazolin-4-one;
2-(2-Methyl-oxazol-4-yl)-6-(3-pyridin-4-yl-propoxy)-3H-quinazolin-4-one;
6-(2-Pyridin-3-yl-ethoxy)-2-thieno[3,2-c]pyridin-6-yl-3H-quinazolin-4-one;
6-(4-Bromo-benzyloxy)-2-thieno[3,2-c]pyridin-6-yl-3H-quinazolin-4-one;
Tert-butyl 3-(4-hydroxy-2-pyrrolo[1,2-c]pyrimidin-3-yl-quinazolin-6-yl)oxyazetidine-1-carboxylate;
6-(Azetidin-3-yloxy)-2-pyrrolo[1,2-c]pyrimidin-3-yl-3H-quinazolin-4-one;
6-(1-Pyrimidin-4-yl-azetidin-3-yloxy)-2-pyrrolo[1,2-c]pyrimidin-3-yl-3H-quinazolin-4-one;
3-(4-Hydroxy-2-thieno[2,3-c]pyridin-5-yl-quinazolin-6-yloxy)-azetidine-1-carboxylic acid tert-butyl ester;
6-(Azetidin-3-yloxy)-2-thieno[2,3-c]pyridin-5-yl-3H-quinazolin-4-one;
6-(1-Propionyl-azetidin-3-yloxy)-2-thieno[2,3-c]pyridin-5-yl-3H-quinazolin-4-one;
6-(Piperidin-4-yloxy)-2-thieno[3,2-c]pyridin-6-yl-3H-quinazolin-4-one;
6-(1-Propionyl-piperidin-4-yloxy)-2-thieno[3,2-c]pyridin-6-yl-3H-quinazolin-4-one;
6-(2-Morpholin-4-yl-ethoxy)-2-pyrrolo[1,2-c]pyrimidin-3-yl-3H-quinazolin-4-one;
6-(2-Methoxy-ethoxy)-2-pyrrolo[1,2-c]pyrimidin-3-yl-3H-quinazolin-4-one;
6-(2-Morpholin-4-yl-ethoxy)-2-thieno[3,2-c]pyridin-6-yl-3H-quinazolin-4-one;
6-(2-Methoxy-ethoxy)-2-thieno[3,2-c]pyridin-6-yl-3H-quinazolin-4-one;
6-(3-Pyridin-3-yl-propoxy)-2-thieno[3,2-c]pyridin-6-yl-3H-quinazolin-4-one;
4-(4-Oxo-2-pyridin-2-yl-3,4-dihydro-quinazolin-6-yloxy)-piperidine-1-carboxylic acid tert-butyl ester;
6-(Piperidin-4-yloxy)-2-pyridin-2-yl-3H-quinazolin-4-one;
6-(1-Acetyl-piperidin-4-yloxy)-2-pyridin-2-yl-3H-quinazolin-4-one;
4-[4-Oxo-2-(4-trifluoromethyl-pyridin-2-yl)-3,4-dihydro-quinazolin-6-yloxymethyl]-piperidine-1-carboxylic acid tert-butyl ester;
6-(Piperidin-4-ylmethoxy)-2-(4-trifluoromethyl-pyridin-2-yl)-3H-quinazolin-4-one;
6-(1-Acetyl-piperidin-4-ylmethoxy)-2-(4-trifluoromethyl-pyridin-2-yl)-3H-quinazolin-4-one;
tert-butyl 4-[(4-oxo-2-thieno[2,3-c]pyridin-5-yl-3H-quinazolin-6-yl)oxymethyl]piperidine-1-carboxylate;
6-(4-piperidylmethoxy)-2-thieno[2,3-c]pyridin-5-yl-3H-quinazolin-4-one;
6-(1-Acetyl-piperidin-4-ylmethoxy)-2-thieno[2,3-c]pyridin-5-yl-3H-quinazolin-4-one;
6-(1-Propionyl-piperidin-4-ylmethoxy)-2-thieno[2,3-c]pyridin-5-yl-3H-quinazolin-4-one;
3-(4-Oxo-2-thieno[2,3-c]pyridin-5-yl-3,4-dihydro-quinazolin-6-yloxy)-pyrrolidine-1-carboxylic acid tert-butyl ester;
6-(Pyrrolidin-3-yloxy)-2-thieno[2,3-c]pyridin-5-yl-3H-quinazolin-4-one;
6-(1-Acetyl-pyrrolidin-3-yloxy)-2-thieno[2,3-c]pyridin-5-yl-3H-quinazolin-4-one;
4-[4-Oxo-2-(4-trifluoromethyl-pyridin-2-yl)-3,4-dihydro-quinazolin-6-yl]-piperazine-1-carboxylic acid tert-butyl ester;
6-Piperazin-1-yl-2-(4-trifluoromethyl-pyridin-2-yl)-3H-quinazolin-4-one;
6-(4-Propionyl-piperazin-1-yl)-2-(4-trifluoromethyl-pyridin-2-yl)-3H-quinazolin-4-one;
4-(4-Oxo-2-thieno[2,3-c]pyridin-5-yl-3,4-dihydro-quinazolin-6-yl)-piperidine-1-carboxylic acid tert-butyl ester;
6-Piperidin-4-yl-2-thieno[2,3-c]pyridin-5-yl-3H-quinazolin-4-one;
6-(1-Acetyl-piperidin-4-yl)-2-thieno[2,3-c]pyridin-5-yl-3H-quinazolin-4-one;
6-[2-(Tetrahydro-pyran-4-yl)-ethoxy]-2-(4-trifluoromethyl-pyridin-2-yl)-3H-quinazolin-4-one;
6-[3-(3-Fluoro-pyridin-4-yl)-propoxy]-2-thieno[2,3-c]pyridin-5-yl-3H-quinazolin-4-one;
6-[3-(4-Methanesulfonyl-phenyl)-propoxy]-2-thieno[2,3-c]pyridin-5-yl-3H-quinazolin-4-one;
6-(3-Pyrazin-2-yl-propoxy)-2-thieno[2,3-c]pyridin-5-yl-3H-quinazolin-4-one;
6-[3-(3-Methoxy-pyridin-4-yl)-propoxy]-2-thieno[2,3-c]pyridin-5-yl-3H-quinazolin-4-one;
6-[3-(2-Methyl-pyridin-4-yl)-propoxy]-2-thieno[2,3-c]pyridin-5-yl-3H-quinazolin-4-one;
6-(3-Oxazol-4-yl-propoxy)-2-thieno[2,3-c]pyridin-5-yl-3H-quinazolin-4-one;
8-Methyl-6-(3-pyridin-4-yl-propoxy)-2-thieno[2,3-c]pyridin-5-yl-3H-quinazolin-4-one;
6-(3-Pyridin-4-yl-propoxy)-2-thieno[2,3-c]pyridin-5-yl-3H-pyrido[3,2-d]pyrimidin-4-one;
6-(3-Pyridin-4-yl-propoxy)-2-(4-trifluoromethyl-pyridin-2-yl)-3H-pyrido[3,2-d]pyrimidin-4-one;
6-(3-Pyridin-4-yl-propoxy)-2-(4-trifluoromethyl-pyridin-2-yl)-3H-pyrido[2,3-d]pyrimidin-4-one;
6-(3-Pyridin-4-yl-propoxy)-2-(4-trifluoromethyl-pyridin-2-yl)-3H-pyrido[3,4-d]pyrimidin-4-one;
6-(3-Pyridin-4-yl-propoxy)-2-thieno[2,3-c]pyridin-5-yl-7-trifluoromethyl-3H-quinazolin-4-one;
5-Chloro-6-(3-pyridin-4-yl-propoxy)-2-thieno[2,3-c]pyridin-5-yl-3H-quinazolin-4-one;
8-Chloro-6-(3-pyridin-4-yl-propoxy)-2-thieno[2,3-c]pyridin-5-yl-3H-quinazolin-4-one;
8-Cyclopropyl-6-(3-pyridin-4-yl-propoxy)-2-thieno[2,3-c]pyridin-5-yl-3H-quinazolin-4-one;
8-Ethyl-6-(3-pyridin-4-yl-propoxy)-2-thieno[2,3-c]pyridin-5-yl-3H-quinazolin-4-one;
8-Fluoro-6-(3-pyridin-4-yl-propoxy)-2-thieno[2,3-c]pyridin-5-yl-3H-quinazolin-4-one;
8-Methyl-6-(tetrahydro-pyran-4-ylmethoxy)-2-thieno[2,3-c]pyridin-5-yl-3H-quinazolin-4-one;
8-Methyl-6-(2-oxetan-3-yl-ethoxy)-2-thieno[2,3-c]pyridin-5-yl-3H-quinazolin-4-one;
8-Methyl-6-[2-(tetrahydro-furan-3-yl)-ethoxy]-2-thieno[2,3-c]pyridin-5-yl-3H-quinazolin-4-one;
8-Methyl-6-[2-(tetrahydro-pyran-4-yl)-ethoxy]-2-thieno[2,3-c]pyridin-5-yl-3H-quinazolin-4-one;
8-Methyl-6-(tetrahydro-furan-3-ylmethoxy)-2-thieno[2,3-c]pyridin-5-yl-3H-quinazolin-4-one;
R-8-Methyl-6-(tetrahydro-furan-3-ylmethoxy)-2-thieno[2,3-c]pyridin-5-yl-3H-quinazolin-4-one;
S-8-Methyl-6-(tetrahydro-furan-3-ylmethoxy)-2-thieno[2,3-c]pyridin-5-yl-3H-quinazolin-4-one;
8-Methyl-6-(1-methyl-6-oxo-piperidin-3-yloxy)-2-thieno[2,3-c]pyridin-5-yl-3H-quinazolin-4-one;
R-8-Methyl-6-((1-methyl-6-oxopiperidin-3-yl)oxy)-2-(thieno[2,3-c]pyridin-5-yl)quinazolin-4(3H)-one;
S-8-Methyl-6-((1-methyl-6-oxopiperidin-3-yl)oxy)-2-(thieno[2,3-c]pyridin-5-yl)quinazolin-4(3H)-one;
8-Methyl-6-(1-propionyl-pyrrolidin-3-yloxy)-2-thieno[2,3-c]pyridin-5-yl-3H-quinazolin-4-one;
R-8-Methyl-6-(1-propionyl-pyrrolidin-3-yloxy)-2-thieno[2,3-c]pyridin-5-yl-3H-quinazolin-4-one;
S-8-Methyl-6-(1-propionyl-pyrrolidin-3-yloxy)-2-thieno[2,3-c]pyridin-5-yl-3H-quinazolin-4-one;
8-Methyl-6-(1-oxetan-3-yl-pyrrolidin-3-yloxy)-2-thieno[2,3-c]pyridin-5-yl-3H-quinazolin-4-one;
R-8-Methyl-6-(1-oxetan-3-yl-pyrrolidin-3-yloxy)-2-thieno[2,3-c]pyridin-5-yl-3H-quinazolin-4-one;
S-8-Methyl-6-(1-oxetan-3-yl-pyrrolidin-3-yloxy)-2-thieno[2,3-c]pyridin-5-yl-3H-quinazolin-4-one;
8-Methyl-6-[2-(2-oxa-7-aza-spiro[3.5]non-7-yl)-ethoxy]-2-thieno[2,3-c]pyridin-5-yl-3H-quinazolin-4-one;
8-methyl-6-(piperidin-4-ylmethoxy)-2-thieno[2,3-c]pyridin-5-yl-3H-quinazolin-4-one;
8-Methyl-6-(1-oxetan-3-yl-piperidin-4-ylmethoxy)-2-thieno[2,3-c]pyridin-5-yl-3H-quinazolin-4-one;
8-Methyl-6-(1-propionyl-piperidin-4-ylmethoxy)-2-thieno[2,3-c]pyridin-5-yl-3H-quinazolin-4-one;
6-(1-Methanesulfonyl-piperidin-4-ylmethoxy)-8-methyl-2-thieno[2,3-c]pyridin-5-yl-3H-quinazolin-4-one;
8-Methyl-6-(2-oxa-7-aza-spiro[3.5]non-7-yl)-2-thieno[2,3-c]pyridin-5-yl-3H-quinazolin-4-one;
8-Methyl-6-(6-oxa-2-aza-spiro[3.4]oct-2-yl)-2-thieno[2,3-c]pyridin-5-yl-3H-quinazolin-4-one;
8-Methyl-6-(3-oxa-9-aza-spiro[5.5]undec-9-yl)-2-thieno[2,3-c]pyridin-5-yl-3H-quinazolin-4-one;
8-Methyl-6-(7-oxa-2-aza-spiro[4.5]dec-2-yl)-2-thieno[2,3-c]pyridin-5-yl-3H-quinazolin-4-one;
8-Methyl-6-(8-oxa-2-aza-spiro[4.5]dec-2-yl)-2-thieno[2,3-c]pyridin-5-yl-3H-quinazolin-4-one;
6-(2-Hydroxy-2-methyl-propylamino)-8-methyl-2-thieno[2,3-c]pyridin-5-yl-3H-quinazolin-4-one;
8-Methyl-6-(2-piperidin-3-yl-ethoxy)-2-thieno[2,3-c]pyridin-5-yl-3H-quinazolin-4-one;
6-[2-(1-Acetyl-piperidin-3-yl)-ethoxy]-8-methyl-2-thieno[2,3-c]pyridin-5-yl-3H-quinazolin-4-one;
6-[2-(4-Acetyl-piperazin-1-yl)-ethoxy]-8-methyl-2-thieno[2,3-c]pyridin-5-yl-3H-quinazolin-4-one;
3-(8-Methyl-4-oxo-2-thieno[2,3-c]pyridin-5-yl-3,4-dihydro-quinazolin-6-yl)-propionaldehyde;
8-Methyl-6-(3-morpholin-4-yl-propyl)-2-thieno[2,3-c]pyridin-5-yl-3H-quinazolin-4-one;
8-Methyl-6-(2-morpholin-4-yl-ethyl)-2-thieno[2,3-c]pyridin-5-yl-3H-quinazolin-4-one;
8-Methyl-6-(3-pyridin-4-yl-propoxy)-2-(4-trifluoromethyl-pyridin-2-yl)-3H-quinazolin-4-one;
8-Methyl-6-(tetrahydro-furan-3-ylmethoxy)-2-(4-trifluoromethyl-pyridin-2-yl)-3H-quinazolin-4-one;
8-Methyl-6-(1-propionyl-azetidin-3-yloxy)-2-(4-trifluoromethyl-pyridin-2-yl)-3H-quinazolin-4-one;
8-Methyl-6-(1-oxetan-3-yl-piperidin-4-yloxy)-2-(4-trifluoromethyl-pyridin-2-yl)-3H-quinazolin-4-one;
8-Methyl-6-(3-oxa-9-aza-spiro[5.5]undec-9-yl)-2-(4-trifluoromethyl-pyridin-2-yl)-3H-quinazolin-4-one;
8-Methyl-6-(3-pyridin-4-yl-propoxy)-2-pyrrolo[1,2-c]pyrimidin-3-yl-3H-quinazolin-4-one;
8-Methyl-2-pyrrolo[1,2-c]pyrimidin-3-yl-6-(tetrahydro-furan-3-ylmethoxy)-3H-quinazolin-4-one;
8-Methyl-6-(3-oxa-9-aza-spiro[5.5]undec-9-yl)-2-pyrrolo[1,2-c]pyrimidin-3-yl-3H-quinazolin-4-one;
8-Methyl-6-(1-oxetan-3-yl-piperidin-4-yloxy)-2-pyrrolo[1,2-c]pyrimidin-3-yl-3H-quinazolin-4-one;
8-Methyl-6-[1-(tetrahydro-pyran-4-yl)-ethoxy]-2-thieno[2,3-c]pyridin-5-yl-3H-quinazolin-4-one;
R-8-Methyl-6-[1-(tetrahydro-pyran-4-yl)-ethoxy]-2-thieno[2,3-c]pyridin-5-yl-3H-quinazolin-4-one;
S-8-Methyl-6-[1-(tetrahydro-pyran-4-yl)-ethoxy]-2-thieno[2,3-c]pyridin-5-yl-3H-quinazolin-4-one;
8-Methyl-6-[1-(tetrahydro-pyran-4-yl)-ethoxy]-2-(4-trifluoromethyl-pyridin-2-yl)-3H-quinazolin-4-one;
R-8-Methyl-6-[1-(tetrahydro-pyran-4-yl)-ethoxy]-2-(4-trifluoromethyl-pyridin-2-yl)-3H-quinazolin-4-one;
S-8-Methyl-6-[1-(tetrahydro-pyran-4-yl)-ethoxy]-2-(4-trifluoromethyl-pyridin-2-yl)-3H-quinazolin-4-one;
6-[(3-fluorotetrahydrofuran-3-yl)methoxy]-8-methyl-2-[4-(trifluoromethyl)-2-pyridyl]-3H-quinazolin-4-one;
8-methyl-6-(3-oxa-9-azaspiro[5.5]undecan-9-yl)-2-thieno[2,3-c]pyridin-5-yl-3-(2-trimethylsilylethoxymethyl)pyrido[3,2-d]pyrimidin-4-one;
8-methyl-6-(morpholinomethyl)-2-thieno[2,3-c]pyridin-5-yl-3H-quinazolin-4-one;
8-methyl-6-(morpholinomethyl)-2-[4-(trifluoromethyl)-2-pyridyl]-3H-quinazolin-4-one;
8-methyl-6-(1-propanoylazetidin-3-yl)oxy-2-thieno[2,3-c]pyridin-5-yl-3H-quinazolin-4-one;
8-methyl-6-(2-morpholinoethyl)-2-[4-(trifluoromethyl)-2-pyridyl]-3H-quinazolin-4-one;
8-Methyl-6-[(1-methyl-6-oxo-3-piperidyl)oxy]-2-pyrrolo[1,2-c]pyrimidin-3-yl-3H-quinazolin-4-one;
8-Methyl-6-(morpholinomethyl)-2-(thieno[3,2-c]pyridin-6-yl)quinazolin-4(3H)-one;
8-Methyl-6-(3-oxa-9-azaspiro[5.5]undecan-9-yl)-2-(thieno[3,2-c]pyridin-6-yl)quinazolin-4(3H)-one;
8-Methyl-6-(2-[1,4]oxazepan-4-yl-ethyl)-2-thieno[2,3-b]pyridin-5-yl-3H-quinazolin-4-one;
8-Methyl-6-(2-[1,4]oxazepan-4-yl-ethyl)-2-thieno[3,2-b]pyridin-6-yl-3H-quinazolin-4-one;
8-Methyl-6-(2-morpholin-4-yl-ethyl)-2-thieno[3,2-c]pyridin-6-yl-3H-quinazolin-4-one;
8-Methyl-6-(2-morpholin-4-yl-ethyl)-2-pyrrolo[1,2-c]pyrimidin-3-yl-3H-quinazolin-4-one;
8-Methyl-6-(morpholinomethyl)-2-pyrrolo[1,2-c]pyrimidin-3-yl-3H-quinazolin-4-one;
8-Methyl-6-(2-morpholino-2-oxoethyl)-2-(thieno[3,2-c]pyridin-6-yl)quinazolin-4(3H)-one;
8-Methyl-6-(2-morpholino-2-oxo-ethyl)-2-thieno[2,3-c]pyridin-5-yl-3H-quinazolin-4-one;
8-Methyl-6-(2-piperidin-1-yl-ethyl)-2-thieno[2,3-b]pyridin-5-yl-3H-quinazolin-4-one;
8-Methyl-6-(1-methyl-6-oxo-piperidin-3-yloxy)-2-thieno[3,2-c]pyridin-6-yl-3H-quinazolin-4-one;
8-Methyl-6-(1-methyl-2-oxo-piperidin-4-ylmethoxy)-2-thieno[2,3-c]pyridin-5-yl-3H-quinazolin-4-one
8-Methyl-6-(1-piperidylmethyl)-2-thieno[2,3-c]pyridin-5-yl-3H-quinazolin-4-one;
8-Methyl-6-(2-morpholino-2-oxo-ethyl)-2-thieno[3,2-c]pyridin-6-yl-3H-quinazolin-4-one;
8-Methyl-6-[(4-methylpiperazin-1-yl)methyl]-2-thieno[2,3-c]pyridin-5-yl-3H-quinazolin-4-one;
8-Methyl-6-(pyrrolidin-1-ylmethyl)-2-thieno[2,3-c]pyridin-5-yl-3H-quinazolin-4-one;
8-Methyl-6-(2-morpholino-2-oxo-ethyl)-2-pyrrolo[1,2-c]pyrimidin-3-yl-3H-quinazolin-4-one;
8-Methyl-6-(morpholine-4-carbonyl)-2-pyrrolo[1,2-c]pyrimidin-3-yl-3H-quinazolin-4-one;
8-Methyl-6-(1-methyl-6-oxo-piperidin-3-yloxy)-2-(4-trifluoromethyl-pyridin-2-yl)-3H-quinazolin-4-one;
8-Methyl-2-thieno[2,3-c]pyridin-5-yl-6-(thiomorpholinomethyl)-3H-quinazolin-4-one;
8-Methyl-6-[2-(1,4-oxazepan-4-yl)-2-oxo-ethyl]-2-thieno[2,3-c]pyridin-5-yl-3H-quinazolin-4-one;
8-Methyl-6-(pyrrolidin-1-ylmethyl)-2-thieno[3,2-c]pyridin-6-yl-3H-quinazolin-4-one;
8-Methyl-6-(1-methyl-5-oxo-pyrrolidin-3-yl)oxy-2-thieno[3,2-c]pyridin-6-yl-3H-quinazolin-4-one;
8-Methyl-6-[(3R)-1-methyl-5-oxo-pyrrolidin-3-yl]oxy-2-thieno[3,2-c]pyridin-6-yl-3H-quinazolin-4-one;
8-Methyl-6-[(3S)-1-methyl-5-oxo-pyrrolidin-3-yl]oxy-2-thieno[3,2-c]pyridin-6-yl-3H-quinazolin-4-one;
Benzyl 3-[(8-methyl-4-oxo-2-thieno[3,2-c]pyridin-6-yl-3H-quinazolin-6-yl)oxy]pyrrolidine-1-carboxylate;
Benzyl (3S)-3-[(8-methyl-4-oxo-2-thieno[3,2-c]pyridin-6-yl-3H-quinazolin-6-yl)oxy]pyrrolidine-1-carboxylate;
Benzyl (3R)-3-[(8-methyl-4-oxo-2-thieno[3,2-c]pyridin-6-yl-3H-quinazolin-6-yl)oxy]pyrrolidine-1-carboxylate;
8-Methyl-6-[2-(4-methyl-3-oxo-piperazin-1-yl)ethyl]-2-thieno[2,3-c]pyridin-5-yl-3H-quinazolin-4-one;
8-Methyl-6-[2-(4-methyl-3-oxo-piperazin-1-yl)ethyl]-2-[4-(trifluoromethyl)-2-pyridyl]-3H-quinazolin-4-one;
8-Methyl-6-[2-(2-oxa-6-azaspiro[3.3]heptan-6-yl)ethyl]-2-thieno[2,3-c]pyridin-5-yl-3H-quinazolin-4-one;
8-Methyl-6-[2-(2-oxa-6-azaspiro[3.3]heptan-6-yl)ethyl]-2-thieno[3,2-c]pyridin-6-yl-3H-quinazolin-4-one;
8-Methyl-6-[(4-methyl-3-oxo-piperazin-1-yl)methyl]-2-thieno[3,2-c]pyridin-6-yl-3H-quinazolin-4-one;
6-(2-((2-Methoxyethyl)(methyl)amino)ethyl)-8-methyl-2-(thieno[3,2-c]pyridin-6-yl)quinazolin-4(3H)-one;
6-(2-(1,1-Dioxidothiomorpholino)ethyl)-8-methyl-2-(thieno[3,2-c]pyridin-6-yl)quinazolin-4(3H)-one;
6-[(1,1-Dioxo-1,4-thiazinan-4-yl)methyl]-8-methyl-2-thieno[3,2-c]pyridin-6-yl-3H-quinazolin-4-one;
6-(((2-Methoxyethyl)(methyl)amino)methyl)-8-methyl-2-(thieno[3,2-c]pyridin-6-yl)quinazolin-4(3H)-one;
6-[(4-Methoxy-1-piperidyl)methyl]-8-methyl-2-thieno[3,2-c]pyridin-6-yl-3H-quinazolin-4-one;
6-[(2,2-Dimethylmorpholin-4-yl)methyl]-8-methyl-2-thieno[3,2-c]pyridin-6-yl-3H-quinazolin-4-one;
8-Chloro-6-(2-morpholinoethyl)-2-(4-(trifluoromethyl)pyridin-2-yl)quinazolin-4(3H)-one;
8-Methyl-6-(2-oxa-7-azaspiro[3.5]nonan-7-ylmethyl)-2-thieno[3,2-c]pyridin-6-yl-3H-quinazolin-4-one;
N,N-Dimethyl-1-((8-methyl-4-oxo-2-(thieno[3,2-c]pyridin-6-yl)-3,4-dihydroquinazolin-6-yl)methyl)piperidine-4-carboxamide;
6-((4-(Methoxymethyl)piperidin-1-yl)methyl)-8-methyl-2-(thieno[3,2-c]pyridin-6-yl)quinazolin-4(3H)-one;
8-Methoxy-6-(2-morpholinoethyl)-2-(4-(trifluoromethyl)pyridin-2-yl)quinazolin-4(3H)-one;
8-Bromo-6-(2-morpholinoethyl)-2-(4-(trifluoromethyl)pyridin-2-yl)quinazolin-4(3H)-one;
6-(2-(2,2-Dimethylmorpholino)ethyl)-8-methyl-2-(4-(trifluoromethyl)pyridin-2-yl)quinazolin-4(3H)-one;
8-Methyl-6-((4-methyl-3-oxopiperazin-1-yl)methyl)-2-(4-(trifluoromethyl)pyridin-2-yl)quinazolin-4(3H)-one;
6-(2-(8-Oxa-3-azabicyclo[3.2.1]octan-3-yl)ethyl)-8-methyl-2-(4-(trifluoromethyl)pyridin-2-yl)quinazolin-4(3H)-one;
6-(2-(3-Oxa-8-azabicyclo[3.2.1]octan-8-yl)ethyl)-8-methyl-2-(4-(trifluoromethyl)pyridin-2-yl)quinazolin-4(3H)-one;
6-(2-(4-Hydroxypiperidin-1-yl)ethyl)-8-methyl-2-(4-(trifluoromethyl)pyridin-2-yl)quinazolin-4(3H)-one;
6-(2-(4,4-Difluoropiperidin-1-yl)ethyl)-8-methyl-2-(4-(trifluoromethyl)pyridin-2-yl)quinazolin-4(3H)-one;
6-(2-(4-Methoxypiperidin-1-yl)ethyl)-8-methyl-2-(4-(trifluoromethyl)pyridin-2-yl)quinazolin-4(3H)-one;
8-Methyl-6-(2-morpholinoethyl)-2-(4-(trifluoromethyl)pyridin-2-yl)pyrido[3,2-d]pyrimidin-4(3H)-one;
and pharmaceutically acceptable salts of any one of the aforementioned compounds.
19. A pharmaceutical composition comprising the compound of any one of items 1 to 18 and a pharmaceutically acceptable excipient.
20. The compound of any one of items 1 to 18 for use as a medicament.
21. The compound of any one of items 1 to 18 or the pharmaceutical composition of item 19 for use in the treatment or prevention of a condition associated with altered glutamatergic signalling and/or functions or a condition which can be affected by alteration of glutamate level or signalling.
22. Use of the compound of any one of items 1 to 18 in the preparation of a medicament for the treatment or prevention of a condition associated with altered glutamatergic signalling and/or functions or a condition which can be affected by alteration of glutamate level or signalling.
23. A method of treating or preventing a condition associated with altered glutamatergic signalling and/or functions or a condition which can be affected by alteration of glutamate level or signalling, the method comprising administering the compound of item 1 to a subject in need thereof.
24. The compound for use according to item 21 or the pharmaceutical composition for use according to item 21 or the use of item 22 or the method of item 23, wherein the condition to be treated or prevented is selected from any one of: epilepsy; dementias and related diseases, including dementias of the Alzheimer's type, Alzheimer's disease, Pick's disease, vascular dementias, Lewy-body disease, dementias due to metabolic, toxic and deficiency diseases, AIDS-dementia complex, Creutzfeld-Jacob disease and atypical subacute spongiform encephalopathy; Parkinsonism and movement disorders, including Parkinson's disease, multiple system atrophy, progressive supranuclear palsy, corticobasal degeneration, hepatolenticular degeneration, chorea, Huntington's disease, hemiballismus, athetosis, dystonias, spasmodic torticollis, occupational movement disorder, Gilles de la Tourette syndrome, tardive or drug induced dyskinesias, levodopa-induced dyskinesia, tremor and myoclonus; motor neuron disease or amyotrophic lateral sclerosis; neurodegenerative and/or hereditary disorders of the nervous system, including spinocerebrellar degenerations, Friedrich's ataxia and other hereditary cerebellar ataxias, predominantly spinal muscular atrophies, hereditary neuropathies, and phakomatoses; disorders of the peripheral nervous system, including trigeminal neuralgia, facial nerve disorders, disorders of the other cranial nerves, nerve root and plexus disorders, mononeuritis, carpal tunnel syndrome, sciatica, hereditary and idiopathic peripheral neuropathies, inflammatory and toxic neuropathies; multiple sclerosis and other autoimmune diseases, including systemic lupus erythematosus and psoriasis; infantile cerebral palsy; hemiplegia, hemiparesis, and other paralytic syndromes; cerebrovascular disorders, including subarachnoid hemorrhage, intracerebral hemorrhage, occlusion and stenosis of precerebral arteries, occlusion of cerebral arteries including thrombosis and embolism, brain ischemia, stroke, transient ischemic attacks, atherosclerosis, cerebrovascular dementias, aneurysms, cerebral deficits due to cardiac bypass surgery and grafting; migraine, including classical migraine and variants, including cluster headache; headache; myoneural disorders including myasthenia gravis, acute muscle spasms, myopathies including muscular dystrophies, mytotonias and familial periodic paralysis; disorders of the eye and visual pathways, including retinal disorders, and visual disturbances; intracranial trauma/injury and their sequels; trauma/injury to nerves and spinal cord and their sequels; poisoning and toxic effects of nonmedicinal substances; accidental poisoning by drugs, medicinal substances and biologicals acting on the central, peripheral and autonomic system; neurological and psychiatric adverse effects of drugs, medicinal and biological substances; disturbance of sphincter control and sexual function; social skill disorders, including autism or autism spectrum disorders, or fragile X syndrome; mental disorders including mental retardation, learning disorders, motor skill disorders, communication disorders, pervasive developmental disorders, attention deficit and disruptive behaviour disorders, feeding and eating disorders, TIC disorders, elimination disorders; delirium and other cognitive disorders; substance related disorders including alcohol-related disorders, nicotine-related disorders, disorders related to cocaine, opioids, cannabis, hallucinogens and other drugs; schizophrenia and other psychotic disorders; mood disorders, including depressive disorders and bipolar disorders; anxiety disorders, including panic disorders, phobias, obsessive-compulsive disorders, stress disorders, generalized anxiety disorders; eating disorders, including anorexia and bulimia; sleep disorders, including dyssomnias, insomnia, hypersomnia, narcolepsy, breathing related sleep disorder, and parasomnias; medication-induced movement disorders including neuroleptic-induced parkinsonism and tardive dyskinesia; endocrine and metabolic diseases including diabetes, disorders of the endocrine glands, hypoglycaemia; acute and chronic pain; nausea and vomiting; irritable bowel syndrome; and cancers.
25. The compound for use according to item 21 or the pharmaceutical composition for use according to item 21 or the use of item 22 or the method of item 23, wherein the condition to be treated or prevented is selected from any one of: dementias and related diseases, including dementias of the Alzheimer's type, Alzheimer's disease, Pick's disease, vascular dementias, Lewy-body disease, dementias due to metabolic, toxic and deficiency diseases, AIDS-dementia complex, Creutzfeld-Jacob disease and atypical subacute spongiform encephalopathy; parkinsonism and movement disorders, including Parkinson's disease, multiple system atrophy, progressive supranuclear palsy, corticobasal degeneration, hepatolenticular degeneration, chorea, Huntington's disease, hemiballismus, athetosis, dystonias, spasmodic torticollis, occupational movement disorder, Gilles de la Tourette syndrome, tardive or drug induced dyskinesias, levodopa-induced dyskinesia, tremor and myoclonus; social skill disorders including autism or autism spectrum disorders, or fragile X syndrome; acute and chronic pain; anxiety disorders, including panic disorders, phobias, obsessive-compulsive disorders, stress disorders and generalized anxiety disorders; schizophrenia and other psychotic disorders; mood disorders, including depressive disorders and bipolar disorders; endocrine and metabolic diseases including diabetes, disorders of the endocrine glands and hypoglycaemia; and cancers.
26. The compound of any one of items 1 to 18 or the pharmaceutical composition of item 19 for use in the treatment or prevention of Parkinson's disease.
27. Use of the compound of any one of items 1 to 28 in the preparation of a medicament for the treatment or prevention of Parkinson's disease.
28. A method of treating or preventing Parkinson's disease, the method comprising administering the compound of item 1 to a subject in need thereof.
29. The compound for use according to any one of items 20, 21 or 24 to 26 or the pharmaceutical composition for use according to item 21 or 24 to 26 or the use of item 22, 24, 25 or 27 or the method of item 23, wherein said compound or said pharmaceutical composition or said medicament is to be administered orally.
30. The compound for use according to any one of items 20, 21, 24 to 26 or 29 or the pharmaceutical composition for use according to any one of items 21, 24 to 26 or 29 or the use of any one of items 22, 24, 25, 27 or 29, wherein said compound or said pharmaceutical composition or said medicament is to be administered to a human subject.
31. The method of item 23, wherein said subject is a human.
32. A method of identifying a test agent that binds to metabotropic glutamate receptor 4 (mGluR4), comprising the following steps:
   (a) contacting mGluR4 with the compound of any one of items 1 to 18, wherein said compound is radio-labeled or fluorescence-labeled, under conditions that permit binding of the compound to mGluR4, thereby generating bound, labeled compound;
   (b) detecting a signal that corresponds to the amount of bound, labeled compound in the absence of test agent;
   (c) contacting the bound, labeled compound with a test agent;
   (d) detecting a signal that corresponds to the amount of bound labeled compound in the presence of test agent; and
   (e) comparing the signal detected in step (d) to the signal detected in step (b) to determine whether the test agent binds to mGluR4.
33. In vitro use of a compound as defined in any one of items 1 to 18 as a positive allosteric modulator of mGluR4.

The invention is also described by the following illustrative FIGURE, which shows:

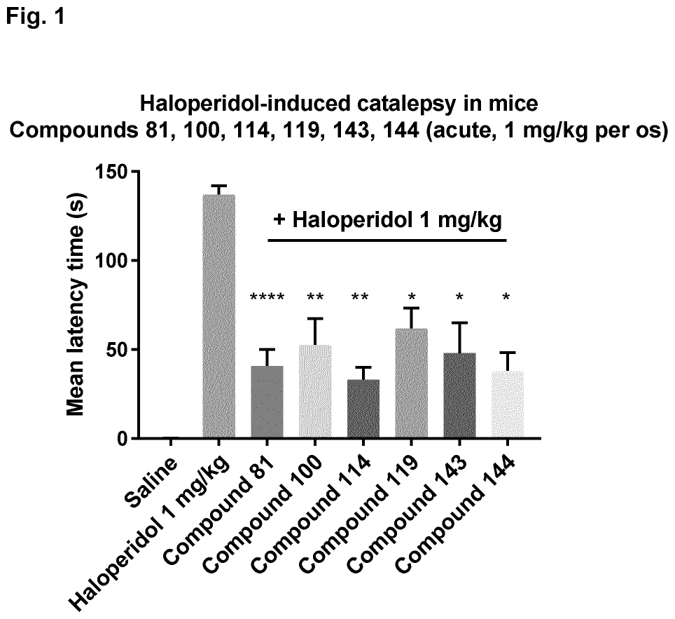

FIG. 1: The anti-cataleptic effect of exemplary compounds of formula (I) was determined in vivo in a haloperidol-induced catalepsy model in the mouse (see section III of the examples). The FIGURE shows the mean time of latency spent on the bar in each group of animals and measured between 135 and 270 min after haloperidol injection. The anti-cataleptic effect of the compounds was compared to vehicle-treated group using ANOVA test followed by the Dunnett's test. Compounds 81, 100, 114, 119, 143 and 144 administered at 1 mg/kg per os 60 minutes after haloperidol injection showed a significant anti-cataleptic effect (with adjusted p values of <0.0001, 0.0065, 0.0066, 0.0307, 0.0176, and 0.0115, respectively).

The invention will now be described by reference to the following examples which are merely illustrative and are not to be construed as a limitation of the scope of the present invention.

The compounds described in the following examples section are defined by their chemical formulae and their corresponding chemical names. In case of conflict between any chemical formula and the corresponding chemical name indicated herein, the present invention relates to both the compound defined by the chemical formula and the compound defined by the chemical name, and particularly relates to the compound defined by the chemical formula.

### EXAMPLES

General Experimental Procedures

All reagents were commercial grade and used without further purification. When required commercially available anhydrous solvents were used. Most reactions were conducted under inert atmosphere (argon). Column chromatography was generally performed with a Biotage Isolera Four apparatus using Biotage KP-Sil cartridges. Thin layer chromatography was carried out using pre-coated silica gel F-254 plates.

¹H NMR spectra were recorded on a Bruker AMX-400 spectrometer. Proton chemical shifts are listed relative to residual CDCl₃ (7.26 ppm), DMSO (2.50 ppm) or D₂O (4.78 ppm). Splitting patterns are designated as s (singlet), d (doublet), dd (doublet-doublet), t (triplet), tt (triplet-triplet), td (triplet-doublet), q (quartet), quint (quintuplet), sex (sextuplet), sept (septuplet), m (multiplet), b (broad).

The HPLC system was a Waters platform with a 2767 sample manager, a 2525 pump, a photodiode array detector (190-400 nm). HPLC is coupled with a Waters Acquity QDa detector. All mass spectra were full-scan experiments (mass range 110-850 amu). Mass spectra were obtained using electro spray ionization. The column used was an XSelect CSH C₁₈ 3.5 μM (4.6×50 mm) in analytical mode and an XSelect CSH prep C₁₈ 5 μM (19×100 mm) in preparative mode. The mobile phase in both cases consisted in an appropriate gradient of A and B. A was water with 0.1% of formic acid and B was acetonitrile with 0.1% of formic acid. Flow rate was 1 mL per min in analytical mode and 25 mL min in preparative mode. All HPLCMS were performed at room temperature. The UPLC system was a Waters Aquity platform with a photodiode array detector (190-400 nm). The column used was an Acquity CSH C₁₈ 1.7 μM (2.1×30 mm). The mobile phase consisted in a gradient of A and B. A was water with 0.025% of TFA and B was acetonitrile with 0.025% of TFA. Flow rate was 0.8 mL per min. All analyses were performed at 55° C. UPLC is coupled with a Waters SQD2 platform. All mass spectra were full-scan experiments (mass range 100-800 amu). Mass spectra were obtained using electro spray ionization.

Melting Points were measured on a Barnstead Electrothermal 9100 and are not corrected.

### I. Synthesis of Selected Compounds of the Invention

The following compounds were synthesized and characterized as outlined below.

Pyrrolo[1,2-c]pyrimidine-3-carboxylic acid, thieno[3,2-c]pyridine-6-carboxylic acid and thieno[2,3-c]pyridine-5-carboxylic acid were prepared according to conditions described in the literature (J. Org. Chem., 1999, 64, 7788-7801; J. Med. Chem. 2006, 49, 4425-4436; and WO 2004/39815).

### Example 1—Synthesis of compound 1 (6-(3-pyridin-4-yl-propoxy)-2-pyrrolo[1,2-c]pyrimidin-3-yl-3H-quinazolin-4-one)

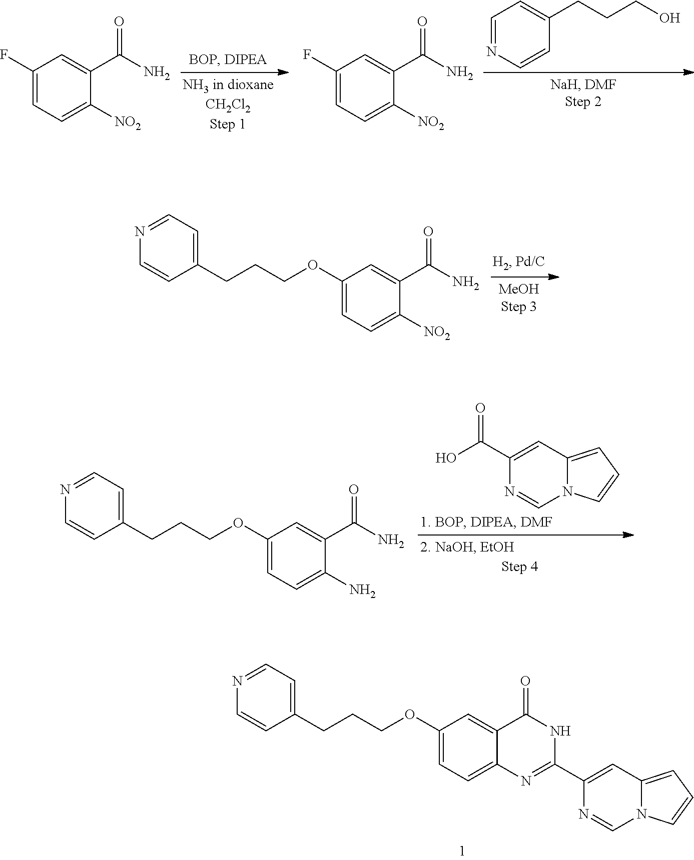

Step 1:

Under inert atmosphere, a solution of 5-fluoro-2-nitrobenzoic acid (2.50 g, 13.5 mmol), ammonia (0.5M in dioxane, 54.0 mL, 27.0 mmol), benzotriazol-1-yloxy-tris(dimethylamino)-phosphonium hexafluorophosphate (8.90 g, 20.3 mmol) and diisopropylethylamine (6.10 mL, 35.1 mmol) in anhydrous dichloromethane (68.0 mL) was stirred for 16 h at room temperature. Then, the mixture was poured into an aqueous saturated solution of ammonium chloride (250 mL) and extracted with dichloromethane (2×200 mL). The combined organic extracts were washed with brine (100 mL), dried over MgSO₄ and concentrated under vacuum. The crude dark solid was purified by flash column chromatography on silica gel using cyclohexane/ethyl acetate as eluent to afford 5-fluoro-2-nitrobenzamide (2.40 g, 13.0 mmol, 96%) as a brown solid.

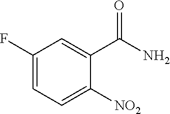 [SMILES: NC(=O)c1cc(F)ccc1[N+](=O)[O-]]

M/Z (M+H)⁺=185.2.

Step 2:

To a suspension of sodium hydride (60% suspension in oil, 0.84 g, 21.7 mmol) in anhydrous DMF (15.0 mL) at 0° C., a solution of 4-pyridinepropanol (1.49 g, 10.8 mmol) in DMF (15.0 mL) was added dropwise under inert atmosphere. After 5 min, at 0° C., a solution of 5-fluoro-2-nitrobenzamide (2.00 g, 10.8 mmol) in DMF (15.0 mL) was added dropwise under vigorous stirring. The resulting reddish mixture was stirred for 1 h at room temperature before being diluted with water (100 mL) and extracted with ethyl acetate (3×300 mL). The combined organic extracts were washed with brine (100 mL), dried over MgSO₄ and concentrated under vacuum. The crude dark oil was purified by flash column chromatography on silica gel using dichloromethane/methanol as eluent to afford 2-nitro-5-(3-pyridin-4-yl-propoxy)-benzamide (2.05 g, 63%) as an orange oil.

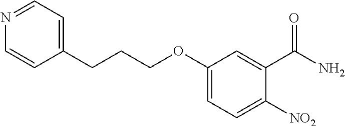 [SMILES: NC(=O)c1cc(OCCCc2ccncc2)ccc1[N+](=O)[O-]]

¹H-NMR (400 MHz, DMSO): 2.09 (m, 2H, CH₂); 2.78 (t, J 7.6 Hz, 2H, CH₂); 4.15 (t, J 6.5 Hz, 2H, CH₂—O); 7.05 (d, J 2.8 Hz, 1H, Ar); 7.14 (dd, J 9.0, 2.8 Hz, 1H, Ar); 7.28 (d, J 5.8 Hz, 2H, Ar); 7.64 (bs, 1H, NH); 8.02 (bs, 1H, NH); 8.04 (d, J 9.0 Hz, 1H, Ar); 8.47 (d, J 5.8 Hz, 2H, Ar). M/Z (M+H)⁺=302.1.

Step 3:

To a solution of 2-nitro-5-(3-pyridin-4-yl-propoxy)-benzamide (2.05 g, 6.80 mmol) in methanol (23.0 mL) and DMF (8.0 mL), 10% palladium on charcoal (1.45 g) was added. The suspension was placed under hydrogen gaz at atmospheric pressure and stirred for 2 h at room temperature. Then, the mixture was filtered through a pad of celite. Methanol was removed under vacuum to give an orange solution which was partitioned between water (50 mL) and ethyl acetate (50 mL) and extracted with ethyl acetate (2×50 mL). The combined organic extracts were washed with brine (100 mL), dried over MgSO₄ and concentrated under vacuum. The crude oil was purified by flash column chromatography on silica gel using dichloromethane/methanol as eluent to afford 2-amino-5-(3-pyridin-4-yl-propoxy)-benzamide (0.96 g, 3.54 mmol, 52%) as a yellow solid.

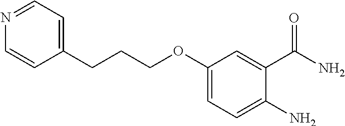 [SMILES: NC(=O)c1cc(OCCCc2ccncc2)ccc1N]

¹H-NMR (400 MHz, DMSO): 1.99 (m, 2H, CH₂); 2.75 (t, J 7.6 Hz, 2H, CH₂); 3.89 (t, J 6.6 Hz, 2H, CH₂—O); 6.12 (bs, 2H, NH₂); 6.63 (d, J 8.9 Hz, 1H, Ar); 6.84 (dd, J 8.9, 2.8 Hz, 1H, Ar); 7.05 (bs, 1H, NH); 7.12 (d, J 2.8 Hz, 1H, Ar); 7.26 (d, J 5.9 Hz, 2H, Ar); 7.67 (bs, 1H, NH); 8.46 (d, J 5.9 Hz, 2H, Ar). M/Z (M+H)⁺=272.2.

Step 4:

A suspension of 2-amino-5-(3-pyridin-4-yl-propoxy)-benzamide (100 mg, 0.37 mmol), pyrrolo[1,2-c]pyrimidine-3-carboxylic acid (70 mg, 0.41 mmol), benzotriazol-1-yloxy-tris(dimethylamino)-phosphonium hexafluorophosphate (245 mg, 0.56 mmol) and diisopropylethylamine (0.20 mL, 1.11 mmol) in anhydrous DMF (1.0 mL) was stirred for 4 h at 70° C. Then, the mixture was poured into ice water (10 mL) to give a grey precipitate which was collected by filtration and triturated with dichloromethane (2×2 mL). The grey solid was suspended in a mixture of sodium hydroxide (5% in water, 0.5 mL) and ethanol (0.5 mL) and heated under reflux for 1 h. Ethanol was removed under vacuum and the resulting solution was poured into a saturated aqueous solution of ammonium chloride (5.0 mL). A brown precipitate formed which was collected by filtration and rinsed several times with water (3.0 mL). Then, the solid was dried overnight under high vacuum in presence of P₂O₅ at 50° C. to afford compound 1 (60 mg, 41%) as a brown powder.

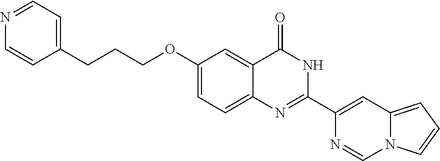 [SMILES: O=c1[nH]c(-c2cc3cccn3cn2)nc2ccc(OCCCc3ccncc3)cc12]

¹H-NMR (400 MHz, DMSO): 2.12 (m, 2H, CH₂); 2.82 (t, J 7.6 Hz, 2H, CH₂); 4.12 (t, J 6.4 Hz, 2H, CH₂—O); 6.85 (d, J 3.4 Hz, 1H, Ar); 7.06 (t, J 2.9 Hz, 1H, Ar); 7.30 (d, J 5.6 Hz, 2H, Ar); 7.48 (dd, J 8.8, 2.9 Hz, 1H, Ar); 7.52 (d, J 2.9 Hz, 1H, Ar); 7.69 (d, J 8.8 Hz, 1H, Ar); 7.89 (s, 1H, Ar); 8.48 (m, 3H, Ar); 9.33 (s, 1H, Ar); NH signal not observed. M/Z (M+H)⁺=398.1. MP=202-206° C.

### Compound 2 (2-Isoquinolin-3-yl-6-(3-pyridin-4-yl-propoxy)-3H-quinazolin-4-one hydrochloride)

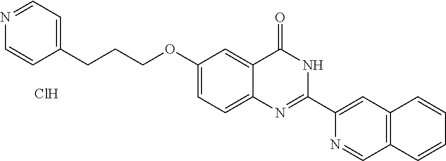 [SMILES: O=c1[nH]c(-c2cc3ccccc3cn2)nc2ccc(OCCCc3ccncc3)cc12 ; Cl]

Compound 2 was prepared according to procedure of example 1, step 4, starting from 2-amino-5-(3-pyridin-4-yl-propoxy)-benzamide and isoquinoline-3-carboxylic acid. The HCl salt was obtained by filtration after addition of an excess of HCl (2N in Et₂O) to a solution of the free base in dichloromethane. Compound 2 was obtained as a white solid in 92% yield.

¹H-NMR (400 MHz, DMSO): 2.37 (m, 2H, CH₂); 3.26 (t, J 7.6 Hz, 2H, CH₂); 4.28 (t, J 6.4 Hz, 2H, CH₂—O); 7.55 (dd, J 8.9, 2.8 Hz, 1H, Ar); 7.69 (d, J 2.8 Hz, 1H, Ar); 7.96 (d, J 8.9 Hz, 1H, Ar); 7.97 (d, J 7.3 Hz, 1H, Ar); 8.05 (t, J 7.3 Hz, 1H, Ar); 8.09 (d, J 6.5 Hz, 2H, Ar); 8.25 (d, J 8.2 Hz, 1H, Ar); 8.36 (d, J 8.2 Hz, 1H, Ar); 8.78 (d, J 6.5 Hz, 2H, Ar); 9.04 (s, 1H, Ar); 9.58 (s, 1H, Ar); NH signal not observed; HCl salt signal not observed. M/Z (M+H)⁺=409.2. MP>250° C.

### Compound 3 (6-(3-Pyridin-4-yl-propoxy)-2-thieno[3,2-c]pyridin-6-yl-3H-quinazolin-4-one hydrochloride)

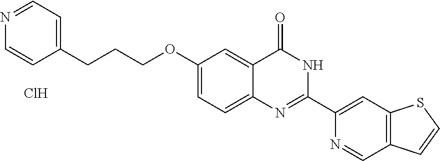 [SMILES: O=c1[nH]c(-c2cc3sccc3cn2)nc2ccc(OCCCc3ccncc3)cc12 ; Cl]

Compound 3 was prepared according to procedure of example 1, step 4, starting from 2-amino-5-(3-pyridin-4-yl-propoxy)-benzamide and thieno[3,2-c]pyridine-6-carboxylic acid. The HCl salt was obtained by filtration after addition of an excess of HCl (2N in Et₂O) to a solution of the free base in dichloromethane and methanol. Compound 3 was obtained as a yellow solid in 93% yield.

¹H-NMR (400 MHz, DMSO): 2.17 (m, 2H, CH₂); 3.06 (t, J 7.6 Hz, 2H, CH₂); 4.13 (t, J 6.4 Hz, 2H, CH₂—O); 7.38 (dd, J 9.0, 3.0 Hz, 1H, Ar); 7.49 (d, J 3.0 Hz, 1H, Ar); 7.70 (m, 2H, Ar); 7.96 (d, J 6.6 Hz, 2H, Ar); 8.06 (d, J 5.5 Hz, 1H, Ar); 8.77 (d, J 6.6 Hz, 2H, Ar); 9.08 (s, 1H, Ar); 9.24 (s, 1H, Ar); NH signal not observed; HCl salt signal not observed. M/Z (M+H)⁺=415.2. MP>250° C.

### Compound 4 (6-(3-Pyridin-4-yl-propoxy)-2-thieno[2,3-c]pyridin-6-yl-3H-quinazolin-4-one hydrochloride)

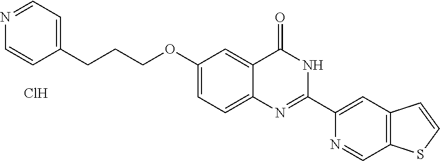 [SMILES: O=c1[nH]c(-c2cc3ccsc3cn2)nc2ccc(OCCCc3ccncc3)cc12 ; Cl]

Compound 4 was prepared according to procedure of example 1, step 4, starting from 2-amino-5-(3-pyridin-4-yl-propoxy)-benzamide and thieno[2,3-c]pyridine-6-carboxylic acid. The HCl salt was obtained by filtration after addition of an excess of HCl (2N in Et₂O) to a solution of the free base in methanol. Compound 4 was obtained as a yellow solid in 73% yield.

¹H-NMR (400 MHz, DMSO): 2.24 (m, 2H, CH₂); 3.13 (t, J 7.6 Hz, 2H, CH₂); 4.20 (t, J 6.4 Hz, 2H, CH₂—O); 7.44 (dd, J 9.0, 2.9 Hz, 1H, Ar); 7.57 (d, J 2.9 Hz, 1H, Ar); 7.78 (d, J 9.0 Hz, 1H, Ar); 7.81 (d, J 5.2 Hz, 1H, Ar); 8.04 (d, J 6.7 Hz, 2H, Ar); 8.31 (d, J 5.2 Hz, 1H, Ar); 8.85 (d, J 6.7 Hz, 2H, Ar); 8.95 (s, 1H, Ar); 9.47 (s, 1H, Ar); NH signal not observed; HCl salt signal not observed. M/Z (M+H)⁺=415.1. MP>250° C.

### Compound 5 (2-Pyridin-2-yl-6-(3-pyridin-4-yl-propoxy)-3H-quinazolin-4-one hydrochloride)

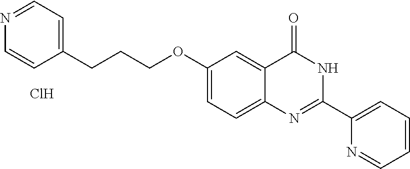 [SMILES: O=c1[nH]c(-c2ccccn2)nc2ccc(OCCCc3ccncc3)cc12 ; Cl]

Compound 5 was prepared according to procedure of example 1, step 4, starting from 2-amino-5-(3-pyridin-4-yl-propoxy)-benzamide and picolinic acid. The HCl salt was obtained by filtration after addition of 1 equivalent of HCl (2N in Et₂O) to a solution of the free base in dichloromethane. Compound 5 was obtained as a yellow solid in 84% yield.

¹H-NMR (400 MHz, DMSO): 2.23 (m, 2H, CH₂); 3.13 (t, J 7.6 Hz, 2H, CH₂); 4.19 (t, J 6.4 Hz, 2H, CH₂—O); 7.43 (dd, J 9.0, 2.9 Hz, 1H, Ar); 7.56 (d, J 2.9 Hz, 1H, Ar); 7.66 (m, 1H, Ar); 7.77 (d, J 9.0 Hz, 1H, Ar); 8.03 (d, J 6.6 Hz, 2H, Ar); 8.08 (t, J 7.7 Hz, 1H, Ar); 8.43 (d, J 7.7 Hz, 1H, Ar); 8.76 (d, J 4.5 Hz, 1H, Ar); 8.85 (d, J 6.6 Hz, 2H, Ar); NH signal not observed; HCl salt signal not observed. M/Z (M+H)⁺=359.2. MP>250° C.

### Reference Compound 6 (2-Pyridin-3-yl-6-(3-pyridin-4-yl-propoxy)-3H-quinazolin-4-one hydrochloride)

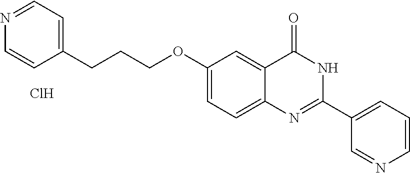 [SMILES: O=c1[nH]c(-c2cccnc2)nc2ccc(OCCCc3ccncc3)cc12 ; Cl]

Compound 6 (reference) was prepared according to procedure of example 1, step 4, starting from 2-amino-5-(3-pyridin-4-yl-propoxy)-benzamide and nicotinic acid. The HCl salt was obtained by filtration after addition of an excess of HCl (2N in Et₂O) to a solution of the free base in dichloromethane. Compound 6 was obtained as a white solid in 60% yield.

¹H-NMR (400 MHz, DMSO): 2.24 (m, 2H, CH₂); 3.12 (t, J 7.6 Hz, 2H, CH₂); 4.20 (t, J 6.4 Hz, 2H, CH₂—O); 7.44 (dd, J 9.0, 2.9 Hz, 1H, Ar); 7.56 (d, J 2.9 Hz, 1H, Ar); 7.77 (d, J 9.0 Hz, 1H, Ar); 7.91 (dd, J 8.1, 5.3 Hz, 1H, Ar); 8.04 (d, J 6.5 Hz, 2H, Ar); 8.46 (m, 3H, Ar); 8.92 (d, J 5.3 Hz, 1H, Ar); 9.41 (s, 1H, Ar); NH signal not observed; HCl salt signal not observed. M/Z (M+H)⁺=359.2. MP>250° C.

### Compound 7 (2-(4-methoxy-pyridin-2-yl)-6-(3-pyridin-4-yl-propoxy)-3H-quinazolin-4-one hydrochloride)

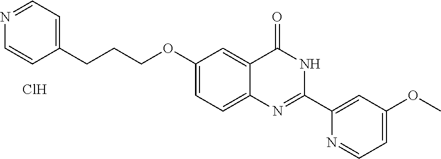 [SMILES: COc1ccnc(-c2nc3ccc(OCCCc4ccncc4)cc3c(=O)[nH]2)c1 ; Cl]

Compound 7 was prepared according to procedure of example 1, step 4, starting from 2-amino-5-(3-pyridin-4-yl-propoxy)-benzamide and 4-methoxypicolinic acid. The HCl salt was obtained by filtration after addition of an excess of HCl (2N in Et₂O) to a solution of the free base in dichloromethane. Compound 7 was obtained as a white solid in 86% yield.

¹H-NMR (400 MHz, DMSO): 2.23 (m, 2H, CH₂); 3.12 (t, J 7.6 Hz, 2H, CH₂); 4.01 (s, 3H, CH₃—O); 4.20 (t, J 6.4 Hz, 2H, CH₂—O); 7.29 (m, 1H, Ar); 7.44 (dd, J 8.8, 2.9 Hz, 1H, Ar); 7.56 (d, J 2.9 Hz, 1H, Ar); 7.78 (d, J 8.8 Hz, 1H, Ar); 8.03 (m, 3H, Ar); 8.60 (d, J 5.8 Hz, 1H, Ar); 8.84 (d, J 6.5 Hz, 2H, Ar); NH signal not observed; HCl salt signal not observed. M/Z (M+H)⁺=389.1. MP>250° C.

### Compound 8 (2-(5-Fluoro-pyridin-2-yl)-6-(3-pyridin-4-yl-propoxy)-3H-quinazolin-4-one hydrochloride)

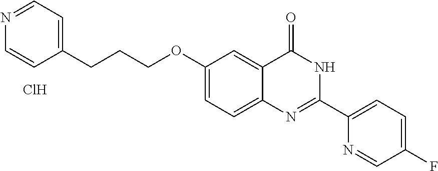 [SMILES: O=c1[nH]c(-c2ccc(F)cn2)nc2ccc(OCCCc3ccncc3)cc12 ; Cl]

Compound 8 was prepared according to the procedure of example 1, step 4, starting from 2-amino-5-(3-pyridin-4-yl-propoxy)-benzamide and 5-fluoropicolinic acid. The crude product was purified by flash column chromatography on silica gel, using dichloromethane/methanol as eluent. The HCl salt was obtained by filtration after addition of an excess of HCl (2N in Et₂O) to a solution of the free base in dichloromethane. Compound 8 was obtained as a yellow solid in 42% yield.

¹H-NMR (400 MHz, DMSO): 2.23 (m, 2H, CH₂); 3.11 (t, J 7.6 Hz, 2H, CH₂); 4.19 (t, J 6.4 Hz, 2H, CH₂—O); 7.43 (dd, J 8.8, 3.0 Hz, 1H, Ar); 7.55 (d, J 3.0 Hz, 1H, Ar); 7.75 (d, J 8.8 Hz, 1H, Ar); 8.00 (m, 3H, Ar); 8.48 (dd, J 8.8, 4.5 Hz, 1H, Ar); 8.75 (d, J 2.8 Hz, 1H, Ar); 8.83 (d, J 6.7 Hz, 2H, Ar); NH signal not observed; HCl salt signal not observed. M/Z (M+H)⁺=377.1. MP>250° C.

### Compound 9 (6-(3-Pyridin-4-yl-propoxy)-2-(4-trifluoromethyl-pyridin-2-yl)-3H-quinazolin-4-one hydrochloride)

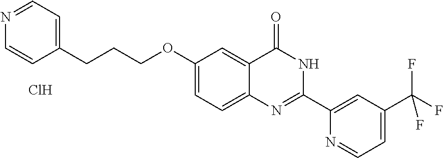 [SMILES: O=c1[nH]c(-c2cc(C(F)(F)F)ccn2)nc2ccc(OCCCc3ccncc3)cc12 ; Cl]

Compound 9 was prepared according to procedure of example 1, step 4, starting from 2-amino-5-(3-pyridin-4-yl-propoxy)-benzamide and 4-trifluoromethylpicolinic acid. The HCl salt was obtained by filtration after addition of an excess of HCl (2N in Et₂O) to a solution of the free base in dichloromethane. Compound 9 was obtained as a white solid in 69% yield.

¹H-NMR (400 MHz, DMSO): 2.23 (m, 2H, CH₂); 3.11 (t, J 7.6 Hz, 2H, CH₂); 4.20 (t, J 6.4 Hz, 2H, CH₂—O); 7.46 (dd, J 8.9, 3.0 Hz, 1H, Ar); 7.57 (d, J 3.0 Hz, 1H, Ar); 7.84 (d, J 8.9 Hz, 1H, Ar); 7.99 (d, J 6.5 Hz, 2H, Ar); 8.04 (d, J 5.1 Hz, 1H, Ar); 8.61 (s, 1H, Ar); 8.82 (d, J 6.5 Hz, 2H, Ar); 9.03 (d, J 5.1 Hz, 1H, Ar); 12.10 (bs, 1H, NH); HCl salt signal not observed. M/Z (M+H)⁺=427.1. MP=239-245° C.

### Compound 10 (6-[3-(4-pyridyl)propoxy]-2-[5-(trifluoromethyl)-2-pyridyl]-3H-quinazolin-4-one hydrochloride)

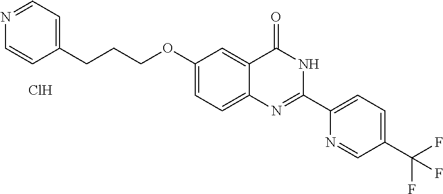 [SMILES: O=c1[nH]c(-c2ccc(C(F)(F)F)cn2)nc2ccc(OCCCc3ccncc3)cc12 ; Cl]

Compound 10 was prepared according to procedure of example 1, step 4, starting from 2-amino-5-(3-pyridin-4-yl-propoxy)-benzamide and 5-trifluoromethylpicolinic acid. The HCl salt was obtained by concentration to dryness and trituration in Et₂O after addition of an excess of HCl in MeOH to a solution of the free base in MeOH. Compound 10 was obtained as a yellow solid in 45% yield.

¹H-NMR (400 MHz, DMSO): 2.22 (tt, J 7.3, 6.1 Hz, 2H, CH₂); 3.08 (t, J 7.3 Hz, 2H, CH₂); 4.20 (t, J 6.1 Hz, 2H, CH₂—O); 7.46 (dd, J 8.8, 3.0 Hz; 1H, Ar); 7.57 (d, J 3.0 Hz, 1H, Ar); 7.79 (d, J 8.8 Hz, 1H, Ar); 7.94 (d, J 6.6 Hz, 2H, Ar); 8.47 (dd, J 8.5, 1.8 Hz, 1H Ar); 8.59 (d, J 8.5 Hz, 1H, Ar); 8.79 (d, J 6.6 Hz, 2H, Ar); 9.11-9.12 (m, 1H, Ar); 12.06 (bs, 1H, NH). HCl salt signal not observed. M/Z (M+H)⁺=427.4. MP>250° C.

### Compound 11 (2-(4-methyl-2-pyridyl)-6-[3-(4-pyridyl)propoxy]-3H-quinazolin-4-one dihydrochloride)

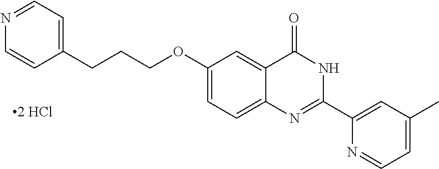 [SMILES: Cc1ccnc(-c2nc3ccc(OCCCc4ccncc4)cc3c(=O)[nH]2)c1 ; "·2 HCl"]

Compound 11 was prepared according to procedure of example 1, step 4, starting from 2-amino-5-(3-pyridin-4-yl-propoxy)-benzamide and 4-methylpyridine-2-carboxylic acid. The HCl salt was obtained by concentration to dryness and trituration in Et₂O after addition of an excess of HCl in MeOH to a solution of the free base in MeOH and dichloromethane. Compound 11 was obtained as a yellow solid in 51% yield.

¹H-NMR (400 MHz, DMSO): 2.23 (tt, J 7.1, 5.9 Hz, 2H, CH₂); 2.47 (s, 3H, CH₃); 3.11 (t, J 7.1 Hz, 2H, CH₂); 4.19 (t, J 5.9 Hz, 2H, CH₂—O); 7.43 (dd, J 8.8, 2.3 Hz; 1H, Ar); 7.48 (d, J 4.5 Hz, 1H, Ar); 7.55 (d, J 2.3 Hz, 1H, Ar); 7.76 (d, J 8.8 Hz, 1H, Ar); 8.01 (d, J 6.2 Hz, 2H Ar); 8.23 (s, 1H, Ar); 8.60 (d, J 4.5 Hz, 1H, Ar); 9.83 (d, J 6.2 Hz, 2H, Ar). NH signal not observed; HCl salt signal not observed. M/Z (M+H)⁺=373.3. MP=175-250° C.

### Compound 12 (2-(6-methyl-2-pyridyl)-6-[3-(4-pyridyl)propoxy]-3H-quinazolin-4-one dihydrochloride)

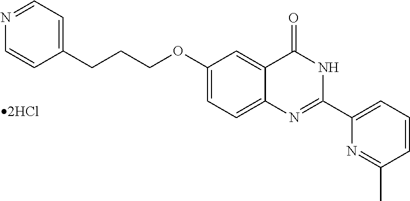 [SMILES: Cc1cccc(-c2nc3ccc(OCCCc4ccncc4)cc3c(=O)[nH]2)n1]

Compound 12 was prepared according to procedure of example 1, step 4, starting from 2-amino-5-(3-pyridin-4-yl-propoxy)-benzamide and 6-methylpyridine-2-carboxylic acid. The HCl salt was obtained by concentration to dryness and trituration in Et₂O after addition of an excess of HCl in MeOH to a solution of the free base in MeOH and dichloromethane. Compound 12 was obtained as a yellow solid in 34% yield.

¹H-NMR (400 MHz, DMSO): 2.27 (tt, J 7.5, 6.2 Hz, 2H, CH₂); 2.62 (s, 3H, CH₃); 3.11 (t, J 7.5 Hz, 2H, CH₂); 4.19 (t, J 6.2 Hz, 2H, CH₂—O); 7.42 (dd, J 8.8, 2.9 Hz, 1H, Ar); 7.50 (d, J 7.7 Hz, 1H, Ar); 7.56 (d, J 2.9 Hz, 1H, Ar); 7.76 (d, J 8.8 Hz, 1H Ar); 7.95 (t, J 7.7 Hz, 1H Ar); 8.02 (d, J 6.6 Hz, 2H, Ar); 8.22 (d, J 7.7 Hz, 1H, Ar); 8.83 (d, J 6.6 Hz, 2H, Ar). NH and HCl salt signals not observed. M/Z (M+H)⁺=373.3. MP>250° C.

### Compound 13 (2-(5-methylpyrazin-2-yl)-6-[3-(4-pyridyl)propoxy]-3H-quinazolin-4-one dihydrochloride)

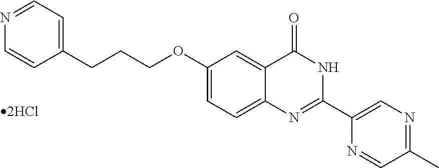 [SMILES: Cc1cnc(-c2nc3ccc(OCCCc4ccncc4)cc3c(=O)[nH]2)cn1]

Compound 13 was prepared according to procedure of example 1, step 4, starting from 2-amino-5-(3-pyridin-4-yl-propoxy)-benzamide and 5-methylpyrazine-2-carboxylic acid. The HCl salt was obtained by concentration to dryness and trituration in Et₂O after addition of an excess of HCl in MeOH to a solution of the free base in MeOH and dichloromethane. Compound 13 was obtained as a yellow solid in 18% yield.

¹H-NMR (400 MHz, DMSO): 2.15 (tt, J 7.5, 6.2 Hz, 2H, CH₂—O); 2.56 (s, 3H, CH₃); 3.02 (t, J 7.5 Hz, 2H, CH₂); 4.12 (t, J 6.2 Hz, 2H, CH₂—O); 7.37 (dd, J 8.9, 2.9 Hz, 1H, Ar); 7.49 (d, J 2.9 Hz, 1H, Ar); 7.71 (d, J 8.9 Hz, 1H, Ar); 7.88 (d, J 5.6 Hz, 2H, Ar); 8.63 (s, 1H, Ar); 8.73 (d, J 5.6 Hz, 2H, Ar); 9.34 (s, 1H, Ar); 12.00 (bs, 1H, NH). HCl salt signal not observed. M/Z (M+H)⁺=374.3. MP>250° C.

### Compound 14 (2-[5-chloro-4-(trifluoromethyl)-2-pyridyl]-6-[3-(4-pyridyl)propoxy]-3H-quinazolin-4-one hydrochloride)

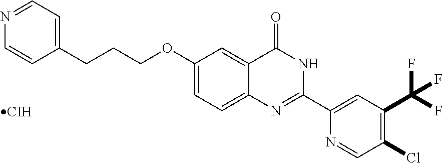

Compound 14 was prepared according to procedure of example 1, step 4, starting from 2-amino-5-(3-pyridin-4-yl-propoxy)-benzam ide and 5-chloro-4-(trifluoromethyl)pyridine-2-carboxylic acid. The HCl salt was obtained by concentration to dryness and trituration in Et₂O after addition of an excess of HCl (1.25M in MeOH) to a solution of the free base in MeOH and dichloromethane. Compound 14 was obtained as a yellow solid in 47% yield.

¹H-NMR (400 MHz, MeOD): 2.35 (tt, J 7.8, 5.9 Hz, 2H, CH₂); 3.24 (t, J 7.8 Hz, 2H, CH₂); 4.24 (t, J 5.9 Hz, 2H, CH₂—O); 7.46 (dd, J 8.9, 2.9 Hz, 1H Ar); 7.64 (d, J 2.9 Hz, 1H, Ar); 7.84 (d, J 8.9 Hz, 1H, Ar); 8.05 (d, J 6.4 Hz, 2H, Ar); 8.75 (d, J 6.4 Hz, 2H, Ar); 8.80 (s, 1H, Ar); 8.97 (s, 1H, Ar). NH and HCl salt signals not observed. M/Z (M+H)⁺=461.2. MP=134-250° C.

### Compound 15 (2-(4-chloro-2-pyridyl)-6-[3-(4-pyridyl)propoxy]-3H-quinazolin-4-one hydrochloride)

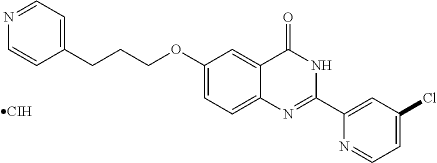

Compound 15 was prepared according to procedure of example 1, step 4, starting from 2-amino-5-(3-pyridin-4-yl-propoxy)-benzamide and lithium 4-chloropyridine-2-carboxylate, and using 3 equivalents of benzotriazol-1-yloxy-tris(dimethylamino)-phosphonium hexafluorophosphate. The HCl salt was obtained by filtration after addition of an excess of HCl in Et₂O to a solution of the free base in dichloromethane. Compound 15 was obtained as a yellow solid in 68% yield.

¹H-NMR (400 MHz, DMSO): 2.22 (tt, J 7.2, 6.2 Hz, 2H, CH₂); 3.09 (t, J 7.2 Hz, 2H, CH₂); 4.19 (t, J 6.2 Hz, 2H, CH₂—O); 7.45 (dd, J 8.8, 3.0 Hz; 1H, Ar); 7.56 (d, J 3.0 Hz, 1H, Ar); 7.77-7.80 (m, 2H, Ar); 7.96 (d, J 6.2 Hz, 2H, Ar); 8.41 (d, J 2.0 Hz, 1H Ar); 8.72 (d, J 5.3 Hz, 1H, Ar); 8.80 (d, J 6.2 Hz, 2H, Ar). NH and HCl salt signals not observed. M/Z (M+H)⁺=393.3. MP=232-241° C.

Lithium 4-chloropyridine-2-carboxylate was prepared as follows:

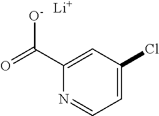

To a suspension of methyl 4-chloropyridine-2-carboxylate (50 mg, 0.29 mmol) in THF (0.5 mL) and water (0.5 mL) was added LiOH (14 mg, 0.58 mmol) and the reaction mixture was stirred overnight at room temperature. Then the reaction mixture was concentrated to dryness to afford the product (quantitative yield).

¹H-NMR (400 MHz, DMSO): 7.53 (dd, J 5.4, 2.3 Hz; 1H, Ar); 7.92 (dd, J 2.3, 0.5 Hz; 1H, Ar); 8.43 (dd, J 5.4, 0.5 Hz; 1H, Ar).

### Compound 16 (2-(4-ethyl-2-pyridyl)-6-[3-(4-pyridyl)propoxy]-3H-quinazolin-4-one dihydrochloride)

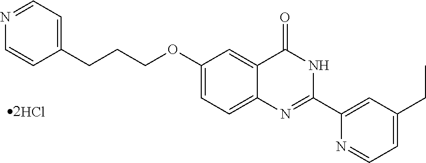 [SMILES: CCc1ccnc(-c2nc3ccc(OCCCc4ccncc4)cc3c(=O)[nH]2)c1 ; "·2HCl"]

Compound 16 was prepared according to procedure of compound 15, starting from 2-amino-5-(3-pyridin-4-yl-propoxy)-benzamide and lithium 4-ethylpyridine-2-carboxylate. The HCl salt was obtained by concentration to dryness after addition of an excess of HCl in Et₂O to a solution of the free base in dichloromethane. Compound 16 was obtained as a brown solid in 38% yield.

¹H-NMR (400 MHz, DMSO): 1.33 (t, J 7.4 Hz, 3H, CH₃); 2.29 (tt, J 7.7, 6.2 Hz, 2H, CH₂); 2.85 (q, J 7.4 Hz, 2H, CH₂); 3.18 (t, J 7.7 Hz, 2H, CH₂); 4.25 (t, J 6.2 Hz, 2H, CH₂—O); 7.49-7.51 (m; 1H, Ar); 7.54-7.62 (m, 2H, Ar); 7.83-7.85 (m, 1H, Ar); 8.09 (d, J 6.3 Hz, 2H, Ar); 8.35 (bs, 1H Ar); 8.68-8.70 (m, 1H, Ar); 8.90 (d, J 5.4 Hz, 2H, Ar). NH and HCl salt signals not observed. M/Z (M+H)⁺=387.3.

Lithium 4-ethylpyridine-2-carboxylate was prepared as follows:

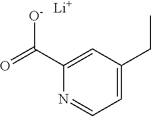

To a suspension of methyl 4-ethylpyridine-2-carboxylate (88 mg, 0.53 mmol) in THF (0.8 mL) and water (0.8 mL) was added LiOH (26 mg, 1.07 mmol) and the reaction mixture was stirred overnight at room temperature. Then the reaction mixture was concentrated to dryness to afford the product (quantitative yield).

¹H-NMR (400 MHz, DMSO): 1.20 (t, J 7.5 Hz, 3H, CH₃); 2.67 (m, 2H, CH₂); 7.28 (bs, 1H, Ar); 7.84 (bs, 1H, Ar); 8.31 (bs, 1H, Ar).

### Compound 17 (6-[3-(4-pyridyl)propoxy]-2-[6-(trifluoromethyl)-2-pyridyl]-3H-quinazolin-4-one hydrochloride)

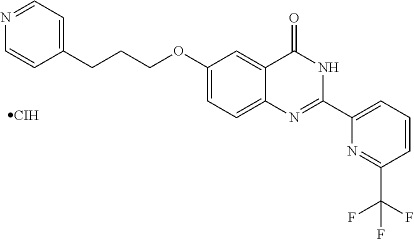 [SMILES: "·CIH" ; O=c1[nH]c(-c2cccc(C(F)(F)F)n2)nc2ccc(OCCCc3ccncc3)cc12]

Compound 17 was prepared according to procedure of example 1, step 4, starting from 2-amino-5-(3-pyridin-4-yl-propoxy)-benzamide and 6-(trifluoromethyl)pyridine-2-carboxylic acid. The HCl salt was obtained by concentration to dryness and trituration in Et₂O after addition of an excess of HCl in MeOH to a solution of the free base in MeOH and dichloromethane. Compound 17 was obtained as a yellow solid in 81% yield.

¹H-NMR (400 MHz, DMSO): 2.22 (tt, J 7.5, 6.2 Hz, 2H, CH₂); 3.08 (t, J 7.5 Hz, 2H, CH₂); 4.20 (t, J 6.2 Hz, 2H, CH₂—O);); 7.45 (dd, J 8.8, 3.0 Hz; 1H, Ar); 7.58 (d, J 3.0 Hz, 1H, Ar); 7.78 (d, J 8.8 Hz, 1H, Ar); 7.95 (d, J 6.7 Hz, 2H, Ar); 8.14 (d, J 7.8 Hz, 1H, Ar); 8.35 (t, J 7.8 Hz, 1H, Ar); 8.64 (d, J 7.8 Hz, 1H, Ar); 8.79 (d, J 6.7 Hz, 2H, Ar); 11.94 (bs, 1H, NH). HCl salt signal not observed. M/Z (M+H)⁺=427.3. MP=238-250° C.

### Compound 18 (2-(4-bromo-2-pyridyl)-6-[3-(4-pyridyl)propoxy]-3H-quinazolin-4-one)

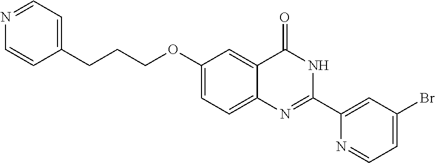 [SMILES: O=c1[nH]c(-c2cc(Br)ccn2)nc2ccc(OCCCc3ccncc3)cc12]

Compound 18 was prepared according to procedure of example 1, step 4, starting from 2-amino-5-(3-pyridin-4-yl-propoxy)-benzamide and 4-bromopyridine-2-carboxylic acid. Compound 18 was obtained as a beige solid in 88% yield.

¹H-NMR (400 MHz, DMSO): 2.10 (tt, J 7.5, 6.3 Hz, 2H, CH₂); 2.81 (t, J 7.5 Hz, 2H, CH₂); 4.04 (t, J 6.3 Hz, 2H, CH₂—O); 7.15 (dd, J 8.8, 3.0 Hz; 1H, Ar); 7.29 (dd, J 5.9 Hz, 2H, Ar); 7.43 (d, J 3.0 Hz, 1H, Ar); 7.53 (d, J 8.8 Hz, 1H, Ar); 7.64 (dd, J 5.3, 2.0 Hz, 1H Ar); 8.46 (d, J 5.9 Hz, 2H, Ar); 8.51 (d, J 5.3 Hz, 1H, Ar); 8.59 (d, J 2.0 Hz, 1H, Ar). NH signal not observed. M/Z (M[⁸¹Br]+H)⁺=439.1. MP>250° C.

### Compound 19 (2-(4-cyclopropyl-2-pyridyl)-6-[3-(4-pyridyl)propoxy]-3H-quinazolin-4-one dihydrochioride)

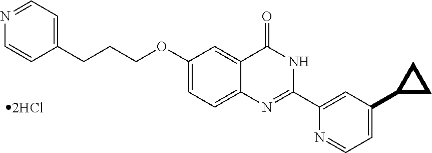

Compound 19 was prepared according to procedure of compound 15, starting from 2-amino-5-(3-pyridin-4-yl-propoxy)-benzamide and lithium 4-cyclopropylpyridine-2-carboxylate. The HCl salt was obtained by filtration after addition of an excess of HCl in Et₂O to a solution of the free base in dichloromethane. Compound 19 was obtained as a green solid in 70% yield.

¹H-NMR (400 MHz, DMSO): 0.88-1.00 (m, 2H, CH₂); 1.17-1.21 (m, 2H, CH₂); 2.12-2.26 (m, 3H, CH₂+CH); 3.10 (t, J 7.2 Hz, 2H, CH₂); 4.18 (t, J 6.1 Hz, 2H, CH₂—O);); 7.28-7.59 (m, 3H, Ar); 7.73-7.83 (m, 1H, Ar); 7.97-8.05 (m, 2H, Ar); 8.09-8.18 (m, 1H, Ar); 8.50-8.60 (m, 1H, Ar); 8.78-8.88 (m, 2H, Ar). NH and HCl salt signals not observed. M/Z (M+H)⁺=399.3. MP=110-156° C.

Lithium 4-cyclopropylpyridine-2-carboxylate was prepared as follows:

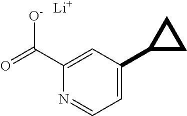

To a suspension of methyl 4-cyclopropylpyridine-2-carboxylate (131 mg, 0.69 mmol) in THF (1.2 mL) and water (1.2 mL), was added LiOH (34 mg, 1.43 mmol) and the reaction mixture was stirred 2 h at room temperature. Next, the reaction mixture was concentrated to dryness to afford the product (142 mg, quantitative yield).

¹H-NMR (400 MHz, DMSO): 0.81 (s, 2H, CH₂); 1.08 (s, 2H, CH₂); 1.99 (s, 1H, CH); 7.14 (bs, 1H, Ar); 7.56 (bs, 1H, Ar); 8.22 (bs, 1H, Ar).

Methyl 4-cyclopropylpyridine-2-carboxylate was prepared as follows:

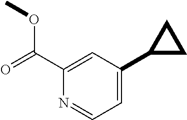

Under inert atmosphere methyl 4-bromopyridine-2-carboxylate (150 mg, 0.69 mmol) was dissolved in dry dioxane (5 mL). Copper iodide (26 mg, 0.14 mmol) and PdCl₂(dppf).CH₂Cl₂ (56 mg, 0.07 mmol) were added followed by cyclopropylzinc bromide (0.5M in THF, 4.0 mL, 2.08 mmol). The reaction mixture was then stirred for 2 h at 80° C. Then the mixture was partitioned between water and ethyl acetate, extracted with ethyl acetate, washed with water and brine, dried over sodium sulfate and concentrated in vacuo to afford methyl 4-cyclopropylpyridine-2-carboxylate (quantitative yield) as a red oil.

¹H-NMR (400 MHz, DMSO): 0.85 (tt, J 4.5, 6.7 Hz, 2H, CH₂); 1.11 (m, 2H, CH₂); 2.07 (m, 1H, CH); 3.86 (s, 3H, CH₃); 7.32 (dd, J 1.7, 5.1 Hz, 1H, Ar); 7.75 (d, J 1.7 Hz, 1H Ar); 8.50 (d, 5.1 Hz, 1H, Ar).

### Reference Compound 20 (2-(3-chlorophenyl)-6-[3-(4-pyridyl)propoxy]-3H-quinazolin-4-one hydrochloride)

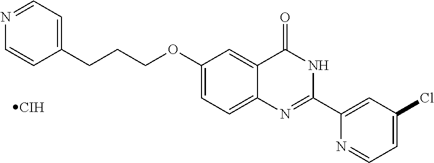

Compound 20 (reference) was prepared according to procedure of example 1, step 4, starting from 2-amino-5-(3-pyridin-4-yl-propoxy)-benzamide and 3-chlorobenzoic acid. The product was purified by column chromatography on silica gel, using dichloromethane/methanol as eluent. The HCl salt was obtained by filtration after addition of an excess of HCl (1.25M in Et₂O) to a solution of the free base in dichloromethane. Compound 20 was obtained as a white solid in 5% yield.

¹H-NMR (400 MHz, DMSO): 2.22 (m, 2H, CH₂); 3.11 (t, J 7.6 Hz, 2H, CH₂); 4.18 (t, J 6.2 Hz, 2H, CH₂—O); 7.41 (dd, J 2.9, 8.8 Hz, 1H, Ar); 7.53 (d, J 2.9 Hz, 1H, Ar); 7.58 (t, 7.9 Hz, 1H, Ar); 7.65 (qd, J 1.1, 8.0H, 1H, Ar); 7.72 (d, J 8.8 Hz, 1H, Ar); 7.99 (d, J 6.4 Hz, 2H, Ar); 8.13 (td, J 1.3, 7.8 Hz, 1H, Ar); 8.23 (t, J 1.8 Hz, 1H, Ar); 8.82 (d, J 6.6 Hz, 2H, Ar); 12.58 (bs, 1H, NH); HCl salt signal not observed. M/Z (M+H)⁺=392. MP>250° C.

### Reference Compound 21 (6-[3-(4-pyridyl)propoxy]-2-[3-(trifluoromethyl)phenyl]-3H-quinazolin-4-one hydrochloride)

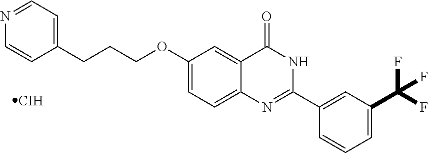

Compound 21 (reference) was prepared according to procedure of example 1, step 4, starting from 2-amino-5-(3-pyridin-4-yl-propoxy)-benzamide and 3-(trifluoromethyl)benzoic acid. The product was purified by column chromatography on silica gel, using dichloromethane/methanol as eluent. The HCl salt was obtained by filtration after addition of an excess of HCl (1.25M in Et₂O) to a solution of the free base in dichloromethane. Compound 21 was obtained as a white solid in 15% yield.

¹H-NMR (400 MHz, DMSO): 2.23 (q, J 6.9 Hz, 2H, CH₂); 3.11 (t, J 7.6 Hz, 2H, CH₂); 4.18 (t, J 6.2 Hz, 2H, CH₂—O); 7.42 (dd, J 3.0, 8.9 Hz, 1H, Ar); 7.54 (d, J 3.0 Hz, 1H, Ar); 7.75 (d, J 8.9 Hz, 1H, Ar); 7.79 (t, J 7.9 Hz, 1H, Ar); 7.94 (d, J 7.8 Hz, 1H, Ar); 8.02 (d, J 6.6 Hz, 2H, Ar); 8.47 (d, J 8.1 Hz, 1H, Ar); 8.52 (s, 1H, Ar); 8.83 (d, J 6.7 Hz, 2H, Ar); 12.73 (bs, 1H, NH); HCl salt signal not observed. M/Z (M+H)⁺=426. MP>250° C.

### Compound 22 (2-(2-Methyl-oxazol-4-yl)-6-(3-pyridin-4-yl-propoxy)-3H-quinazolin-4-one hydrochloride)

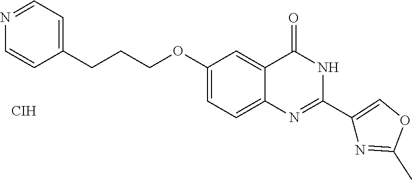 [SMILES: "CIH" ; Cc1nc(-c2nc3ccc(OCCCc4ccncc4)cc3c(=O)[nH]2)co1]

Compound 22 was prepared according to procedure of example 1, step 4, starting from 2-amino-5-(3-pyridin-4-yl-propoxy)-benzamide and 2-methyloxazole-4-carboxylic acid. The HCl salt was obtained by concentration after addition of an excess of HCl (2N in Et₂O) to a solution of the free base in MeOH. Compound 22 was obtained as a yellow solid in 83% yield.

¹H-NMR (400 MHz, DMSO): 2.22 (m, 2H, CH₂); 2.54 (s, 3H, CH₃); 3.10 (t, J 7.6 Hz, 2H, CH₂); 4.17 (t, J 6.4 Hz, 2H, CH₂—O); 7.40 (dd, J 8.9, 2.9 Hz, 1H, Ar); 7.51 (d, J 2.9 Hz, 1H, Ar); 7.68 (d, J 8.9 Hz, 1H, Ar); 8.03 (d, J 6.7 Hz, 2H, Ar); 8.84 (m, 3H, Ar); NH signal not observed; HCl salt signal not observed. M/Z (M+H)⁺=363.1. MP>250° C.

### Example 2—Synthesis of compound 23 (6-(2-Pyridin-3-yl-ethoxy)-2-thieno[3,2-c]pyridin-6-yl-3H-quinazolin-4-one hydrochloride)

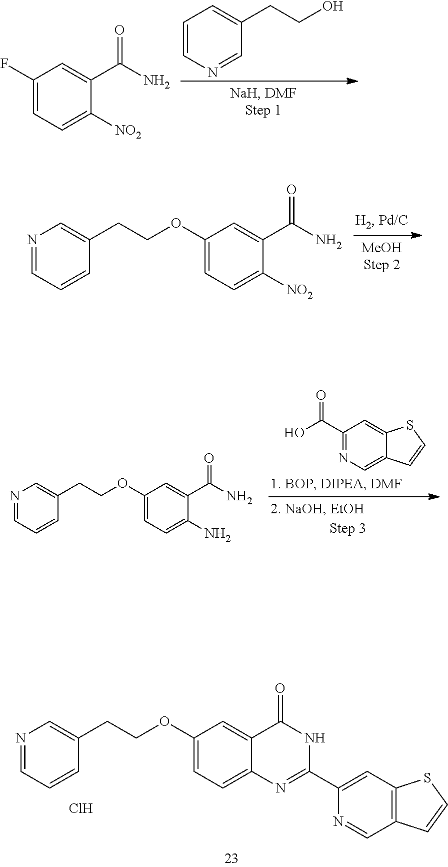

Step 1:

2-Nitro-5-(2-pyridin-3-yl-ethoxy)-benzamide was prepared according to procedure of example 1, step 2, starting from 2-nitro-5-fluorobenzamide and 2-pyridin-3-yl-ethanol. It was obtained as a white powder in 31% yield.

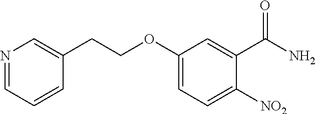 [SMILES: NC(=O)c1cc(OCCc2cccnc2)ccc1[N+](=O)[O-]]

¹H-NMR (400 MHz, CDCl₃): 3.11 (t, J 6.6 Hz, 2H, CH₂); 4.40 (t, J 6.6 Hz, 2H, CH₂-0); 7.07 (d, J 2.7 Hz, 1H, Ar); 7.15 (dd, J 9.0, 2.7 Hz, 1H, Ar); 7.35 (dd, J 7.8, 4.8 Hz, 1H, Ar); 7.64 (bs, 1H, NH); 7.78 (d, J 7.8 Hz, 1H, Ar); 8.01 (bs, 1H, NH); 8.03 (d, J 9.0 Hz, 1H, Ar); 8.45 (dd, J 4.8, 1.7 Hz, 1H, Ar); 8.56 (d, J 1.7 Hz, 1H, Ar). M/Z (M+H)⁺=288.1.

Step 2:

2-Amino-5-(2-pyridin-3-yl-ethoxy)-benzamide was prepared according to procedure of example 1, step 3, and isolated as a beige solid in 86% yield.

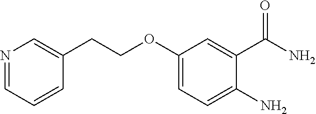 [SMILES: NC(=O)c1cc(OCCc2cccnc2)ccc1N]

¹H-NMR (400 MHz, CDCl₃): 3.01 (t, J 6.6 Hz, 2H, CH₂—C); 4.12 (t, J 6.6 Hz, 2H, CH₂—O); 6.15 (bs, 2H, NH₂); 6.63 (d, J 8.8 Hz, 1H, Ar); 6.83 (dd, J 8.8, 2.9 Hz, 1H, Ar); 7.04 (bs, 1H, NH); 7.11 (d, J 2.9 Hz, 1H, Ar); 7.35 (dd, J 7.7, 4.8 Hz, 1H, Ar); 7.71 (bs, 1H, NH); 7.74 (d, J 7.7 Hz, 1H, Ar); 8.44 (dd, J 4.8, 1.7 Hz, 1H, Ar); 8.54 (d, J 1.7 Hz, 1H, Ar). M/Z (M+H)⁺=258.1.

Step 3:

Compound 23 was prepared according to procedure of example 1, step 4, starting from 2-amino-5-(2-pyridin-3-yl-ethoxy)-benzamide and thieno[3,2-c]pyridine-6-carboxylic acid. The HCl salt was obtained by concentration after addition of an excess of HCl (2N in Et₂O) to a solution of the free base in MeOH. Compound 23 was obtained as a yellow solid in 80% yield.

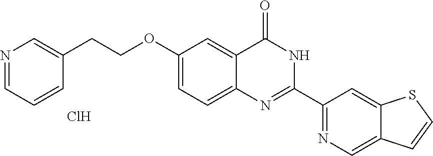 [SMILES: O=c1[nH]c(-c2cc3sccc3cn2)nc2ccc(OCCc3cccnc3)cc12 ; Cl]

¹H-NMR (400 MHz, DMSO): 3.49 (t, J 6.0 Hz, 2H, CH₂); 4.54 (t, J 6.0 Hz, 2H, CH₂—O); 7.57 (dd, J 9.0, 2.9 Hz, 1H, Ar); 7.75 (d, J 2.9 Hz, 1H, Ar); 7.87 (d, J 5.4 Hz, 1H, Ar); 7.94 (d, J 9.0 Hz, 1H, Ar); 8.13 (dd, J 8.1, 5.7 Hz, 1H, Ar); 8.19 (d, J 5.4 Hz, 1H, Ar); 8.76 (d, J 8.1 Hz, 1H, Ar); 8.81 (d, J 5.7 Hz, 1H, Ar); 8.97 (s, 1H, Ar); 9.20 (s, 1H, Ar); 9.43 (s, 1H, Ar); NH signal not observed; HCl salt signal not observed. M/Z (M+H)⁺=401.0. MP>250° C.

### Example 3—Synthesis of compound 24 (6-(4-Bromo-benzyloxy)-2-thieno[3,2-c]pyridin-6-yl-3H-quinazolin-4-one)

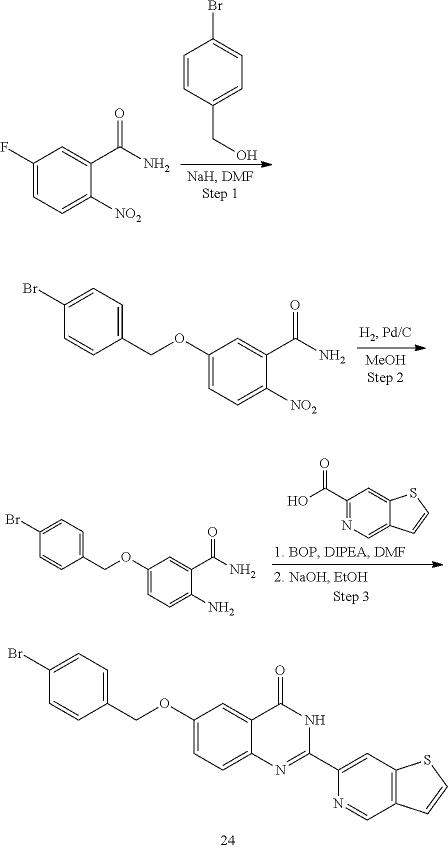 [SMILES: NC(=O)c1cc(F)ccc1[N+](=O)[O-] ; OCc1ccc(Br)cc1 ; NC(=O)c1cc(OCc2ccc(Br)cc2)ccc1[N+](=O)[O-] ; NC(=O)c1cc(OCc2ccc(Br)cc2)ccc1N ; O=C(O)c1cc2sccc2cn1 ; O=c1[nH]c(-c2cc3sccc3cn2)nc2ccc(OCc3ccc(Br)cc3)cc12]

Step 1:

2-Nitro-5-(4-Bromo-benzyloxy)benzamide was prepared according to procedure of example 1, step 2, starting from 2-nitro-5-fluorobenzamide and 4-bromobenzyl alcohol. It was obtained as a yellow solid in 63% yield.

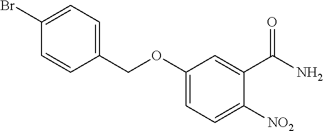 [SMILES: NC(=O)c1cc(OCc2ccc(Br)cc2)ccc1[N+](=O)[O-]]

M/Z (M[⁷⁹Br]+H)⁺=351.0.

Step 2:

2-Amino-5-(4-Bromo-benzyloxy)benzamide was prepared according to procedure of example 1, step 3, and isolated as a pale yellow solid in 55% yield.

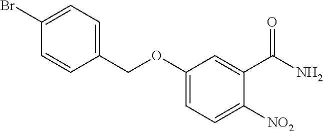 [SMILES: NC(=O)c1cc(OCc2ccc(Br)cc2)ccc1[N+](=O)[O-]]

M/Z (M[⁷⁹Br]+H)⁺=321.0.

Step 3:

Compound 24 was prepared according to procedure of example 1, step 4, starting from 2-amino-5-(4-Bromo-benzyloxy)benzamide and thieno[3,2-c]pyridine-6-carboxylic acid. Compound 24 was obtained as a beige solid in 69% yield.

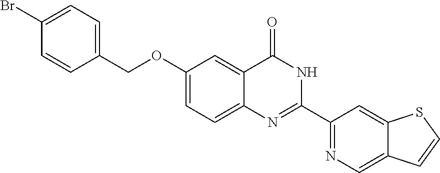 [SMILES: O=c1[nH]c(-c2cc3sccc3cn2)nc2ccc(OCc3ccc(Br)cc3)cc12]

¹H-NMR (400 MHz, DMSO): 5.16 (s, 2H, CH₂—O); 7.24 (dd, J 8.8, 2.9 Hz, 1H, Ar); 7.47 (d, J 8.5 Hz, 2H, Ar); 7.53 (d, J 2.9 Hz, 1H, Ar); 7.57 (d, J 8.8 Hz, 1H, Ar); 7.61 (d, J 8.5 Hz, 2H, Ar); 7.67 (d, J 5.4 Hz, 1H, Ar); 7.91 (d, J 5.4 Hz, 1H, Ar); 9.05 (s, 1H, Ar); 9.18 (s, 1H, Ar); NH signal not observed. M/Z (M[⁷⁹Br]+H)⁺=364.0. MP>250° C.

### Example 4—Synthesis of compounds 25 (tert-butyl 3-(4-hydroxy-2-pyrrolo[1,2-c]pyrimidin-3-yl-quinazolin-6-yl)oxyazetidine-1-carboxylate), 26 (6-(azetidin-3-yloxy)-2-pyrrolo[1,2-c]pyrimidin-3-yl-3H-quinazolin-4-one hydrochloride) and 27 (6-(1-Pyrimidin-4-yl-azetidin-3-yloxy)-2-pyrrolo[1,2-c]pyrimidin-3-yl-3H-quinazolin-4-one)

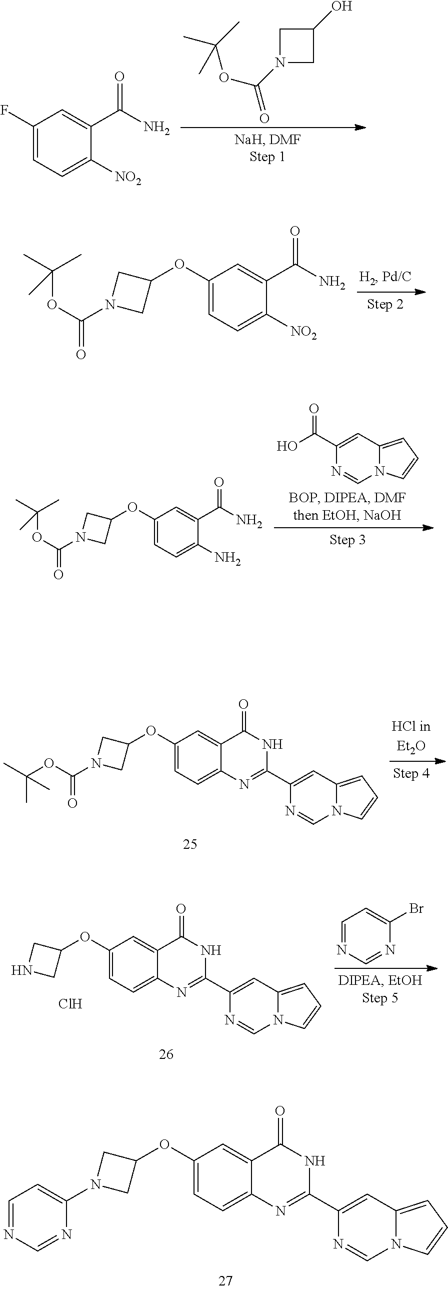

Step 1:

3-(4-Nitro-3-carbamoyl-phenoxy)-azetidine-1-carboxylic acid tert-butyl ester was prepared according to procedure of example 1, step 2, starting from 2-nitro-5-fluorobenzamide and 1-boc-3-hydroxyazetidine. It was obtained as a yellow oil in 94% yield.

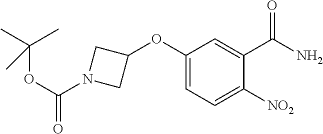 [SMILES: CC(C)(C)OC(=O)N1CC(Oc2ccc([N+](=O)[O-])c(C(N)=O)c2)C1]

M/Z (M+Na)⁺=360.1.

Step 2:

3-(4-Amino-3-carbamoyl-phenoxy)-azetidine-1-carboxylic acid tert-butyl ester was prepared according to procedure of example 1, step 3, and isolated as a pale yellow solid in 94% yield. It was taken crude to the next step.

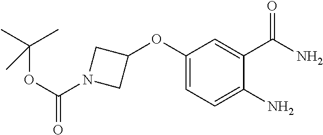 [SMILES: CC(C)(C)OC(=O)N1CC(Oc2ccc(N)c(C(N)=O)c2)C1]

¹H-NMR (400 MHz, DMSO): 1.38 (s, 9H, tert-butyl); 3.76 (m, 2H, 2 CH); 4.24 (m, 2H, 2 CH); 4.87 (m, 1H, CH); 6.21 (bs, 2H, NH₂); 6.65 (d, J 8.8 Hz, 1H, Ar); 6.78 (dd, J 8.8, 2.8 Hz, 1H, Ar); 6.97 (d, J 2.8 Hz, 1H, Ar); 7.08 (bs, 1H, NH); 7.74 (bs, 1H, NH). M/Z (M+Na)⁺=330.1.

Step 3:

3-(4-Hydroxy-2-pyrrolo[1,2-c]pyrimidin-3-yl-quinazolin-6-yloxy)-azetidine-1-carboxylic acid tert-butyl ester 25 was prepared according to procedure of example 1, step 4, starting from 3-(4-Amino-3-carbamoyl-phenoxy)-azetidine-1-carboxylic acid tert-butyl ester and pyrrolo[1,2-c]pyrimidine-3-carboxylic acid. It was obtained as a green solid in 50% yield.

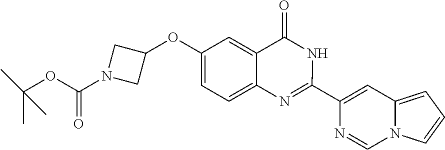 [SMILES: CC(C)(C)OC(=O)N1CC(Oc2ccc3nc(-c4cc5cccn5cn4)[nH]c(=O)c3c2)C1]

¹H-NMR (400 MHz, DMSO): 1.45 (s, 9H, tert-butyl); 3.91 (m, 2H, 2 CH); 4.40 (m, 2H, 2 CH); 5.21 (m, 1H, CH); 6.90 (d, J 3.8 Hz, 1H, Ar); 7.11 (dd, J 3.8, 2.7 Hz, 1H, Ar); 7.38 (d, J 2.9 Hz, 1H, Ar); 7.47 (dd, J 8.8, 2.9 Hz, 1H, Ar); 7.76 (d, J 8.8 Hz, 1H, Ar); 7.94 (d, J 2.7 Hz, 1H, Ar); 8.54 (s, 1H, Ar); 9.37 (s, 1H, Ar); 11.11 (bs, 1H, NH). M/Z (M+H)⁺=434.1. MP>250° C.

Step 4:

At 0° C., to a solution of 3-(4-hydroxy-2-pyrrolo[1,2-c]pyrimidin-3-yl-quinazolin-6-yloxy)-azetidine-1-carboxylic acid tert-butyl ester 25 (135 mg, 0.31 mmol) in dichloromethane (2.0 mL), a solution of HCl (2N in Et₂O, 1.55 mL) was added dropwise. The reaction mixture was stirred for 2 h at room temperature before a dark precipitate was collected by filtration, triturated in dichloromethane and dried under vacuum. 6-(azetidin-3-yloxy)-2-pyrrolo[1,2-c]pyrimidin-3-yl-quinazolin-4-ol hydrochloride 26 (148 mg, quantitative yield) was obtained with 70% purity (UV of LC/MS) and was taken crude to next step without purification.

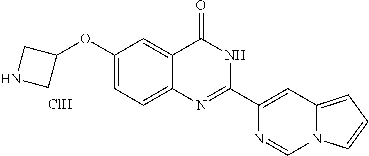 [SMILES: O=c1[nH]c(-c2cc3cccn3cn2)nc2ccc(OC3CNC3)cc12 ; Cl]

M/Z (M+H)⁺=334.1.

Step 5:

Under inert atmosphere, a suspension of 6-(azetidin-3-yloxy)-2-pyrrolo[1,2-c]pyrimidin-3-yl-quinazolin-4-ol hydrochloride 26 (148 mg, 0.40 mmol), 4-bromopyrimidine hydrochloride (156 mg, 0.80 mmol) and diisopropylethylamine (0.30 mL, 1.60 mmol) in ethanol (2.2 mL) was stirred at room temperature for 20 h. The solvent was removed under vacuum and the crude dark oil was purified by flash column chromatography on silica gel using dichloromethane/methanol as eluent to afford compound 27 (30 mg, 18%) as a yellow solid.

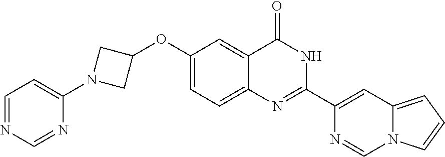 [SMILES: O=c1[nH]c(-c2cc3cccn3cn2)nc2ccc(OC3CN(c4ccncn4)C3)cc12]

¹H-NMR (400 MHz, DMSO): 4.10 (m, 2H, 2 CH); 4.58 (m, 2H, 2 CH); 5.39 (m, 1H, CH); 6.50 (d, J 5.9 Hz, 1H, Ar); 6.86 (d, J 6.7 Hz, 1H, Ar); 7.06 (m, 1H, Ar); 7.48 (m, 2H, Ar); 7.75 (d, J 8.2 Hz, 1H, Ar); 7.89 (s, 1H, Ar); 8.20 (d, J 5.9 Hz, 1H, Ar); 8.51 (m, 2H, Ar); 9.31 (s, 1H, Ar); 11.16 (bs, 1H, NH). M/Z (M+H)⁺=412.1. MP>250° C.

### Example 5—Synthesis of compounds 28 (3-(4-Hydroxy-2-thieno[2,3-c]pyridin-5-yl-quinazolin-6-yloxy)-azetidine-1-carboxylic acid tert-butyl ester), 29 (6-(azetidin-3-yloxy)-2-thieno[2,3-c]pyridin-5-yl-quinazolin-4-ol 2,2,2-trifluoroacetate) and 30 (6-(1-Propionyl-azetidin-3-yloxy)-2-thieno[2,3-c]pyridin-5-yl-3H-quinazolin-4-one)

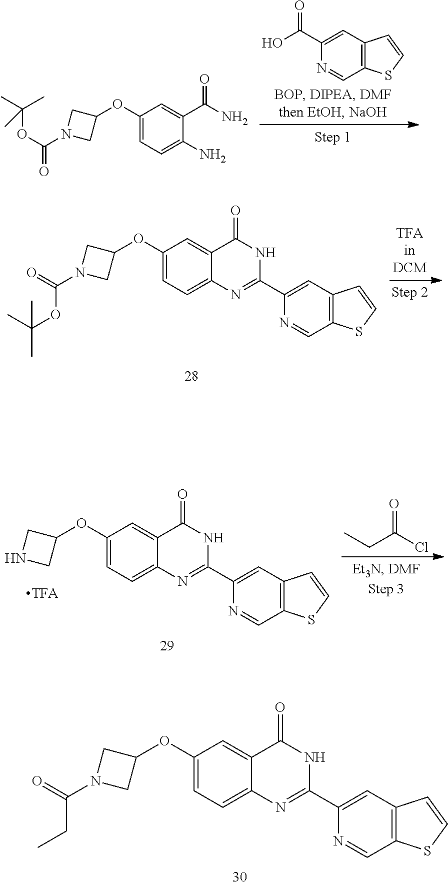

Step 1:

3-(4-Hydroxy-2-thieno[2,3-c]pyridin-5-yl-quinazolin-6-yloxy)-azetidine-1-carboxylic acid tert-butyl ester 28 was prepared according to procedure of example 1, step 4, starting from 3-(4-amino-3-carbamoyl-phenoxy)-azetidine-1-carboxylic acid tert-butyl ester (440 mg, 0.94 mmol) and thieno[2,3-c]pyridine-5-carboxylic acid (252 mg, 1.41 mmol). It was obtained as a white solid (430 mg, quantitative yield).

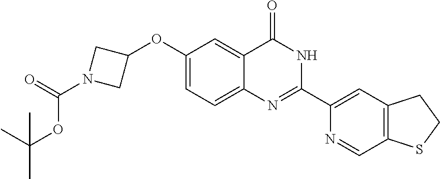 [SMILES: CC(C)(C)OC(=O)N1CC(Oc2ccc3nc(-c4cc5c(cn4)SCC5)[nH]c(=O)c3c2)C1]

M/Z (M+H)⁺=451.0.

Step 2:

At 0° C., to a solution of 3-(4-Hydroxy-2-thieno[2,3-c]pyridin-5-yl-quinazolin-6-yloxy)-azetidine-1-carboxylic acid tert-butyl ester 28 (430 mg, 0.95 mmol) in dichloromethane (5.0 mL), TFA (750 μL, 9.60 mmol) was added dropwise. The reaction mixture was stirred for 2 h at room temperature before being concentrated to dryness under vacuum. Trituration in diethyl ether afforded 6-(azetidin-1-ium-3-yloxy)-2-thieno[2,3-c]pyridin-5-yl-quinazolin-4-ol 2,2,2-trifluoroacetate 29 (quantitative yield) as a yellow solid.

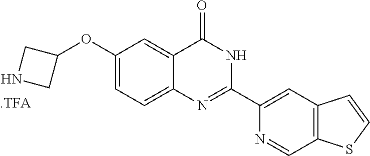 [SMILES: O=c1[nH]c(-c2cc3ccsc3cn2)nc2ccc(OC3CNC3)cc12]

M/Z (M+H)⁺=350.9.

Step 3:

At 0° C., to a mixture of 6-(azetidin-3-yloxy)-2-thieno[2,3-c]pyridin-5-yl-quinazolin-4-ol 2,2,2-trifluoroacetate 29 (200 mg, 0.43 mmol) and triethylamine (180 μL, 1.29 mmol) in DMF (4.5 mL) propionyl chloride (40 μL, 0.43 mmol) was added dropwise. The reaction mixture was stirred for 16 h at room temperature before being poured into ice water (20 mL). The resulting beige precipitate was collected by filtration and purified by column chromatography on silia gel, using dichloromethane/methanol as eluent to afford compound 30 (69 mg, 39%) as a white solid.

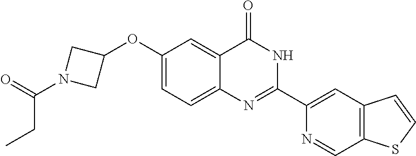 [SMILES: CCC(=O)N1CC(Oc2ccc3nc(-c4cc5ccsc5cn4)[nH]c(=O)c3c2)C1]

¹H-NMR (400 MHz, DMSO): 0.97 (t, J 7.5 Hz, 3H, ethyl); 2.12 (q, J 7.5 Hz, 2H, ethyl); 3.86 (m, 1H, CH); 4.14 (m, 1H, CH); 4.34 (m, 1H, CH); 4.62 (m, 1H, CH); 5.23 (m, 1H, CH); 7.40 (d, J 2.9 Hz, 1H, Ar); 7.48 (dd, J 8.8, 2.9 Hz, 1H, Ar); 7.80 (m, 2H, Ar); 8.27 (d, J 5.4 Hz, 1H, Ar); 8.93 (s, 1H, Ar); 9.43 (s, 1H, Ar); 11.81 (s, 1H, NH). M/Z (M+H)⁺=406.9. MP>250° C.

### Example 6—Synthesis of compound 31 (6-(Piperidin-4-yloxy)-2-thieno[3,2-c]pyridin-6-yl-3H-quinazolin-4-one) and 32 (6-(1-Propionyl-piperidin-4-yloxy)-2-thieno[3,2-c]pyridin-6-yl-3H-quinazolin-4-one)

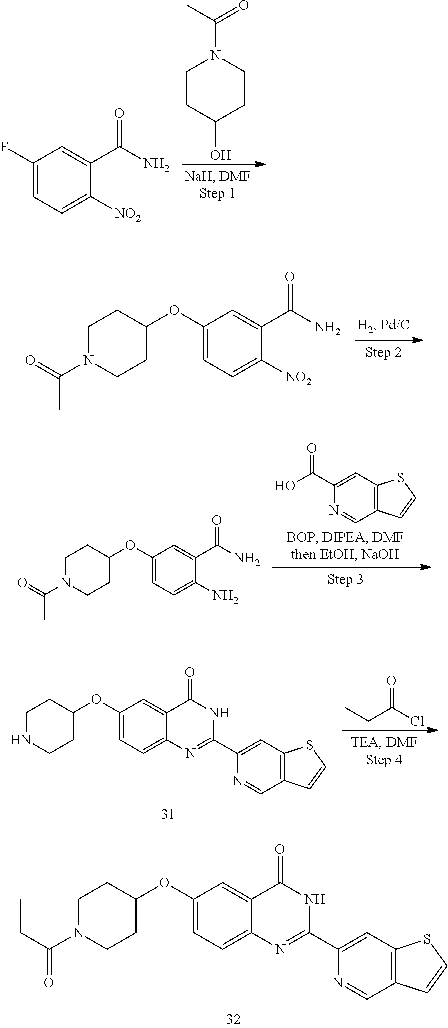

Step 1:

5-(1-Acetyl-piperidin-4-yloxy)-2-nitro-benzamide was prepared according to procedure of example 1, step 2, starting from 2-nitro-5-fluorobenzamide and 1-acetyl-4-hydroxypiperidine. It was obtained as a yellow oil in 33% yield.

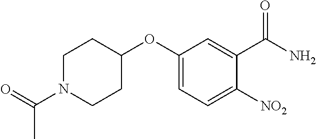 [SMILES: CC(=O)N1CCC(Oc2ccc([N+](=O)[O-])c(C(N)=O)c2)CC1]

M/Z (M+H)⁺=308.1.

Step 2:

5-(1-Acetyl-piperidin-4-yloxy)-2-amino-benzamide was prepared according to procedure of example 2, step 3, and isolated as a yellow solid in 85% yield. It was used in the next step without purification.

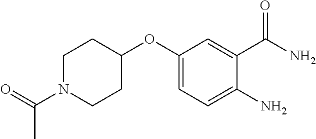 [SMILES: CC(=O)N1CCC(Oc2ccc(N)c(C(N)=O)c2)CC1]

M/Z (M+Na)⁺=300.1.

Step 3:

6-(Piperidin-4-yloxy)-2-thieno[3,2-c]pyridin-6-yl-quinazolin-4-ol was prepared according to procedure of example 1, step 4, starting from 5-(1-acetyl-piperidin-4-yloxy)-2-amino-benzamide and thieno[3,2-c]pyridine-6-carboxylic acid. Compound 31 was obtained as a yellow solid in 88% yield.

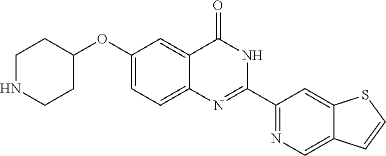 [SMILES: O=c1[nH]c(-c2cc3sccc3cn2)nc2ccc(OC3CCNCC3)cc12]

M/Z (M+Na)⁺=379.1.

Step 4:

Compound 32 was prepared according to procedure of example 5, step 3, starting from compound 31 and was obtained as a white solid in 35% yield.

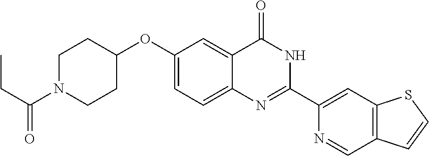 [SMILES: CCC(=O)N1CCC(Oc2ccc3nc(-c4cc5sccc5cn4)[nH]c(=O)c3c2)CC1]

¹H-NMR (400 MHz, DMSO): 1.06 (t, J 7.4 Hz, 3H, ethyl); 1.68 (m, 2H, 2 CH); 2.07 (m, 2H, 2 CH); 2.41 (q, J 7.4 Hz, 2H, ethyl); 3.45 (m, 2H, 2 CH); 3.77 (m, 1H, CH); 3.95 (m, 1H, CH); 4.90 (m, 1H, CH); 7.58 (dd, J 8.9, 2.9 Hz, 1H, Ar); 7.71 (d, J 2.9 Hz, 1H, Ar); 7.82 (d, J 5.4 Hz, 1H, Ar); 7.83 (d, J 8.9 Hz, 1H, Ar); 8.17 (d, J 5.4 Hz, 1H, Ar); 9.20 (s, 1H, Ar); 9.36 (s, 1H, Ar); 11.83 (s, 1H, NH). M/Z (M+H)⁺=435.0. MP>250° C.

### Example 7—Synthesis of compound 33 (6-(2-Morpholin-4-yl-ethoxy)-2-pyrrolo[1,2-c]pyrimidin-3-yl-3H-quinazolin-4-one hydrochloride)

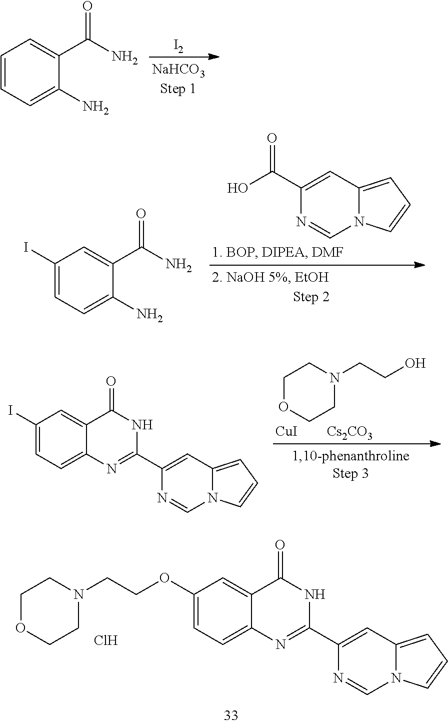

Step 1:

To a suspension of anthranilamide (2.2 g, 16.4 mmol) in an aqueous solution of sodium bicarbonate (5%, 0.1M, 165 mL) was added iodine (4.6 g, 18.0 mmol). The reaction mixture was stirred for 16 h at room temperature before being poured into an aqueous saturated solution of sodium sulfite (300 mL) and extracted with ethyl acetate (3×300 mL). The combined organic extracts were washed with brine (100 mL), dried over MgSO₄ and concentrated under vacuum. Purification by flash column chromatography on silica gel using ethyl acetate/cyclohexane as eluent afforded 2-amino-5-iodo-benzamide (2.7 g, 63%) as a beige solid.

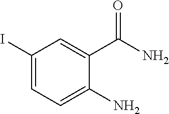 [SMILES: NC(=O)c1cc(I)ccc1N]

¹H-NMR (400 MHz, DMSO): 6.54 (d, J 8.7 Hz, 1H, Ar); 6.70 (bs, 2H, NH₂); 7.13 (bs, 1H, NH); 7.38 (dd, J 8.7, 2.0 Hz, 1H, Ar); 7.80 (d, J 2.0 Hz, 1H, Ar); 7.81 (bs, 1H, NH). M/Z (M+H)⁺=263.0.

Step 2:

6-Iodo-2-pyrrolo[1,2-c]pyrimidin-3-yl-quinazolin-4-ol was prepared according to procedure of example 1, step 4, starting from 2-amino-5-iodo-benzamide and pyrrolo[1,2-c]pyrimidine-3-carboxylic acid. It was obtained as a green solid in 70% yield.

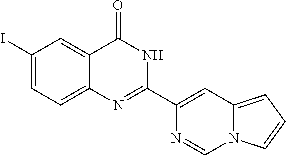 [SMILES: O=c1[nH]c(-c2cc3cccn3cn2)nc2ccc(I)cc12]

¹H-NMR (400 MHz, DMSO): 6.67 (d, J 3.8 Hz, 1H, Ar); 6.99 (dd, J 3.8, 2.7 Hz, 1H, Ar); 7.42 (d, J 8.6 Hz, 1H, Ar); 7.78 (dd, J 8.6, 2.2 Hz, 1H, Ar); 7.85 (d, J 2.7 Hz, 1H, Ar); 8.33 (d, J 2.2 Hz, 1H, Ar); 8.53 (s, 1H, Ar); 9.20 (s, 1H, Ar). M/Z (M+H)⁺=289.0.

Step 3:

Under inert atmosphere, in a sealed tube, a suspension of 6-iodo-2-pyrrolo[1,2-c]pyrimidin-3-yl-quinazolin-4-ol (50 mg, 0.13 mmol), copper iodide (5 mg, 0.03 mmol), 1,10-phenanthroline (9 mg, 0.05 mmol) and cesium carbonate (84 mg, 0.26 mmol) in 4-(2-hydroxyethyl)morpholine (1.0 mL) was heated at 120° C. for 24 h. After cooling to room temperature, the mixture was poured into ice water (10 mL) and the resulting dark precipitate was collected by filtration. It was dissolved in DMSO (5 mL) and purified by preparative HPLC. The HCl salt was obtained by concentration after addition of an excess of HCl (2N in Et₂O) to a solution of the free base in MeOH to give compound 33 (9 mg, 16%) as a brown solid.

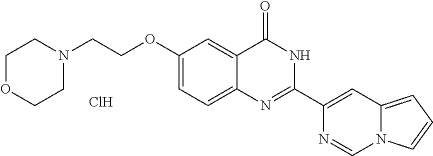 [SMILES: O=c1[nH]c(-c2cc3cccn3cn2)nc2ccc(OCCN3CCOCC3)cc12 ; Cl]

¹H-NMR (400 MHz, DMSO): 3.22 (m, 2H, CH₂); 3.52 (m, 2H, CH₂); 3.61 (m, 2H, CH₂); 3.85 (m, 2H, CH₂); 3.96 (m, 2H, CH₂); 4.59 (m, 2H, CH₂); 6.88 (bs, 1H, Ar); 7.08 (m, 1H, Ar); 7.53 (bs, 1H, Ar); 7.63 (m, 1H, Ar); 7.76 (bs, 1H, Ar); 7.92 (bs, 1H, Ar); 8.53 (s, 1H, Ar); 9.36 (s, 1H, Ar); 11.30 (bs, NH); HCl salt signal not observed. M/Z (M+H)⁺=392.0. MP=165-173° C.

### Compound 34 (6-(2-Methoxy-ethoxy)-2-pyrrolo[1,2-c]pyrimidin-3-yl-3H-quinazolin-4-one)

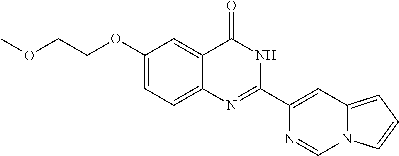 [SMILES: COCCOc1ccc2nc(-c3cc4cccn4cn3)[nH]c(=O)c2c1]

Compound 34 was prepared according to procedure of example 7, step 3, starting from 6-iodo-2-pyrrolo[1,2-c]pyrimidin-3-yl-quinazolin-4-ol and 2-methoxyethanol. It was purified by flash column chromatography on silica gel, using dichloromethane/methanol as eluent. Compound 34 was obtained as a yellow solid in 10% yield.

¹H-NMR (400 MHz, DMSO): 3.33 (s, 3H, CH₃); 3.72 (m, 2H, CH₂); 4.24 (m, 2H, CH₂); 6.86 (d, J 3.8 Hz, 1H, Ar); 7.07 (dd, J 3.8, 2.7 Hz, 1H, Ar); 7.46 (dd, J 8.8, 2.9 Hz, 1H, Ar); 7.55 (d, J 2.9 Hz, 1H, Ar); 7.68 (d, J 8.8 Hz, 1H, Ar); 7.89 (d, J 2.7 Hz, 1H, Ar); 8.49 (s, 1H, Ar); 9.33 (s, 1H, Ar); 11.46 (bs, 1H, NH). M/Z (M+H)⁺=337.1. MP=181-187° C.

### Compound 35 (6-(2-Morpholin-4-yl-ethoxy)-2-thieno[3,2-c]pyridin-6-yl-3H-quinazolin-4-one hydrochloride)

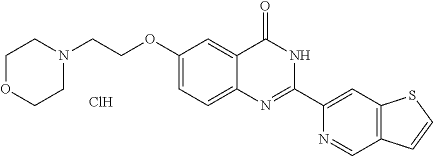 [SMILES: O=c1[nH]c(-c2cc3sccc3cn2)nc2ccc(OCCN3CCOCC3)cc12 ; Cl]

Compound 35 was prepared according to procedure of example 7, starting from thieno[3,2-c]pyridine-6-carboxylic acid and 2-amino-5-iodo-benzamide in step 2. The HCl salt was obtained by concentration after addition of an excess of HCl (2N in Et₂O) to a solution of the free base in MeOH. Compound 35 was obtained as a white solid in 42% yield.

¹H-NMR (400 MHz, DMSO): 3.24 (m, 2H, CH₂); 3.55 (m, 2H, CH₂); 3.63 (m, 2H, CH₂); 3.82 (m, 2H, CH₂); 3.99 (m, 2H, CH₂); 4.61 (m, 2H, CH₂); 7.57 (d, J 8.6 Hz, 1H, Ar); 7.68 (s, 1H, Ar); 7.77 (d, J 5.0 Hz, 1H, Ar); 7.82 (d, J 8.6 Hz, 1H, Ar); 8.13 (d, J 5.0 Hz, 1H, Ar); 9.16 (s, 1H, Ar); 9.32 (s, 1H, Ar); 11.15 (bs, 1H, NH); HCl salt signal not observed. M/Z (M+H)⁺=409.0. MP>250° C.

### Compound 36 (6-(2-Methoxy-ethoxy)-2-thieno[3,2-c]pyridin-6-yl-3H-quinazolin-4-one)

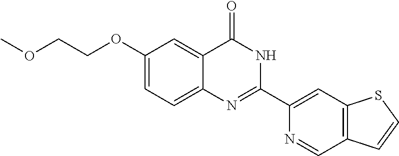 [SMILES: COCCOc1ccc2nc(-c3cc4sccc4cn3)[nH]c(=O)c2c1]

Compound 36 was prepared according to procedure of example 7, step 3, starting from 6-iodo-2-thieno[3,2-c]pyridin-6-yl-quinazolin-4-ol and 2-methoxyethanol. It was purified by flash column chromatography on silica gel using dichloromethane/methanol as eluent. Compound 36 was obtained as a white solid in 22% yield.

¹H-NMR (400 MHz, DMSO): 3.37 (s, 3H, CH₃); 3.75 (m, 2H, CH₂); 4.28 (m, 2H, CH₂); 7.49 (dd, J 9.0, 2.9 Hz, 1H, Ar); 7.63 (d, J 2.9 Hz, 1H, Ar); 7.75 (d, J 5.4 Hz, 1H, Ar); 7.77 (d, J 9.0 Hz, 1H, Ar); 8.07 (d, J 5.4 Hz, 1H, Ar); 9.11 (s, 1H, Ar); 9.29 (s, 1H, Ar); 11.41 (bs, 1H, NH). M/Z (M+H)⁺=354.1. MP=209-213° C.

### Compound 37 (6-(3-Pyridin-3-yl-propoxy)-2-thieno[3,2-c]pyridin-6-yl-3H-quinazolin-4-one hydrochloride)

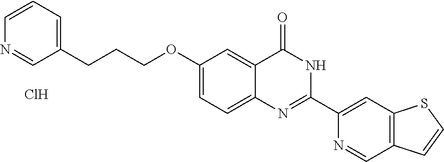 [SMILES: O=c1[nH]c(-c2cc3sccc3cn2)nc2ccc(OCCCc3cccnc3)cc12 ; Cl]

Compound 37 was prepared according to procedure of example 7, step 3, starting from 6-iodo-2-thieno[3,2-c]pyridin-6-yl-quinazolin-4-ol and 3-pyridinepropanol. It was purified by preparative HPLC and the HCl salt was obtained by filtration after addition of an excess of HCl (2N in Et₂O) to a solution of the product in dichloromethane. Compound 37 was obtained as an orange solid in 14% yield.

¹H-NMR (400 MHz, DMSO): 2.20 (m, 2H, CH₂); 3.04 (t, J 7.5 Hz, 2H, CH₂); 4.19 (t, J 6.2 Hz, 2H, CH₂—O); 7.44 (dd, J 8.8, 2.7 Hz, 1H, Ar); 7.57 (d, J 2.7 Hz, 1H, Ar); 7.77 (m, 2H, Ar); 8.03 (dd, J 8.0, 5.6 Hz, 1H, Ar); 8.12 (d, J 5.4 Hz, 1H, Ar); 8.57 (d, J 8.0 Hz, 1H, Ar); 8.80 (d, J 5.4 Hz, 1H, Ar); 8.91 (s, 1H, Ar); 9.15 (s, 1H, Ar); 9.31 (s, 1H, Ar); NH signal not observed; HCl salt signal not observed. M/Z (M+H)⁺=415.0. MP=149-154° C.

### Example 8—Synthesis of reference compound 38 (3-Methyl-6-(3-pyridin-4-yl-propoxy)-2-thieno[3,2-c]pyridin-6-yl-quinazolin-4-one)

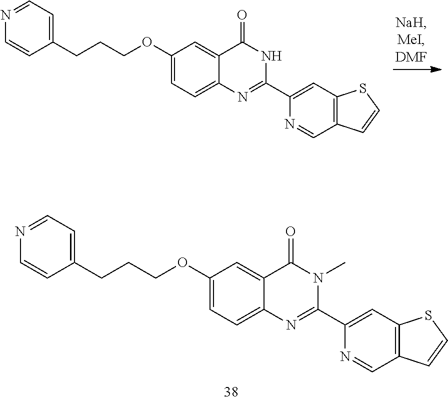 [SMILES: O=c1[nH]c(-c2cc3sccc3cn2)nc2ccc(OCCCc3ccncc3)cc12 ; Cn1c(-c2cc3sccc3cn2)nc2ccc(OCCCc3ccncc3)cc2c1=O]

At 0° C., to a suspension of sodium hydride (60% dispersion in oil, 24 mg, 0.60 mmol) in DMF (0.6 mL) was added dropwise a solution of compound 3 (50 mg, 0.12 mmol) in DMF (0.6 mL). The reaction mixture was stirred for 15 min at 0° C. before addition of iodomethane (60 μL, 0.97 mmol). Then, the reaction mixture was stirred for 1 h at room temperature before addition of water (10 mL). The resulting orange precipitate was collected by filtration and purified by flash column chromatography on silica gel, using dichloromethane/methanol as eluent. The HCl salt was obtained by filtration after addition of an excess of HCl (2N in Et₂O) to a solution of the product in dichloromethane to give reference compound 38 (11 mg, 21%) as a yellow solid.

¹H-NMR (400 MHz, DMSO): 2.23 (m, 2H, CH₂); 3.12 (t, J 7.3 Hz, 2H, CH₂); 3.49 (s, 3H, CH₃); 4.20 (t, J 6.2 Hz, 2H, CH₂—O); 7.43 (dd, J 8.8, 2.9 Hz, 1H, Ar); 7.57 (d, J 2.9 Hz, 1H, Ar); 7.68 (d, J 8.8 Hz, 1H, Ar); 7.76 (d, J 5.5 Hz, 1H, Ar); 8.02 (d, J 6.6 Hz, 2H, Ar); 8.10 (d, J 5.5 Hz, 1H, Ar); 8.60 (s, 1H, Ar); 8.83 (d, J 6.6 Hz, 2H, Ar); 9.28 (s, 1H, Ar); HCl salt signal not observed. M/Z (M+H)⁺=429.1. MP>250° C.

### Example 9—Synthesis of compounds 39 (4-(4-Oxo-2-pyridin-2-yl-3,4-dihydro-quinazolin-6-yloxy)-piperidine-1-carboxylic acid tert-butyl ester), 40 (6-(piperidin-4-yloxy)-2-pyridin-2-yl-3H-quinazolin-4-one hydrochloride) and 41 (6-(1-Acetyl-piperidin-4-yloxy)-2-pyridin-2-yl-3H-quinazolin-4-one)

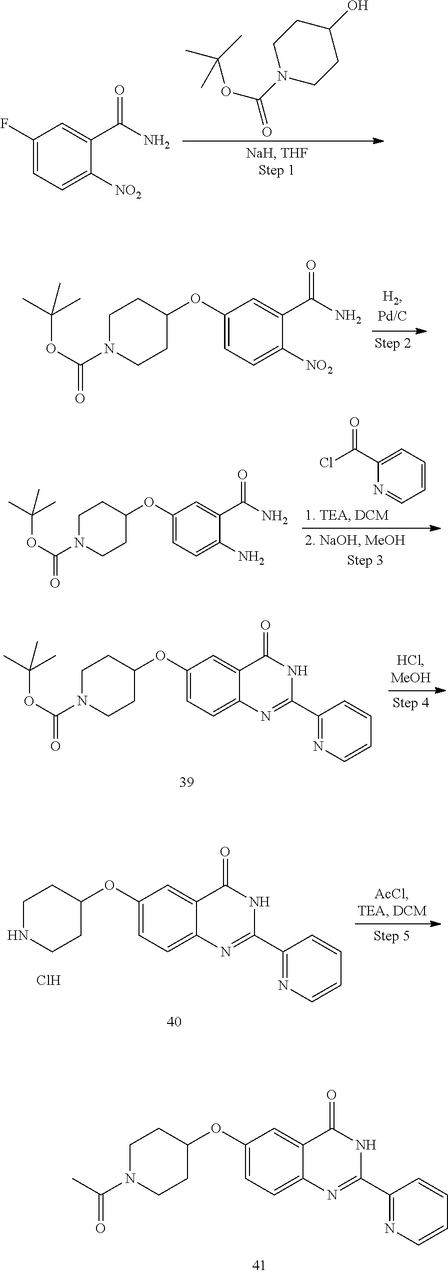

Step 1:

To a solution of 5-fluoro-2-nitrobenzamide (1.00 g, 5.43 mmol) and 1-boc-4-hydroxypiperidine (1.64 g, 8.15 mmol) in dry THF (30 mL) was added a suspension of sodium hydride (60% dispersion in oil, 869 mg, 21.7 mmol). The yellow suspension was stirred at room temperature for 1 day before being poured onto aqueous ammonium chloride (100 mL) and extracted with ethyl acetate (3×50 mL). The combined organic phase was washed with brine (50 mL), dried over anhydrous sodium sulfate, filtered and concentrated under reduced pressure. The residue was purified by column chromatography on silica gel, using hexane/ethyl acetate as eluent, to give 4-(3-carbamoyl-4-nitro-phenoxy)-piperidine-1-carboxylic acid tert-butyl ester (603 mg, 30%) as a white solid.

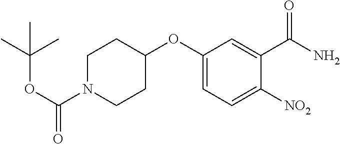 [SMILES: CC(C)(C)OC(=O)N1CCC(Oc2ccc([N+](=O)[O-])c(C(N)=O)c2)CC1]

M/Z (M−C₄H₇)⁺=310.

Step 2:

A solution of 4-(3-carbamoyl-4-nitro-phenoxy)-piperidine-1-carboxylic acid tert-butyl ester (525 mg, 1.44 mmol) in methanol (60 mL) was pumped through a H-Cube instrument containing 10% palladium on charcoal CatCart and full hydrogen flow generated in the chamber of H-Cube by electrolysis of water. The flow rate was set to 1 mL/min and the temperature to 60° C. After 20 min all the reaction mixture had passed through the H-Cube. The CatCart was washed with methanol for 10 min. The fractions were concentrated under reduced pressure to give 4-(4-amino-3-carbamoyl-phenoxy)-piperidine-1-carboxylic acid tert-butyl ester (552 mg, quantitative yield) as a colorless oil.

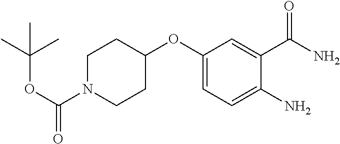 [SMILES: CC(C)(C)OC(=O)N1CCC(Oc2ccc(N)c(C(N)=O)c2)CC1]

M/Z (M−C₄H₇)⁺=280.

Step 3:

To a solution of 4-(4-amino-3-carbamoyl-phenoxy)-piperidine-1-carboxylic acid tert-butyl ester (482 mg, 1.44 mmol,) in dry dichloromethane (10 mL) was added triethylamine (801 μL, 5.75 mmol) and picolinoyl chloride hydrochloride (384 mg, 2.16 mmol) and the colorless solution was stirred at room temperature for 3 h. The reaction mixture was concentrated under reduced pressure and purified by column chromatography on silica gel, using hexane/ethyl acetate as eluent to give 4-{3-carbamoyl-4-[(pyridine-2-carbonyl)-amino]-phenoxy}-piperidine-1-carboxylic acid tert-butyl ester (571 mg, 87%) as a colorless oil.

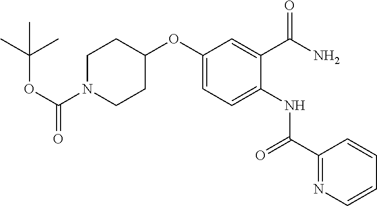 [SMILES: CC(C)(C)OC(=O)N1CCC(Oc2ccc(NC(=O)c3ccccn3)c(C(N)=O)c2)CC1]

M/Z (M−C₄H₇)⁺=310.

To a solution of 4-{3-carbamoyl-4-[(pyridine-2-carbonyl)-amino]-phenoxy}-piperidine-1-carboxylic acid tert-butyl ester (520 mg, 1.18 mmol) in methanol (1 mL) was added a 1M aqueous solution of sodium hydroxide (5 mL, 5.00 mmol) and the white suspension was heated to reflux for 1 h. The tan solution was cooled to room temperature and water (5 mL) was added to the white suspension. The precipitate was filtered, washed with water and dried under reduce pressure to give compound 39 4-(4-oxo-2-pyridin-2-yl-3,4-dihydro-quinazolin-6-yloxy)-piperidine-1-carboxylic acid tert-butyl ester (268 mg, 54%) as a white solid.

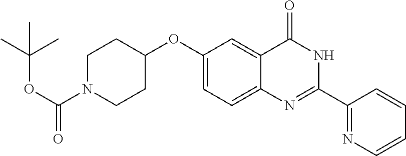 [SMILES: CC(C)(C)OC(=O)N1CCC(Oc2ccc3nc(-c4ccccn4)[nH]c(=O)c3c2)CC1]

M/Z (M+H)⁺=423.

Step 4:

To a suspension of 4-(4-oxo-2-pyridin-2-yl-3,4-dihydro-quinazolin-6-yloxy)-piperidine-1-carboxylic acid tert-butyl ester 39 (250 mg, 0.59 mmol) in dry methanol (5 mL) was added a solution of hydrogen chloride (4 M in dioxane, 4.44 mL, 17.75 mmol) and the cloudy yellow solution was stirred at room temperature overnight. Diethyl ether was added to the yellow suspension and the precipitate was collected by filtration, washed with diethyl ether and dried under reduced pressure to give 6-(piperidin-4-yloxy)-2-pyridin-2-yl-3H-quinazolin-4-one hydrochloride 40 (266 mg, quantitative yield) as a yellow solid.

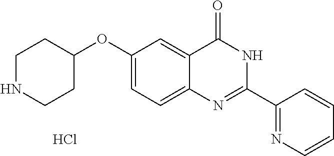 [SMILES: O=c1[nH]c(-c2ccccn2)nc2ccc(OC3CCNCC3)cc12 ; Cl]

M/Z (M+H)⁺=323.

Step 5:

At 0° C., to a solution of 6-(piperidin-4-yloxy)-2-pyridin-2-yl-3H-quinazolin-4-one hydrochloride 40 (234 mg, 0.59 mmol) and triethylamine (330 μL, 2.37 mmol) in dry dichloromethane (15 mL) was added dropwise a solution of acetyl chloride (46 μL, 0.65 mmol) in dry dichloromethane (5 mL) and the white suspension was stirred at room temperature for 2 h. The reaction mixture was poured in aqueous HCl (0.2N, 50 mL), extracted with dichloromethane (3×25 mL), the combined organic phase was dried over anhydrous sodium sulfate, filtered and concentrated under reduced pressure to give compound 41 (189 mg, 87%) as a white solid.

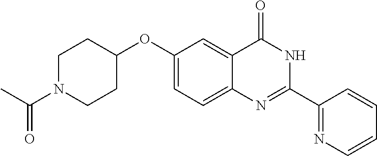 [SMILES: CC(=O)N1CCC(Oc2ccc3nc(-c4ccccn4)[nH]c(=O)c3c2)CC1]

¹H-NMR (400 MHz, DMSO): 1.55 (m, 1H, CH); 1.68 (m, 1H, CH); 1.99 (m, 5H, CH₃+2 CH); 3.26 (m, 1H, CH); 3.40 (ddd, J 13.3, 8.0, 3.4 Hz, 1H, CH); 3.71 (m, 1H, CH); 3.85 (m, 1H, CH); 4.84 (ddd, J 11.7, 7.9, 3.8 Hz, 1H, CH); 7.52 (dd, J 8.9, 3.0 Hz, 1H, Ar); 7.63 (m, 2H, Ar); 7.77 (d, J 8.9 Hz, 1H, Ar); 8.06 (td, J 7.7, 1.7 Hz, 1H, Ar); 8.42 (dt, J 8.0, 0.9 Hz, 1H, Ar); 8.75 (ddd, J 4.8, 1.6, 0.9 Hz, 1H, Ar); 11.74 (s, 1H, NH). M/Z (M+H)⁺=365.

### Example 10—Synthesis of compounds 42 (4-[4-Oxo-2-(4-trifluoromethyl-pyridin-2-yl)-3,4-dihydro-quinazolin-6-yloxymethyl]-piperidine-1-carboxylic acid tert-butyl ester), 43 (6-(piperidin-4-ylmethoxy)-2-(4-trifluoromethyl-pyridin-2-yl)-3H-quinazolin-4-one hydrochloride) and 44 (6-(1-Acetyl-piperidin-4-ylmethoxy)-2-(4-trifluoromethyl-pyridin-2-yl)-3H-quinazolin-4-one)

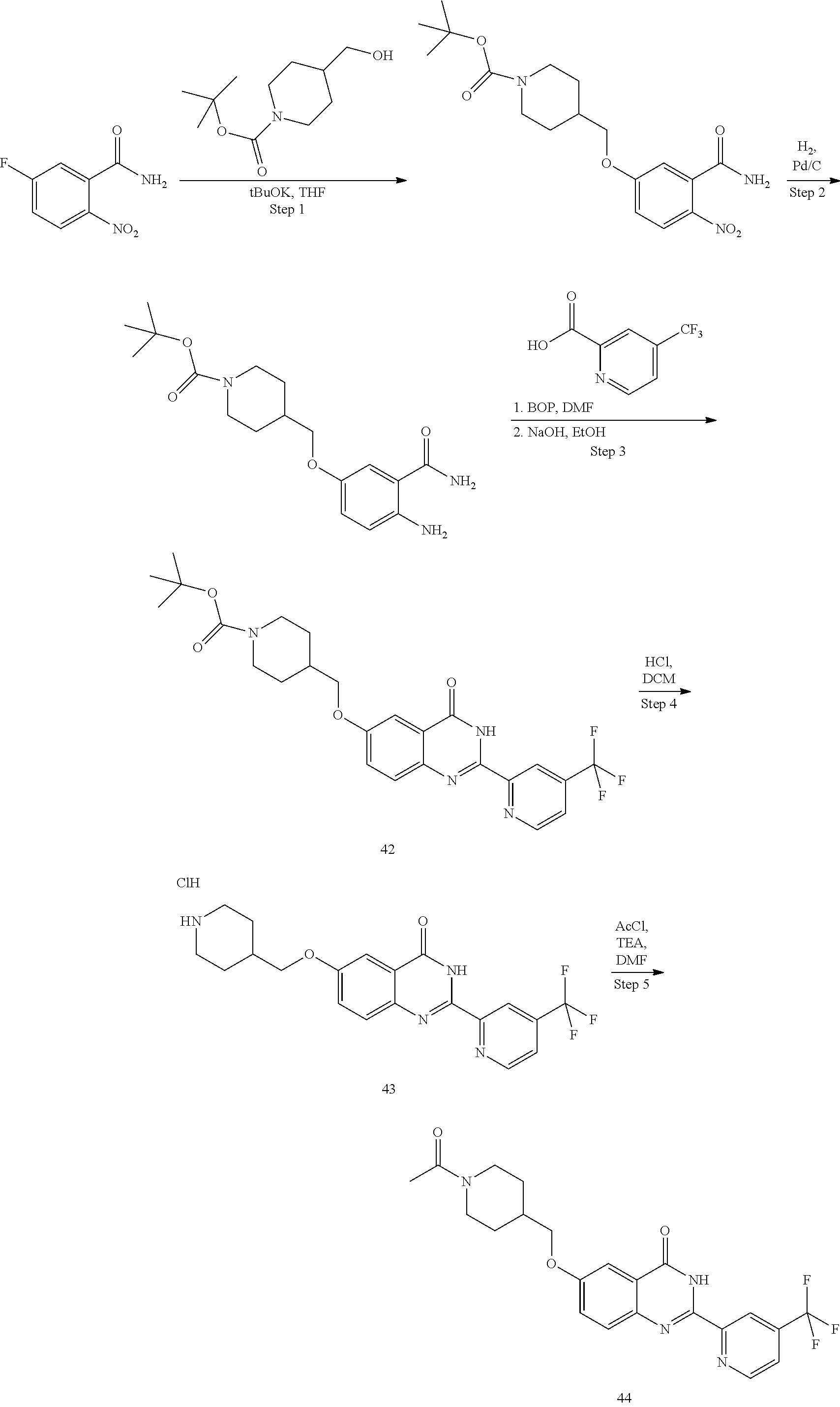

Step 1:

At 0° C., to a solution of 1-boc-4-piperidinemethanol (1.40 g, 6.52 mmol) in dry THF (30 mL) was added potassium tert-butoxide (1.60 g, 12.0 mmol) portionwise. The yellow suspension was stirred at 0° C. for 15 minutes before addition of 5-fluoro-2-nitrobenzamide (1.00 g, 5.43 mmol). The reaction mixture was stirred at room temperature for 20 minutes before being poured onto aqueous ammonium chloride (100 mL) and extracted with dichloromethane (3×50 mL). The combined organic phase was washed with brine (50 mL), dried over anhydrous sodium sulfate, filtered and concentrated under reduced pressure. The crude residue was purified by column chromatography on silica gel, using hexane/ethyl acetate as eluent, to give 4-(3-carbamoyl-4-nitro-phenoxymethyl)-piperidine-1-carboxylic acid tert-butyl ester (1.80 g, 87%) as a white solid.

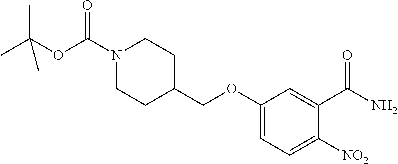 [SMILES: CC(C)(C)OC(=O)N1CCC(COc2ccc([N+](=O)[O-])c(C(N)=O)c2)CC1]

M/Z (M+Na)⁺=402.0.

Step 2:

A suspension of 4-(3-carbamoyl-4-nitro-phenoxymethyl)-piperidine-1-carboxylic acid tert-butyl ester (1.80 g, 4.74 mmol) and 10% palladium on charcoal (505 mg, 0.47 mmol) in ethanol (50 mL) was placed under hydrogen atmosphere (5 bars) and stirred at room temperature for 3 h. The reaction mixture was filtered through celite and the filtrate was concentrated to dryness to give 4-(4-amino-3-carbamoyl-phenoxymethyl)-piperidine-1-carboxylic acid tert-butyl ester (1.75 g, quantitative yield) as a brown solid.

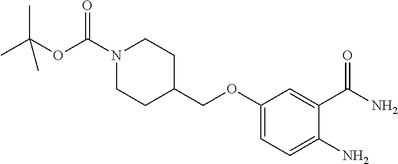 [SMILES: CC(C)(C)OC(=O)N1CCC(COc2ccc(N)c(C(N)=O)c2)CC1]

M/Z (M+Na)⁺=372.5.

Step 3:

Compound 42 was prepared according to procedure of example 1, step 4, starting from 4-(4-amino-3-carbamoyl-phenoxymethyl)-piperidine-1-carboxylic acid tert-butyl ester (400 mg, 1.14 mmol) and 4-(trifluoromethyl)pyridine-2-carboxylic acid (241 mg, 1.26 mmol). It was obtained as a beige solid (580 mg, 91%).

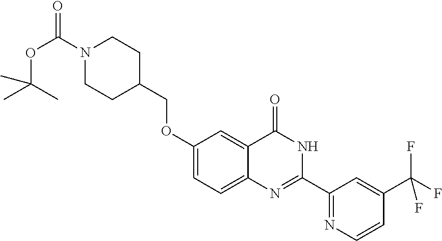 [SMILES: CC(C)(C)OC(=O)N1CCC(COc2ccc3nc(-c4cc(C(F)(F)F)ccn4)[nH]c(=O)c3c2)CC1]

M/Z (M+H)⁺=505.0.

Step 4:

Compounds 43 was prepared according to procedure of example 4, step 4 starting from compound 42 (300 mg, 0.59 mmol). Trituration in Et₂O afforded compound 43 (280 mg, quantitative yield) as a yellow solid.

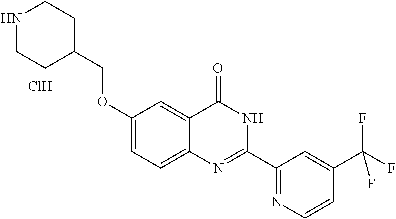 [SMILES: O=c1[nH]c(-c2cc(C(F)(F)F)ccn2)nc2ccc(OCC3CCNCC3)cc12 ; Cl]

M/Z (M+H)⁺=404.9.

Step 5:

At 0° C., to a solution of compound 43 (110 mg, 0.25 mmol) and triethylamine (100 μL, 0.75 mmol) in dry DMF (2.5 mL) was added dropwise a solution of acetyl chloride (27 μL, 0.38 mmol). The reaction mixture was stirred at room temperature for 16 h before being poured in water (50 mL) and extracted with dichloromethane (3×25 mL). The combined organic extract was dried over anhydrous sodium sulfate, filtered and concentrated under reduced pressure. Purification by column chromatography on silica gel, using dichloromethane/methanol as eluent afforded compound 44 (40 mg, 36%) as a beige solid.

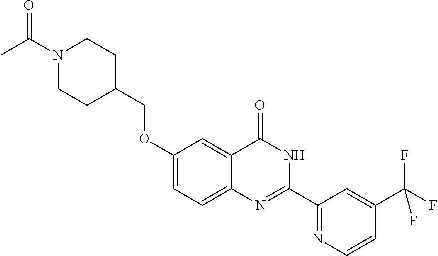 [SMILES: CC(=O)N1CCC(COc2ccc3nc(-c4cc(C(F)(F)F)ccn4)[nH]c(=O)c3c2)CC1]

¹H-NMR (400 MHz, DMSO): 1.17 (m, 1H, CH); 1.30 (m, 1H, CH); 1.82 (m, 1H, CH); 2.04 (m, 1H, CH); 2.00 (s, 3H, CH₃); 2.06 (m, 1H, CH); 2.57 (m, 1H, CH); 3.07 (m, 1H, CH); 3.86 (m, 1H, CH); 4.01 (d, J 6.4 Hz, 2H, CH₂—O); 4.42 (m, 1H, CH); 7.49 (dd, J 9.0, 3.0 Hz, 1H, Ar); 7.58 (d, J 3.0 Hz, 1H, Ar); 7.82 (d, J 9.0 Hz, 1H, Ar); 8.03 (dd, J 5.1, 1.1 Hz, 1H, Ar); 8.60 (s, 1H, Ar); 9.02 (d, J 5.1 Hz, 1H, Ar); 12.09 (br s, 1H, NH). M/Z (M+H)⁺=447.0. MP=197-199° C.

### Compound 45 (tert-butyl 4-[(4-oxo-2-thieno[2,3-c]pyridin-5-yl-3H-quinazolin-6-yl)oxymethyl]piperidine-1-carboxylate)

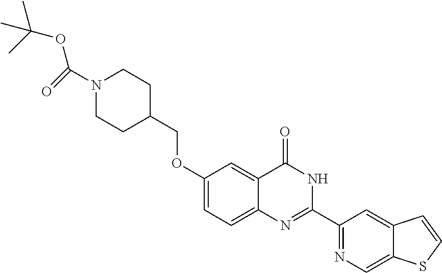 [SMILES: CC(C)(C)OC(=O)N1CCC(COc2ccc3nc(-c4cc5ccsc5cn4)[nH]c(=O)c3c2)CC1]

Compound 45 was prepared according to procedure of example 10, step 1 to 3, starting from thieno[2,3-c]pyridine-5-carboxylic acid in step 3. It was obtained as a beige solid.

M/Z (M+H)⁺=493.0

### Compound 46 (6-(4-piperidylmethoxy)-2-thieno[2,3-c]pyridin-5-yl-3H-quinazolin-4-one hydrochloride)

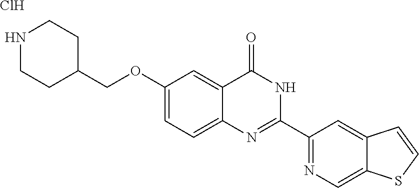 [SMILES: Cl ; O=c1[nH]c(-c2cc3ccsc3cn2)nc2ccc(OCC3CCNCC3)cc12]

Compound 46 was prepared from compound 45 according to procedure of example 10, step 4. It was obtained as a yellow solid (130 mg, quantitative yield).

M/Z (M+H)⁺=392.9

### Compound 47 (6-(1-Acetyl-piperidin-4-ylmethoxy)-2-thieno[2,3-c]pyridin-5-yl-3H-quinazolin-4-one)

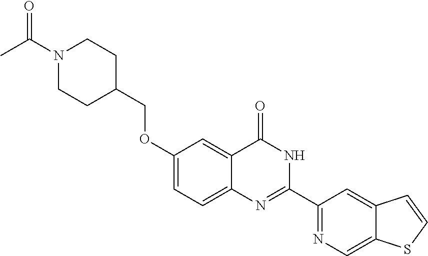 [SMILES: CC(=O)N1CCC(COc2ccc3nc(-c4cc5ccsc5cn4)[nH]c(=O)c3c2)CC1]

Compound 47 was prepared according to procedure of example 10, step 5, starting from 46 to give the product as a white solid (21 mg, 33%).

¹H-NMR (400 MHz, DMSO): 1.23 (m, 2H, 2 CH); 1.83 (m, 2H, 2 CH); 2.00 (s, 3H, CH₃); 2.06 (m, 1H, CH); 2.57 (m, 1H, CH); 3.07 (m, 1H, CH); 3.86 (m, 1H, CH); 4.01 (d, J 6.4 Hz, 2H, CH₂—O); 4.42 (m, 1H, CH); 7.49 (dd, J 8.8, 2.9 Hz, 1H, Ar); 7.58 (d, J 2.9 Hz, 1H, Ar); 7.76 (d, J 8.8 Hz, 1H, Ar); 7.79 (d, J 5.4 Hz, 1H, Ar); 8.28 (d, J 5.4 Hz, 1H, Ar); 8.93 (m, 1H, Ar); 9.44 (m, 1H, Ar); 11.76 (s, 1H, NH). M/Z (M+H)⁺=435.0. MP>250° C.

### Compound 48 (6-(1-Propionyl-piperidin-4-ylmethoxy)-2-thieno[2,3-c]pyridin-5-yl-3H-quinazolin-4-one)

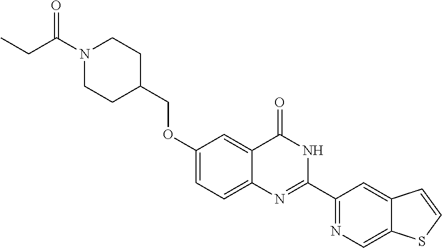 [SMILES: CCC(=O)N1CCC(COc2ccc3nc(-c4cc5ccsc5cn4)[nH]c(=O)c3c2)CC1]

Compound 48 was prepared according to procedure of example 10, starting from propionyl chloride and compound 46 in step 5 to give the product as a white solid (34 mg, 52%).

¹H-NMR (400 MHz, DMSO): 0.99 (t, J 7.5 Hz, 3H, ethyl); 1.22 (m, 2H, 2 CH); 1.83 (m, 2H, 2 CH); 2.07 (m, 1H, CH); 2.32 (q, J 7.4 Hz, 2H, ethyl); 2.60 (m, 1H, CH); 3.03 (m, 1H, CH); 3.90 (m, 1H, CH); 4.01 (d, J 6.4 Hz, 2H, CH₂—O); 4.44 (m, 1H, CH); 7.48 (dd, J 9.0, 2.9 Hz, 1H, Ar); 7.58 (d, J 2.9 Hz, 1H, Ar); 7.76 (d, J 9.0 Hz, 1H, Ar); 7.79 (d, J 5.4 Hz, 1H, Ar); 8.28 (d, J 5.4 Hz, 1H, Ar); 8.93 (m, 1H, Ar); 9.44 (m, 1H, Ar); 11.76 (s, 1H, NH). M/Z (M+H)⁺=449.0. MP=234-239° C.

### Example 11—Synthesis of compounds 49 (3-(4-Oxo-2-thieno[2,3-c]pyridin-5-yl-3,4-dihydro-quinazolin-6-yloxy)-pyrrolidine-1-carboxylic acid tert-butyl ester), 50 (6-(Pyrrolidin-3-yloxy)-2-thieno[2,3-c]pyridin-5-yl-3H-quinazolin-4-one hydrochloride) and 51 (6-(1-Acetyl-pyrrolidin-3-yloxy)-2-thieno[2,3-c]pyridin-5-yl-3H-quinazolin-4-one)

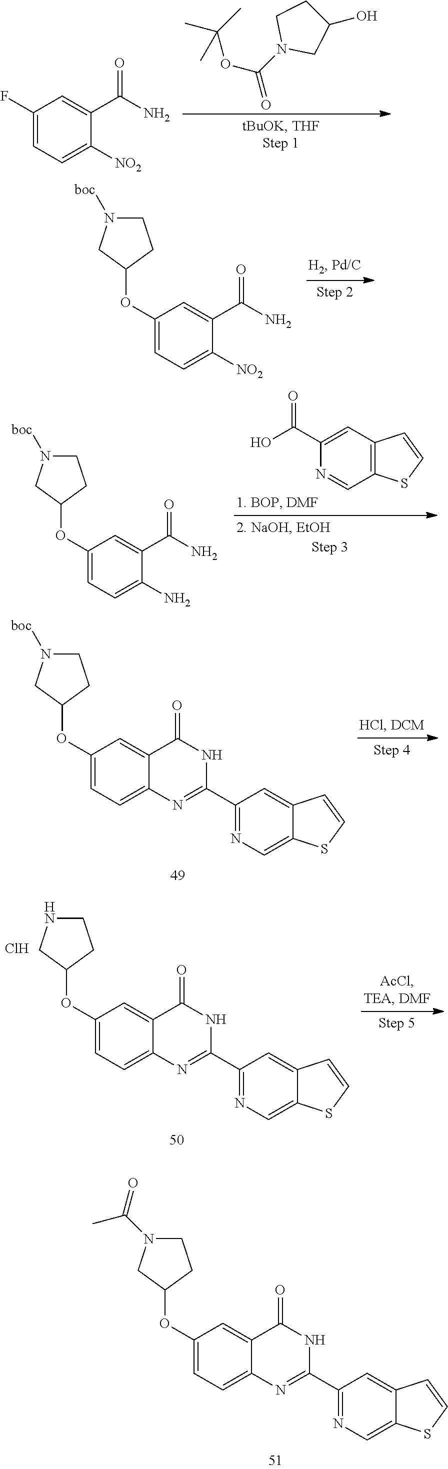

Step 1:

3-(3-Carbamoyl-4-nitro-phenoxy)-pyrrolidine-1-carboxylic acid tert-butyl ester was prepared according to procedure of example 10, step 1, starting from 1-boc-3-pyrrolidinol (610 mg, 3.26 mmol) and 5-fluoro-2-nitrobenzamide (500 mg, 2.71 mmol). It was obtained as a beige solid (700 mg, 73%).

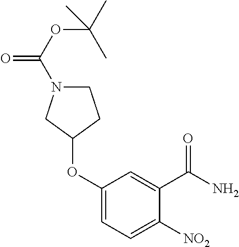 [SMILES: CC(C)(C)OC(=O)N1CCC(Oc2ccc([N+](=O)[O-])c(C(N)=O)c2)C1]

M/Z (M+Na)⁺=374.0.

Step 2:

3-(4-Amino-3-carbamoyl-phenoxy)-pyrrolidine-1-carboxylic acid tert-butyl ester was prepared according to procedure of example 10, step 2, starting from 3-(3-carbamoyl-4-nitro-phenoxy)-pyrrolidine-1-carboxylic acid tert-butyl ester (700 mg, 1.99 mmol). It was purified by column chromatography on silica gel, using hexane/ethyl acetate as eluent, to give 3-(4-amino-3-carbamoyl-phenoxy)-pyrrolidine-1-carboxylic acid tert-butyl ester as a yellow solid (700 mg, quantitative yield).

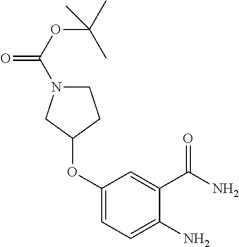 [SMILES: CC(C)(C)OC(=O)N1CCC(Oc2ccc(N)c(C(N)=O)c2)C1]

M/Z (M+Na)⁺=344.0.

Step 3:

Compound 49 was prepared according to procedure of example 1, step 4, starting from 3-(4-amino-3-carbamoyl-phenoxy)-pyrrolidine-1-carboxylic acid tert-butyl ester (350 mg, 1.09 mmol) and thieno[2,3-c]pyridine-5-carboxylic acid (240 mg, 1.20 mmol). It was obtained as a beige solid (317 mg, 62%).

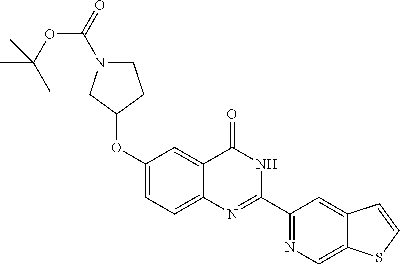 [SMILES: CC(C)(C)OC(=O)N1CCC(Oc2ccc3nc(-c4cc5ccsc5cn4)[nH]c(=O)c3c2)C1]

M/Z (M+H)⁺=465.0.

Step 4:

Compound 50 was prepared according to procedure of example 10, step 4, starting from compound 49 (277 mg, 0.60 mmol). It was obtained as a yellow solid (247 mg, quantitative yield).

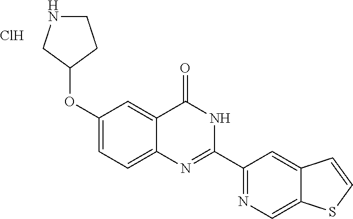 [SMILES: Cl ; O=c1[nH]c(-c2cc3ccsc3cn2)nc2ccc(OC3CCNC3)cc12]

M/Z (M+H)⁺=365.0.

Step 5:

Compound 51 was prepared according to procedure of example 10, step 5, starting from compound 50 (100 mg, 0.25 mmol). It was obtained as a white solid (50 mg, 50%).

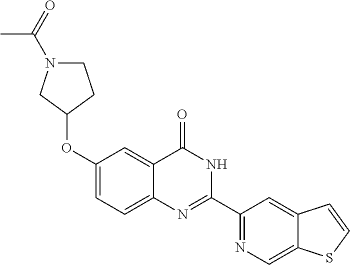 [SMILES: CC(=O)N1CCC(Oc2ccc3nc(-c4cc5ccsc5cn4)[nH]c(=O)c3c2)C1]

¹H-NMR (400 MHz, DMSO): 1.99 (s, 3H, CH₃); 2.21 (m, 2H, 2 CH); 3.58 (m, 2H, 2 CH); 3.67 (m, 2H, 2 CH); 5.22 (m, 1H, CH-0); 7.48 (dd, J 9.0, 2.9 Hz, 1H, Ar); 7.60 (d, J 2.9 Hz, 1H, Ar); 7.77 (m, 2H, Ar); 8.28 (d, J 5.4 Hz, 1H, Ar); 8.93 (m, 1H, Ar); 9.44 (m, 1H, Ar); 11.79 (s, 1H, NH). M/Z (M+H)⁺=407.0. MP>250° C.

### Example 12—Synthesis of compounds 52 (4-[4-Oxo-2-(4-trifluoromethyl-pyridin-2-yl)-3,4-dihydro-quinazolin-6-yl]-piperazine-1-carboxylic acid tert-butyl ester), 53 (6-Piperazin-1-yl-2-(4-trifluoromethyl-pyridin-2-yl)-3H-quinazolin-4-one hydrochloride) and 54 (6-(4-Propionyl-piperazin-1-yl)-2-(4-trifluoromethyl-pyridin-2-yl)-3H-quinazolin-4-one)

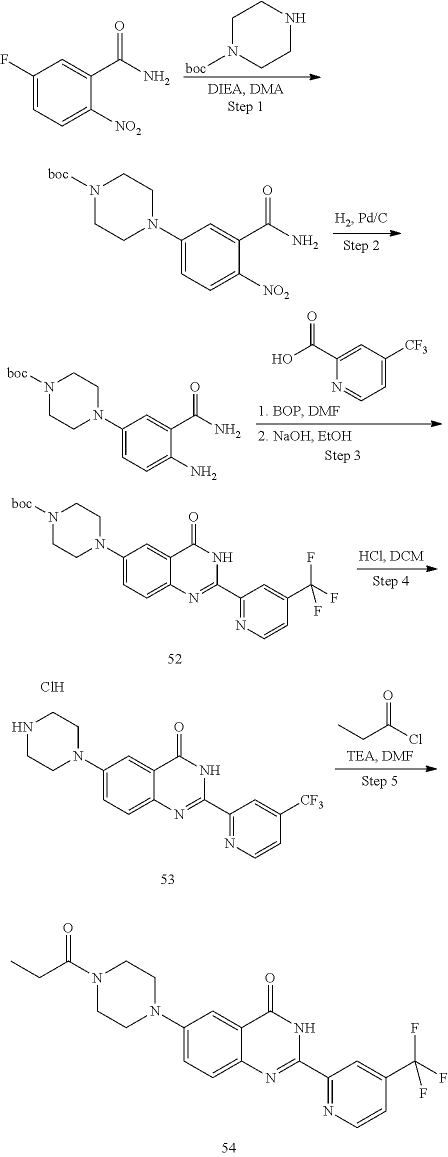

Step 1:

A solution of 5-fluoro-2-nitrobenzamide (300 mg, 1.63 mmol), 1-boc-piperazine (364 mg, 1.95 mmol) and diisopropylethylamine (620 μL, 3.58 mmol) in DMA (16 mL) was heated at 130° C. for 16 h. After cooling to room temperature, the reaction mixture was poured into an aqueous solution of ammonium chloride (200 mL) and extracted with ethyl acetate (200 mL). The combined organic extract was washed with brine (50 mL), dried over sodium sulfate and concentrated under vacuum. Purification by column chromatography on silica gel, using hexane/ethyl acetate as eluent, afforded 4-(3-carbamoyl-4-nitro-phenyl)-piperazine-1-carboxylic acid tert-butyl ester as a yellow solid (600 mg, quantitative yield).

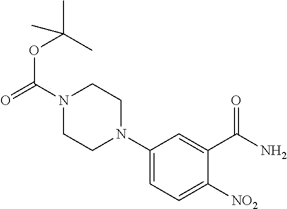 [SMILES: CC(C)(C)OC(=O)N1CCN(c2ccc([N+](=O)[O-])c(C(N)=O)c2)CC1]

M/Z (M+Na)⁺=372.9.

Step 2:

4-(4-Amino-3-carbamoyl-phenyl)-piperazine-1-carboxylic acid tert-butyl ester was prepared according to procedure of example 10, step 2, starting from 4-(3-carbamoyl-4-nitro-phenyl)-piperazine-1-carboxylic acid tert-butyl ester. Purification by column chromatography on silica gel, using hexane/ethyl acetate as eluent, afforded the product as a yellow solid (468 mg, 67%).

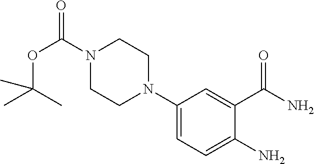 [SMILES: CC(C)(C)OC(=O)N1CCN(c2ccc(N)c(C(N)=O)c2)CC1]

M/Z (M+H)⁺=320.1.

Step 3:

Compound 52 was prepared according to procedure of example 1, step 4, starting from 4-(4-amino-3-carbamoyl-phenyl)-piperazine-1-carboxylic acid tert-butyl ester (390 mg, 1.15 mmol) and 4-(trifluoromethyl)pyridine-2-carboxylic acid (241 mg, 1.26 mmol). It was obtained as a yellow solid (400 mg, 86%).

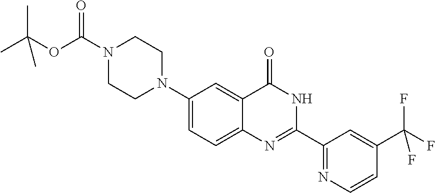 [SMILES: CC(C)(C)OC(=O)N1CCN(c2ccc3nc(-c4cc(C(F)(F)F)ccn4)[nH]c(=O)c3c2)CC1]

M/Z (M+H)⁺=476.2.

Step 4:

Compound 53 was prepared according to procedure of example 10, step 4, starting from 4-[4-oxo-2-(4-trifluoromethyl-pyridin-2-yl)-3,4-dihydro-quinazolin-6-yl]-piperazine-1-carboxylic acid tert-butyl ester 52 (400 mg, 0.84 mmol). It was obtained as a red solid (400 mg, quantitative yield).

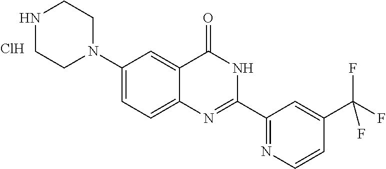 [SMILES: Cl ; O=c1[nH]c(-c2cc(C(F)(F)F)ccn2)nc2ccc(N3CCNCC3)cc12]

M/Z (M+H)⁺=376.0.

Step 5:

Compound 54 was prepared according to procedure of example 10, step 5, starting from compound 53 (100 mg, 0.24 mmol) and propionyl chloride (32 μL, 0.36 mmol) to afford the product as a yellow solid (65 mg, 62%).

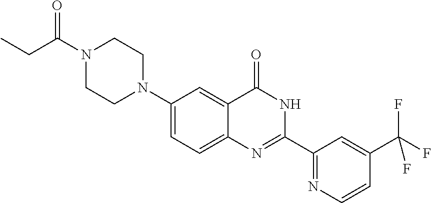 [SMILES: CCC(=O)N1CCN(c2ccc3nc(-c4cc(C(F)(F)F)ccn4)[nH]c(=O)c3c2)CC1]

¹H-NMR (400 MHz, DMSO): 1.02 (t, J 7.3 Hz, 3H, ethyl); 2.38 (q, J 7.3 Hz, 2H, ethyl); 3.37 (m, 4H, 2 CH₂); 3.64 (m, 4H, 2 CH₂); 7.50 (d, J 2.7 Hz, 1H, Ar); 7.63 (dd, J 2.7, 9.4 Hz, 1H, Ar); 7.77 (d, J 9.4 Hz, 1H, Ar); 8.00 (d, J 5.0 Hz, 1H, Ar); 8.59 (s, 1H, Ar); 9.01 (d, J 5.0 Hz, 1H, Ar); 11.89 (s, 1H, NH). M/Z (M+H)⁺=432.0. MP>250° C.

### Example 13—Synthesis of compounds 55 (4-(4-oxo-2-thieno[2,3-c]pyridin-5-yl-3,4-dihydro-quinazolin-6-yl)-piperidine-1-carboxylic acid tert-butyl ester), 56 (6-Piperidin-4-yl-2-thieno[2,3-c]pyridin-5-yl-3H-quinazolin-4-one hydrochloride) and 57 (6-(1-acetyl-piperidin-4-yl)-2-thieno[2,3-c]pyridin-5-yl-3H-quinazolin-4-one)

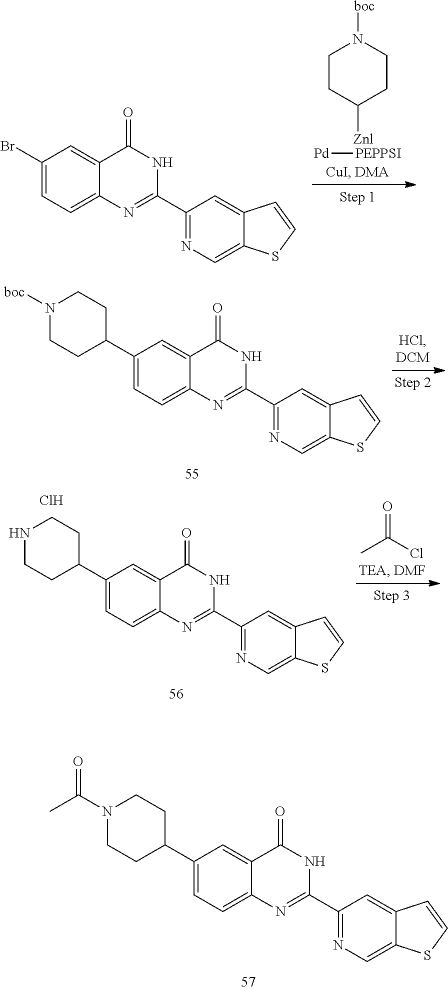

Step 1:

Under inert atmosphere, at 0° C., to a suspension of zinc dust (126 mg, 8.24 mmol) in dry DMA (0.7 mL) were added trimethylsilylchloride (28 μL, 0.22 mmol) and 1,2-dibromoethane (20 μL, 0.23 mmol) successively. The resulting slurry was stirred at room temperature for 15 minutes before addition of a solution of tert-butyl-4-iodopiperidine-1-carboxylate (770 mg, 2.47 mmol) in dry DMA (2.1 mL). The reaction mixture was stirred at room temperature for 30 minutes. Then, a solution of 6-bromo-2-thieno[2,3-c]pyridin-5-yl-3H-quinazolin-4-one (350 mg, 0.98 mmol) in dry DMA (9.0 mL), copper iodide (19 mg, 0.10 mmol) and Pd-PEPPSI-IPentCI-o-pinacoline (41 mg, 0.05 mmol) were added to the reaction mixture, which was then heated at 80° C. for 16 h. The reaction mixture was poured into cold water (50 mL) and the resulting grey precipitate was collected by filtration. Purification by column chromatography on silica gel, using hexane/ethyl acetate as eluent, afforded 4-(4-oxo-2-thieno[2,3-c]pyridin-5-yl-3,4-dihydro-quinazolin-6-yl)-piperidine-1-carboxylic acid tert-butyl ester 55 (350 mg, 77%) as a white solid.

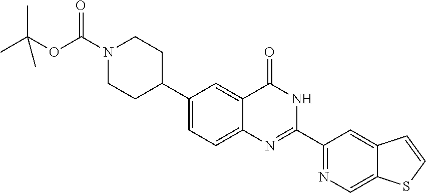 [SMILES: CC(C)(C)OC(=O)N1CCC(c2ccc3nc(-c4cc5ccsc5cn4)[nH]c(=O)c3c2)CC1]

M/Z (M+H)⁺=463.0.

Step 2:

Compound 56 was prepared according to procedure of example 10, step 4, starting from compound 55 (350 mg, 0.76 mmol). It was obtained as a brown solid (150 mg, 50%).

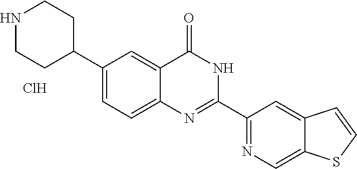 [SMILES: O=c1[nH]c(-c2cc3ccsc3cn2)nc2ccc(C3CCNCC3)cc12 ; Cl]

M/Z (M+H)⁺=362.9.

Step 3:

Compound 57 was prepared according to procedure of example 10, step 5, starting from compound 55 (75 mg, 0.19 mmol) and acetyl chloride (20 μL, 0.28 mmol) to afford the product as a beige solid (15 mg, 20%).

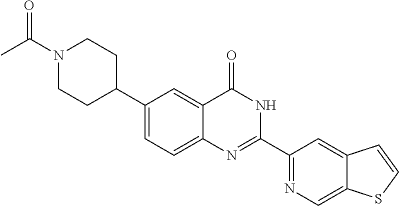 [SMILES: CC(=O)N1CCC(c2ccc3nc(-c4cc5ccsc5cn4)[nH]c(=O)c3c2)CC1]

¹H-NMR (400 MHz, DMSO): 1.50 (m, 1H, CH); 1.69 (m, 1H, CH); 1.88 (m, 2H, 2 CH); 2.05 (s, 3H, CH₃); 2.63 (m, 1H, CH); 2.98 (m, 1H, CH); 3.17 (m, 1H, CH); 3.96 (m, 1H, CH); 4.57 (m, 1H, CH); 7.78 (m, 3H, Ar); 8.03 (d, J 1.8 Hz, 1H, Ar); 8.29 (d, J 5.3 Hz, 1H, Ar); 8.97 (s, 1H, Ar); 9.45 (s, 1H, Ar); 11.77 (s, 1H, NH). M/Z (M+H)⁺=404.9. MP=240-247° C.

### Example 14—Synthesis of compound 58 (6-[2-(Tetrahydro-pyran-4-yl)-ethoxy]-2-(4-trifluoromethyl-pyridin-2-yl)-3H-quinazolin-4-one)

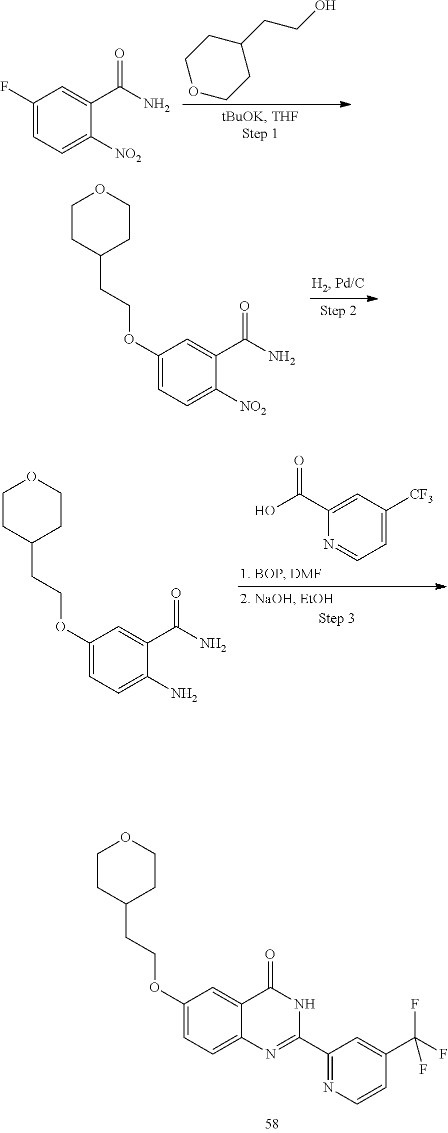

Step 1:

2-nitro-5-[2-(tetrahydro-pyran-4-yl)-ethoxy]-benzamide was prepared according to procedure of example 10, step 1, starting from 2-(tetrahydro-2H-pyran-4-yl)ethan-1-ol and 5-fluoro-2-nitrobenzamide (200 mg, 1.09 mmol). The product (273 mg, 85%) was obtained without purification as an orange oil.

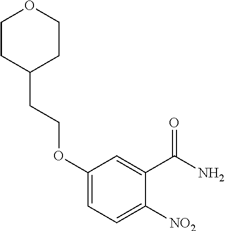 [SMILES: NC(=O)c1cc(OCCC2CCOCC2)ccc1[N+](=O)[O-]]

M/Z (M+H)⁺=295.0.

Step 2:

2-Amino-5-[2-(tetrahydro-pyran-4-yl)-ethoxy]-benzamide (273 mg, 0.93 mmol) was prepared according to procedure of example 10, step 2, starting from 2-nitro-5-[2-(tetrahydro-pyran-4-yl)-ethoxy]-benzamide (246 mg, quantitative yield).

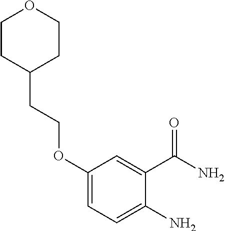 [SMILES: NC(=O)c1cc(OCCC2CCOCC2)ccc1N]

M/Z (M+H)⁺=265.0.

Step 3:

Compound 58 was prepared according to procedure of example 1, step 4, starting from 2-amino-5-[2-(tetrahydro-pyran-4-yl)-ethoxy]-benzamide (60 mg, 0.23 mmol) and 4-(trifluoromethyl)pyridine-2-carboxylic acid (48 mg, 0.25 mmol). It was obtained as a yellow solid (32 mg, 44%).

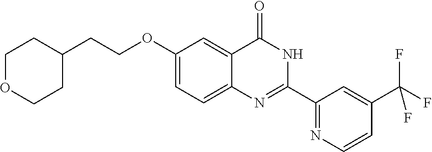 [SMILES: O=c1[nH]c(-c2cc(C(F)(F)F)ccn2)nc2ccc(OCCC3CCOCC3)cc12]

¹H-NMR (400 MHz, DMSO): 1.25 (m, 2H, 2 CH); 1.68 (m, 5H, 5CH); 3.29 (m, 2H, 2 CH); 3.84 (m, 2H, 2 CH); 4.12 (t, J 6.2 Hz, 2H, CH₂—O); 7.27 (dd, J 8.8, 2.8 Hz, 1H, Ar); 7.50 (d, J 2.8 Hz, 1H, Ar); 7.65 (d, J 8.8 Hz, 1H, Ar); 7.84 (d, J 5.0 Hz, 1H, Ar); 8.65 (s, 1H, Ar); 8.95 (d, J 5.0 Hz, 1H, Ar). NH signal not observed. M/Z (M+H)⁺=420. MP>250° C.

### Example 15—Synthesis of compound 59 (6-[3-(3-Fluoro-pyridin-4-yl)-propoxy]-2-thieno[2,3-c]pyridin-5-yl-3H-quinazolin-4-one)

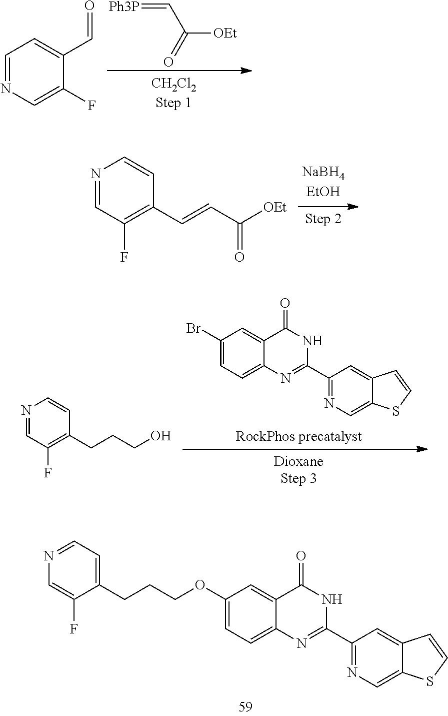

Step 1:

To a solution of 3-fluoro-4-pyridinecarbaldehyde (500 mg, 3.99 mmol) in dichloromethane (20 mL) was added (carbethoxymethylene)triphenylphosphorane (1.5 g, 4.40 mmol) and the reaction mixture was stirred at room temperature for 16 h. The reaction mixture was concentrated under reduced pressure and purified by column chromatography on silica gel, using hexane/ethyl acetate as eluent, to give 3-(3-fluoro-pyridin-4-yl)-acrylic acid ethyl ester (760 mg, 97%) as a white solid.

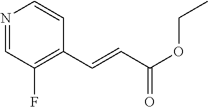

M/Z (M+H)⁺=195.8.

Step 2:

Under dry conditions, at 0° C., to a solution of 3-(3-fluoro-pyridin-4-yl)-acrylic acid ethyl ester (760 mg, 3.89 mmol) in ethanol (20 mL) was added sodium borohydride (1.47 g, 38.9 mmol). The reaction mixture was stirred at room temperature for 16 h. A second addition of sodium borohydride (1.47 g, 38.9 mmol) was realized to obtain a complete conversion. The reaction mixture was poured in ice water (50 mL) and extracted with dichloromethane (3×25 mL). The combined organic phase was dried over anhydrous sodium sulfate, filtered and concentrated under vacuum. Purification by column chromatography on silica gel, using hexane/ethyl acetate as eluent, afforded 3-(3-fluoro-pyridin-4-yl)-propan-1-ol (272 mg, 45%) as a colorless oil.

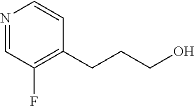 [SMILES: OCCCc1ccncc1F]

M/Z (M+H)⁺=155.9.

Step 3:

Under inert atmosphere, a mixture of 6-bromo-2-thieno[3,2-c]pyridin-6-yl-quinazolin-4-one (125 mg, 0.35 mmol), 3-(3-fluoro-pyridin-4-yl)-propan-1-ol (271 mg, 1.74 mmol), cesium carbonate (340 mg, 1.05 mmol) and RockPhos precatalyst (30 mg, 0.04 mmol) in dioxane (3.5 mL) was heated at 100° C. for 20 h in a sealed reactor. After cooling to room temperature, the reaction mixture was poured in water (20 mL) and extracted with dichloromethane (3×25 mL). The combined organic extract was dried over anhydrous sodium sulfate, filtered and concentrated under vacuum. Purification by column chromatography on silica gel, using dichloromethane/methanol as eluent, afforded compound 59 (20 mg, 13%) as a yellow solid.

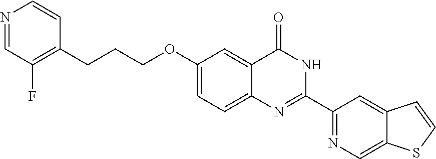 [SMILES: O=c1[nH]c(-c2cc3ccsc3cn2)nc2ccc(OCCCc3ccncc3F)cc12]

¹H-NMR (400 MHz, DMSO): 2.12 (m, 2H, CH₂); 2.88 (t, J 7.5 Hz, 2H, CH₂); 4.17 (t, J 6.1 Hz, 2H, CH₂—O); 7.45 (m, 2H, Ar); 7.56 (d, J 2.8 Hz, 1H, Ar); 7.76 (d, J 9.0 Hz, 1H, Ar); 7.79 (d, J 5.4 Hz, 1H, Ar); 8.24 (d, J 5.4 Hz, 1H, Ar); 8.35 (d, J 4.8 Hz, 1H, Ar); 8.48 (d, J 1.5 Hz, 1H, Ar); 8.93 (s, 1H, Ar); 9.44 (s, 1H, Ar); 11.76 (s, 1H, NH). M/Z (M+H)⁺=433.0. MP=240-247° C.

### Compound 60 (6-[3-(4-Methanesulfonyl-phenyl)-propoxy]-2-thieno[2,3-c]pyridin-5-yl-3H-quinazolin-4-one)

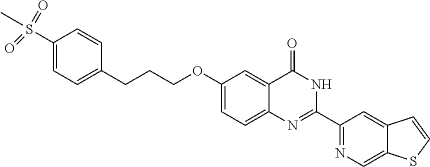 [SMILES: CS(=O)(=O)c1ccc(CCCOc2ccc3nc(-c4cc5ccsc5cn4)[nH]c(=O)c3c2)cc1]

Compound 60 was prepared according to procedure of example 15, starting from 4-(methylsulfonyl)benzaldehyde in step 1. It was purified by preparative HPLC to give a beige solid.

¹H-NMR (400 MHz, DMSO): 2.14 (m, 2H, CH₂); 2.92 (t, J 7.5 Hz, 2H, CH₂); 3.19 (s, 3H, CH₃); 4.15 (t, J 6.2 Hz, 2H, CH₂—O); 7.49 (dd, J 8.8, 2.9 Hz, 1H, Ar); 7.55 (m, 3H, Ar); 7.76 (d, J 8.8 Hz, 1H, Ar); 7.79 (d, J 5.2 Hz, 1H, Ar); 7.85 (m, 2H, Ar); 8.28 (d, J 5.2 Hz, 1H, Ar); 8.93 (s, 1H, Ar); 9.44 (s, 1H, Ar); 11.76 (s, 1H, NH). M/Z (M+H)⁺=492.1. MP=236-237° C.

### Compound 61 (6-(3-Pyrazin-2-yl-propoxy)-2-thieno[2,3-c]pyridin-5-yl-3H-quinazolin-4-one hydrochloride)

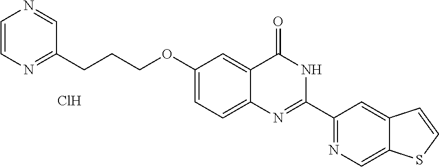 [SMILES: O=c1[nH]c(-c2cc3ccsc3cn2)nc2ccc(OCCCc3cnccn3)cc12 ; Cl]

Compound 61 was prepared according to procedure of example 15, starting from pyrazine-2-carbaldehyde in step 1. It was purified by preparative HPLC and the HCl salt was obtained by concentration to dryness after addition of an excess of HCl (1.2N in MeOH) to a solution of the product in methanol. Compound 61 was obtained as a yellow solid.

¹H-NMR (400 MHz, DMSO): 2.26 (m, 2H, CH₂); 3.02 (t, J 7.3 Hz, 2H, CH₂); 4.23 (t, J 6.3 Hz, 2H, CH₂—O); 7.44 (dd, J 9.0, 3.0 Hz, 1H, Ar); 7.61 (d, J 3.0 Hz, 1H, Ar); 7.77 (m, 2H, Ar); 8.24 (d, J 5.3 Hz, 1H, Ar); 8.46 (d, J 2.5 Hz, 1H, Ar); 8.55 (m, 1H, Ar); 8.60 (m, 1H, Ar); 8.94 (s, 1H, Ar); 9.41 (s, 1H, Ar); HCl salt signal not observed; NH signal not observed. M/Z (M+H)⁺=416.0. MP=230-240° C.

### Compound 62 (6-[3-(3-Methoxy-pyridin-4-yl)-propoxy]-2-thieno[2,3-c]pyridin-5-yl-3H-quinazolin-4-one hydrochloride)

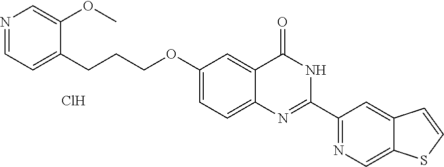 [SMILES: COc1cnccc1CCCOc1ccc2nc(-c3cc4ccsc4cn3)[nH]c(=O)c2c1 ; Cl]

Compound 62 was prepared according to procedure of example 15, starting from 3-methoxypyridine-4-carbaldehyde in step 1. It was purified by column chromatography on silica gel, using dichloromethane/methanol as eluent, and the HCl salt was obtained by filtration after addition of an excess of HCl (2N in Et₂O) to a solution of the product in dichloromethane. Compound 62 was obtained as a yellow solid.

¹H-NMR (400 MHz, DMSO): 2.16 (m, 2H, CH₂); 3.00 (t, J 7.2 Hz, 2H, CH₂); 4.00 (s, 3H, CH₃-0); 4.18 (t, J 6.1 Hz, 2H, CH₂—O); 7.43 (dd, J 8.8, 3.0 Hz, 1H, Ar); 7.55 (d, J 3.0 Hz, 1H, Ar); 7.77 (d, J 8.8 Hz, 1H, Ar); 7.79 (d, J 5.3 Hz, 1H, Ar); 7.93 (d, J 5.6 Hz, 1H, Ar); 8.29 (d, J 5.3 Hz, 1H, Ar); 8.51 (d, J 5.6 Hz, 1H, Ar); 8.58 (s, 1H, Ar); 8.93 (s, 1H, Ar); 9.45 (s, 1H, Ar); HCl salt signal not observed; NH signal not observed. M/Z (M+H)⁺=445.1. MP=250-251° C.

### Example 16—Synthesis of compound 63 (6-[3-(2-Methyl-pyridin-4-yl)-propoxy]-2-thieno[2,3-c]pyridin-5-yl-3H-quinazolin-4-one hydrochloride)

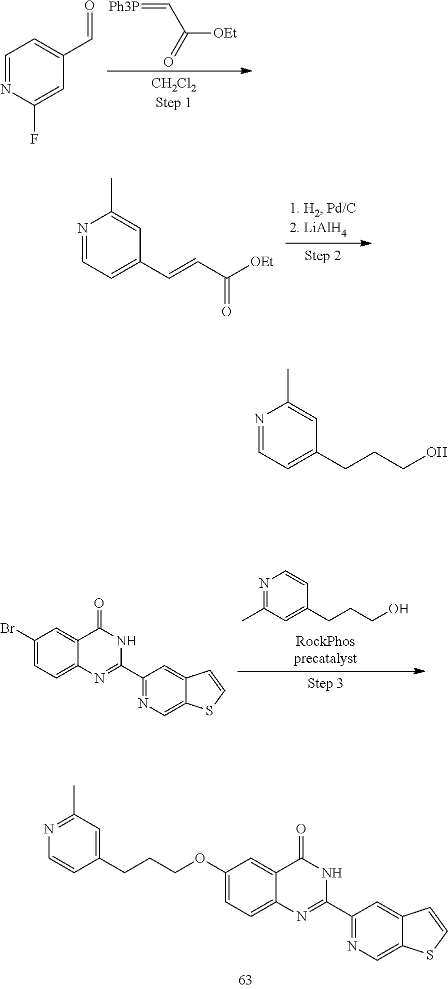

Step 1:

3-(2-Methyl-pyridin-4-yl)-acrylic acid ethyl ester was prepared according to procedure of example 15, step 1 and obtained as a yellow solid in quantitative yield.

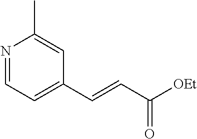

M/Z (M+H)⁺=191.8.

Step 2:

A suspension of 3-(2-methyl-pyridin-4-yl)-acrylic acid ethyl ester (785 mg, 4.13 mmol) and 10% palladium on charcoal (439 mg, 0.41 mmol) in ethanol (21 mL) was placed under hydrogen atmosphere (5 bars) and stirred for 1 h at room temperature. The reaction mixture was filtered through celite and concentrated to dryness to give a yellow oil. At 0° C., the crude yellow oil was dissolved in THF (20 mL), and a solution of lithium aluminium hydride (2M in THF, 2.9 mL, 5.86 mmol) was added dropwise. The reaction mixture was stirred at 0° C. for 1 h before being hydrolyzed by an aqueous solution of NaOH (3N, 0.5 mL). The resulting precipitate was filtered off, washed with dichloromethane, and the filtrate was concentrated to dryness to give 3-(2-methyl-pyridin-4-yl)-propan-1-ol (560 mg, 95%) as a brown oil.

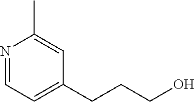

M/Z (M+H)⁺=152.0.

Step 3:

Compound 63 was prepared according to procedure of example 15, step 3, starting from 3-(2-methyl-pyridin-4-yl)-propan-1-ol (380 mg, 2.51 mmol) and 6-bromo-2-thieno[2,3-c]pyridin-5-yl-3H-quinazolin-4-one (150 mg, 0.42 mmol). It was purified by preparative HPLC and the HCl salt was obtained by concentration to dryness after addition of an excess of HCl (1.2N in MeOH) to a solution of the product in methanol. Compound 63 was obtained as a beige solid in 33% yield.

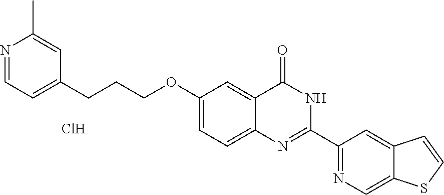 [SMILES: Cc1cc(CCCOc2ccc3nc(-c4cc5ccsc5cn4)[nH]c(=O)c3c2)ccn1 ; Cl]

¹H-NMR (400 MHz, DMSO): 2.23 (m, 2H, CH₂); 2.69 (s, 3H, CH₃); 3.04 (t, J 7.6 Hz, 2H, CH₂); 4.22 (t, J 6.2 Hz, 2H, CH₂—O); 7.45 (dd, J 8.8, 2.9 Hz, 1H, Ar); 7.60 (d, J 2.9 Hz, 1H, Ar); 7.70 (m, 1H, Ar); 7.77 (m, 3H, Ar); 8.25 (d, J 5.4 Hz, 1H, Ar); 8.59 (d, J 5.9 Hz, 1H, Ar); 8.93 (s, 1H, Ar); 9.41 (s, 1H, Ar); HCl salt signal not observed; NH signal not observed. M/Z (M+H)⁺=428.9. MP>250° C.

### Compound 64 (6-(3-Oxazol-4-yl-propoxy)-2-thieno[2,3-c]pyridin-5-yl-3H-quinazolin-4-one)

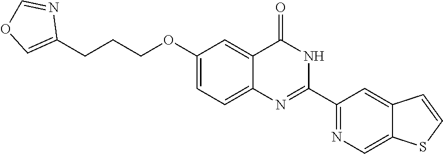 [SMILES: O=c1[nH]c(-c2cc3ccsc3cn2)nc2ccc(OCCCc3cocn3)cc12]

Compound 64 was prepared according to procedure of example 16, starting from 4-oxazole-carbaldehyde in step 1. It was purified by column chromatography on silica gel, using dichloromethane/methanol as eluent, to give a beige solid in 27% yield.

¹H-NMR (400 MHz, DMSO): 2.12 (m, 2H, CH₂); 2.71 (t, J 7.5 Hz, 2H, CH₂); 4.22 (t, J 6.4 Hz, 2H, CH₂—O); 7.48 (dd, J 9.0, 2.9 Hz, 1H, Ar); 7.61 (d, J 2.9 Hz, 1H, Ar); 7.77 (m, 2H, Ar); 7.82 (m, 1H, Ar); 8.17 (s, 1H, Ar); 8.24 (d, J 5.4 Hz, 1H, Ar); 8.94 (s, 1H, Ar); 9.40 (s, 1H, Ar); 11.38 (m, 1H, NH). M/Z (M+H)⁺=404.9. MP=197-199° C.

### Example 17—Synthesis of compound 65 (8-Methyl-6-(3-pyridin-4-yl-propoxy)-2-thieno[2,3-c]pyridin-5-yl-3H-quinazolin-4-one hydrochloride)

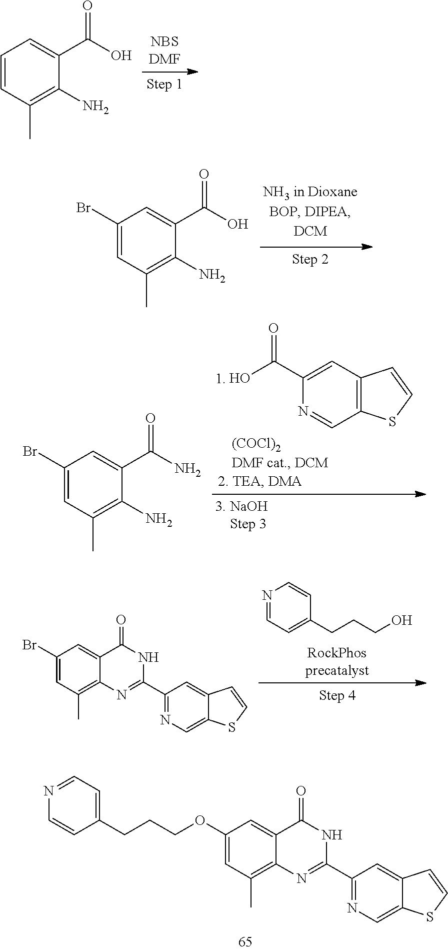

Step 1:

To a solution of 2-amino-3-methylbenzoic acid (500 mg, 3.31 mmol) in DMF (33 mL) was added N-bromosuccinimide (618 mg, 3.47 mmol). The reaction mixture was stirred at room temperature for 1 h before being poured in water (50 mL) and extracted with ethyl acetate (3×50 mL). The combined organic phase was dried over anhydrous sodium sulfate, filtered and concentrated under vacuum to give 2-amino-5-bromo-3-methyl-benzoic acid (760 ma, quantitative yield) as a brown solid.

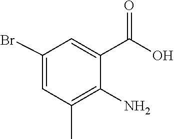

M/Z (M[⁷⁹Br]+H)⁺=230.0.

Step 2:

2-Amino-5-bromo-3-methyl-benzamide was prepared according to procedure of example 1, step 1, starting from 2-amino-5-bromo-3-methyl-benzoic acid (720 mg, 3.13 mmol) to afford 2-amino-5-bromo-3-methyl-benzamide (570 mg, 79%) as a beige solid.

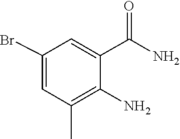

M/Z (M[⁷⁹Br]+H)⁺=229.0.

Step 3:

At 0° C., oxalyl chloride (3.2 mL, 37.2 mmol) and then DMF (46 μL, 0.60 mmol) were added dropwise to a solution of thieno[2,3-c]pyridine-5-carboxylic acid (3.58 g, 20.00 mmol) in dichloromethane (200 mL). The reaction mixture was stirred at room temperature for 1 h before being concentrated to dryness, and co-evaporated twice with toluene. The crude acyl chloride was dissolved in dimethylacetamide (144 mL), then triethylamine (5.2 mL, 37.2 mmol) and 2-amino-5-bromo-3-methyl-benzamide (2.84 g, 12.4 mmol) were added and the reaction mixture was stirred at room temperature for 1 h. Then aqueous NaOH (1 N, 74.4 mL) was added and the reaction mixture was heated at 100° C. for 1 h. The suspension was then allowed to cool down to room temperature and an aqueous saturated solution of NH₄Cl (150 mL) was slowly added. The resulting beige solid was collected by filtration and rinsed thoroughly with water. It was then dried in vacuo over P₂O₅ for 2 days to afford 6-bromo-8-methyl-2-thieno[2,3-c]pyridin-5-yl-3H-quinazolin-4-one (2.53 g, 55%) as a beige solid.

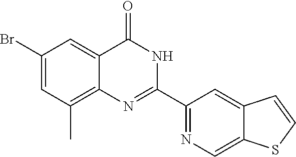 [SMILES: Cc1cc(Br)cc2c(=O)[nH]c(-c3cc4ccsc4cn3)nc12]

M/Z (M[⁷⁹Br]+H)⁺=372.0.

Step 4:

8-Methyl-6-(3-pyridin-4-yl-propoxy)-2-thieno[2,3-c]pyridin-5-yl-3H-quinazolin-4-one was obtained according to the procedure of example 15, step 3, starting from 6-bromo-8-methyl-2-thieno[2,3-c]pyridin-5-yl-3H-quinazolin-4-one (100 mg, 0.27 mmol) and 4-pyridinepropanol (220 mg, 1.61 mmol). It was purified by column chromatography on silica gel, using dichloromethane/methanol as eluent, and the HCl salt was obtained by concentration to dryness after addition of an excess of HCl (2N in Et₂O) to a solution of the product in dichloromethane. Compound 65 was obtained as a yellow solid in 28% yield.

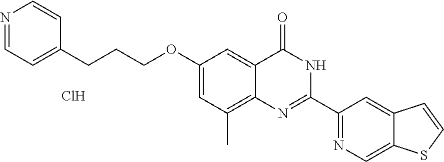 [SMILES: Cc1cc(OCCCc2ccncc2)cc2c(=O)[nH]c(-c3cc4ccsc4cn3)nc12 ; Cl]

¹H-NMR (400 MHz, DMSO): 2.22 (m, 2H, CH₂); 2.68 (s, 3H, CH₃); 3.11 (t, J 7.2 Hz, 2H, CH₂); 4.16 (t, J 6.4 Hz, 2H, CH₂—O); 7.32 (d, J 2.4 Hz, 1H, Ar); 7.41 (d, J 2.4 Hz, 1H, Ar); 7.81 (d, J 5.4 Hz, 1H, Ar); 8.01 (d, J 6.8 Hz, 2H, Ar); 8.29 (d, J 5.4 Hz, 1H, Ar); 8.83 (d, J 6.8 Hz, 2H, Ar); 8.97 (s, 1H, Ar); 9.44 (s, 1H, Ar); 11.77 (s, 1H, NH); HCl salt signal not observed. M/Z (M+H)⁺=429.5. MP>250° C.

### Example 18—Synthesis of compound 66 (6-(3-Pyridin-4-yl-propoxy)-2-thieno[2,3-c]pyridin-5-yl-3H-pyrido[3,2-d]pyrimidin-4-one hydrochloride)

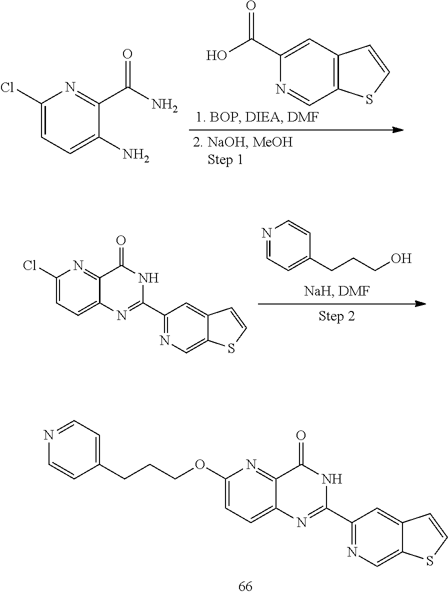 [SMILES: NC(=O)c1nc(Cl)ccc1N ; O=C(O)c1cc2ccsc2cn1 ; O=c1[nH]c(-c2cc3ccsc3cn2)nc2ccc(Cl)nc12 ; OCCCc1ccncc1 ; O=c1[nH]c(-c2cc3ccsc3cn2)nc2ccc(OCCCc3ccncc3)nc12]

Step 1:

6-Chloro-2-thieno[2,3-c]pyridin-5-yl-3H-pyrido[3,2-d]pyrimidin-4-one (225 mg, 0.71 mmol) was prepared according to procedure of example 1, step 4, starting from 3-amino-6-chloropyridine-2-carboxamide (130 mg, 0.76 mmol) and thieno[3,2-c]pyridine-6-carboxylic acid (204 mg, 1.14 mmol). It was obtained as a beige solid in 93% yield.

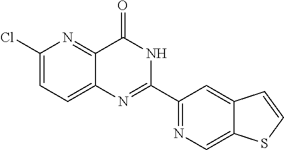 [SMILES: O=c1[nH]c(-c2cc3ccsc3cn2)nc2ccc(Cl)nc12]

M/Z (M[³⁵Cl]+H)⁺=315.0.

Step 2:

At 0° C., sodium hydride (60% dispersion in oil, 86 mg, 2.14 mmol) was added dropwise to a solution of 4-pyridine-propanol (234 mg, 1.70 mmol) in DMF (4.0 mL). The reaction mixture was stirred at 0° C. for 30 minutes before addition of 6-chloro-2-thieno[2,3-c]pyridin-5-yl-3H-pyrido[3,2-d]pyrimidin-4-one (224 mg, 0.71 mmol). The reaction mixture was heated at 100° C. for 2 h before being poured into a cold aqueous solution of ammonium chloride (40 mL). The brown precipitate was collected by filtration and purified by column chromatography on silica gel, using dichloromethane/methanol as eluent. HCl salt was obtained by concentration to dryness after addition of an excess of HCl (2N in Et₂O) to a solution of the product in dichloromethane to afford compound 66 (46 mg, 14%) as a yellow solid.

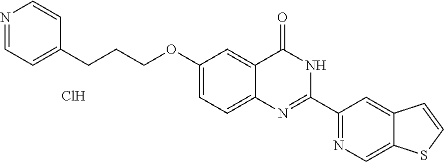 [SMILES: O=c1[nH]c(-c2cc3ccsc3cn2)nc2ccc(OCCCc3ccncc3)cc12 ; Cl]

¹H-NMR (400 MHz, DMSO): 2.25 (m, 2H, CH₂); 3.11 (t, J 7.5 Hz, 2H, CH₂); 4.48 (t, J 6.5 Hz, 2H, CH₂—O); 7.28 (d, J 8.9 Hz, 1H, Ar); 7.80 (d, J 5.4 Hz, 1H, Ar); 8.08 (m, 2H, Ar); 8.13 (d, J 8.9 Hz, 1H, Ar); 8.30 (d, J 5.4 Hz, 1H, Ar); 8.84 (m, 2H, Ar); 8.92 (s, 1H, Ar); 9.46 (m, 1H, Ar); HCl salt signal not observed; NH signal not observed. M/Z (M+H)⁺=416.0. MP=231-238° C.

### Compound 67 (6-(3-Pyridin-4-yl-propoxy)-2-(4-trifluoromethyl-pyridin-2-yl)-3H-pyrido[3,2-d]pyrimidin-4-one hydrochloride)

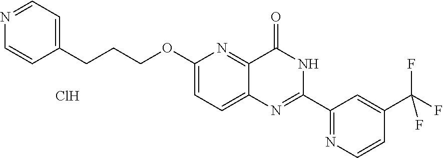 [SMILES: O=c1[nH]c(-c2cc(C(F)(F)F)ccn2)nc2ccc(OCCCc3ccncc3)nc12 ; Cl]

Compound 67 was prepared according to procedure of example 17, starting from 4-(trifluoromethyl)pyridine-2-carboxylic acid in step 1 and using BOP/DIEA instead of oxalyl chloride in step 3 (cf. procedure of example 19, step 2). It was obtained as a white solid.

¹H-NMR (400 MHz, DMSO): 2.25 (m, 2H, CH₂); 3.11 (t, J 7.5 Hz, 2H, CH₂); 4.48 (t, J 6.5 Hz, 2H, CH₂—O); 7.30 (d, J 8.9 Hz, 1H, Ar); 8.05 (m, 1H, Ar); 8.08 (m, 2H, Ar); 8.20 (d, J 8.9 Hz, 1H, Ar); 8.60 (m, 1H, Ar); 8.84 (m, 2H, Ar); 9.04 (m, 1H, Ar); 12.34 (bs, 1H, NH); HCl salt signal not observed. M/Z (M+H)⁺=428.0. MP>250° C.

### Example 19—Synthesis of compound 68 (6-(3-Pyridin-4-yl-propoxy)-2-(4-trifluoromethyl-pyridin-2-yl)-3H-pyrido[2,3-d]pyrimidin-4-one hydrochloride)

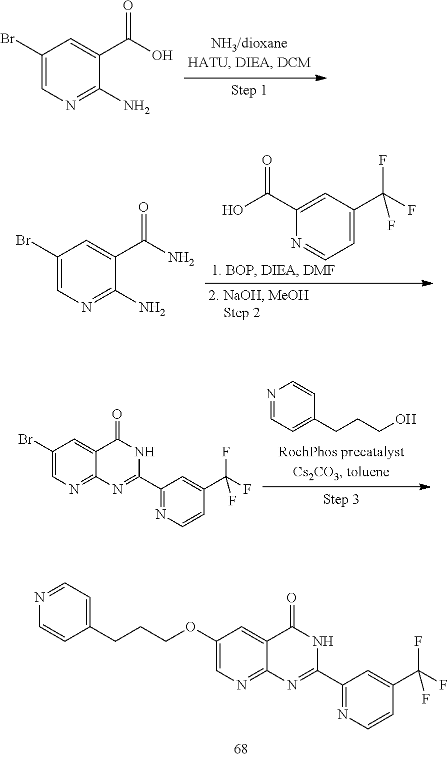 [SMILES: Nc1ncc(Br)cc1C(=O)O ; NC(=O)c1cc(Br)cnc1N ; O=C(O)c1cc(C(F)(F)F)ccn1 ; O=c1[nH]c(-c2cc(C(F)(F)F)ccn2)nc2ncc(Br)cc12 ; OCCCc1ccncc1 ; O=c1[nH]c(-c2cc(C(F)(F)F)ccn2)nc2ncc(OCCCc3ccncc3)cc12]

Step 1:

A solution of 5-bromo-2-aminonicotinic acid (630 mg, 2.90 mmol), ammonia (0.5M in dioxane, 12.0 mL, 5.80 mmol), O-(7-azabenzotriazol-1-yl)-N,N,N′,N′-tetramethyluronium hexafluorophosphate (1.65 g, 4.35 mmol) and diisopropylethylamine (1.30 mL, 7.54 mmol) in anhydrous dichloromethane (15.0 mL) was stirred for 16 h at room temperature. The reaction mixture was poured into aqueous ammonium chloride (70 mL) and extracted with ethyl acetate (2×100 mL).

The combined organic extracts were washed with brine (100 mL), dried over MgSO₄ and concentrated under vacuum. Purification by column chromatography on silica gel, using hexane/ethyl acetate as eluent, afforded 2-amino-5-bromo-nicotinamide (426 mg, 69%) as a yellow solid.

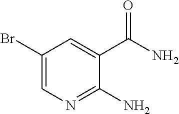 [SMILES: NC(=O)c1cc(Br)cnc1N]

M/Z (M[⁷⁹Br]+H)⁺=212.2.

Step 2:

6-Bromo-2-(4-trifluoromethyl-pyridin-2-yl)-3H-pyrido[2,3-d]pyrimidin-4-one was prepared according to procedure of example 1, step 4, starting from 2-amino-5-bromo-nicotinamide (213 mg, 0.99 mmol) and 4-(trifluoromethyl)pyridine-2-carboxylic acid (208 mg, 1.09 mmol). Purification by column chromatography on silica gel, using hexane/ethyl acetate as eluent, afforded the product (112 mg, 30%) as a beige solid.

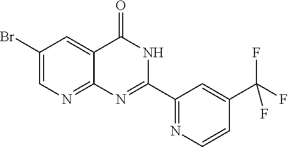 [SMILES: O=c1[nH]c(-c2cc(C(F)(F)F)ccn2)nc2ncc(Br)cc12]

M/Z (M[⁷⁹Br]+H)⁺=371.0.

Step 3:

Compound 68 was obtained according to the procedure of example 15, step 3, starting from 6-bromo-2-(4-trifluoromethyl-pyridin-2-yl)-3H-pyrido[2,3-d]pyrimidin-4-one (42 mg, 0.11 mmol) and 4-pyridinepropanol (91 mg, 0.66 mmol), and using toluene instead of dioxane for step 3. The HCl salt was obtained by concentration to dryness after addition of an excess of HCl (1.2N in MeOH) to a solution of the pure product in methanol to give compound 68 (9 mg, 18%) as a yellow solid.

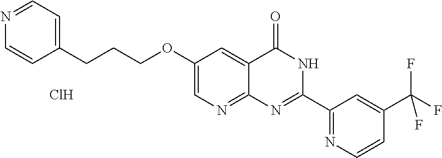 [SMILES: O=c1[nH]c(-c2cc(C(F)(F)F)ccn2)nc2ncc(OCCCc3ccncc3)cc12 ; Cl]

¹H-NMR (400 MHz, DMSO): 2.24 (m, 2H, CH₂); 3.12 (t, J 7.5 Hz, 2H, CH₂); 4.27 (t, J 6.1 Hz, 2H, CH₂—O); 7.94 (d, J 3.3 Hz, 1H, Ar); 8.03 (m, 2H, Ar); 8.07 (d, J 5.0 Hz, 1H, Ar); 8.63 (s, 1H, Ar); 8.71 (d, J 3.3 Hz, 1H, Ar); 8.83 (m, 2H, Ar); 9.06 (d, J 5.0 Hz, 1H, Ar); 12.47 (bs, 1H, NH); HCl salt signal not observed. M/Z (M+H)⁺=428.0. MP>250° C.

### Example 20—Synthesis of compound 69 (6-(3-Pyridin-4-yl-propoxy)-2-(4-trifluoromethyl-pyridin-2-yl)-3H-pyrido[3,4-d]pyrimidin-4-one hydrochloride)

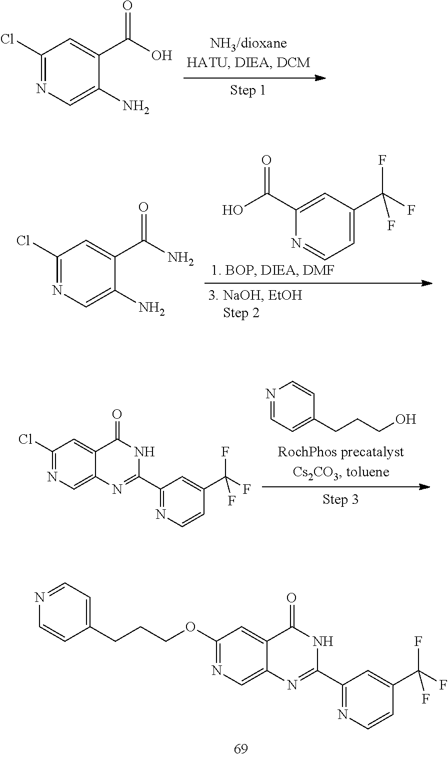 [SMILES: Nc1cnc(Cl)cc1C(=O)O ; NC(=O)c1cc(Cl)ncc1N ; O=C(O)c1cc(C(F)(F)F)ccn1 ; O=c1[nH]c(-c2cc(C(F)(F)F)ccn2)nc2cnc(Cl)cc12 ; OCCCc1ccncc1 ; O=c1[nH]c(-c2cc(C(F)(F)F)ccn2)nc2cnc(OCCCc3ccncc3)cc12]

Step 1:

5-Amino-2-chloro-isonicotinamide was prepared according to procedure of example 19, step 1, starting from 5-amino-2-chloro-isonicotic acid (370 mg, 2.14 mmol) to afford the product (313 mg, 85%) as a yellow solid.

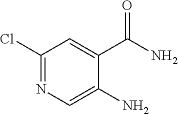 [SMILES: NC(=O)c1cc(Cl)ncc1N]

M/Z (M[³⁵Cl]+H)⁺=172.3.

Step 2:

6-Chloro-2-(4-trifluoromethyl-pyridin-2-yl)-3H-pyrido[3,4-d]pyrimidin-4-one was prepared according to procedure of example 1, step 4, starting from 5-amino-2-chloro-isonicotinamide (160 mg, 0.93 mmol) and 4-(trifluoromethyl)pyridine-2-carboxylic acid (195 mg, 1.02 mmol). Purification by column chromatography on silice gel, using hexane/ethyl acetate as eluent, afforded the product (118 mg, 37%) as a white solid.

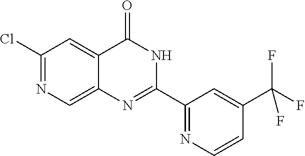 [SMILES: O=c1[nH]c(-c2cc(C(F)(F)F)ccn2)nc2cnc(Cl)cc12]

M/Z (M[³⁵Cl]+H)⁺=326.9.

Step 3:

Compound 69 was obtained according to the procedure of example 19, step 3, starting from 6-chloro-2-(4-trifluoromethyl-pyridin-2-yl)-3H-pyrido[3,4-d]pyrimidin-4-one (46 mg, 0.14 mmol) and 4-pyridinepropanol (115 mg, 0.84 mmol) to give compound 69 (16 mg, 25%) as a yellow solid.

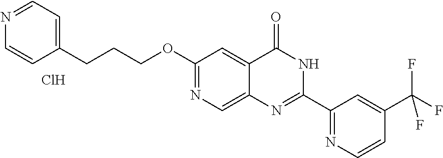 [SMILES: O=c1[nH]c(-c2cc(C(F)(F)F)ccn2)nc2cnc(OCCCc3ccncc3)cc12 ; Cl]

¹H-NMR (400 MHz, DMSO): 2.21 (m, 2H, CH₂); 3.07 (t, J 7.6 Hz, 2H, CH₂); 4.42 (t, J 6.3 Hz, 2H, CH₂—O); 7.31 (d, J 0.7 Hz, 1H, Ar); 7.94 (m, 2H, Ar); 8.06 (m, 1H, Ar); 8.61 (m, 1H, Ar); 8.79 (m, 2H, Ar); 8.92 (d, J 0.7 Hz, 1H, Ar); 9.04 (d, J 5.0 Hz, 1H, Ar); 12.35 (bs, 1H, NH); HCl salt signal not observed. M/Z (M+H)⁺=428.0. MP=224-228° C.

### Example 21—Synthesis of compound 70 (6-(3-Pyridin-4-yl-propoxy)-2-thieno[2,3-c]pyridin-5-yl-7-trifluoromethyl-3H-quinazolin-4-one hydrochloride)

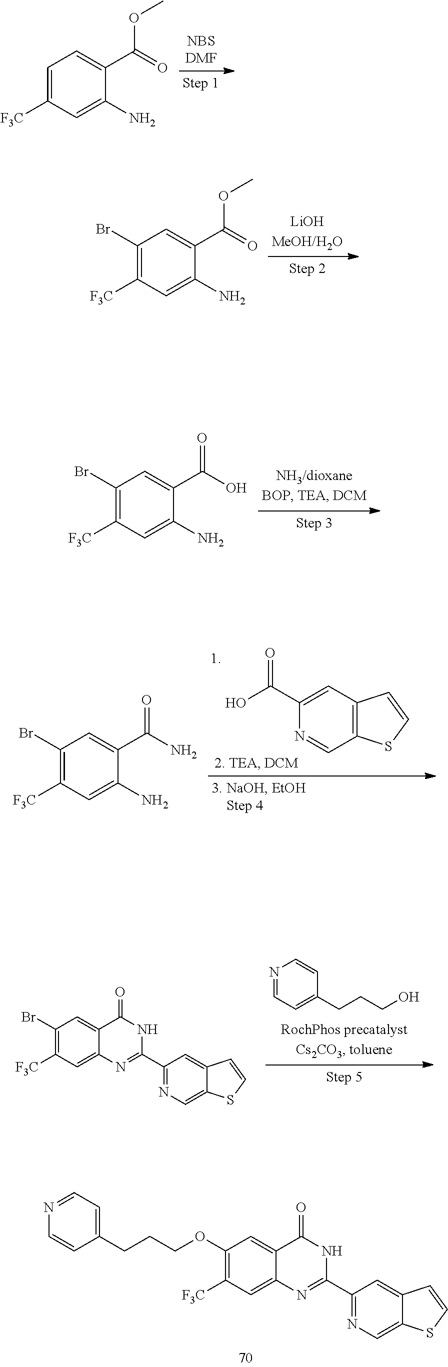

Step 1:

N-bromosuccinimide (427 mg, 2.40 mmol) was added to a solution of methyl 2-amino-4-(trifluoromethyl)benzoate (500 mg, 2.28 mmol) in DMF (23 mL). The reaction mixture was stirred at room temperature for 16 h before being poured into aqueous potassium carbonate (100 mL). The resulting precipitate was collected by filtration and dried under vacuum to give 2-amino-5-bromo-4-methyl-benzoic acid methyl ester (615 mg, 82%) as a beige solid.

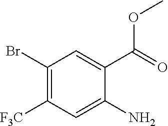 [SMILES: COC(=O)c1cc(Br)c(C(F)(F)F)cc1N]

M/Z (M[⁷⁹Br]+H)⁺=298.0.

Step 2:

Lithium hydroxide (145 mg, 6.04 mmol) was added to a suspension of 2-amino-5-bromo-4-methyl-benzoic acid methyl ester (600 mg, 2.01 mmol) in methanol (3.0 mL) and water (3.0 mL). The reaction mixture was heated at 50° C. for 1 h before being diluted in cold water, acidified to pH=1 with aqueous HCl (1N), and extracted with dichloromethane. The combined organic extract was washed with brine, dried over sodium sulfate and concentrated to dryness to afford 2-amino-5-bromo-4-methyl-benzoic acid (525 mg, 92%) as a beige solid.

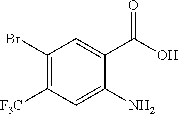 [SMILES: Nc1cc(C(F)(F)F)c(Br)cc1C(=O)O]

M/Z (M[⁷⁹Br]+H)⁺=284.0.

Step 3:

2-Amino-5-bromo-4-trifluoromethyl-benzamide was prepared according to procedure of example 1, step 1, starting from 2-amino-5-bromo-4-methyl-benzoic acid (525 mg, 1.85 mmol) and using triethylamine instead of diisopropylethylamine as a base. The product (428 mg, 82%) was obtained as a beige solid.

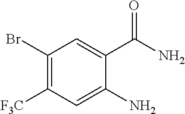 [SMILES: NC(=O)c1cc(Br)c(C(F)(F)F)cc1N]

M/Z (M[⁷⁹Br]+H)⁺=283.0.

Step 4:

6-Bromo-2-thieno[2,3-c]pyridin-5-yl-7-trifluoromethyl-3H-quinazolin-4-one was prepared according to procedure of example 17, step 3, starting from 2-amino-5-bromo-4-trifluoromethyl-benzamide (260 mg, 0.92 mmol) and thieno[3,2-c]pyridine-6-carboxylic acid (370 mg, 1.84 mmol). Purification by trituration in dichloromethane afforded the product as a brown solid (230 mg, 59%).

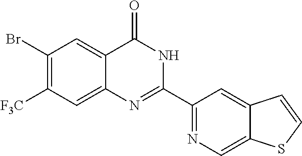 [SMILES: O=c1[nH]c(-c2cc3ccsc3cn2)nc2cc(C(F)(F)F)c(Br)cc12]

M/Z (M[⁷⁹Br]+H)⁺=426.0.

Step 5:

Compound 70 was obtained according to the procedure of example 19, step 3, starting from 6-bromo-2-thieno[2,3-c]pyridin-5-yl-7-trifluoromethyl-3H-quinazolin-4-one (100 mg, 0.25 mmol) and 4-pyridinepropanol (208 mg, 1.52 mmol) to give compound 70 (46 mg, 35%) as a beige solid.

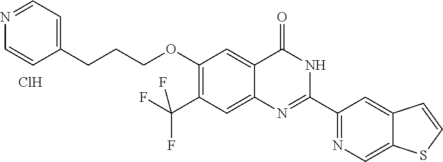 [SMILES: O=c1[nH]c(-c2cc3ccsc3cn2)nc2cc(C(F)(F)F)c(OCCCc3ccncc3)cc12 ; Cl]

¹H-NMR (400 MHz, DMSO): 2.25 (m, 2H, CH₂); 3.10 (t, J 7.6 Hz, 2H, CH₂); 4.33 (t, J 6.3 Hz, 2H, CH₂—O); 7.79 (m, 2H, Ar); 7.99 (m, 2H, Ar); 8.06 (m, 1H, Ar); 8.30 (d, J 5.4 Hz, 1H, Ar); 8.83 (m, 2H, Ar); 8.94 (s, 1H, Ar); 9.47 (s, 1H, Ar); HCl salt signal not observed; NH signal not observed. M/Z (M+H)⁺=483.0. MP>250° C.

### Example 22—Synthesis of compound 71 (5-Chloro-6-(3-pyridin-4-yl-propoxy)-2-thieno[2,3-c]pyridin-5-yl-3H-quinazolin-4-one hydrochloride)

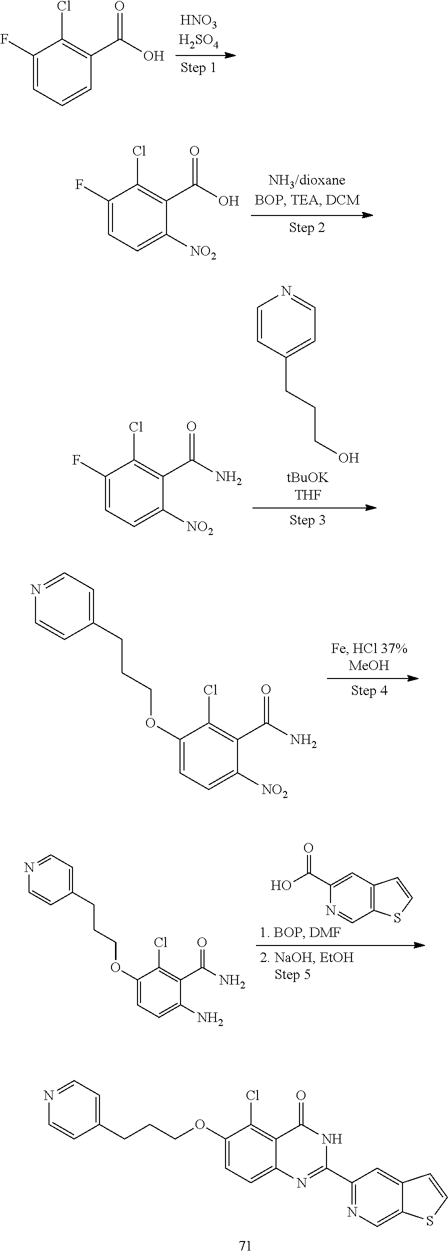

Step 1:

At 0° C., concentrated nitric acid (240 μL, 5.67 mmol) was slowly added to a solution of 2-chloro-3-fluorobenzoic acid (900 mg, 5.16 mmol) in concentrated sulfuric acid (50 mL). The reaction mixture was stirred at room temperature for 1 h before being poured into ice and water (100 mL) and extracted with dichloromethane (3×50 mL). The combined organic extract was washed with brine, dried over sodium sulfate and concentrated to dryness to give 2-chloro-3-fluoro-6-nitro-benzoic acid (1.2 g, 65% purity of the desired regioisomer) as a beige solid.

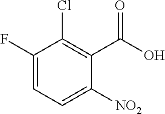 [SMILES: O=C(O)c1c([N+](=O)[O-])ccc(F)c1Cl]

M/Z (M[³⁵Cl]+H)⁺=219.5.

Step 2:

2-Chloro-3-fluoro-6-nitro-benzamide was obtained according to the procedure of example 21, step 3, starting from 2-chloro-3-fluoro-6-nitro-benzoic acid (1.1 g, 5.16 mmol) to afford the product (400 mg, 35%) as a yellow solid.

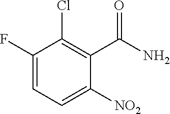 [SMILES: NC(=O)c1c([N+](=O)[O-])ccc(F)c1Cl]

M/Z (M[³⁵Cl]+H)⁺=218.5.

Step 3:

2-Chloro-6-nitro-3-(3-pyridin-4-yl-propoxy)-benzamide was prepared according to procedure of example 10, step 1, starting from 2-chloro-3-fluoro-6-nitro-benzamide (400 mg, 1.83 mmol) and 4-pyridine-propanol (251 mg, 1.83 mmol) to afford the product (320 mg, 52%) as a yellow solid.

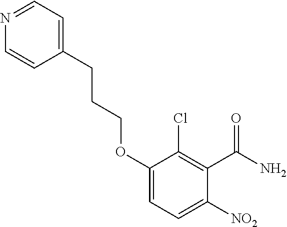 [SMILES: NC(=O)c1c([N+](=O)[O-])ccc(OCCCc2ccncc2)c1Cl]

M/Z (M[³⁵Cl]+H)⁺=335.5.

Step 4:

At 0° C., iron dust (299 mg, 5.36 mmol) was added to a solution of 2-chloro-6-nitro-3-(3-pyridin-4-yl-propoxy)-benzamide (300 mg, 0.89 mmol) in methanol (9.0 mL). An aqueous solution of HCl (37%, 2.0 mL) was added dropwise and the reaction mixture was stirred at room temperature for 1 h. At 0° C., the reaction mixture was neutralized with aqueous potassium carbonate and extracted twice with dichloromethane. The combined organic extract was dried over sodium sulfate, filtered and concentrated under vacuum. Purification by column chromatography on silica gel, using dichloromethane/methanol as eluent, afforded 6-amino-2-chloro-3-(3-pyridin-4-yl-propoxy)-benzamide (125 mg, 46%) as a beige solid.

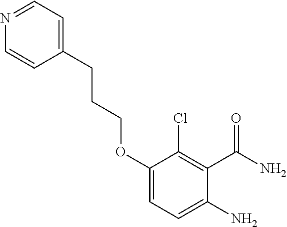 [SMILES: NC(=O)c1c(N)ccc(OCCCc2ccncc2)c1Cl]

M/Z (M[³⁵Cl]+H)⁺=305.5.

Step 5:

Compound 71 was prepared according to procedure of example 17, step 3, starting from 6-amino-2-chloro-3-(3-pyridin-4-yl-propoxy)-benzamide (125 mg, 0.41 mmol) and thieno[3,2-c]pyridine-6-carboxylic acid (120 mg, 0.61 mmol). It was purified by column chromatography on silica gel, using dichloromethane/methanol as eluent, and the HCl salt was obtained by filtration after addition of an excess of HCl (2N in Et₂O) to a solution of the product in dichloromethane to give compound 71 (64 mg, 35%) as a yellow solid.

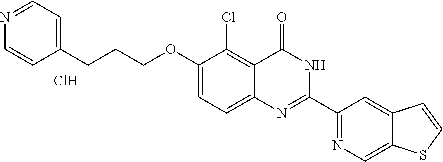 [SMILES: O=c1[nH]c(-c2cc3ccsc3cn2)nc2ccc(OCCCc3ccncc3)c(Cl)c12 ; Cl]

¹H-NMR (400 MHz, DMSO): 2.32 (m, 2H, CH₂); 3.20 (t, J 7.6 Hz, 2H, CH₂); 4.31 (t, J 6.3 Hz, 2H, CH₂—O); 7.82 (m, 3H, Ar); 8.05 (m, 2H, Ar); 8.36 (d, J 5.4 Hz, 1H, Ar); 8.88 (m, 2H, Ar); 8.97 (s, 1H, Ar); 9.52 (s, 1H, Ar); HCl salt signal not observed; NH signal not observed. M/Z (M[³⁵Cl]+H)⁺=449.5. MP>250° C.

### Example 23—Synthesis of compound 72 (8-Chloro-6-(3-pyridin-4-yl-propoxy)-2-thieno[2,3-c]pyridin-5-yl-3H-quinazolin-4-one hydrochloride)

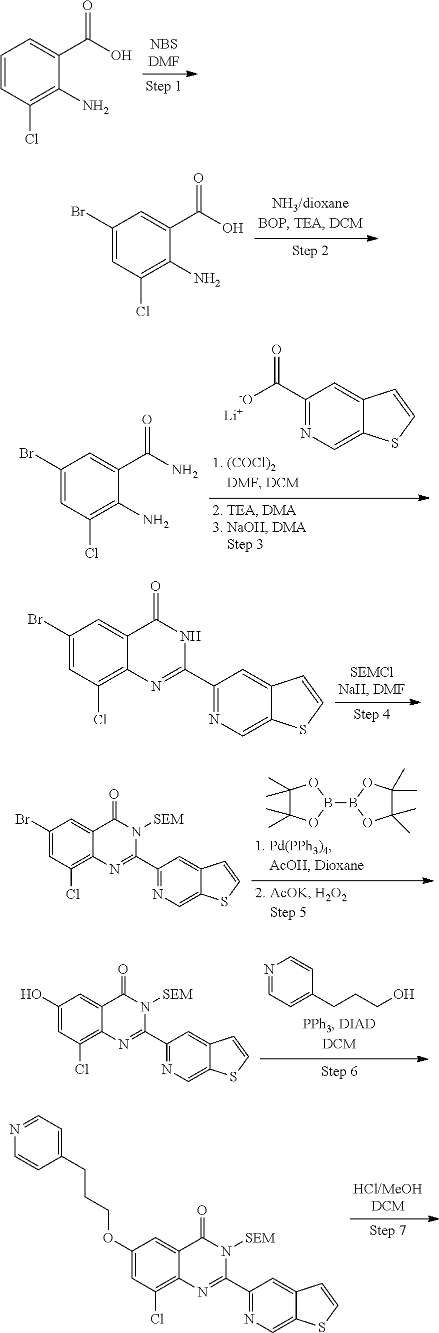

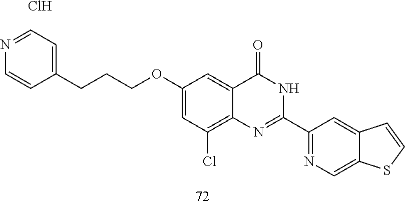 [SMILES: Cl ; O=c1[nH]c(-c2cc3ccsc3cn2)nc2c(Cl)cc(OCCCc3ccncc3)cc12]

Step 1:

N-bromosuccinimide (1.09 g, 6.12 mmol) was added to a solution of 2-amino-3-chloro-benzoic acid (1.0 g, 5.82 mmol) in DMF (30 mL). The reaction mixture was stirred at room temperature for 1 h before being poured into water and extracted twice with ethyl acetate. The combined organic extracts were dried over MgSO₄, filtered and concentrated under vacuum to give 2-amino-5-bromo-3-chloro-benzoic acid (2.9 g, quantitative yield) as a beige solid.

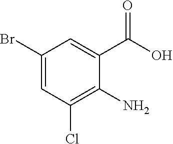 [SMILES: Nc1c(Cl)cc(Br)cc1C(=O)O]

M/Z (M[³⁵Cl][⁷⁹Br]+H)⁺=252.4.

Step 2:

2-Amino-5-bromo-3-chloro-benzamide was obtained according to procedure of example 21, step 3, starting from 2-amino-5-bromo-3-chloro-benzoic acid (1.46 g; 5.82 mmol). Purification by column chromatography on silica gel, using hexane/ethyl acetate as eluent, afforded the product (815 mg, 56%) as a white solid.

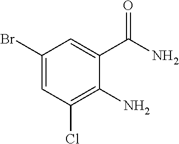 [SMILES: NC(=O)c1cc(Br)cc(Cl)c1N]

M/Z (M[³⁵Cl][⁷⁹Br]+H)⁺=251.5.

Step 3:

Under inert atmosphere, to a solution of lithium thieno[2,3-c]pyridine-5-carboxylate (790 mg, 3.93 mmol) in dichloromethane (20 mL) and DMF (15 μL) was added dropwise oxalyl chloride (515 μL, 5.89 mmol) and the reaction mixture was stirred for 30 min at room temperature. It was then concentrated to dryness and co-evaporated twice with toluene. The resulting solid residue was dissolved in DMA (20 mL) together with 2-amino-5-bromo-3-chloro-benzamide (490 mg, 1.96 mmol). Triethylamine (821 μL, 5.89 mmol) was added and the reaction mixture was stirred 1 h at room temperature. Then NaOH 1N in water (11.8 mL, 11.78 mmol) was added and the mixture was stirred for 1 h at 110° C. After cooling down to room temperature the resulting precipitate was filtrated, triturated in water and dried in vacuo to afford the product as a white solid (510 mg, 66%).

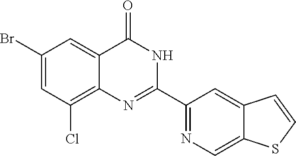 [SMILES: O=c1[nH]c(-c2cc3ccsc3cn2)nc2c(Cl)cc(Br)cc12]

M/Z (M[³⁵Cl][⁷⁹Br]+H)⁺=426.0.

Step 4:

A suspension of 6-bromo-8-chloro-2-thieno[2,3-c]pyridin-5-yl-3H-quinazolin-4-one (540 mg, 1.38 mmol) in dry DMF (14 mL) was sonicated for 1 minute before slow addition of sodium hydride (60% suspension in oil, 110 mg, 2.75 mmol). The reaction mixture was stirred for 5 minutes at room temperature and 2-(trimethylsilyl)ethoxymethyl chloride (0.73 mL, 4.13 mmol) was slowly added. The reactor was sealed, sonicated for 10 minutes and the reaction mixture was stirred for 16 h at room temperature before being poured into ice and aqueous sodium bicarbonate and extracted twice with ethyl acetate. The combined organic extracts were washed with brine, dried over MgSO₄, filtered and concentrated under vacuum. Purification by column chromatography on silica gel, using hexane/ethyl acetate as eluent, afforded 6-bromo-8-chloro-2-thieno[2,3-c]pyridin-5-yl-3-(2-trimethylsilanyl-ethoxymethyl)-3H-quinazolin-4-one (575 mg, 80%) as a beige solid.

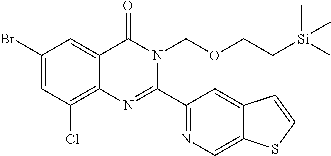 [SMILES: C[Si](C)(C)CCOCn1c(-c2cc3ccsc3cn2)nc2c(Cl)cc(Br)cc2c1=O]

M/Z (M[³⁵Cl][⁷⁹Br]+H)⁺=524.5.

Step 5:

Under inert atmosphere, a suspension of 6-bromo-8-chloro-2-thieno[2,3-c]pyridin-5-yl-3-(2-trimethylsilanyl-ethoxymethyl)-3H-quinazolin-4-one (500 mg, 0.96 mmol), bispinacolatodiboron (422 mg, 1.43 mmol), potassium acetate (282 mg, 2.87 mmol) and Pd(PPh₃)₄ (111 mg, 0.10 mmol) in dioxane (10 mL) was stirred at 100° C. for 3 h. After cooling to room temperature, acetic acid (0.33 mL, 5.74 mmol) and hydrogen peroxide (30% in water, 0.18 mL, 5.74 mmol) were added and the reaction mixture was stirred for 48 h at room temperature, before being poured into aqueous sodium bicarbonate and extracted twice with ethyl acetate. The combined organic extracts were washed with brine, dried over MgSO₄, filtered and concentrated under vacuum. Purification by column chromatography on silica gel, using hexane/ethyl acetate as eluent, afforded 8-chloro-6-hydroxy-2-thieno[2,3-c]pyridin-5-yl-3-(2-trimethylsilanyl-ethoxymethyl)-3H-quinazolin-4-one (242 mg, 50%) as a white solid.

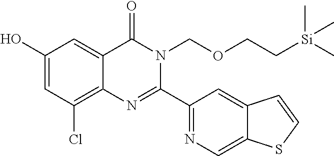 [SMILES: C[Si](C)(C)CCOCn1c(-c2cc3ccsc3cn2)nc2c(Cl)cc(O)cc2c1=O]

M/Z (M[³⁵Cl]+H)⁺=460.6.

Step 6:

In a sealed vial, a suspension of 8-chloro-6-hydroxy-2-thieno[2,3-c]pyridin-5-yl-3-(2-trimethylsilanyl-ethoxymethyl)-3H-quinazolin-4-one (140 mg, 0.30 mmol), triphenylphosphine (160 mg, 0.61 mmol), 4-pyridinepropanol (83 mg, 0.61 mmol) and diisopropyl azodicarboxylate (0.12 mL, 0.61 mmol) in dichloromethane (3.0 mL) was heated at 40° C. for 16 h. After cooling to room temperature, the reaction mixture was treated with aqueous sodium bicarbonate and extracted twice with dichloromethane. The combined organic extracts were washed with brine, dried over MgSO₄, filtered and concentrated under vacuum. Purification by column chromatography on silica gel, using dichloromethane/methanol as eluent, afforded 8-chloro-6-(3-pyridin-4-yl-propoxy)-2-thieno[2,3-c]pyridin-5-yl-3-(2-trimethylsilanyl-ethoxymethyl)-3H-quinazolin-4-one (151 mg, quantitative yield) as a white solid.

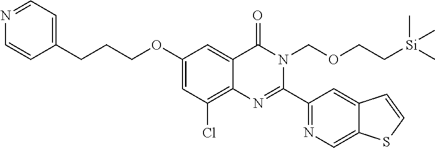 [SMILES: C[Si](C)(C)CCOCn1c(-c2cc3ccsc3cn2)nc2c(Cl)cc(OCCCc3ccncc3)cc2c1=O]

M/Z (M[³⁵Cl]+H)⁺=579.6.

Step 7:

In a sealed vial, to a solution of 8-chloro-6-(3-pyridin-4-yl-propoxy)-2-thieno[2,3-c]pyridin-5-yl-3-(2-trimethylsilanyl-ethoxymethyl)-3H-quinazolin-4-one (40 mg, 0.07 mmol) in dichloromethane (1.8 mL) was added HCl (1.2N solution in MeOH, 0.7 mL). The reaction mixture was heated at 50° C. for 2 h before being concentrated under vacuum. The solid residue was triturated in dichloromethane and diethyl ether to afford compound 72 (18 mg, 53%) as a white solid.

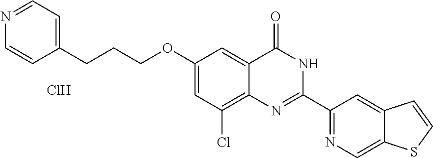 [SMILES: O=c1[nH]c(-c2cc3ccsc3cn2)nc2c(Cl)cc(OCCCc3ccncc3)cc12 ; Cl]

¹H-NMR (400 MHz, DMSO): 2.21 (m, 2H, CH₂); 3.08 (t, J 7.4 Hz, 2H, CH₂); 4.20 (t, J 6.1 Hz, 2H, CH₂—O); 7.54 (d, J 2.9 Hz, 1H, Ar); 7.64 (d, J 2.9 Hz, 1H, Ar); 7.84 (d, J 5.4 Hz, 1H, Ar); 7.96 (d, J 6.4 Hz, 2H, Ar); 8.30 (d, J 5.4 Hz, 1H, Ar); 8.81 (d, J 6.1 Hz, 2H, Ar); 8.94 (s, 1H, Ar); 9.46 (s, 1H, Ar); 12.03 (s, 1H, NH); HCl salt signal not observed. M/Z (M[³⁵Cl]+H)⁺=449.6. MP>250° C.

### Example 24—Synthesis of compound 73 (8-Cyclopropyl-6-(3-pyridin-4-yl-propoxy)-2-thieno[2,3-c]pyridin-5-yl-3H-quinazolin-4-one hydrochloride)

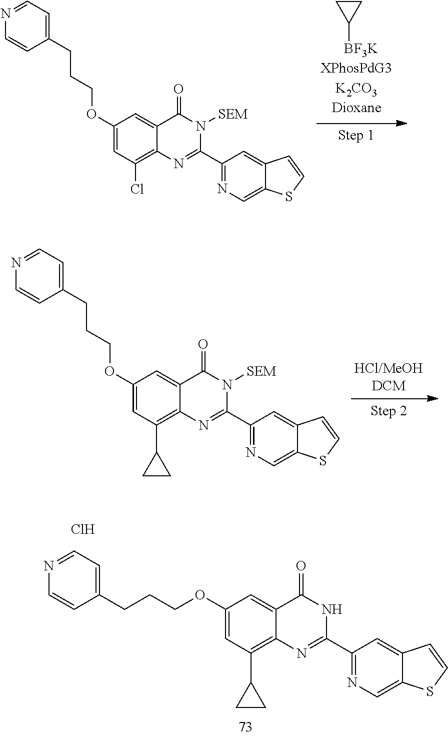

Step 1:

Under inert atmosphere, a mixture of 8-chloro-6-(3-pyridin-4-yl-propoxy)-2-thieno[2,3-c]pyridin-5-yl-3-(2-trimethylsilanyl-ethoxymethyl)-3H-quinazolin-4-one (84 mg, 0.14 mmol), potassium cyclopropyltrifluoroborate (64 mg, 0.43 mmol), potassium carbonate (60 mg, 0.43 mmol) and XPhos precatalyst generation 3 (12 mg, 0.014 mmol) in water (140 μL) and dioxane (1.4 mL) was heated at 80° C. for 1 h. After cooling to room temperature, the reaction mixture was treated with water and extracted twice with ethyl acetate. The combined organic extracts were washed with brine, dried over MgSO₄, filtered and concentrated under vacuum. Purification by column chromatography on silica gel, using cyclohexane/ethyl acetate as eluent, afforded 8-cyclopropyl-6-(3-pyridin-4-yl-propoxy)-2-thieno[2,3-c]pyridin-5-yl-3-(2-trimethylsilanyl-ethoxymethyl)-3H-quinazolin-4-one (42 mg, 49%) as a white solid.

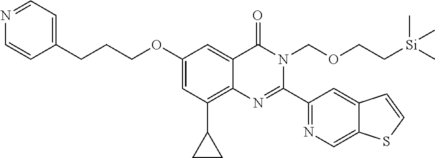 [SMILES: C[Si](C)(C)CCOCn1c(-c2cc3ccsc3cn2)nc2c(C3CC3)cc(OCCCc3ccncc3)cc2c1=O]

M/Z (M+H)⁺=585.7.

Step 2:

Compound 73 was prepared according to procedure of example 23, step 7, starting from 8-cyclopropyl-6-(3-pyridin-4-yl-propoxy)-2-thieno[2,3-c]pyridin-5-yl-3-(2-trimethylsilanyl-ethoxymethyl)-3H-quinazolin-4-one (40 mg, 0.07 mmol) to afford the product as a yellow solid (10 mg, 30%).

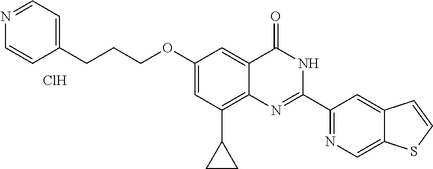 [SMILES: O=c1[nH]c(-c2cc3ccsc3cn2)nc2c(C3CC3)cc(OCCCc3ccncc3)cc12 ; Cl]

¹H-NMR (400 MHz, DMSO): 0.89 (m, 2H, cyclopropyl); 1.18 (m, 2H, cyclopropyl); 2.20 (m, 2H, CH₂); 3.09 (t, J 7.4 Hz, 2H, CH₂); 3.19 (m, 1H, cyclopropyl); 4.15 (t, J 6.1 Hz, 2H, CH₂—O); 6.79 (d, J 2.9 Hz, 1H, Ar); 7.36 (d, J 2.9 Hz, 1H, Ar); 7.79 (d, J 5.4 Hz, 1H, Ar); 8.00 (d, J 6.4 Hz, 2H, Ar); 8.28 (d, J 5.4 Hz, 1H, Ar); 8.83 (d, J 6.5 Hz, 2H, Ar); 8.98 (s, 1H, Ar); 9.44 (s, 1H, Ar); 11.76 (s, 1H, NH); HCl salt signal not observed. M/Z (M+H)⁺=455.7. MP>250° C.

### Example 25—Synthesis of compound 74 (8-Ethyl-6-(3-pyridin-4-yl-propoxy)-2-thieno[2,3-c]pyridin-5-yl-3H-quinazolin-4-one hydrochloride)

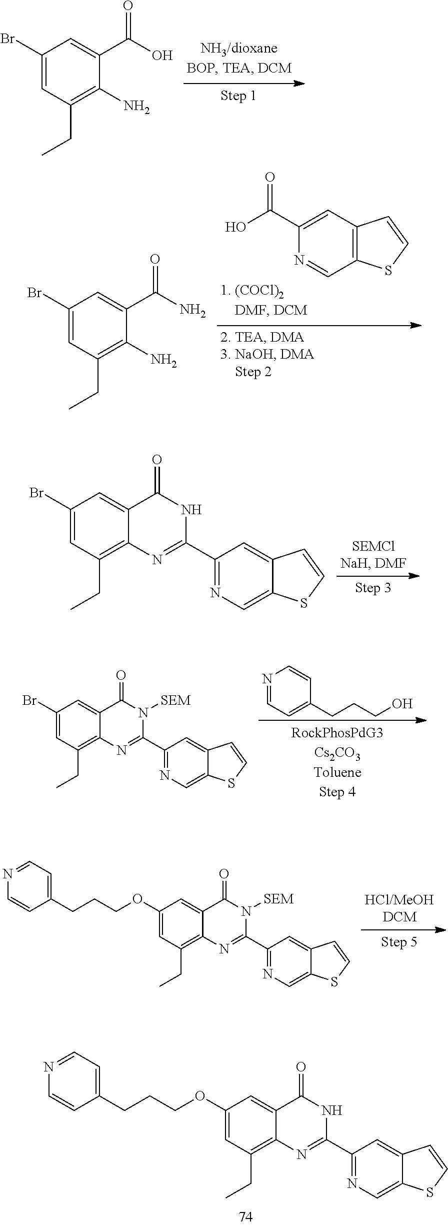

Step 1:

2-Amino-5-bromo-3-ethyl-benzamide was obtained according to the procedure of example 21, step 3, starting from 2-amino-5-bromo-3-ethyl-benzoic acid (310 mg, 1.27 mmol) to afford the product (268 mg, 87%) as a brown solid.

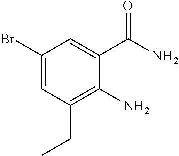

M/Z (M[⁷⁹Br]+H)⁺=243.5.

Step 2:

6-Bromo-8-ethyl-2-thieno[2,3-c]pyridin-5-yl-3H-quinazolin-4-one was prepared according to procedure of example 23, step 3, starting from 2-amino-5-bromo-3-ethyl-benzamide (260 mg, 1.07 mmol) and thieno[3,2-c]pyridine-6-carboxylic acid (396 mg, 1.58 mmol) to afford the product as a beige solid (346 mg, 84%).

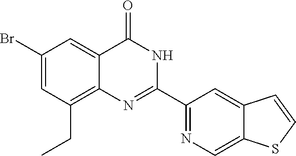 [SMILES: CCc1cc(Br)cc2c(=O)[nH]c(-c3cc4ccsc4cn3)nc12]

M/Z (M[⁷⁹Br]+H)⁺=386.3.

Step 3:

6-Bromo-8-ethyl-2-thieno[2,3-c]pyridin-5-yl-3-(2-trimethylsilanyl-ethoxymethyl)-3H-quinazolin-4-one was prepared according to procedure of example 23, step 4, starting from 6-bromo-8-ethyl-2-thieno[2,3-c]pyridin-5-yl-3H-quinazolin-4-one (340 mg, 0.88 mmol) to afford the product (73 mg, 16%) as a white solid.

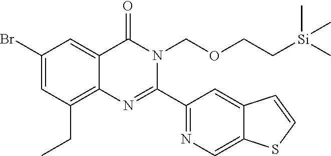 [SMILES: CCc1cc(Br)cc2c(=O)n(COCC[Si](C)(C)C)c(-c3cc4ccsc4cn3)nc12]

M/Z (M[⁷⁹Br]+H)⁺=516.6.

Step 4:

8-Ethyl-6-(3-pyridin-4-yl-propoxy)-2-thieno[2,3-c]pyridin-5-yl-3-(2-trimethylsilanyl-ethoxymethyl)-3H-quinazolin-4-one was obtained according to the procedure of example 19, step 3, starting from 6-bromo-8-ethyl-2-thieno[2,3-c]pyridin-5-yl-3-(2-trimethylsilanyl-ethoxymethyl)-3H-quinazolin-4-one (130 mg, 0.25 mmol) and 4-pyridinepropanol (104 mg, 0.75 mmol) to afford the product (91 mg, 63%) as a yellow solid.

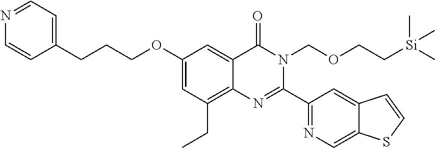 [SMILES: CCc1cc(OCCCc2ccncc2)cc2c(=O)n(COCC[Si](C)(C)C)c(-c3cc4ccsc4cn3)nc12]

M/Z (M+H)⁺=573.7.

Step 5:

Compound 74 was prepared according to procedure of example 23, step 7, starting from 8-ethyl-6-(3-pyridin-4-yl-propoxy)-2-thieno[2,3-c]pyridin-5-yl-3-(2-trimethylsilanyl-ethoxymethyl)-3H-quinazolin-4-one (120 mg, 0.21 mmol) to afford the product as a yellow solid (70 mg, 57%).

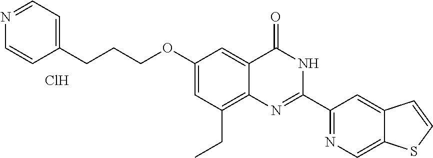 [SMILES: CCc1cc(OCCCc2ccncc2)cc2c(=O)[nH]c(-c3cc4ccsc4cn3)nc12 ; Cl]

¹H-NMR (400 MHz, DMSO): 1.33 (t, J 7.6 Hz, 3H, ethyl); 2.22 (m, 2H, CH₂); 3.11 (t, J 7.6 Hz, 2H, CH₂); 3.16 (q, J 7.6 Hz, 2H, ethyl); 4.17 (t, J 6.1 Hz, 2H, CH₂—O); 7.28 (d, J 2.0 Hz, 1H, Ar); 7.41 (d, J 2.0 Hz, 1H, Ar); 7.82 (d, J 5.3 Hz, 1H, Ar); 8.03 (d, J 6.0 Hz, 2H, Ar); 8.29 (d, J 5.3 Hz, 1H, Ar); 8.84 (d, J 6.0 Hz, 2H, Ar); 8.93 (s, 1H, Ar); 9.44 (s, 1H, Ar); 11.76 (s, 1H, NH); HCl salt signal not observed. M/Z (M+H)⁺=443.6. MP>250° C.

### Compound 75 (8-Fluoro-6-(3-pyridin-4-yl-propoxy)-2-thieno[2,3-c]pyridin-5-yl-3H-quinazolin-4-one)

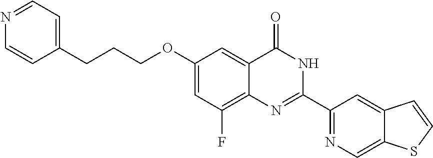 [SMILES: O=c1[nH]c(-c2cc3ccsc3cn2)nc2c(F)cc(OCCCc3ccncc3)cc12]

Compound 75 was prepared according to procedure of example 25, step 1-4, starting from 2-amino-5-bromo-3-fluoro-benzoic acid in step 1, followed by procedure of example 36, step 7. Purification by preparative HPLC afforded compound 75 as a white solid.

¹H-NMR (400 MHz, DMSO): 2.11 (m, 2H, CH₂); 2.80 (t, J 7.4 Hz, 2H, CH₂); 4.15 (t, J 6.3 Hz, 2H, CH₂—O); 7.29 (d, J 5.4 Hz, 2H, Ar); 7.40 (d, J 2.8 Hz, 1H, Ar); 7.44 (dd, J 11.7, 2.8 Hz, 1H, Ar); 7.82 (d, J 5.4 Hz, 1H, Ar); 8.28 (d, J 5.4 Hz, 1H, Ar); 8.47 (d, J 5.4 Hz, 2H, Ar); 8.92 (m, 1H, Ar); 9.44 (m, 1H, Ar); 11.94 (bs, 1H, NH). M/Z (M+H)⁺=433.6.

### Example 26—Synthesis of compound 76 (8-Methyl-6-(tetrahydro-pyran-4-ylmethoxy)-2-thieno[2,3-c]pyridin-5-yl-3H-quinazolin-4-one)

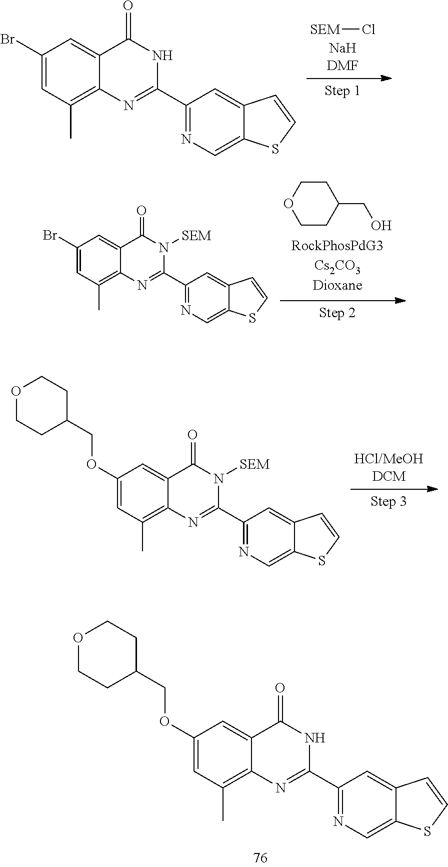 [SMILES: Cc1cc(Br)cc2c(=O)[nH]c(-c3cc4ccsc4cn3)nc12 ; C[Si](C)(C)CCOCCl ; Cc1cc(Br)cc2c(=O)n(COCC[Si](C)(C)C)c(-c3cc4ccsc4cn3)nc12 ; OCC1CCOCC1 ; Cc1cc(OCC2CCOCC2)cc2c(=O)n(COCC[Si](C)(C)C)c(-c3cc4ccsc4cn3)nc12 ; Cc1cc(OCC2CCOCC2)cc2c(=O)[nH]c(-c3cc4ccsc4cn3)nc12]

Step 1:

6-Bromo-8-methyl-2-thieno[2,3-c]pyridin-5-yl-3-(2-trimethylsilanyl-ethoxymethyl)-3H-quinazolin-4-one was prepared according to the procedure of example 23, step 4, starting from 6-bromo-8-methyl-2-thieno[2,3-c]pyridin-5-yl-3H-quinazolin-4-one (from example 17, 825 mg, 2.29 mmol) to afford the product (820 mg, 74%) as a white solid.

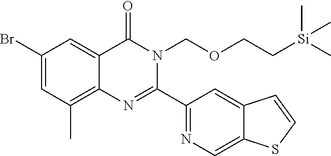 [SMILES: Cc1cc(Br)cc2c(=O)n(COCC[Si](C)(C)C)c(-c3cc4ccsc4cn3)nc12]

M/Z (M[⁷⁹Br]+H)⁺=502.5.

Step 2:

8-Methyl-6-(tetrahydro-pyran-4-ylmethoxy)-2-thieno[2,3-c]pyridin-5-yl-3-(2-trimethylsilanyl-ethoxymethyl)-3H-quinazolin-4-one was prepared according to the procedure of example 15, step 3, starting from 6-bromo-8-methyl-2-thieno[2,3-c]pyridin-5-yl-3-(2-trimethylsilanyl-ethoxymethyl)-3H-quinazolin-4-one (75 mg, 0.15 mmol) and 4-(hydroxymethyl)tetrahydropyran (104 mg, 0.89 mmol) to afford the product (43 mg, 54%) as a white solid.

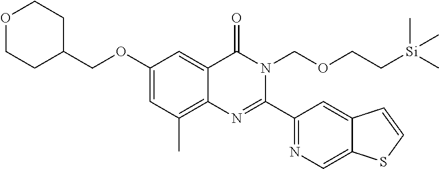 [SMILES: Cc1cc(OCC2CCOCC2)cc2c(=O)n(COCC[Si](C)(C)C)c(-c3cc4ccsc4cn3)nc12]

M/Z (M+H)⁺=538.7.

Step 3:

Compound 76 was prepared according to procedure of example 23, step 7, starting from 8-methyl-6-(tetrahydro-pyran-4-ylmethoxy)-2-thieno[2,3-c]pyridin-5-yl-3-(2-trimethylsilanyl-ethoxymethyl)-3H-quinazolin-4-one (40 mg, 0.07 mmol) to afford the product as a yellow solid (27 mg, 81%).

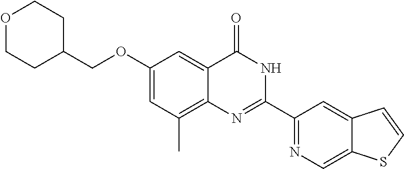 [SMILES: Cc1cc(OCC2CCOCC2)cc2c(=O)[nH]c(-c3cc4ccsc4cn3)nc12]

¹H-NMR (400 MHz, DMSO): 1.38 (m, 2H, 2 CH); 1.71 (m, 2H, 2 CH); 2.05 (m, 1H, CH); 2.68 (s, 3H, CH₃); 3.35 (m, 2H, 2 CH); 3.90 (m, 2H, 2 CH); 3.96 (d, J 6.4 Hz, 2H, CH₂—O); 7.37 (d, J 2.6 Hz, 1H, Ar); 7.42 (d, J 2.6 Hz, 1H, Ar); 7.80 (d, J 5.4 Hz, 1H, Ar); 8.28 (d, J 5.4 Hz, 1H, Ar); 8.96 (s, 1H, Ar); 9.43 (s, 1H, Ar); 11.71 (s, 1H, NH). M/Z (M+H)⁺=408.5.

### Compound 77 (8-Methyl-6-(2-oxetan-3-yl-ethoxy)-2-thieno[2,3-c]pyridin-5-yl-3H-quinazolin-4-one)

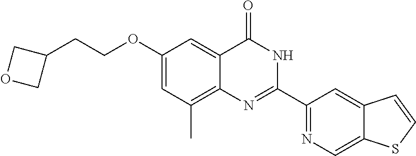 [SMILES: Cc1cc(OCCC2COC2)cc2c(=O)[nH]c(-c3cc4ccsc4cn3)nc12]

Compound 77 was prepared according to procedure of example 26, step 1 and 2, starting from 2-(oxetan-3-yl)ethanol in step 2, followed by procedure of example 30, step 2, to afford the product as a white solid.

¹H-NMR (400 MHz, DMSO): 2.13 (m, 2H, 2 CH); 2.67 (s, 3H, CH₃); 3.17 (m, 1H, CH); 4.07 (t, J 6.3 Hz, 2H, CH₂); 4.39 (t, J 6.3 Hz, 2H, CH₂); 4.70 (dd, J 7.9, 5.9 Hz, 2H, 2 CH); 7.33 (d, J 2.8 Hz, 1H, Ar); 7.40 (d, J 2.8 Hz, 1H, Ar); 7.80 (d, J 5.4 Hz, 1H, Ar); 8.28 (d, J 5.4 Hz, 1H, Ar); 8.96 (s, 1H, Ar); 9.43 (s, 1H, Ar); 11.71 (s, 1H, NH). M/Z (M+H)⁺=394.5.

### Compound 78 (8-Methyl-6-[2-(tetrahydro-furan-3-yl)-ethoxy]-2-thieno[2,3-c]pyridin-5-yl-3H-quinazolin-4-one)

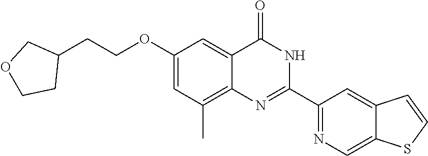 [SMILES: Cc1cc(OCCC2CCOC2)cc2c(=O)[nH]c(-c3cc4ccsc4cn3)nc12]

Compound 78 was prepared according to procedure of example 26, starting from 2-(tetrahydro-furan-3-yl)-ethanol in step 2 to afford the product as a yellow solid.

¹H-NMR (400 MHz, DMSO): 1.57 (m, 1H, CH); 1.84 (m, 2H, 2 CH); 2.06 (m, 1H, CH); 2.33 (m, 1H, CH); 2.68 (s, 3H, CH₃); 3.34 (t, J 7.8 Hz, 1H, CH); 3.64 (m, 1H, CH); 3.75 (ddd, J 8.3, 8.3, 4.5 Hz, 1H, CH); 3.85 (dd, J 8.3, 7.5 Hz, 1H, CH); 4.11 (m, 2H, 2 CH); 7.36 (d, J 2.8 Hz, 1H, Ar); 7.42 (d, J 2.8 Hz, 1H, Ar); 7.80 (d, J 5.4 Hz, 1H, Ar); 8.27 (d, J 5.4 Hz, 1H, Ar); 8.95 (s, 1H, Ar); 9.42 (s, 1H, Ar); 11.70 (s, 1H, NH). M/Z (M+H)⁺=408.6.

### Compound 79 (8-Methyl-6-[2-(tetrahydro-pyran-4-yl)-ethoxy]-2-thieno[2,3-c]pyridin-5-yl-3H-quinazolin-4-one)

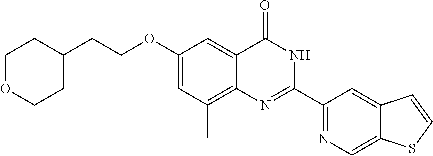 [SMILES: Cc1cc(OCCC2CCOCC2)cc2c(=O)[nH]c(-c3cc4ccsc4cn3)nc12]

Compound 79 was prepared according to procedure of example 26, starting from 2-(tetrahydro-2H-pyran-4-yl)ethan-1-ol in step 2 to afford the product as a yellow solid.

¹H-NMR (400 MHz, DMSO): 1.26 (m, 2H, 2 CH); 1.66 (m, 2H, 2 CH); 1.72 (m, 2H, 2 CH); 2.68 (s, 3H, CH₃); 3.30 (m, 1H, CH); 3.84 (m, 4H, 2 CH₂); 4.14 (t, J 6.3 Hz, 2H, CH₂); 7.37 (d, J 2.9 Hz, 1H, Ar); 7.43 (d, J 2.9 Hz, 1H, Ar); 7.80 (d, J 5.4 Hz, 1H, Ar); 8.29 (d, J 5.4 Hz, 1H, Ar); 8.96 (s, 1H, Ar); 9.44 (s, 1H, Ar); 11.74 (s, 1H, NH). M/Z (M+H)⁺=422.6.

### Example 27—Synthesis of compound 80 (8-Methyl-6-(tetrahydro-furan-3-ylmethoxy)-2-thieno[2,3-c]pyridin-5-yl-3H-quinazolin-4-one)

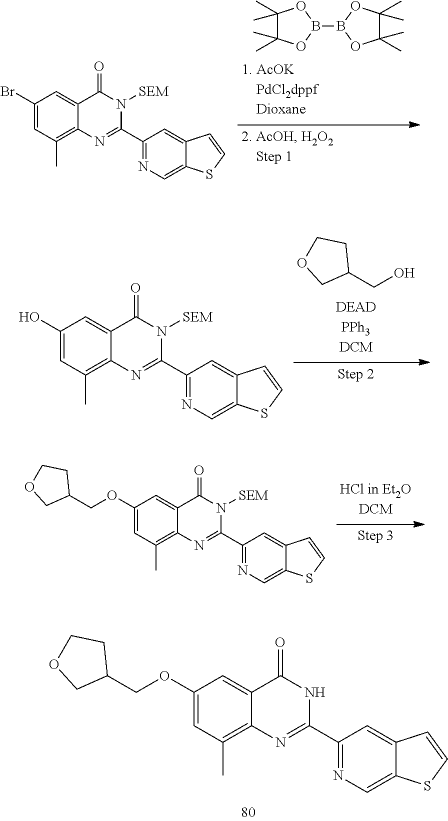

Step 1:

6-Hydroxy-8-methyl-2-thieno[2,3-c]pyridin-5-yl-3-(2-trimethylsilanyl-ethoxymethyl)-3H-quinazolin-4-one was prepared according to procedure of example 23, step 5, starting from 6-bromo-8-methyl-2-thieno[2,3-c]pyridin-5-yl-3-(2-trimethylsilanyl-ethoxymethyl)-3H-quinazolin-4-one (1.0 g, 1.99 mmol) and using PdCl₂.dppf as a catalyst instead of Pd(PPh₃)₄. The product (667 mg, 76%) was obtained as a white solid.

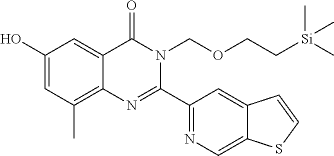 [SMILES: Cc1cc(O)cc2c(=O)n(COCC[Si](C)(C)C)c(-c3cc4ccsc4cn3)nc12]

M/Z (M+H)⁺=440.7.

Step 2:

8-Methyl-6-(tetrahydro-furan-3-ylmethoxy)-2-thieno[2,3-c]pyridin-5-yl-3-(2-trimethylsilanyl-ethoxymethyl)-3H-quinazolin-4-one was prepared according to procedure of example 23, step 6, starting from 6-hydroxy-8-methyl-2-thieno[2,3-c]pyridin-5-yl-3-(2-trimethylsilanyl-ethoxymethyl)-3H-quinazolin-4-one (100 mg, 0.23 mmol) and tetrahydro-3-furanmethanol (46 mg, 0.45 mmol), and using DEAD instead of DIAD. The product (62 mg, 52%) was obtained as a white solid.

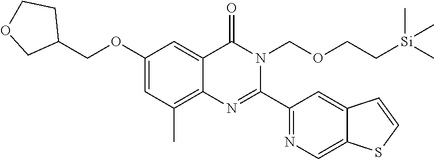 [SMILES: Cc1cc(OCC2CCOC2)cc2c(=O)n(COCC[Si](C)(C)C)c(-c3cc4ccsc4cn3)nc12]

M/Z (M+H)⁺=524.7.

Step 3:

Compound 80 was prepared according to procedure of example 23, step 7, starting from 8-methyl-6-(tetrahydro-furan-3-ylmethoxy)-2-thieno[2,3-c]pyridin-5-yl-3-(2-trimethylsilanyl-ethoxymethyl)-3H-quinazolin-4-one (180 mg, 0.34 mmol) and using HCl 2N in diethyl ether instead of HCl in methanol and purifying the product by trituration in ethanol and diethyl ether. The product was obtained as a yellow solid (47 mg, quantitative yield).

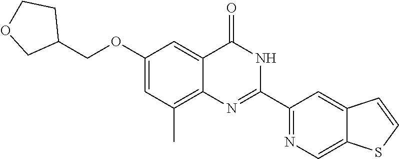 [SMILES: Cc1cc(OCC2CCOC2)cc2c(=O)[nH]c(-c3cc4ccsc4cn3)nc12]

¹H-NMR (400 MHz, DMSO): 1.73 (m, 1H, CH); 2.05 (m, 1H, CH); 2.70 (m, 1H, CH); 2.68 (s, 3H, CH₃); 3.58 (dd, J 8.5, 5.6 Hz, 1H, CH); 3.68 (m, 1H, CH); 3.81 (m, 2H, 2 CH); 4.05 (m, 2H, 2 CH); 7.38 (d, J 2.8 Hz, 1H, Ar); 7.44 (d, J 2.8 Hz, 1H, Ar); 7.80 (d, J 5.4 Hz, 1H, Ar); 8.28 (d, J 5.4 Hz, 1H, Ar); 8.96 (s, 1H, Ar); 9.43 (s, 1H, Ar); 11.73 (s, 1H, NH). M/Z (M+H)⁺=394.5.

### Compound 80-R (R-8-Methyl-6-(tetrahydro-furan-3-ylmethoxy)-2-thieno[2,3-c]pyridin-5-yl-3H-quinazolin-4-one)

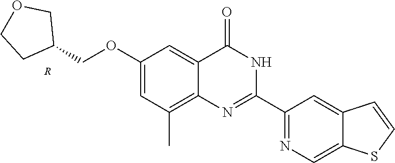

Compound 80-R was prepared according to procedure of example 27, starting from (S)-tetrahydrofuran-3-ylmethanol in step 2 to afford the product as a white solid after trituration in methanol and diethyl ether.

¹H-NMR (400 MHz, DMSO): 1.73 (m, 1H, CH); 2.05 (m, 1H, CH); 2.68 (s, 3H, CH₃); 2.72 (m, 1H, CH); 3.58 (dd, J 8.5, 5.6 Hz, 1H, CH); 3.69 (m, 1H, CH); 3.81 (m, 2H, 2 CH); 4.05 (m, 2H, 2 CH); 7.38 (d, J 2.8 Hz, 1H, Ar); 7.44 (d, J 2.8 Hz, 1H, Ar); 7.80 (d, J 5.4 Hz, 1H, Ar); 8.28 (d, J 5.4 Hz, 1H, Ar); 8.96 (s, 1H, Ar); 9.43 (s, 1H, Ar); 11.73 (s, 1H, NH). M/Z (M+H)⁺=394.0.

### Compound 80-S (S-8-Methyl-6-(tetrahydro-furan-3-ylmethoxy)-2-thieno[2,3-c]pyridin-5-yl-3H-quinazolin-4-one)

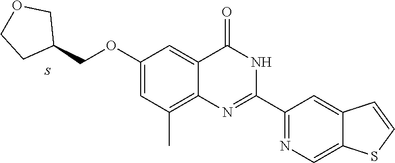

Compound 80-S was prepared according to procedure of example 27, starting from (R)-tetrahydrofuran-3-ylmethanol in step 2 to afford the product as a white solid after trituration in methanol and diethyl ether.

¹H-NMR (400 MHz, DMSO): 1.73 (m, 1H, CH); 2.05 (m, 1H, CH); 2.68 (s, 3H, CH₃); 2.72 (m, 1H, CH); 3.58 (dd, J 8.5, 5.6 Hz, 1H, CH); 3.69 (m, 1H, CH); 3.81 (m, 2H, 2 CH); 4.05 (m, 2H, 2 CH); 7.38 (d, J 2.8 Hz, 1H, Ar); 7.44 (d, J 2.8 Hz, 1H, Ar); 7.80 (d, J 5.4 Hz, 1H, Ar); 8.28 (d, J 5.4 Hz, 1H, Ar); 8.96 (s, 1H, Ar); 9.43 (s, 1H, Ar); 11.72 (s, 1H, NH). M/Z (M+H)⁺=394.0.

### Compound 81 (8-Methyl-6-(1-methyl-6-oxo-piperidin-3-yloxy)-2-thieno[2,3-c]pyridin-5-yl-3H-quinazolin-4-one)

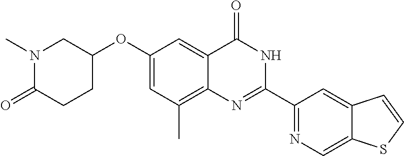 [SMILES: Cc1cc(OC2CCC(=O)N(C)C2)cc2c(=O)[nH]c(-c3cc4ccsc4cn3)nc12]

Compound 81 was prepared according to procedure of example 27, step 1 and 2, starting from 5-hydroxy-1-methyl-piperidin-2-one in step 2, followed by procedure of example 30, step 2. Purification by preparative HPLC afforded the product as a white solid.

¹H-NMR (400 MHz, DMSO): 2.05-2.12 (m, 2H, CH₂); 2.24-2.43 (m, 2H, CH₂); 2.69 (s, 3H, CH₃); 2.83 (s, 3H, CH₃); 3.44 (dd, J 13.3, 3.7 Hz, 1H, CH₂); 3.69 (dd, J 13.3, 3.9 Hz, 1H, CH₂); 5.04 (quin, J 3.9 Hz, 1H, CH—O); 7.44 (dd, J 2.9, 0.8 Hz, 1H, Ar); 7.52 (d, J 2.9 Hz, 1H, Ar); 7.81 (dd, J 5.3, 0.5 Hz, 1H, Ar); 8.28 (d, J 5.3 Hz, 1H, Ar); 8.97 (d, J 0.9 Hz, 1H, Ar); 9.44 (bs, 1H, Ar); 11.75 (s, 1H, NH). M/Z (M+H)⁺=421.7. MP=221-225° C.

### Compound 81-E1 ((R or S)-8-Methyl-6-((1-methyl-6-oxopiperidin-3-yl)oxy)-2-(thieno[2,3-c]pyridin-5-yl)quinazolin-4(3H)-one)

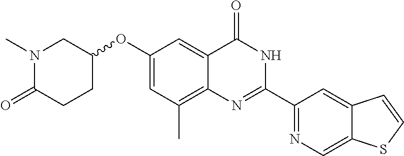 [SMILES: Cc1cc(OC2CCC(=O)N(C)C2)cc2c(=O)[nH]c(-c3cc4ccsc4cn3)nc12]

Compound 81-E1 was obtained by chiral separation of compound 81 on a CHIRALPAK_{®}IA column (5 μm 250×4.6 mm) using carbon dioxide/(ethanol+1% diethylamine) 60/40 as an eluant and isolating the first eluting enantiomer. Purification by trituration in diethyl ether afforded the product as a beige solid.

¹H-NMR (400 MHz, DMSO): 2.04-2.13 (m, 2H, CH₂); 2.24-2.43 (m, 2H, CH₂); 2.68 (s, 3H, CH₃); 2.83 (s, 3H, CH₃—N); 3.44 (dd, J 13.3, 3.6 Hz, 1H, CH₂); 3.69 (dd, J 13.3, 3.9 Hz, 1H, CH₂); 5.04 (quin, J 3.9 Hz, 1H, CH—O); 7.44 (d, J 2.3 Hz, 1H, Ar); 7.51 (d, J 2.9 Hz, 1H, Ar); 7.81 (d, J 5.3 Hz, 1H, Ar); 8.29 (d, J 5.3 Hz, 1H, Ar); 8.96 (s, 1H, Ar); 9.43 (bs, 1H, Ar); 11.74 (s, 1H, NH). M/Z (M+H)⁺=421.8. MP=240-245° C.

### Compound 81-E2 ((R or S)-8-Methyl-6-((1-methyl-6-oxopiperidin-3-yl)oxy)-2-(thieno[2,3-c]pyridin-5-yl)quinazolin-4(3H)-one)

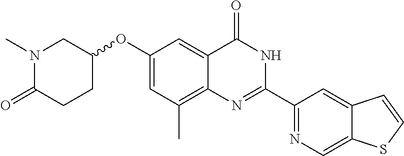 [SMILES: Cc1cc(OC2CCC(=O)N(C)C2)cc2c(=O)[nH]c(-c3cc4ccsc4cn3)nc12]

Compound 81-E2 was obtained by chiral separation of compound 81 on a CHIRALPAK_{®}IA column (5 μm 250×4.6 mm) using carbon dioxide/(ethanol+1% diethylamine) 60/40 as an eluant and isolating the second eluting enantiomer. Purification by column chromatography on silica gel, using dichloromethane/methanol as eluent, afforded the product as an off-white solid.

¹H-NMR (400 MHz, DMSO): 2.04-2.13 (m, 2H, CH₂); 2.24-2.43 (m, 2H, CH₂); 2.68 (s, 3H, CH₃); 2.83 (s, 3H, CH₃—N); 3.44 (dd, J 13.3, 3.6 Hz, 1H, CH₂); 3.69 (dd, J 13.3, 3.9 Hz, 1H, CH₂); 5.04 (quin, J 3.9 Hz, 1H, CH—O); 7.44 (dd, J 0.6, 2.9 Hz, 1H, Ar); 7.51 (d, J 2.9 Hz, 1H, Ar); 7.81 (dd, J 0.4, 5.3 Hz, 1H, Ar); 8.29 (d, J 5.3 Hz, 1H, Ar); 8.96 (d, J 1.0 Hz, 1H, Ar); 9.44 (bs, 1H, Ar); 11.74 (s, 1H, NH). M/Z (M+H)⁺=421.8. MP=245-250° C.

### Compound 82-R (R-8-Methyl-6-(1-propionyl-pyrrolidin-3-yloxy)-2-thieno[2,3-c]pyridin-5-yl-3H-quinazolin-4-one)

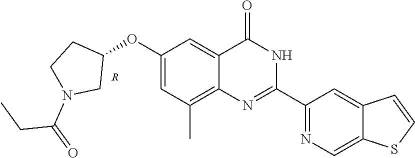

Compound 82-R was prepared according to procedure of example 27, step 1 and 2, starting from (S)-1-(3-hydroxy-pyrrolidin-1-yl)-propan-1-one in step 2, followed by procedure of example 36, step 7. Purification by column chromatography on silica gel, using dichloromethane/methanol as eluent, afforded the product as a yellow solid.

¹H-NMR (400 MHz, DMSO): 1.00 (t, J 7.4 Hz, 3H, ethyl); 2.13 (m, 1H, CH); 2.24 (q, J 7.4 Hz, 2H, ethyl); 2.30 (m, 1H, CH); 2.36 (m, 1H, CH); 2.68 (s, 3H, CH₃); 3.37 (m, 1H, CH); 3.60 (m, 3H, 3CH); 5.18 (m, 1H, CH); 7.40 (d, J 2.9 Hz, 1H, Ar); 7.43 (d, J 2.9 Hz, 1H, Ar); 7.80 (d, J 5.4 Hz, 1H, Ar); 8.28 (d, J 5.4 Hz, 1H, Ar); 8.96 (s, 1H, Ar); 9.43 (s, 1H, Ar); 11.74 (s, 1H, NH). M/Z (M+H)⁺=435.0.

(S)-1-(3-Hydroxy-pyrrolidin-1-yl)-propan-1-one was prepared as follows:

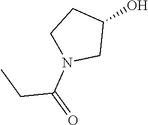

To a solution of (S)-3-hydroxypyrrolidine (500 mg, 5.74 mmol) and diisopropylethylamine (2.3 mL, 13.2 mmol) in diethyl ether (28 mL) was added propionyl chloride (0.50 mL, 5.74 mmol) and the reaction mixture was stirred at room temperature for 16 h. After centrifugation, the supernatant was concentrated under vacuum to give the product (464 mg, 56%) as a yellow oil.

¹H-NMR (400 MHz, DMSO): 0.97 (t, J 7.4 Hz, 3H, ethyl); 1.74 (m, 1H, CH); 1.91 (m, 1H, CH); 2.20 (q, J 7.4 Hz, 2H, ethyl); 3.25 (m, 2H, 2 CH); 3.46 (m, 2H, 2 CH); 4.26 (m, 1H, CH); 4.91 (d, J 3.6 Hz, 1H, OH).

### Compound 82-S (S-8-Methyl-6-(1-propionyl-pyrrolidin-3-yloxy)-2-thieno[2,3-c]pyridin-5-yl-3H-quinazolin-4-one)

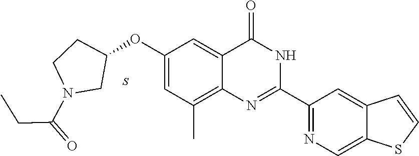

Compound 82-S was prepared according to procedure of compound 82-R, starting from (R)-1-(3-hydroxy-pyrrolidin-1-yl)-propan-1-one in step 2, to afford the product as a white solid.

¹H-NMR (400 MHz, DMSO): 1.00 (t, J 7.4 Hz, 3H, ethyl); 2.11 (m, 1H, CH); 2.13 (m, 1H, CH); 2.24 (q, J 7.4 Hz, 2H, ethyl); 2.30 (m, 1H, CH); 2.68 (s, 3H, CH₃); 3.37 (m, 1H, CH); 3.60 (m, 3H, 3CH); 5.18 (m, 1H, CH); 7.39 (d, J 2.9 Hz, 1H, Ar); 7.43 (d, J 2.9 Hz, 1H, Ar); 7.80 (d, J 5.4 Hz, 1H, Ar); 8.28 (d, J 5.4 Hz, 1H, Ar); 8.96 (s, 1H, Ar); 9.43 (s, 1H, Ar); 11.74 (s, 1H, NH). M/Z (M+H)⁺=435.4.

(R)-1-(3-Hydroxy-pyrrolidin-1-yl)-propan-1-one (314 mg, 38%) was prepared using procedure of (S)-1-(3-hydroxy-pyrrolidin-1-yl)-propan-1-one and starting from (R)-3-hydroxypyrrolidine

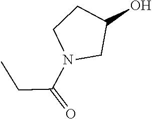

¹H-NMR (400 MHz, DMSO): 0.97 (t, J 7.4 Hz, 3H, ethyl); 1.82 (m, 1H, CH); 1.92 (m, 1H, CH); 2.18 (q, J 7.4 Hz, 2H, ethyl); 3.25 (m, 2H, 2 CH); 3.46 (m, 2H, 2 CH); 4.22 (m, 1H, CH); 4.86 (d, J 3.6 Hz, 1H, OH).

### Compound 83-R (R-8-Methyl-6-(1-oxetan-3-yl-pyrrolidin-3-yloxy)-2-thieno[2,3-c]pyridin-5-yl-3H-quinazolin-4-one)

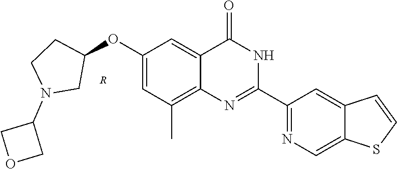

Compound 83-R was prepared according to procedure of compound 82-R, starting from (S)-1-oxetan-3-yl-pyrrolidin-3-ol in step 2, to afford the product as a white solid.

¹H-NMR (400 MHz, DMSO): 1.89 (m, 1H, CH); 2.33 (m, 1H, CH); 2.52 (m, 1H, CH); 2.68 (s, 3H, CH₃); 2.72 (m, 2H, 2 CH); 2.90 (m, 1H, Ar); 3.66 (m, 1H, CH); 4.47 (dt, J 9.9, 5.9 Hz, 2H, 2 CH); 4.58 (dt, J 6.5, 1.7 Hz, 2H, 2 CH); 5.05 (m, 1H, CH); 7.35 (m, 2H, Ar); 7.80 (d, J 5.4 Hz, 1H, Ar); 8.28 (d, J 5.4 Hz, 1H, Ar); 8.96 (s, 1H, Ar); 9.43 (s, 1H, Ar); 11.72 (s, 1H, NH). M/Z (M+H)⁺=435.0.

(S)-1-oxetan-3-yl-pyrrolidin-3-ol was prepared as follows:

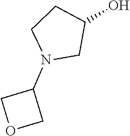

To a solution of (S)-3-hydroxypyrrolidine (500 mg, 5.74 mmol) and 3-oxetanone (404 μL, 6.31 mmol) in THF (28 mL) was added sodium triacetoxyborohydride (1.82 g, 8.61 mmol) and the reaction mixture was stirred at room temperature for 16 h. After filtration with dichloromethane, the supernatant was concentrated under vacuum and purified by column chromatography on silica gel, using dichloromethane/methanol as eluent to give the product (525 mg, 64%) as a yellow oil.

¹H-NMR (400 MHz, DMSO): 1.55 (m, 1H, CH); 1.96 (m, 1H, CH); 2.25 (m, 1H, CH); 2.40 (m, 1H, CH); 2.52 (m, 1H, CH); 2.67 (m, 1H, CH); 3.56 (m, 1H, CH); 4.19 (m, 1H, CH); 4.42 (m, 2H, 2 CH); 4.54 (t, J 6.5 Hz, 2H, 2 CH); 4.70 (d, J 4.6 Hz, 1H, OH).

### Compound 83-S (S-8-Methyl-6-(1-oxetan-3-yl-pyrrolidin-3-yloxy)-2-thieno[2,3-c]pyridin-5-yl-3H-quinazolin-4-one)

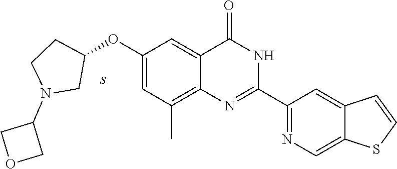

Compound 83-S was prepared according to procedure of compound 82-R, starting from (R)-1-oxetan-3-yl-pyrrolidin-3-ol in step 2, to afford the product as a white solid.

¹H-NMR (400 MHz, DMSO): 1.89 (m, 1H, CH); 2.33 (m, 1H, CH); 2.52 (m, 1H, CH); 2.68 (s, 3H, CH₃); 2.74 (m, 2H, 2 CH); 2.90 (m, 1H, Ar); 3.67 (m, 1H, CH); 4.48 (dt, J 9.9, 5.9 Hz, 2H, 2 CH); 4.58 (dt, J 6.5, 1.7 Hz, 2H, 2 CH); 5.05 (m, 1H, CH); 7.35 (m, 2H, Ar); 7.79 (d, J 5.4 Hz, 1H, Ar); 8.28 (d, J 5.4 Hz, 1H, Ar); 8.95 (s, 1H, Ar); 9.42 (s, 1H, Ar); 11.71 (s, 1H, NH). M/Z (M+H)⁺=435.4.

(R)-1-oxetan-3-yl-pyrrolidin-3-ol (406 mg, 49%) was prepared using procedure of (S)-1-oxetan-3-yl-pyrrolidin-3-ol and starting from (R)-3-hydroxypyrrolidine.

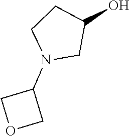

¹H-NMR (400 MHz, DMSO): 1.54 (m, 1H, CH); 1.95 (m, 1H, CH); 2.25 (m, 1H, CH); 2.40 (m, 1H, CH); 2.52 (m, 1H, CH); 2.67 (m, 1H, CH); 3.56 (m, 1H, CH); 4.19 (m, 1H, CH); 4.42 (m, 2H, 2 CH); 4.54 (t, J 6.5 Hz, 2H, 2 CH); 4.70 (d, J 4.6 Hz, 1H, OH).

### Compound 84 (8-Methyl-6-[2-(2-oxa-7-aza-spiro[3.5]non-7-yl)-ethoxy]-2-thieno[2,3-c]pyridin-5-yl-3H-quinazolin-4-one)

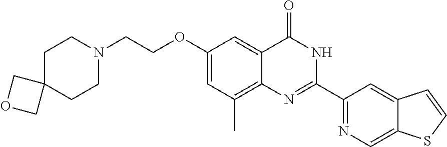 [SMILES: Cc1cc(OCCN2CCC3(CC2)COC3)cc2c(=O)[nH]c(-c3cc4ccsc4cn3)nc12]

Compound 84 was prepared according to procedure of example 27, step 1 and 2, starting from 2-(2-oxa-7-aza-spiro[3.5]non-7-yl)-ethanol in step 2, followed by procedure of example 30, step 2, to afford the product as a white solid.

¹H-NMR (400 MHz, CDCl₃): 1.53 (m, 4H, 2 CH₂); 2.06 (m, 3H, 3CH); 2.67 (m, 5H, 5CH); 2.99 (m, 2H, 2 CH); 4.36 (m, 5H, CH₃+2 CH); 7.19 (m, 1H, Ar); 7.50 (m, 2H, Ar); 7.78 (d, J 5.4 Hz, 1H, Ar); 8.92 (s, 1H, Ar); 9.09 (s, 1H, Ar); 10.98 (s, 1H, NH). M/Z (M+H)⁺=463.7.

2-(2-Oxa-7-aza-spiro[3.5]non-7-yl)-ethanol was prepared as follows:

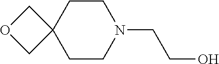 [SMILES: OCCN1CCC2(CC1)COC2]

In a sealed vial, a suspension of 2-oxa-7-aza-spiro[3,5]nonane oxalic acid (100 mg, 0.46 mmol), potassium carbonate (115 mg, 0.84 mmol) and bromoethanol (30 μL, 0.42 mmol) in dry acetonitrile (1.5 mL) was heated at 90° C. for 16 h. After cooling to room temperature, the suspension was filtered off and the filtrate was concentrated under vacuum to give the product (80 mg, quantitative yield) as a yellow oil.

¹H-NMR (400 MHz, CDCl₃): 1.83 (m, 2H, 2 CH); 2.01 (m, 3H, 3CH); 2.59 (bs, 1H, OH); 2.63 (m, 2H, 2 CH); 3.38 (m, 2H, 2 CH); 3.69 (m, 1H, CH); 3.79 (m, 1H, CH); 4.22 (m, 1H, CH); 4.41 (m, 4H, 2 CH₂).

### Example 28—Synthesis of compounds 85 (8-methyl-6-(piperidin-4-ylmethoxy)-2-thieno[2,3-c]pyridin-5-yl-3H-quinazolin-4-one) and 86 (8-Methyl-6-(1-oxetan-3-yl-piperidin-4-ylmethoxy)-2-thieno[2,3-c]pyridin-5-yl-3H-quinazolin-4-one)

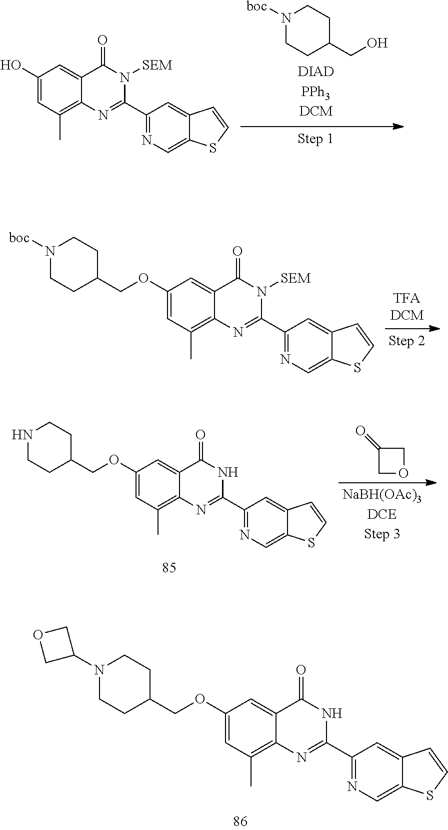

Step 1:

4-[8-Methyl-4-oxo-2-thieno[2,3-c]pyridin-5-yl-3-(2-trimethylsilanyl-ethoxymethyl)-3,4-dihydro-quinazolin-6-yloxymethyl]-piperidine-1-carboxylic acid tert-butyl ester was prepared according to procedure of example 23, step 6, starting from 6-hydroxy-8-methyl-2-thieno[2,3-c]pyridin-5-yl-3-(2-trimethylsilanyl-ethoxymethyl)-3H-quinazolin-4-one (325 mg, 0.74 mmol) and N-boc-4-piperidinemethanol (239 mg, 1.11 mmol) to afford the product (186 mg, 39%) as a colorless oil.

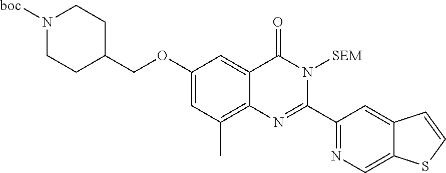

M/Z (M+H)⁺=637.8.

Step 2:

At 0° C., to a solution of 4-[8-Methyl-4-oxo-2-thieno[2,3-c]pyridin-5-yl-3-(2-trimethylsilanyl-ethoxymethyl)-3,4-dihydro-quinazolin-6-yloxymethyl]-piperidine-1-carboxylic acid tert-butyl ester (166 mg, 0.26 mmol) in dichloromethane (1.5 mL) was added TFA (0.40 mL, 5.20 mmol). The reaction mixture was stirred for 4 h at room temperature before being neutralized with aqueous potassium carbonate and extracted twice with ethyl acetate. The combined organic extracts were washed with brine, dried over MgSO₄, filtered and concentrated under vacuum to give compound 85 (84 mg, 79%) as a beige solid.

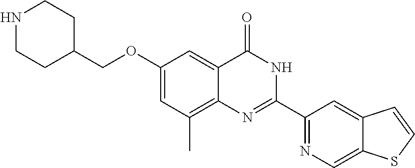 [SMILES: Cc1cc(OCC2CCNCC2)cc2c(=O)[nH]c(-c3cc4ccsc4cn3)nc12]

M/Z (M+H)⁺=407.6.

Step 3:

To a solution compound 85 (40 mg, 0.10 mmol) and 3-oxetanone (18 mg, 0.24 mmol) in dry 1,2-dichloroethane (1.4 mL) was added sodium triacetoxyborohydride (62 mg, 0.29 mmol). The reaction mixture was stirred for 17 h at room temperature before being treated with aqueous sodium carbonate and extracted with dichloromethane. The combined organic extracts were washed with brine, dried over MgSO₄, filtered and concentrated under vacuum. Purification by column chromatography on silica gel, using dichloromethane/methanol as eluent, afforded compound 86 as a white solid (10 mg, 21%).

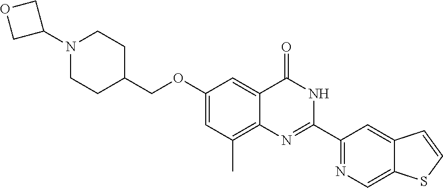 [SMILES: Cc1cc(OCC2CCN(C3COC3)CC2)cc2c(=O)[nH]c(-c3cc4ccsc4cn3)nc12]

¹H-NMR (400 MHz, DMSO): 1.38 (m, 2H, 2 CH); 1.80 (m, 4H, 2 CH₂); 2.69 (s, 3H, CH₃); 2.76 (m, 1H, CH); 3.41 (m, 2H, 2 CH); 3.97 (d, J 5.9 Hz, 2H, CH₂—O); 4.44 (m, 2H, 2 CH); 4.54 (m, 2H, 2 CH); 7.38 (d, J 2.8 Hz, 1H, Ar); 7.43 (d, J 2.8 Hz, 1H, Ar); 7.81 (d, J 5.4 Hz, 1H, Ar); 8.28 (d, J 5.4 Hz, 1H, Ar); 8.97 (s, 1H, Ar); 9.44 (s, 1H, Ar); 11.76 (s, 1H, NH). M/Z (M+H)⁺=463.7.

### Example 29—Synthesis of compound 87 (8-Methyl-6-(1-propionyl-piperidin-4-ylmethoxy)-2-thieno[2,3-c]pyridin-5-yl-3H-quinazolin-4-one)

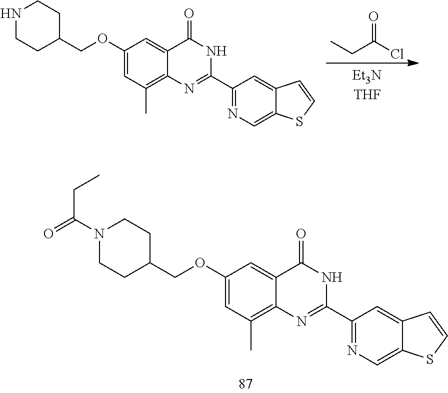

To a solution of compound 85 (40 mg, 0.10 mmol) in dry THF (4.0 mL) were added triethylamine (42 μL, 0.30 mmol) and propionyl chloride (13 μL, 0.15 mmol). The reaction mixture was stirred for 1 h at room temperature before being treated with water and extracted twice with ethyl acetate. The combined organic extracts were washed with brine, dried over MgSO₄, filtered and concentrated under vacuum. Purification by column chromatography on silica gel, using dichloromethane/methanol as eluent, afforded compound 87 as a white solid (10 mg, 21%).

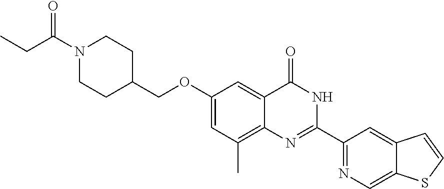 [SMILES: CCC(=O)N1CCC(COc2cc(C)c3nc(-c4cc5ccsc5cn4)[nH]c(=O)c3c2)CC1]

¹H-NMR (400 MHz, DMSO): 0.99 (t, J 7.3 Hz, 3H, ethyl); 1.21 (m, 4H, 2 CH₂); 1.84 (m, 2H, 2 CH); 2.33 (q, J 7.3 Hz, 2H, ethyl); 2.69 (s, 3H, CH₃); 2.76 (m, 1H, CH); 3.90 (m, 1H, CH); 3.98 (d, J 6.1 Hz, 2H, CH₂—O); 4.44 (m, 1H, CH); 7.38 (d, J 2.6 Hz, 1H, Ar); 7.43 (d, J 2.6 Hz, 1H, Ar); 7.81 (d, J 5.4 Hz, 1H, Ar); 8.28 (d, J 5.4 Hz, 1H, Ar); 8.97 (s, 1H, Ar); 9.43 (s, 1H, Ar); 11.72 (s, 1H, NH). M/Z (M+H)⁺=463.7.

### Compound 88 (6-(1-Methanesulfonyl-piperidin-4-ylmethoxy)-8-methyl-2-thieno[2,3-c]pyridin-5-yl-3H-quinazolin-4-one)

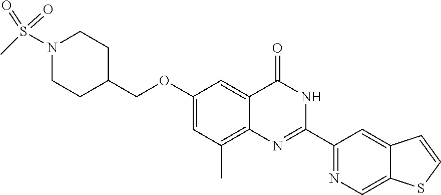 [SMILES: Cc1cc(OCC2CCN(S(C)(=O)=O)CC2)cc2c(=O)[nH]c(-c3cc4ccsc4cn3)nc12]

Compound 88 was prepared according to procedure of example 29, starting from methane sulfonyl chloride instead of propionyl chloride, to afford compound 88 as a yellow solid.

¹H-NMR (400 MHz, DMSO): 1.20 (m, 1H, CH); 1.40 (m, 2H, 2 CH); 1.92 (m, 2H, 2 CH); 2.68 (s, 3H, CH₃); 2.76 (m, 2H, 2 CH); 2.86 (s, 3H, CH₃); 3.61 (m, 2H, 2 CH); 4.01 (d, J 6.1 Hz, 2H, CH₂O); 7.38 (d, J 2.8 Hz, 1H, Ar); 7.43 (d, J 2.8 Hz, 1H, Ar); 7.82 (d, J 5.4 Hz, 1H, Ar); 8.28 (d, J 5.4 Hz, 1H, Ar); 8.98 (s, 1H, Ar); 9.45 (s, 1H, Ar); 11.74 (s, 1H, NH). M/Z (M+H)⁺=485.7.

### Example 30—Synthesis of compound 89 (8-Methyl-6-(2-oxa-7-aza-spiro[3.5]non-7-yl)-2-thieno[2,3-c]pyridin-5-yl-3H-quinazolin-4-one)

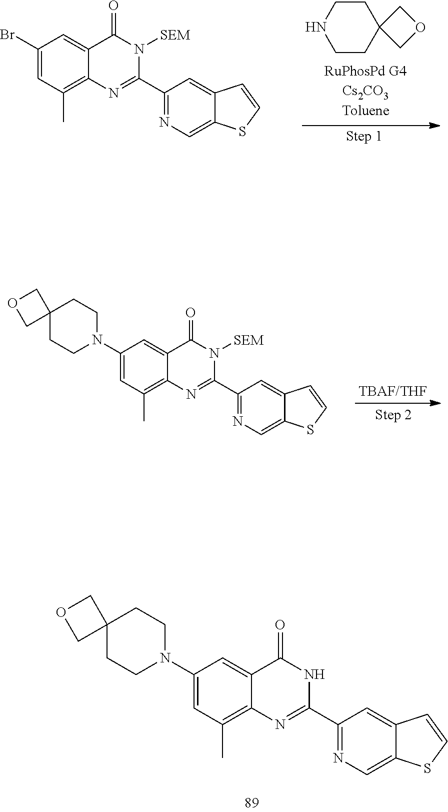 [SMILES: Cc1cc(Br)cc2c(=O)n(COCC[Si](C)(C)C)c(-c3cc4ccsc4cn3)nc12 ; C1CC2(CCN1)COC2 ; Cc1cc(N2CCC3(CC2)COC3)cc2c(=O)n(COCC[Si](C)(C)C)c(-c3cc4ccsc4cn3)nc12 ; Cc1cc(N2CCC3(CC2)COC3)cc2c(=O)[nH]c(-c3cc4ccsc4cn3)nc12]

Step 1:

Under inert atmosphere, to a suspension of 6-bromo-8-methyl-2-thieno[2,3-c]pyridin-5-yl-3-(2-trimethylsilanyl-ethoxymethyl)-3H-quinazolin-4-one (200 mg, 0.40 mmol), cesium carbonate (648 mg, 1.99 mmol) and 2-oxa-7-aza-spiro[3.5]nonane (173 mg, 0.80 mmol) in dry toluene (4.0 mL) was added RuPhos precatalyst generation 4 (34 mg, 0.04 mmol) and the reaction mixture was heated at 90° C. for 16 h. After cooling to room temperature, the mixture was treated with water and extracted twice with ethyl acetate. The combined organic extracts were washed with brine, dried over MgSO₄, filtered and concentrated under vacuum. Purification by column chromatography on silica gel, using cyclohexane/ethyl acetate as eluent, afforded 8-methyl-6-(2-oxa-7-aza-spiro[3.5]non-7-yl)-2-thieno[2,3-c]pyridin-5-yl-3-(2-trimethylsilanyl-ethoxymethyl)-3H-quinazolin-4-one (182 mg, 83%) as a yellow solid.

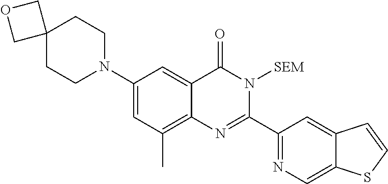 [SMILES: Cc1cc(N2CCC3(CC2)COC3)cc2c(=O)n(COCC[Si](C)(C)C)c(-c3cc4ccsc4cn3)nc12]

M/Z (M+H)⁺=549.8.

Step 2:

To a solution of 8-methyl-6-(2-oxa-7-aza-spiro[3.5]non-7-yl)-2-thieno[2,3-c]pyridin-5-yl-3-(2-trimethylsilanyl-ethoxymethyl)-3H-quinazolin-4-one (179 mg, 0.33 mmol) in THF (1.6 mL) was added tetrabutylmammonium fluoride 1M in THF (1.6 mL, 1.63 mmol) and the reaction mixture was stirred for 23 h at 70° C. After cooling to room temperature, the mixture was diluted with ethyl acetate and washed twice with water. The organic layer was then washed with brine, dried over MgSO₄, filtered and concentrated under vacuum. Purification by trituration in ethanol and diethyl ether afforded compound 89 as a beige solid (101 mg, 74%).

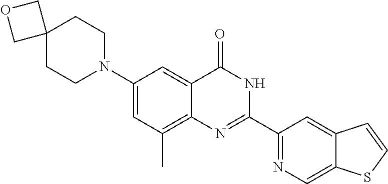 [SMILES: Cc1cc(N2CCC3(CC2)COC3)cc2c(=O)[nH]c(-c3cc4ccsc4cn3)nc12]

¹H-NMR (400 MHz, DMSO): 1.91 (m, 4H, 2 CH₂); 2.66 (s, 3H, CH₃); 3.24 (m, 4H, 2 CH₂); 4.36 (s, 4H, 2 CH₂); 7.34 (d, J 2.5 Hz, 1H, Ar); 7.50 (d, J 2.5 Hz, 1H, Ar); 7.79 (d, J 5.4 Hz, 1H, Ar); 8.27 (d, J 5.4 Hz, 1H, Ar); 8.92 (s, 1H, Ar); 9.41 (s, 1H, Ar); 11.55 (s, 1H, NH). M/Z (M+H)⁺=418.9.

### Compound 90 (8-Methyl-6-(6-oxa-2-aza-spiro[3.4]oct-2-yl)-2-thieno[2,3-c]pyridin-5-yl-3H-quinazolin-4-one)

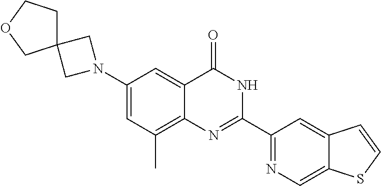 [SMILES: Cc1cc(N2CC3(CCOC3)C2)cc2c(=O)[nH]c(-c3cc4ccsc4cn3)nc12]

Compound 90 was prepared according to procedure of example 30, starting from 6-oxa-2-azaspiro[3.4]octane hemioxalate in step 1, to afford the product as a beige solid.

¹H-NMR (400 MHz, DMSO): 2.18 (t, J 6.9 Hz, 2H, 2 CH); 2.66 (s, 3H, CH₃); 3.76 (t, J 6.9 Hz, 2H, 2 CH); 3.84 (s, 2H, 2 CH); 3.93 (AB system, J 7.5 Hz, 4H, 2 CH₂); 6.90 (d, J 2.6 Hz, 1H, Ar); 6.93 (d, J 2.6 Hz, 1H, Ar); 7.78 (d, J 5.4 Hz, 1H, Ar); 8.26 (d, J 5.4 Hz, 1H, Ar); 8.91 (s, 1H, Ar); 9.40 (s, 1H, Ar); 11.49 (s, 1H, NH). M/Z (M+H)⁺=405.0.

### Compound 91 (8-Methyl-6-(3-oxa-9-aza-spiro[5.5]undec-9-yl)-2-thieno[2,3-c]pyridin-5-yl-3H-quinazolin-4-one)

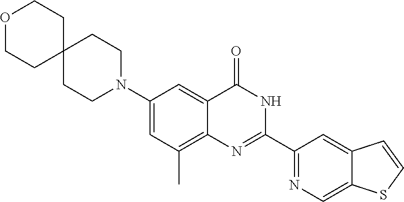 [SMILES: Cc1cc(N2CCC3(CCOCC3)CC2)cc2c(=O)[nH]c(-c3cc4ccsc4cn3)nc12]

Compound 91 was prepared according to procedure of example 30, starting from 3-oxa-9-azaspiro[5.5]undecane in step 1, to afford the product as a beige solid.

¹H-NMR (400 MHz, DMSO): 1.47 (t, J 5.4 Hz, 4H, 2 CH₂); 1.62 (dd, J 6.8, 4.4 Hz, 4H, 2 CH₂); 2.65 (s, 3H, CH₃); 3.29 (dd, J 6.8, 4.4 Hz, 4H, 2 CH₂); 3.58 (t, J 5.4 Hz, 4H, 2 CH₂); 7.33 (d, J 2.6 Hz, 1H, Ar); 7.47 (d, J 2.6 Hz, 1H, Ar); 7.77 (d, J 5.4 Hz, 1H, Ar); 8.24 (d, J 5.4 Hz, 1H, Ar); 8.91 (s, 1H, Ar); 9.39 (s, 1H, Ar); 11.51 (s, 1H, NH); M/Z (M+H)⁺=447.7.

### Compound 92 (8-Methyl-6-(7-oxa-2-aza-spiro[4.5]dec-2-yl)-2-thieno[2,3-c]pyridin-5-yl-3H-quinazolin-4-one)

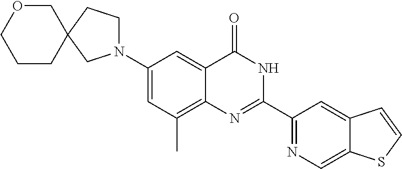 [SMILES: Cc1cc(N2CCC3(CCCOC3)C2)cc2c(=O)[nH]c(-c3cc4ccsc4cn3)nc12]

Compound 92 was prepared according to procedure of example 30, starting from 7-oxa-2-azaspiro[4.5]decane in step 1. Purification by column chromatography on silica gel, using cyclohexane/ethyl acetate as eluent, and trituration in diethyl ether afforded the product as a white solid.

¹H-NMR (400 MHz, DMSO): 1.62 (m, 2H, 2 CH); 1.69 (m, 2H, 2 CH); 1.79 (m, 1H, CH); 1.96 (m, 1H, CH); 2.67 (s, 3H, CH₃); 3.12 (d, J 10.0 Hz, 1H, CH); 3.40 (m, 5H, 5CH); 3.54 (m, 1H, CH); 3.65 (m, 1H, CH); 6.95 (d, J 2.6 Hz, 1H, Ar); 7.07 (d, J 2.6 Hz, 1H, Ar); 7.78 (d, J 5.4 Hz, 1H, Ar); 8.25 (d, J 5.4 Hz, 1H, Ar); 8.90 (s, 1H, Ar); 9.39 (s, 1H, Ar); 11.40 (s, 1H, NH). M/Z (M+H)⁺=432.8.

### Compound 93 (8-Methyl-6-(8-oxa-2-aza-spiro[4.5]dec-2-yl)-2-thieno[2,3-c]pyridin-5-yl-3H-quinazolin-4-one)

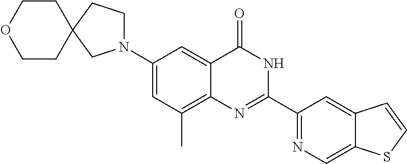 [SMILES: Cc1cc(N2CCC3(CCOCC3)C2)cc2c(=O)[nH]c(-c3cc4ccsc4cn3)nc12]

Compound 93 was prepared according to procedure of example 30, starting from 8-oxa-2-azaspiro[4.5]decane in step 1, to afford the product as a yellow solid.

¹H-NMR (400 MHz, DMSO): 1.58 (m, 4H, 2 CH₂); 1.94 (t, J 7.0 Hz, 2H, 2 CH); 2.67 (s, 3H, CH₃); 3.29 (s, 2H, CH₂); 3.43 (t, J 7.0 Hz, 2H, 2 CH); 3.64 (m, 4H, 2 CH₂); 6.96 (d, J 2.6 Hz, 1H, Ar); 7.07 (d, J 2.6 Hz, 1H, Ar); 7.78 (d, J 5.4 Hz, 1H, Ar); 8.25 (d, J 5.4 Hz, 1H, Ar); 8.90 (s, 1H, Ar); 9.39 (s, 1H, Ar); 11.39 (s, 1H, NH). M/Z (M+H)⁺=432.6.

### Compound 94 (6-(2-Hydroxy-2-methyl-propylamino)-8-methyl-2-thieno[2,3-c]pyridin-5-yl-3H-quinazolin-4-one)

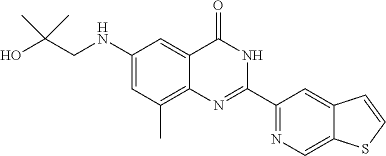 [SMILES: Cc1cc(NCC(C)(C)O)cc2c(=O)[nH]c(-c3cc4ccsc4cn3)nc12]

Compound 94 was prepared according to procedure of example 30, starting from 1-amino-2-methyl-2-propanol and BrettPhos precatalyst generation 1 in step 1. The product was obtained as a yellow solid.

¹H-NMR (400 MHz, DMSO): 1.20 (s, 6H, 2 CH₃); 2.61 (s, 3H, CH₃); 3.06 (d, J 5.7 Hz, 2H, CH₂); 4.51 (s, 1H, OH); 5.95 (t, J 5.7 Hz, 1H, NH); 7.07 (d, J 2.6 Hz, 1H, Ar); 7.20 (d, J 2.6 Hz, 1H, Ar); 7.78 (d, J 5.4 Hz, 1H, Ar); 8.25 (d, J 5.4 Hz, 1H, Ar); 8.90 (s, 1H, Ar); 9.39 (s, 1H, Ar); 11.34 (s, 1H, NH). M/Z (M+H)⁺=381.5.

### Example 31—Synthesis of compounds 95 (8-Methyl-6-(2-piperidin-3-yl-ethoxy)-2-thieno[2,3-c]pyridin-5-yl-3H-quinazolin-4-one) and 96 (6-[2-(1-Acetyl-piperidin-3-yl)-ethoxy]-8-methyl-2-thieno[2,3-c]pyridin-5-yl-3H-quinazolin-4-one)

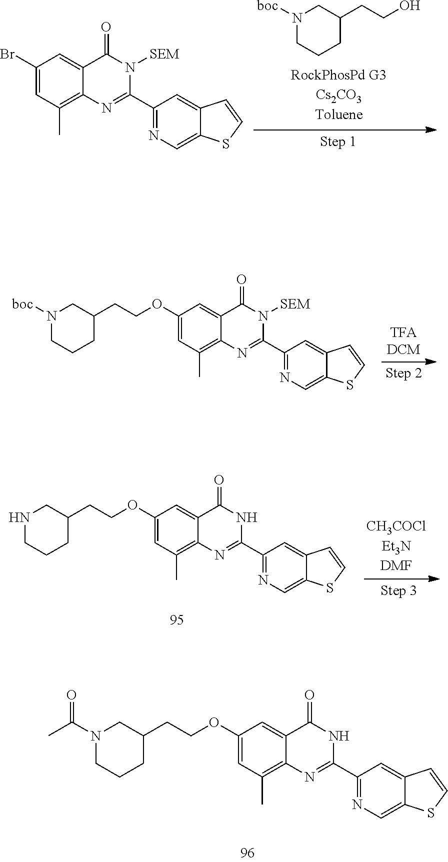

Step 1:

3-{2-[8-methyl-4-oxo-2-thieno[2,3-c]pyridin-5-yl-3-(2-trimethylsilanyl-ethoxymethyl)-3,4-dihydro-quinazolin-6-yloxy]ethyl}-piperidine-1-carboxylic acid tert-butyl ester was prepared according to procedure of example 19, step 3 starting from 6-bromo-8-methyl-2-thieno[2,3-c]pyridin-5-yl-3-(2-trimethylsilanyl-ethoxymethyl)-3H-quinazolin-4-one (75 mg, 0.15 mmol) and 1-N-boc-piperidine-3-ethanol (103 mg, 0.48 mmol), to afford the product (71 mg, 73%) as a yellow solid.

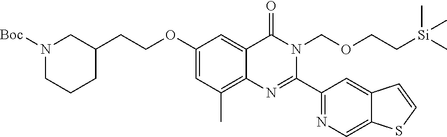 [SMILES: Cc1cc(OCCC2CCCN(C(=O)OC(C)(C)C)C2)cc2c(=O)n(COCC[Si](C)(C)C)c(-c3cc4ccsc4cn3)nc12]

M/Z (M+H)⁺=651.7.

Step 2:

Compound 95 was prepared according to procedure of example 28, step 2, starting from 3-{2-[8-methyl-4-oxo-2-thieno[2,3-c]pyridin-5-yl-3-(2-trimethylsilanyl-ethoxymethyl)-3,4-dihydro-quinazolin-6-yloxy]-ethyl}-piperidine-1-carboxylic acid tert-butyl ester. Purification by trituration in a solution of dichloromethane/methanol (9:1) afforded the product (37 mg, 81%) as an orange solid.

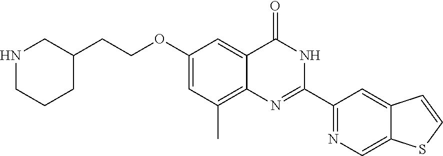 [SMILES: Cc1cc(OCCC2CCCNC2)cc2c(=O)[nH]c(-c3cc4ccsc4cn3)nc12]

M/Z (M+H)⁺=421.6.

Step 3:

Compound 96 was prepared according to procedure of example 29, starting from compound 95 and using acetyl chloride instead of propionyl chloride. Purification by trituration in diethyl ether afforded the product as a yellow solid.

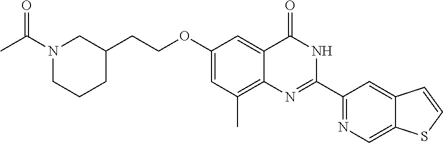 [SMILES: CC(=O)N1CCCC(CCOc2cc(C)c3nc(-c4cc5ccsc5cn4)[nH]c(=O)c3c2)C1]

¹H-NMR (400 MHz, DMSO): 1.17 (m, 2H, 2 CH); 1.26 (m, 1H, CH); 1.73 (m, 4H, 2 CH₂); 1.98 (s, 3H, CH₃); 2.68 (s, 3H, CH₃); 3.05 (m, 2H, 2 CH); 3.67 (m, 1H, CH); 4.15 (m, 3H, 3CH); 7.37 (d, J 2.6 Hz, 1H, Ar); 7.43 (d, J 2.6 Hz, 1H, Ar); 7.80 (d, J 5.4 Hz, 1H, Ar); 8.28 (d, J 5.4 Hz, 1H, Ar); 8.96 (s, 1H, Ar); 9.44 (s, 1H, Ar); 11.71 (s, 1H, NH). M/Z (M+H)⁺=463.7.

### Example 32—Synthesis of compound 97 (6-[2-(4-acetyl-piperazin-1-yl)-ethoxy]-8-methyl-2-thieno[2,3-c]pyridin-5-yl-3H-quinazolin-4-one hydrochloride)

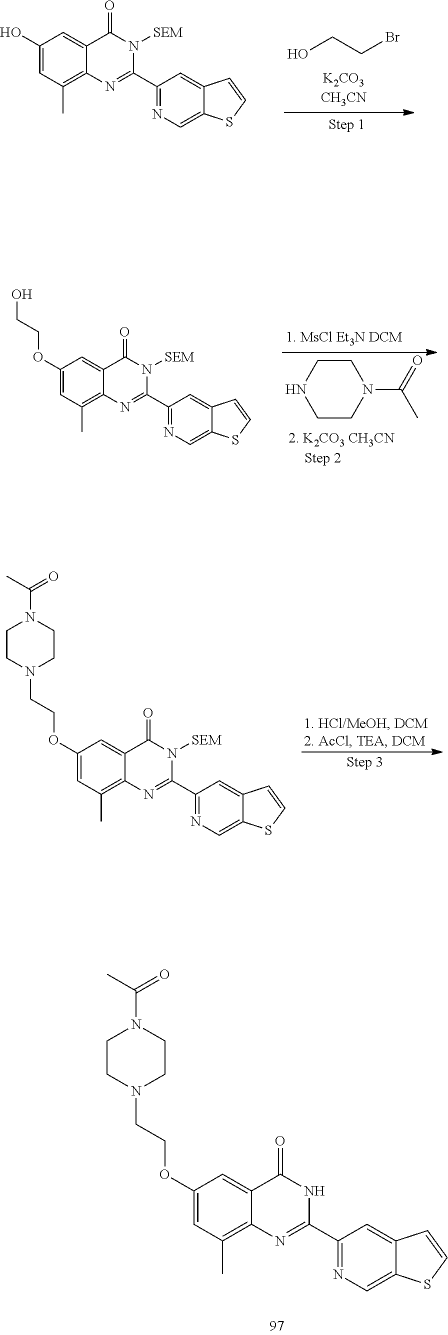

Step 1:

A suspension of 6-hydroxy-8-methyl-2-thieno[2,3-c]pyridin-5-yl-3-(2-trimethylsilanyl-ethoxymethyl)-3H-quinazolin-4-one (62 mg, 0.14 mmol), potassium carbonate (58 mg, 0.42 mmol) and 2-bromo-1-ethanol (45 μL, 0.63 mmol) in dry acetonitrile (2.0 mL) was heated at 100° C. for 16 h. After cooling to room temperature, the mixture was filtered off and the filtrate was concentrated under vacuum. Purification by column chromatography on silica gel, using cyclohexane/ethyl acetate as eluent, afforded 6-(2-hydroxy-ethoxy)-8-methyl-2-thieno[2,3-c]pyridin-5-yl-3-(2-trimethylsilanyl-ethoxymethyl)-3H-quinazolin-4-one (67 mg, quantitative yield) as a yellow oil.

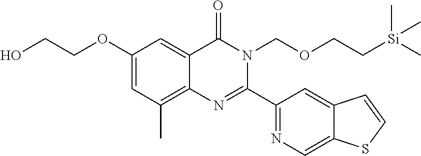 [SMILES: Cc1cc(OCCO)cc2c(=O)n(COCC[Si](C)(C)C)c(-c3cc4ccsc4cn3)nc12]

M/Z (M+H)⁺=484.6.

Step 2:

At 0° C., to a solution of 6-(2-hydroxy-ethoxy)-8-methyl-2-thieno[2,3-c]pyridin-5-yl-3-(2-trimethylsilanyl-ethoxymethyl)-3H-quinazolin-4-one (67 mg, 0.14 mmol) and triethylamine (38 μL, 0.27 mmol) in dichloromethane (2.0 mL) was added methane sulfonyl chloride (13 μL, 0.17 mmol). The reaction mixture was stirred for 1 h at room temperature before being treated with aqueous sodium carbonate and extracted with dichloromethane The combined organic extracts were washed with brine, dried over MgSO₄, filtered and concentrated under vacuum to give the mesylated intermediate as a crude yellow oil. M/Z (M+H)⁺=562.5.

A suspension of the crude oil, potassium carbonate (57 mg, 0.41 mmol) and 1-acetylpiperazine (27 mg, 0.21 mmol) in dry acetonitrile (2.0 mL) was heated at 100° C. for 48 h. After cooling to room temperature, the reaction mixture was treated with water and extracted twice with ethyl acetate. The combined organic extracts were washed with brine, dried over MgSO₄, filtered and concentrated under vacuum. Purification by column chromatography on silica gel, using dichloromethane/methanol as eluent, afforded 6-[2-(4-acetyl-piperazin-1-yl)-ethoxy]-8-methyl-2-thieno[2,3-c]pyridin-5-yl-3-(2-trimethylsilanyl-ethoxymethyl)-3H-quinazolin-4-one (11 mg, 13%) as a yellow oil.

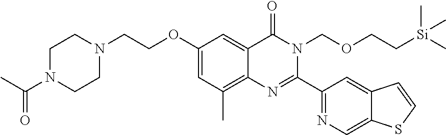 [SMILES: CC(=O)N1CCN(CCOc2cc(C)c3nc(-c4cc5ccsc5cn4)n(COCC[Si](C)(C)C)c(=O)c3c2)CC1]

M/Z (M+H)⁺=594.8.

Step 3:

Compound 97 was prepared according to procedure of example 9, step 4 & 5, starting from 6-[2-(4-acetyl-piperazin-1-yl)-ethoxy]-8-methyl-2-thieno[2,3-c]pyridin-5-yl-3-(2-trimethylsilanyl-ethoxymethyl)-3H-quinazolin-4-one (11 mg, 0.02 mmol), to afford the product as a beige solid (4 mg, 42%).

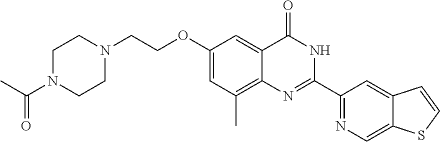 [SMILES: CC(=O)N1CCN(CCOc2cc(C)c3nc(-c4cc5ccsc5cn4)[nH]c(=O)c3c2)CC1]

¹H-NMR (400 MHz, DMSO): 1.99 (s, 3H, CH₃); 2.46 (m, 4H, 2 CH₂); 2.69 (s, 3H, CH₃); 2.79 (t, J 5.5 Hz, 2H, CH₂); 3.44 (m, 4H, 2 CH₂); 4.23 (t, J 5.5 Hz, 2H, CH₂); 7.39 (d, J 2.8 Hz, 1H, Ar); 7.46 (d, J 2.8 Hz, 1H, Ar); 7.83 (d, J 5.4 Hz, 1H, Ar); 8.27 (d, J 5.4 Hz, 1H, Ar); 9.00 (s, 1H, Ar); 9.44 (s, 1H, Ar); 11.74 (s, 1H, NH). M/Z (M+H)⁺=464.6.

### Example 33—Synthesis of compounds 98 (3-(8-methyl-4-oxo-2-thieno[2,3-c]pyridin-5-yl-3,4-dihydro-quinazolin-6-yl)-propionaldehyde) and 99 (8-Methyl-6-(3-morpholin-4-yl-propyl)-2-thieno[2,3-c]pyridin-5-yl-3H-quinazolin-4-one hydrochloride)

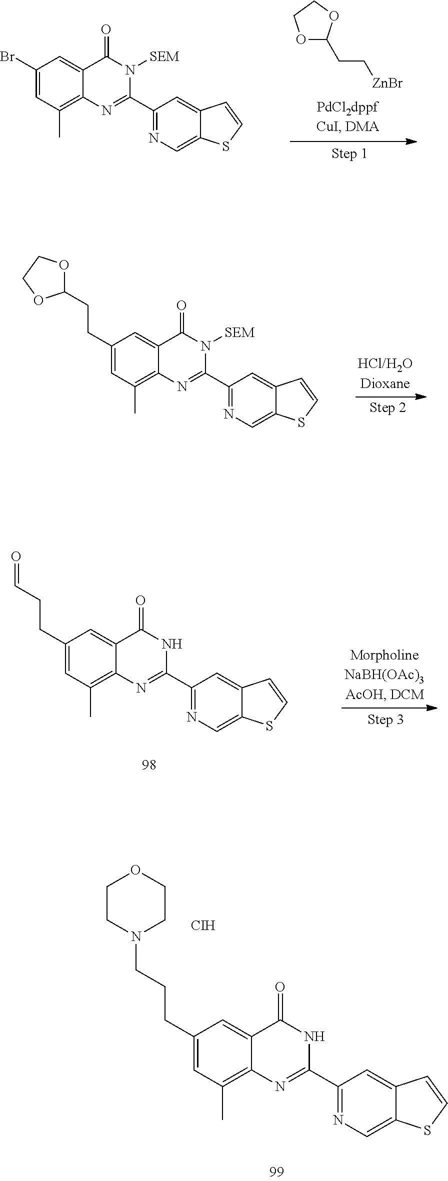

Step 1:

Under inert atmosphere, to a suspension of 6-bromo-8-methyl-2-thieno[2,3-c]pyridin-5-yl-3-(2-trimethylsilanyl-ethoxymethyl)-3H-quinazolin-4-one (50 mg, 0.10 mmol), copper iodide (2 mg, 0.01 mmol) and 2-(1,3-dioxolan-2-yl)ethylzinc bromide (0.5M solution in THF, 0.30 mL, 0.15 mmol) in dry DMA (0.7 mL) was added PdCl₂(dppf).CH₂Cl₂ (4 mg, 0.005 mmol) and the reaction mixture was heated at 80° C. for 16 h. After cooling to room temperature, the reaction mixture was treated with water and extracted twice with ethyl acetate. The combined organic extracts were washed with brine, dried over MgSO₄, filtered and concentrated under vacuum. Purification by column chroimatography on silica gel, using cyclohexane/ethyl acetate as eluent, afforded 6-(2-[1,3]dioxolan-2-yl-ethyl)-8-methyl-2-thieno[2,3-c]pyridin-5-yl-3-(2-trimethylsilanyl-ethoxymethyl)-3H-quinazolin-4-one (43 mg, 83%) as a brown oil.

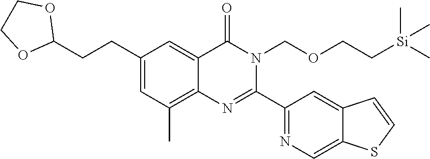 [SMILES: Cc1cc(CCC2OCCO2)cc2c(=O)n(COCC[Si](C)(C)C)c(-c3cc4ccsc4cn3)nc12]

M/Z (M+H)⁺=524.7.

Step 2:

To a solution of 6-(2-[1,3]dioxolan-2-yl-ethyl)-8-methyl-2-thieno[2,3-c]pyridin-5-yl-3-(2-trimethylsilanyl-ethoxymethyl)-3H-quinazolin-4-one (43 mg, 0.08 mmol) in dioxane (0.7 mL) was added aqueous HCl (3N, 0.2 mL) and the reaction mixture was heated at 70° C. for 3 h. After cooling to room temperature, the reaction mixture was neutralized with aqueous sodium bicarbonate and extracted twice with ethyl acetate. The combined organic extracts were washed with brine, dried over MgSO₄, filtered and concentrated under vacuum to give 3-(8-methyl-4-oxo-2-thieno[2,3-c]pyridin-5-yl-3,4-dihydro-quinazolin-6-yl)-propionaldehyde 98 (25 mg, 87%) as a brown solid.

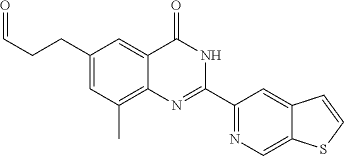 [SMILES: Cc1cc(CCC=O)cc2c(=O)[nH]c(-c3cc4ccsc4cn3)nc12]

M/Z (M+H)⁺=350.5.

Step 3:

To a suspension of compound 98 (25 mg, 0.07 mmol), morpholine (12 μL, 0.14 mmol) and acetic acid (41 μL, 0.007 mmol) in dichloromethane (7.0 mL) was added sodium triacetoxyborohydride (22 mg, 0.11 mmol). The reaction mixture was stirred for 16 h at room temperature before being treated with water and extracted with dichloromethane. The combined organic extracts were washed with brine, dried over MgSO₄, filtered and concentrated under vacuum. Purification by column chromatography on silica gel, using dichloromethane/methanol as eluent, followed by salt formation using HCl in diethyl ether afforded compound 99 (13 mg, 40%) as a beige solid.

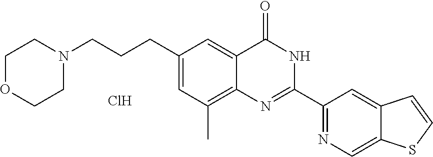 [SMILES: Cc1cc(CCCN2CCOCC2)cc2c(=O)[nH]c(-c3cc4ccsc4cn3)nc12 ; Cl]

¹H-NMR (400 MHz, DMSO): 2.10 (m, 2H, 2 CH); 2.70 (s, 3H, CH₃); 2.78 (t, J 7.4 Hz, 2H, CH₂); 3.08 (m, 4H, 2 CH₂); 3.44 (m, 2H, CH₂); 3.77 (t, J 11.5 Hz, 2H, CH₂); 3.95 (m, 2H, CH₂); 7.64 (s, 1H, Ar); 7.82 (d, J 5.4 Hz, 1H, Ar); 7.90 (s, 1H, Ar); 8.30 (d, J 5.4 Hz, 1H, Ar); 9.00 (s, 1H, Ar); 9.45 (s, 1H, Ar); 10.68 (s, 1H, HCl salt); 11.77 (s, 1H, NH). M/Z (M+H)⁺=421.6.

### Example 34—Synthesis of compound 100 (8-methyl-6-(2-morpholin-4-yl-ethyl)-2-thieno[2,3-c]pyridin-5-yl-3H-quinazolin-4-one hydrochloride)

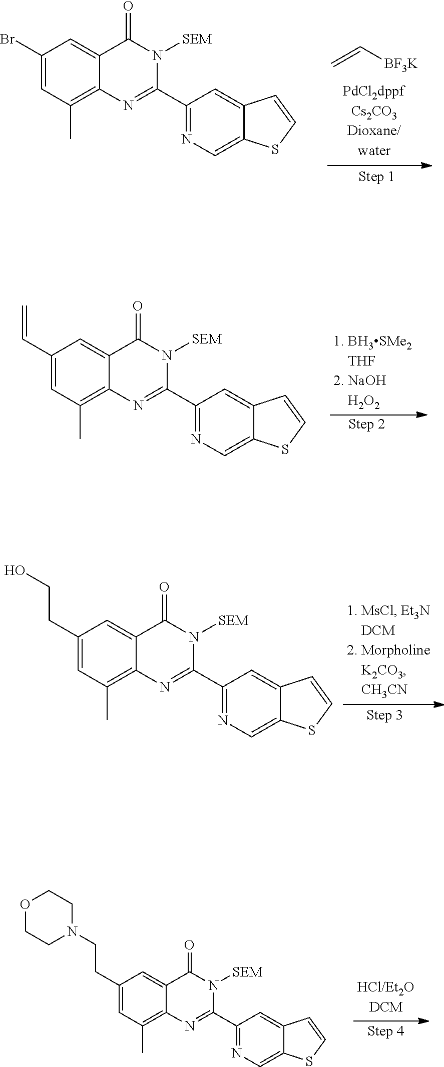

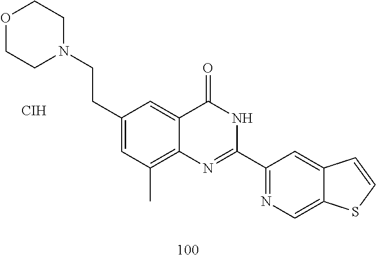 [SMILES: Cc1cc(CCN2CCOCC2)cc2c(=O)[nH]c(-c3cc4ccsc4cn3)nc12 ; "CIH"]

Step 1:

Under inert atmosphere, to a suspension of 6-bromo-8-methyl-2-thieno[2,3-c]pyridin-5-yl-3-(2-trimethylsilanyl-ethoxymethyl)-3H-quinazolin-4-one (389 mg, 0.77 mmol), potassium vinyltrifluoroborate (206 mg, 1.54 mmol) and cesium carbonate (753 mg, 2.31 mmol) in dioxane (7.7 mL) and water (0.40 mL) was added PdCl₂(dppf).CH₂Cl₂ (63 mg, 0.08 mmol). The reaction mixture was heated at 80° C. for 4 h. After cooling to room temperature, the reaction mixture was treated with water and extracted twice with ethyl acetate. The combined organic extracts were washed with brine, dried over MgSO₄, filtered and concentrated under vacuum. Purification by column chromatography on silica gel, using cyclohexane/ethyl acetate as eluent, afforded 8-methyl-2-thieno[2,3-c]pyridin-5-yl-3-(2-trimethylsilanyl-ethoxymethyl)-6-vinyl-3H-quinazolin-4-one (280 mg, 68%) as a white solid.

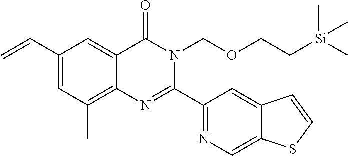 [SMILES: C=Cc1cc(C)c2nc(-c3cc4ccsc4cn3)n(COCC[Si](C)(C)C)c(=O)c2c1]

M/Z (M+H)⁺=450.1.

Step 2:

To a solution of 8-methyl-2-thieno[2,3-c]pyridin-5-yl-3-(2-trimethylsilanyl-ethoxymethyl)-6-vinyl-3H-quinazolin-4-one (280 mg, 0.62 mmol) in dry THF (6.2 mL) was added borane dimethyl sulfide complex (0.12 mL, 1.23 mmol) and the reaction mixture was stirred at room temperature for 16 h. At 0° C., aqueous sodium hydroxide (1.5N, 31.5 mL), then 30% hydrogen peroxide (21.0 mL) were successively added and the reaction mixture was stirred at room temperature for 2 h, before being extracted twice with dichloromethane. The combined organic extracts were washed with brine, dried over MgSO₄, filtered and concentrated under vacuum. Purification by column chromatography on silica gel, using dichloromethane/methanol as eluent, afforded 6-(2-hydroxy-ethyl)-8-methyl-2-thieno[2,3-c]pyridin-5-yl-3-(2-trimethylsilanyl-ethoxymethyl)-3H-quinazolin-4-one (110 mg, 38%) as a white solid.

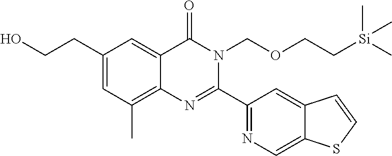 [SMILES: Cc1cc(CCO)cc2c(=O)n(COCC[Si](C)(C)C)c(-c3cc4ccsc4cn3)nc12]

M/Z (M+H)⁺=468.0.

Step 3:

8-Methyl-6-(2-morpholin-4-yl-ethyl)-2-thieno[2,3-c]pyridin-5-yl-3-(2-trimethylsilanyl-ethoxymethyl)-3H-quinazolin-4-one was prepared according to procedure of example 32, step 2, starting from 6-(2-hydroxy-ethyl)-8-methyl-2-thieno[2,3-c]pyridin-5-yl-3-(2-trimethylsilanyl-ethoxymethyl)-3H-quinazolin-4-one (110 mg, 0.24 mmol) and morpholine, to afford the product (48 mg, 37%) as a white solid.

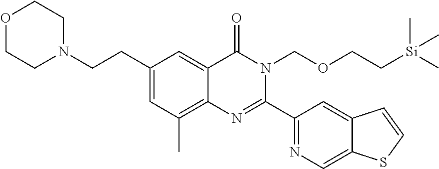 [SMILES: Cc1cc(CCN2CCOCC2)cc2c(=O)n(COCC[Si](C)(C)C)c(-c3cc4ccsc4cn3)nc12]

M/Z (M+H)⁺=537.6.

Step 4:

Compound 100 was prepared according to procedure of example 27, step 3, starting from 8-methyl-6-(2-morpholin-4-yl-ethyl)-2-thieno[2,3-c]pyridin-5-yl-3-(2-trimethylsilanyl-ethoxymethyl)-3H-quinazolin-4-one (48 mg, 0.09 mmol), to afford the product as a beige solid (25 mg, 69%).

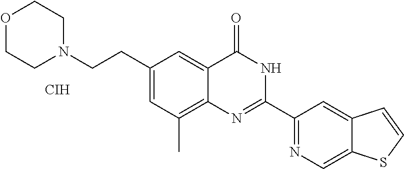

¹H-NMR (400 MHz, DMSO): 2.71 (s, 3H, CH₃); 3.13 (m, 2H, 2 CH); 3.22 (m, 2H, 2 CH); 3.42 (m, 2H, 2 CH); 3.52 (m, 2H, 2 CH); 3.83 (m, 2H, 2 CH); 4.01 (m, 2H, 2 CH); 7.67 (s, 1H, Ar); 7.82 (d, J 5.4 Hz, 1H, Ar); 7.96 (s, 1H, Ar); 8.30 (d, J 5.4 Hz, 1H, Ar); 9.00 (s, 1H, Ar); 9.46 (s, 1H, Ar); 11.00 (s, 1H, HCl salt); 11.84 (s, 1H, NH). M/Z (M+H)⁺=407.4.

### Example 35—Synthesis of compound 101 (8-Methyl-6-(3-pyridin-4-yl-propoxy)-2-(4-trifluoromethyl-pyridin-2-yl)-3H-quinazolin-4-one hydrochloride)

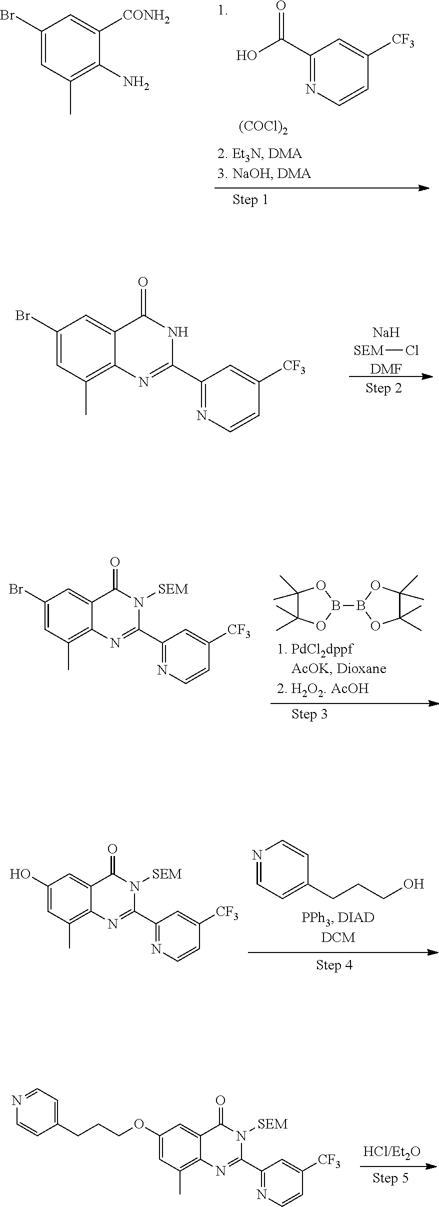

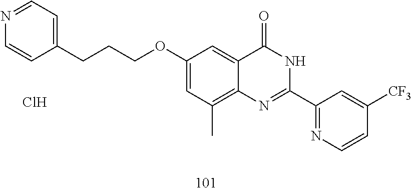 [SMILES: Cc1cc(OCCCc2ccncc2)cc2c(=O)[nH]c(-c3cc(C(F)(F)F)ccn3)nc12 ; Cl]

Step 1:

6-Bromo-8-methyl-2-(4-trifluoromethyl-pyridin-2-yl)-3H-quinazolin-4-one was prepared according to procedure of example 23, step 3, starting from 2-amino-5-bromo-3-methylbenzamide (700 mg, 3.06 mmol) and 4-trifluoromethyl-pyridine-2-carboxylic acid (167 mg, 0.87 mmol) to afford the product as a beige solid (687 mg, 58%).

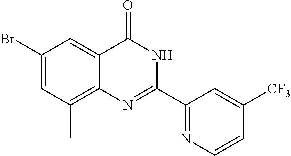 [SMILES: Cc1cc(Br)cc2c(=O)[nH]c(-c3cc(C(F)(F)F)ccn3)nc12]

M/Z (M[⁷⁹Br]+H)⁺=384.4.

Step 2:

6-Bromo-8-methyl-2-(4-trifluoromethyl-pyridin-2-yl)-3-(2-trimethylsilanyl-ethoxymethyl)-3H-quinazolin-4-one was prepared according to procedure of example 23, step 4, starting from 6-bromo-8-methyl-2-(4-trifluoromethyl-pyridin-2-yl)-3H-quinazolin-4-one (717 mg, 1.87 mmol) to afford the product (739 mg, 77%) as a white solid.

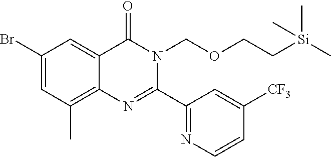 [SMILES: Cc1cc(Br)cc2c(=O)n(COCC[Si](C)(C)C)c(-c3cc(C(F)(F)F)ccn3)nc12]

M/Z (M[⁷⁹Br]+H)⁺=514.5.

Step 3:

6-Hydroxy-8-methyl-2-(4-trifluoromethyl-pyridin-2-yl)-3-(2-trimethylsilanyl-ethoxymethyl)-3H-quinazolin-4-one was prepared according to procedure of example 27, step 1, starting from 6-bromo-8-methyl-2-(4-trifluoromethyl-pyridin-2-yl)-3-(2-trimethylsilanyl-ethoxymethyl)-3H-quinazolin-4-one (109 mg, 0.21 mmol) to afford the product (88 mg, 93%) as a yellow solid.

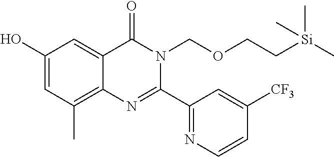 [SMILES: Cc1cc(O)cc2c(=O)n(COCC[Si](C)(C)C)c(-c3cc(C(F)(F)F)ccn3)nc12]

M/Z (M+H)⁺=452.6.

Step 4:

8-Methyl-6-(3-pyridin-4-yl-propoxy)-2-(4-trifluoromethyl-pyridin-2-yl)-3-(2-trimethylsilanyl-ethoxymethyl)-3H-quinazolin-4-one was prepared according to procedure of example 23, step 6, starting from 6-hydroxy-8-methyl-2-(4-trifluoromethyl-pyridin-2-yl)-3-(2-trimethylsilanyl-ethoxymethyl)-3H-quinazolin-4-one (88 mg, 0.20 mmol) to afford the product (116 mg, quantitative yield) as a white solid.

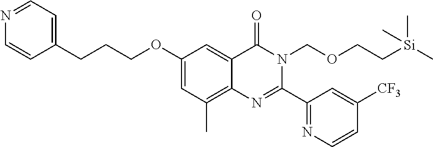 [SMILES: Cc1cc(OCCCc2ccncc2)cc2c(=O)n(COCC[Si](C)(C)C)c(-c3cc(C(F)(F)F)ccn3)nc12]

M/Z (M+H)⁺=571.7.

Step 5:

Compound 101 was prepared according to procedure of example 27, step 3, starting from 8-methyl-6-(3-pyridin-4-yl-propoxy)-2-(4-trifluoromethyl-pyridin-2-yl)-3-(2-trimethylsilanyl-ethoxymethyl)-3H-quinazolin-4-one (30 mg, 0.05 mmol) to afford the product (12 mg, 50%) as a white solid.

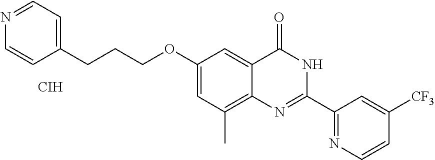

¹H-NMR (400 MHz, DMSO): 2.11 (m, 2H, CH₂); 2.66 (s, 3H, CH₃); 2.81 (dd, J 7.5, 6.3 Hz, 2H, CH₂); 4.12 (t, J 6.3 Hz, 2H, CH₂—O); 7.29 (m, 2H, Ar); 7.39 (d, J 2.9 Hz, 1H, Ar); 7.42 (d, J 2.9 Hz, 1H, Ar); 8.02 (d, J 5.0 Hz, 1H, Ar); 8.47 (m, 2H, Ar); 8.64 (s, 1H, Ar); 9.02 (d, J 5.0 Hz, 1H, Ar); 12.06 (s, 1H, NH); HCl salt signal not observed. M/Z (M+H)⁺=441.6.

### Compound 102 (8-Methyl-6-(tetrahydro-furan-3-ylmethoxy)-2-(4-trifluoromethyl-pyridin-2-yl)-3H-quinazolin-4-one)

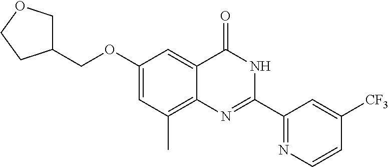 [SMILES: Cc1cc(OCC2CCOC2)cc2c(=O)[nH]c(-c3cc(C(F)(F)F)ccn3)nc12]

Compound 102 was prepared according to procedure of example 27 step 1 and 2, starting from 8-methyl-6-(3-pyridin-4-yl-propoxy)-2-(4-trifluoromethyl-pyridin-2-yl)-3-(2-trimethylsilanyl-ethoxymethyl)-3H-quinazolin-4-one in step 2, followed by procedure of example 30 step 2 to afford the product as a yellow solid.

¹H-NMR (400 MHz, DMSO): 1.73 (m, 1H, CH); 2.05 (m, 1H, CH); 2.65 (s, 3H, CH₃); 2.69 (m, 1H, CH); 3.58 (m, 1H, CH); 3.68 (m, 1H, CH); 3.80 (m, 2H, 2 CH); 4.05 (m, 2H, 2 CH); 7.39 (d, J 2.9 Hz, 1H, Ar); 7.44 (d, J 2.9 Hz, 1H, Ar); 8.01 (d, J 5.0 Hz, 1H, Ar); 8.63 (s, 1H, Ar); 9.01 (d, J 5.0 Hz, 1H, Ar); 12.03 (s, 1H, NH). M/Z (M+H)⁺=405.9.

### Compound 103 (8-Methyl-6-(1-propionyl-azetidin-3-yloxy)-2-(4-trifluoromethyl-pyridin-2-yl)-3H-quinazolin-4-one)

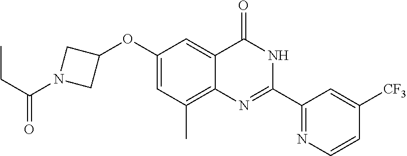 [SMILES: CCC(=O)N1CC(Oc2cc(C)c3nc(-c4cc(C(F)(F)F)ccn4)[nH]c(=O)c3c2)C1]

Compound 103 was prepared according to procedure of example 35, step 1-4, starting from 1-(3-hydroxy-azetidin-1-yl)-propan-1-one in step 4, followed by procedure of example 36, step 7. Purification by trituration in diethyl ether afforded the product as a yellow solid.

¹H-NMR (400 MHz, DMSO): 0.97 (t, J 7.5 Hz, 3H, ethyl); 2.11 (q, J 7.5 Hz, 2H, ethyl); 2.66 (s, 3H, CH₃); 3.84 (dd, J 10.5, 3.5 Hz, 1H, CH); 4.12 (dd, J 9.3, 3.5 Hz, 1H, CH); 4.33 (dd, J 10.5, 6.5 Hz, 1H, CH); 4.62 (dd, J 9.3, 6.5 Hz, 1H, CH); 5.21 (m, 1H, CH); 7.25 (d, J 2.9 Hz, 1H, Ar); 7.38 (d, J 2.9 Hz, 1H, Ar); 8.02 (d, J 5.2 Hz, 1H, Ar); 8.63 (s, 1H, Ar); 9.02 (d, J 5.2 Hz, 1H, Ar); 12.10 (s, 1H, NH). M/Z (M+H)⁺=433.0.

1-(3-Hydroxy-azetidin-1-yl)-propan-1-one was prepared in 18% yield using procedure of (S)-1-(3-hydroxy-pyrrolidin-1-yl)-propan-1-one of compound 82 and starting from 3-hydroxyazetidine

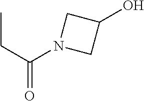

¹H-NMR (400 MHz, DMSO): 0.94 (t, J 7.5 Hz, 3H, ethyl); 2.03 (q, J 7.5 Hz, 2H, ethyl); 3.54 (dd, J 9.8, 4.3 Hz, 1H, CH); 3.80 (dd, J 8.5, 4.3 Hz, 1H, CH); 3.99 (dd, J 9.8, 6.8 Hz, 1H, CH); 4.24 (m, 1H, CH); 4.42 (m, 1H, CH); 5.65 (d, J 5.9 Hz, 1H, OH).

### Compound 104 (8-Methyl-6-(1-oxetan-3-yl-piperidin-4-yloxy)-2-(4-trifluoromethyl-pyridin-2-yl)-3H-quinazolin-4-one)

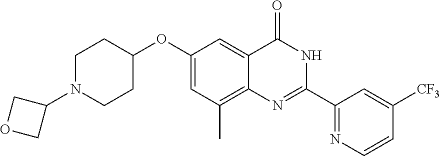 [SMILES: Cc1cc(OC2CCN(C3COC3)CC2)cc2c(=O)[nH]c(-c3cc(C(F)(F)F)ccn3)nc12]

Compound 104 was prepared according to procedure of example 35, step 1-4, starting from 1-oxetan-3-yl-piperidin-4-ol in step 4, followed by procedure of example 36, step 7. Purification by trituration in diethyl ether afforded the product as a white solid.

¹H-NMR (400 MHz, DMSO): 1.77 (m, 2H, 2 CH); 2.03 (m, 2H, 2 CH); 2.29 (m, 2H, 2 CH); 2.63 (m, 2H, 2 CH); 2.68 (s, 3H, CH₃); 3.57 (m, 1H, CH); 4.49 (m, 2H, 2 CH); 4.58 (m, 3H, 3CH); 7.39 (d, J 2.8 Hz, 1H, Ar); 7.50 (d, J 2.8 Hz, 1H, Ar); 7.96 (d, J 5.2 Hz, 1H, Ar); 8.64 (s, 1H, Ar); 9.02 (d, J 5.2 Hz, 1H, Ar); 11.47 (s, 1H, NH). M/Z (M+H)⁺=461.0.

1-Oxetan-3-yl-piperidin-4-ol was prepared in 94% yield using procedure of (S)-1-oxetan-3-yl-pyrrolidin-3-ol in compound 83-R and starting from 4-hydroxypiperidine

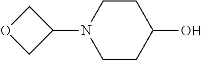 [SMILES: OC1CCN(C2COC2)CC1]

¹H-NMR (400 MHz, DMSO): 1.38 (m, 2H, 2 CH); 1.70 (m, 2H, 2 CH); 1.84 (m, 2H, 2 CH); 2.46 (m, 2H, 2 CH); 3.33 (m, 1H, CH); 3.44 (m, 1H, CH); 4.38 (t, J 6.0 Hz, 2H, 2 CH); 4.49 (t, J 6.5 Hz, 2H, 2 CH); 4.54 (d, J 4.2 Hz, 1H, OH).

### Compound 105 (8-Methyl-6-(3-oxa-9-aza-spiro[5.5]undec-9-yl)-2-(4-trifluoromethyl-pyridin-2-yl)-3H-quinazolin-4-one)

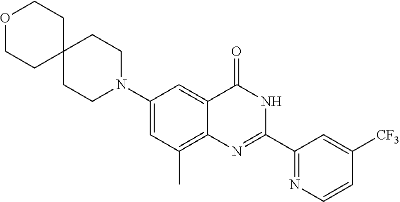 [SMILES: Cc1cc(N2CCC3(CCOCC3)CC2)cc2c(=O)[nH]c(-c3cc(C(F)(F)F)ccn3)nc12]

Compound 105 was prepared according to procedure of example 30, starting from 6-bromo-8-methyl-2-(4-trifluoromethyl-pyridin-2-yl)-3-(2-trimethylsilanyl-ethoxymethyl)-3H-quinazolin-4-one and 3-oxa-9-azaspiro[5.5]undecane in step 1 to afford the product as a yellow solid.

¹H-NMR (400 MHz, DMSO): 1.48 (t, J 5.4 Hz, 4H, 2 CH₂); 1.63 (dd, J 6.8, 4.4 Hz, 4H, 2 CH₂); 2.64 (s, 3H, CH₃); 3.30 (dd, J 6.8, 4.4 Hz, 4H, 2 CH₂); 3.59 (t, J 5.4 Hz, 4H, 2 CH₂); 7.34 (d, J 2.6 Hz, 1H, Ar); 7.50 (d, J 2.6 Hz, 1H, Ar); 7.98 (d, J 5.1 Hz, 1H, Ar); 8.61 (s, 1H, Ar); 8.99 (d, J 5.1 Hz, 1H, Ar); 11.81 (s, 1H, NH). M/Z (M+H)⁺=459.7.

### Compound 106 (8-methyl-6-(3-pyridin-4-yl-propoxy)-2-pyrrolo[1,2-c]pyrimidin-3-yl-3H-quinazolin-4-one hydrochloride)

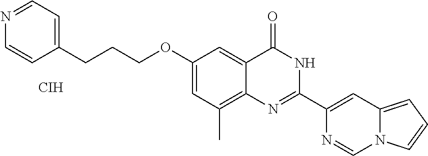

Compound 106 was prepared according to procedure of example 35, starting from pyrrolo[1,2-c]pyrimidine-3-carboxylic acid and 2-amino-5-bromo-3-methylbenzamide in step 1. Purification by column chromatography on silica gel, using dichloromethane/methanol as eluent, and salt formation using HCl in diethyl ether afforded the product as a brown solid.

¹H-NMR (400 MHz, DMSO): 2.21 (m, 2H, CH₂); 2.63 (s, 3H, CH₃); 3.10 (t, J 7.6 Hz, 2H, CH₂); 4.14 (t, J 6.2 Hz, 2H, CH₂—O); 6.87 (d, J 3.8 Hz, 1H, Ar); 7.07 (dd, J 3.8, 2.8 Hz, 1H, Ar); 7.28 (d, J 2.9 Hz, 1H, Ar); 7.37 (d, J 2.9 Hz, 1H, Ar); 7.89 (s, 1H, Ar); 8.01 (d, J 6.6 Hz, 2H, Ar); 8.52 (s, 1H, Ar); 8.83 (d, J 6.6 Hz, 2H, Ar); 9.34 (s, 1H, HCl salt); 11.43 (s, 1H, NH). M/Z (M+H)⁺=411.9.

### Compound 107 (8-methyl-2-pyrrolo[1,2-c]pyrimidin-3-yl-6-(tetrahydro-furan-3-ylmethoxy)-3H-quinazolin-4-one)

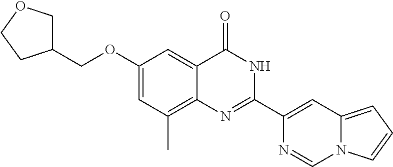 [SMILES: Cc1cc(OCC2CCOC2)cc2c(=O)[nH]c(-c3cc4cccn4cn3)nc12]

Compound 107 was prepared according to procedure of example 35, starting from pyrrolo[1,2-c]pyrimidine-3-carboxylic acid in step 1 and (tetrahydro-furan-3-yl)-methanol in step 4 to afford the product as a yellow solid.

¹H-NMR (400 MHz, DMSO): 1.72 (m, 1H, CH); 2.04 (m, 1H, CH); 2.63 (s, 3H, CH₃); 2.69 (m, 1H, CH); 3.57 (m, 1H, CH); 3.69 (m, 1H, CH); 3.80 (m, 2H, 2 CH); 4.04 (m, 2H, 2 CH); 6.87 (d, J 3.7 Hz, 1H, Ar); 7.07 (dd, J 3.7, 2.8 Hz, 1H, Ar); 7.34 (d, J 2.9 Hz, 1H, Ar); 7.40 (d, J 2.9 Hz, 1H, Ar); 7.89 (d, J 2.8 Hz, 1H, Ar); 8.51 (s, 1H, Ar); 9.33 (s, 1H, Ar); 11.41 (s, 1H, NH). M/Z (M+H)⁺=376.9.

### Compound 108 (8-methyl-6-(3-oxa-9-aza-spiro[5.5]undec-9-yl)-2-pyrrolo[1,2-c]pyrimidin-3-yl-3H-quinazolin-4-one)

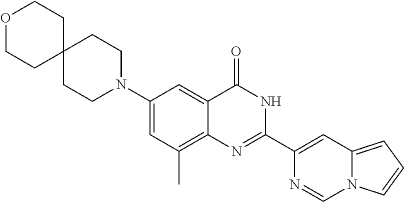 [SMILES: Cc1cc(N2CCC3(CCOCC3)CC2)cc2c(=O)[nH]c(-c3cc4cccn4cn3)nc12]

Compound 108 was prepared according to procedure of example 30, starting from 6-bromo-8-methyl-2-pyrrolo[1,2-c]pyrimidin-3-yl-3-(2-trimethylsilanyl-ethoxymethyl)-3H-quinazolin-4-one and 3-oxa-9-azaspiro[5.5]undecane to afford the product as a yellow solid.

¹H-NMR (400 MHz, DMSO): 1.48 (t, J 5.4 Hz, 4H, 2 CH₂); 1.64 (dd, J 6.8, 4.4 Hz, 4H, 2 CH₂); 2.62 (s, 3H, CH₃); 3.30 (dd, J 6.8, 4.4 Hz, 4H, 2 CH₂); 3.59 (t, J 5.4 Hz, 4H, 2 CH₂); 6.85 (d, J 3.7 Hz, 1H, Ar); 7.06 (dd, J 3.7, 2.8 Hz, 1H, Ar); 7.32 (d, J 2.9 Hz, 1H, Ar); 7.47 (d, J 2.9 Hz, 1H, Ar); 7.89 (d, J 2.8 Hz, 1H, Ar); 8.48 (s, 1H, Ar); 9.33 (s, 1H, Ar); 11.22 (s, 1H, NH). M/Z (M+H)⁺=430.1.

### Compound 109 (8-methyl-6-(1-oxetan-3-yl-piperidin-4-yloxy)-2-pyrrolo[1,2-c]pyrimidin-3-yl-3H-quinazolin-4-one)

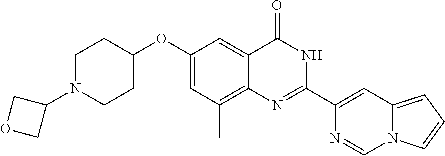 [SMILES: Cc1cc(OC2CCN(C3COC3)CC2)cc2c(=O)[nH]c(-c3cc4cccn4cn3)nc12]

Compound 109 was prepared according to procedure of compound 104, starting from 6-hydroxy-8-methyl-2-pyrrolo[1,2-c]pyrimidin-3-yl-3-(2-trimethylsilanyl-ethoxymethyl)-3H-quinazolin-4-one. Purification by column chromatography on silica gel, using dichloromethane/methanol as eluent, and trituration in diethyl ether afforded the product as a beige solid.

¹H-NMR (400 MHz, DMSO): 1.70 (m, 2H, 2 CH); 2.00 (m, 2H, 2 CH); 2.16 (m, 2H, 2 CH); 2.55 (m, 2H, 2 CH); 2.62 (s, 3H, CH₃); 3.44 (m, 1H, CH); 4.44 (t, J 6.1 Hz, 2H, 2 CH); 4.54 (m, 3H, 3CH); 6.87 (d, J 3.9 Hz, 1H, Ar); 7.07 (dd, J 3.9, 2.6 Hz, 1H, Ar); 7.35 (d, J 2.8 Hz, 1H, Ar); 7.40 (d, J 2.8 Hz, 1H, Ar); 7.89 (d, J 2.6 Hz, 1H, Ar); 8.51 (s, 1H, Ar); 9.33 (s, 1H, Ar); 11.40 (s, 1H, NH). M/Z (M+H)⁺=432.5.

### Compound 110-R (R-8-Methyl-6-[1-(tetrahydro-pyran-4-yl)-ethoxy]-2-thieno[2,3-c]pyridin-5-yl-3H-quinazolin-4-one)

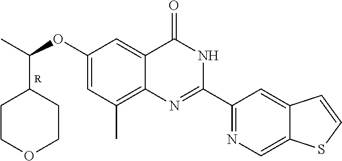

Compound 110-R was prepared according to procedure of example 27, step 1 and 2, starting from (1S)-1-(oxan-4-yl)ethan-1-ol in step 2, followed by procedure of example 36, step 7. Purification by trituration in diethyl ether afforded the product as a beige solid.

¹H-NMR (400 MHz, DMSO): 1.26 (d, J 6.1 Hz, 3H, CH₃); 1.34 (m, 1H, CH); 1.43 (m, 1H, CH); 1.58 (m, 1H, CH); 1.77 (m, 1H, CH); 1.84 (m, 1H, CH); 2.68 (s, 3H, CH₃); 3.33 (m, 1H, CH); 3.28 (m, 1H, CH); 3.90 (m, 2H, 2 CH); 4.42 (p, J 6.1 Hz, 1H, CH); 7.36 (d, J 2.7 Hz, 1H, Ar); 7.43 (d, J 2.7 Hz, 1H, Ar); 7.80 (d, J 5.4 Hz, 1H, Ar); 8.28 (d, J 5.4 Hz, 1H, Ar); 8.96 (s, 1H, Ar); 9.43 (s, 1H, Ar); 11.70 (s, 1H, NH). M/Z (M+H)⁺=422.7.

### Compound 110-S (S-8-Methyl-6-[1-(tetrahydro-pyran-4-yl)-ethoxy]-2-thieno[2,3-c]pyridin-5-yl-3H-quinazolin-4-one)

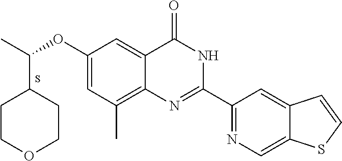

Compound 110-S was prepared according to procedure of compound 110-R, starting from (1R)-1-(oxan-4-yl)ethan-1-ol in step 2 to afford the product as a beige solid.

¹H-NMR (400 MHz, DMSO): 1.26 (d, J 6.1 Hz, 3H, CH₃); 1.39 (sd, J 4.0, 12.0 Hz 2H, CH₂); 1.56-1.61 (m, 1H, CH₂); 1.74-1.80 (m, 1H, CH₂); 1.81-1.88 (m, 1H, CH); 2.68 (s, 3H, CH₃); 3.28 (brs, 1H, CH₂—O); 3.34 (bs, 1H, CH₂—O); 3.90 (d, J 3.9, 11.0 Hz, 2H, CH₂—O); 4.41 (quint, J 6.0 Hz, 1H, CH—O); 7.36 (d, J 2.3 Hz, 1H, Ar); 7.43 (d, J 2.8 Hz, 1H, Ar); 7.81 (d, J 5.4 Hz, 1H, Ar); 8.28 (d, J 5.4 Hz, 1H, Ar); 8.96 (d, J 0.9 Hz, 1H, Ar); 9.43 (bs, 1H, Ar); 11.70 (s, 1H, NH). M/Z (M+H)⁺=422.6. MP=150-180° C.

### Compound 111-R (R-8-Methyl-6-[1-(tetrahydro-pyran-4-yl)-ethoxy]-2-(4-trifluoromethyl-pyridin-2-yl)-3H-quinazolin-4-one)

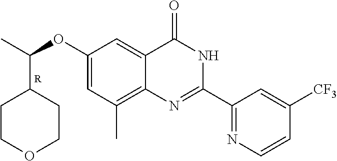

Compound 111-R was prepared according to procedure of compound 110-R, starting from (1S)-1-(oxan-4-yl)ethan-1-ol and 6-hydroxy-8-methyl-2-(4-trifluoromethyl-pyridin-2-yl)-3-(2-trimethylsilanyl-ethoxymethyl)-3H-quinazolin-4-one in step 2, to afford the product as a beige solid.

¹H-NMR (400 MHz, DMSO): 1.26 (d, J 6.1 Hz, 3H, CH₃); 1.34 (m, 1H, CH); 1.43 (m, 1H, CH); 1.57 (m, 1H, CH); 1.76 (m, 1H, CH); 1.84 (m, 1H, CH); 2.64 (s, 3H, CH₃); 3.33 (m, 1H, CH); 3.28 (m, 1H, CH); 3.90 (m, 2H, 2 CH); 4.43 (p, J 6.1 Hz, 1H, CH); 7.37 (d, J 2.7 Hz, 1H, Ar); 7.43 (d, J 2.7 Hz, 1H, Ar); 8.01 (d, J 5.1 Hz, 1H, Ar); 8.62 (s, 1H, Ar); 9.01 (d, J 5.1 Hz, 1H, Ar); 12.00 (s, 1H, NH). M/Z (M+H)⁺=434.7.

### Compound 111-S (S-8-Methyl-6-[1-(tetrahydro-pyran-4-yl)-ethoxy]-2-(4-trifluoromethyl-pyridin-2-yl)-3H-quinazolin-4-one)

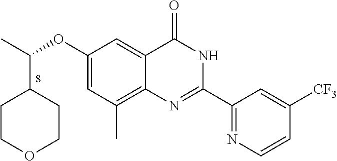

Compound 111-S was prepared according to procedure compound 111-R, starting from (1R)-1-(oxan-4-yl)ethan-1-ol in step 2, to afford the product as a beige solid.

¹H-NMR (400 MHz, DMSO): 1.26 (d, J 6.2 Hz, 3H, CH₃); 1.38 (sd, J 4.6, 12.3 Hz, 2H, CH₂); 1.55-1.60 (m, 1H, CH₂); 1.74-1.79 (m, 1H, CH₂); 1.80-1.88 (m, 1H, CH); 2.64 (s, 3H, CH₃); 3.28 (brs, 1H, CH₂—O); 3.33 (brs, 1H, CH₂—O); 3.87-3.92 (m, 2H, CH₂—O); 4.43 (quint, J 6.16 Hz, 1H, CH—O); 7.37 (d, J 2.8 Hz, 1H, Ar); 7.43 (d, J 2.8 Hz, 1H, Ar); 8.0.1 (dd, J 1.0, 2.0 Hz, 1H, Ar); 8.63 (s, 1H, Ar); 9.01 (d, J 5.1 Hz, 1H, Ar); 12.01 (s, 1H, NH). M/Z (M+H)⁺=434.6. MP=120-132° C.

### Compound 112 (6-[(3-fluorotetrahydrofuran-3-yl)methoxy]-8-methyl-2-[4-(trifluoromethyl)-2-pyridyl]-3H-quinazolin-4-one)

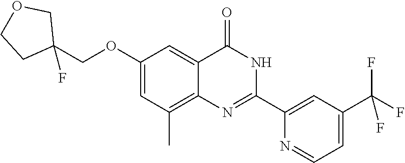 [SMILES: Cc1cc(OCC2(F)CCOC2)cc2c(=O)[nH]c(-c3cc(C(F)(F)F)ccn3)nc12]

Compound 112 was prepared according to procedure of compound 110-R, starting from (3-fluorotetrahydrofuran-3-yl)methanol and 6-hydroxy-8-methyl-2-(4-trifluoromethyl-pyridin-2-yl)-3-(2-trimethylsilanyl-ethoxymethyl)-3H-quinazolin-4-one in step 2, to afford the product as a beige solid.

¹H-NMR (400 MHz, DMSO): 2.2 (t, J 7.1 Hz, 1H, CH₂); 2.24-2.29 (m, 1H, CH₂); 2.66 (s, 3H, CH₃); 3.82-4.03 (m, 4H, CH₂—O); 4.40-4.53 (m, 2H, CH₂—O); 7.44 (d, J 2.9 Hz, 1H, Ar); 7.50 (d, J 2.9 Hz, 1H, Ar); 8.02 (dd, 1.4, 5.1 Hz, 1H, Ar); 8.64 (s, 1H, Ar); 9.02 (d, J 5.1 Hz, 1H, Ar); 12.07 (s, 1H, NH). M/Z (M+H)⁺=424.6. MP=188-195° C.

### Example 36—Synthesis of compound 113 (8-methyl-6-(3-oxa-9-azaspiro[5.5]undecan-9-yl)-2-thieno[2,3-c]pyridin-5-yl-3-(2-trimethylsilylethoxymethyl)pyrido[3,2-d]pyrimidin-4-one)

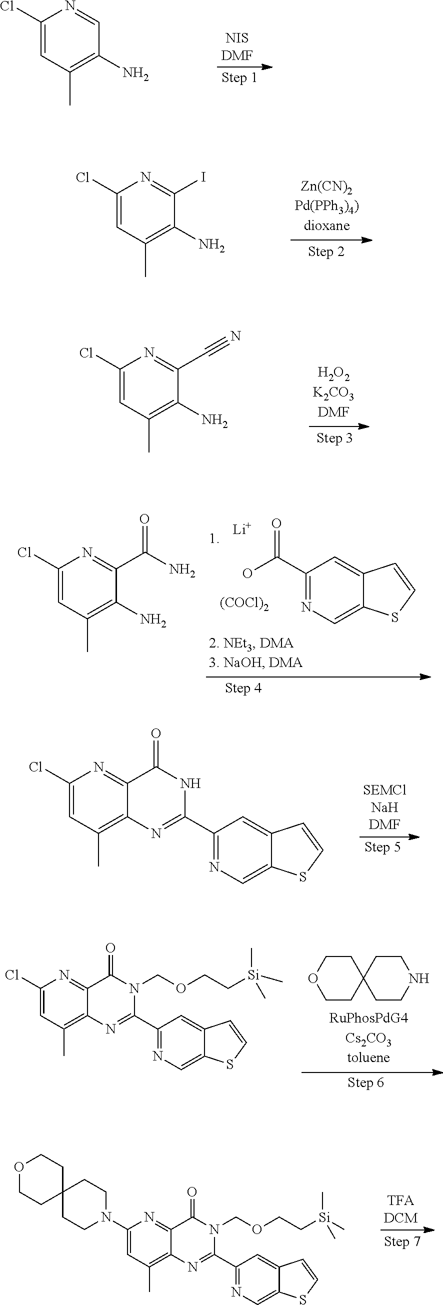

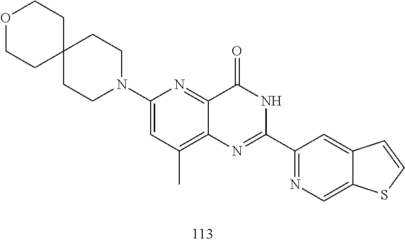 [SMILES: Cc1cc(N2CCC3(CCOCC3)CC2)nc2c(=O)[nH]c(-c3cc4ccsc4cn3)nc12]

Step 1:

Under inert atmosphere, to a solution of 6-chloro-4-methyl-pyridin-3-amine (200 mg, 1.40 mmol) in dry DMF (7 mL) was added N-iodo-succinimide (346 mg, 1.54 mmol). The reaction mixture was stirred overnight at room temperature, then water (70 mL) was added. The precipitate was filtered, washed with water and dried overnight under high vacuum in presence of P₂O₅ at 50° C. to afford 6-chloro-2-iodo-4-methyl-pyridin-3-amine (265 mg) as a brown solid. The filtrate was then extracted with dichloromethane (2×30 mL), dried over MgSO₄ and concentrated under vacuum. Water was added to the resulting crude residue to precipitate the product which was filtered and dried overnight under high vacuum in presence of P₂O₅ at 50° C. to afford more 6-chloro-2-iodo-4-methyl-pyridin-3-amine (160 mg) as an orange solid. In total 425 mg of 6-chloro-2-iodo-4-methyl-pyridin-3-amine were obtained (quantitative yield).

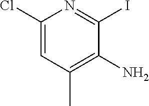

M/Z (M+H)⁺=269.3.

Step 2:

Under inert atmosphere to a suspension of 6-chloro-2-iodo-4-methyl-pyridin-3-amine (265 mg, 0.99 mmol) in dry dioxane (5 mL) were added zinc cyanide (116 mg, 0.99 mmol) and Pd(PPh₃)₄ (110 mg, 0.01 mmol) and the mixture was heated 5 days at 90° C. Then a saturated aqueous solution of sodium bicarbonate (100 mL) was added to the reaction mixture which was extracted with dichloromethane (2×100 mL), washed with brine, dried over MgSO₄ and concentrated under vacuum. Purification by column chromatography on silica gel, using dichloromethane/methanol as eluent afforded desired 3-amino-6-chloro-4-methyl-pyridine-2-carbonitrile (101 mg, 61%) as a beige solid as well as 3-amino-6-chloro-4-methyl-pyridine-2-carboxamide (68 mg) as a brown solid.

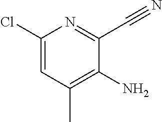

M/Z (M+H)⁺=168.

Step 3:

Under atmosphere, to a solution of 3-amino-6-chloro-4-methyl-pyridine-2-carbonitrile (98 mg, 0.58 mmol) in dry DMF (3 mL), were added H₂O₂ (30% aq. solution, 208 μL, 2.32 mmol) and potassium carbonate (32 mg, 0.23 mmol) and the reaction mixture was stirred at room temperature for 2 days. Then an aqueous saturated solution of ammonium chloride (30 mL) was added to the reaction mixture which was extracted with ethyl acetate (2×30 mL), washed with brine, dried over MgSO₄ and concentrated under vacuum. Purification by column chromatography on silica gel, together with the 68 mg of 3-amino-6-chloro-4-methyl-pyridine-2-carboxamide from step 2, using dichloromethane/methanol as eluent afforded 3-amino-6-chloro-4-methyl-pyridine-2-carboxamide (193 mg, 99%) as a beige solid.

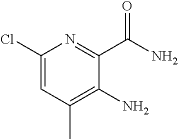

M/Z (M+H)⁺=186.

Step 4:

6-chloro-8-methyl-2-thieno[2,3-c]pyridin-5-yl-3H-pyrido[3,2-d]pyrimidin-4-one was prepared according to procedure of example 23, step 3, starting from 3-amino-6-chloro-4-methyl-pyridine-2-carboxamide (190 mg, 1.03 mmol) and thieno[3,2-c]pyridine-6-carboxylic acid (381 mg, 2.06 mmol) to afford 6-chloro-8-methyl-2-thieno[2,3-c]pyridin-5-yl-3H-pyrido[3,2-d]pyrimidin-4-one (226 mg, 67%) as a beige powder.

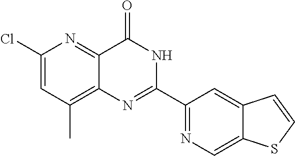 [SMILES: Cc1cc(Cl)nc2c(=O)[nH]c(-c3cc4ccsc4cn3)nc12]

M/Z (M+H)⁺=329.

Step 5:

6-chloro-8-methyl-2-thieno[2,3-c]pyridin-5-yl-3-(2-trimethylsilylethoxymethyl)pyrido [3,2-d]pyrimidin-4-one was prepared according to procedure of example 23, step 4, starting from 6-chloro-8-methyl-2-thieno[2,3-c]pyridin-5-yl-3H-pyrido[3,2-d]pyrimidin-4-one (225 mg, 0.68 mmol) to afford the product (243 mg, 78%) as an orange oil.

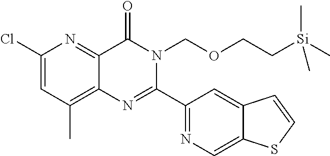 [SMILES: Cc1cc(Cl)nc2c(=O)n(COCC[Si](C)(C)C)c(-c3cc4ccsc4cn3)nc12]

Step 6:

8-methyl-6-(3-oxa-9-azaspiro[5.5]undecan-9-yl)-2-thieno[2,3-c]pyridin-5-yl-3-(2-trimethylsilylethoxymethyl)pyrido[3,2-d]pyrimidin-4-one was prepared according to procedure of example 30, step 1, starting from 6-chloro-8-methyl-2-thieno[2,3-c]pyridin-5-yl-3H-pyrido[3,2-d]pyrimidin-4-one (240 mg, 0.52 mmol) and 3-oxa-9-azaspiro[5.5]undecane (161 mg, 1.04 mmol). Purification by column chromatography on silica gel, using dichloromethane/methanol as eluent afforded the product (122 mg, 41%) as a yellow oil.

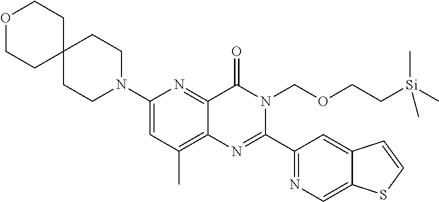 [SMILES: Cc1cc(N2CCC3(CCOCC3)CC2)nc2c(=O)n(COCC[Si](C)(C)C)c(-c3cc4ccsc4cn3)nc12]

M/Z (M+H)⁺=578.8.

Step 7:

To a solution of 6-chloro-8-methyl-2-thieno[2,3-c]pyridin-5-yl-3H-pyrido[3,2-d]pyrimidin-4-one (120 mg, 0.21 mmol) in dichloromethane (1.1 mL) was added TFA (242 μL, 3.15 mmol) and the reaction mixture was stirred at room temperature for 24 h. Then the reaction was treated with aqueous sodium bicarbonate, extracted twice with dichloromethane, washed with brine, dried over MgSO₄ and evaporated to dryness. Purification by column chromatography on silica gel, using dichloromethane/methanol as eluent, followed by recrystallization in DMSO, afforded compound 113 (27 mg, 29%) as a yellow solid.

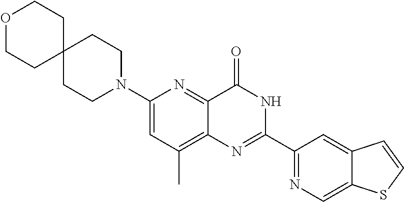 [SMILES: Cc1cc(N2CCC3(CCOCC3)CC2)nc2c(=O)[nH]c(-c3cc4ccsc4cn3)nc12]

¹H-NMR (400 MHz, DMSO): 1.50 (t, J 5.3 Hz, 4H, CH₂); 1.55-1.58 (m, 4H, CH₂); 2.73 (s, 3H, CH₃); 3.60 (t, J 5.3 Hz, 4H, CH₂—N); 3.68-3.71 (m, 4H, CH₂—O); 7.34 (s, 1H, Ar); 7.78 (d, J 5.4 Hz, 1H, Ar); 8.27 (d, J 5.4 Hz, 1H, Ar); 8.89 (d, J 0.9 Hz, 1H, Ar); 9.41 (bs, 1H, Ar); 11.66 (s, 1H, NH). M/Z (M+H)⁺=448.7. MP>250° C.

### Example 37—Synthesis of compound 114 (8-methyl-6-(morpholinomethyl)-2-thieno[2,3-c]pyridin-5-yl-3H-quinazolin-4-one hydrochloride)

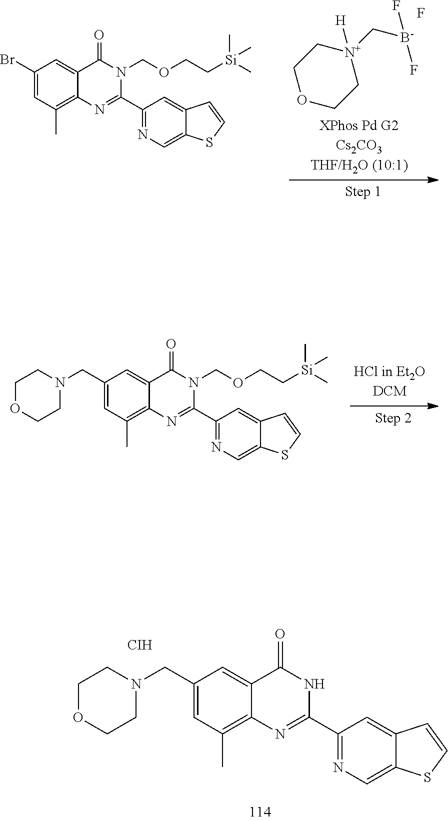

Step 1:

Under inert atmosphere, a mixture of 6-bromo-8-methyl-2-thieno[2,3-c]pyridin-5-yl-3-(2-trimethylsilanyl-ethoxymethyl)-3H-quinazolin-4-one (from example 26, 150 mg, 0.30 mmol), morpholinium-4-yl-methyl)trifluoroborate internal salt (50 mg, 0.30 mmol), cesium carbonate (292 mg, 0.90 mmol) and XPhos Pd G2 (12 mg, 0.02 mmol) in degassed THF (3 mL) and water (0.3 mL) was heated at 95° C. for 20 h in a sealed tube. After cooling to room temperature, the reaction mixture was poured in water and extracted twice with ethyl acetate. The combined organic extracts were washed with brine, dried over MgSO₄, filtered and concentrated under vacuum. Purification by column chromatography on silica gel, using ethyl acetate/cyclohexane as eluent, afforded 8-methyl-6-(morpholinomethyl)-2-thieno[2,3-c]pyridin-5-yl-3-(2-trimethylsilylethoxymethyl)quinazolin-4-one (108 mg, 69%) as a yellow oil.

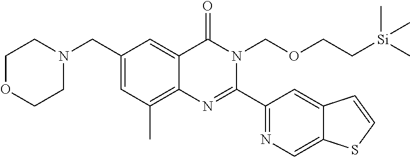 [SMILES: Cc1cc(CN2CCOCC2)cc2c(=O)n(COCC[Si](C)(C)C)c(-c3cc4ccsc4cn3)nc12]

M/Z (M+H)⁺=523.7.

Step 2:

Compound 114 was prepared according to procedure of example 27, step 3, starting from 8-methyl-6-(morpholinomethyl)-2-thieno[2,3-c]pyridin-5-yl-3-(2-trimethylsilylethoxymethyl)quinazolin-4-one (105 mg, 0.20 mmol) to afford the product as a yellow solid (78 mg, 90%) after trituration in diethyl ether.

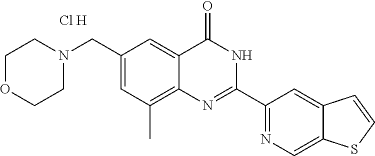

¹H-NMR (400 MHz, DMSO): 2.72 (s, 3H, CH₃); 3.07-3.21 (m, 2H, CH₂—N); 3.23-3.34 (d, 2H, J 12.2 Hz, CH₂—N); 3.76 (t, J 11.8 Hz, 2H, CH₂—O); 3.95 (d, J 12.1 Hz, 2H, CH₂—O); 4.47 (d, J 4.0 Hz, 2H, CH₂—N); 7.83 (dd, J 0.5, 5.4 Hz, 1H, Ar); 8.24 (d, J 1.6 Hz, 1H, Ar); 8.31 (d, J 5.4 Hz, 1H, Ar); 9.04 (d, J 0.9 Hz, 1H, Ar); 9.47 (s, 1H, Ar); 10.97 (bs, 1H, HCl salt); 11.99 (bs, 1H, NH). M/Z (M+H)⁺=393.7. MP>250° C.

### Compound 115 (8-methyl-6-(morpholinomethyl)-2-[4-(trifluoromethyl)-2-pyridyl]-3H-quinazolin-4-one hydrochloride)

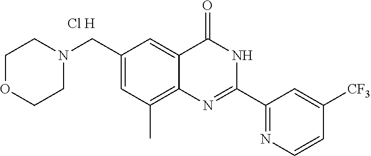

Compound 115 was prepared according to procedure of example 37, starting from (6-bromo-3,8-dimethyl-2-(4-methyl-2-pyridyl)quinazolin-4-one in step 1, to afford the product as a white solid.

¹H-NMR (400 MHz, DMSO): 2.70 (s, 3H, CH₃); 3.08-3.22 (m, 2H, CH₂—N); 3.23-3.29 (m, 2H, CH₂—N); 3.7 (t, J 12.2 Hz, 2H, CH₂—O); 3.97 (d, J 12.8 Hz, 2H, CH₂—O); 4.49 (d, J 4.2 Hz, 2H, CH₂—N); 7.94 (s, 1H, Ar); 8.08 (dd, J 1.2, 5.1 Hz, 1H, Ar); 8.26 (s, 1H, Ar); 8.69 (s, 1H, Ar); 9.06 (d, J 5.0 Hz, 1H, Ar); 10.54 (bs, 1H, HCl salt); 12.35 (s, 1H, NH). M/Z (M+H)⁺=405.7. MP>250° C.

### Compound 116 (8-methyl-6-(1-propanoylazetidin-3-yl)oxy-2-thieno[2,3-c]pyridin-5-yl-3H-quinazolin-4-one)

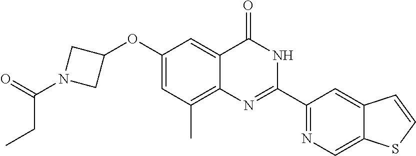 [SMILES: CCC(=O)N1CC(Oc2cc(C)c3nc(-c4cc5ccsc5cn4)[nH]c(=O)c3c2)C1]

Compound 116 was prepared according to procedure of example 27, step 1 and 2, starting from 1-(3-hydroxyazetidin-1-yl)propan-1-one in step 2, followed by procedure of example 36, step 7. Purification by column chromatography on silica gel, using dichloromethane/methanol as eluent, afforded the product as a beige solid.

¹H-NMR (400 MHz, DMSO): 0.98 (t, J 7.5 Hz, 3H, CH₃); 2.12 (q, J 7.5 Hz, 2H, CH₂); 2.69 (s, 3H, CH₃); 3.84 (dd, J 10.5, 3.4 Hz, 1H, CH₂—N); 4.12 (dd, J 9.6, 3.4 Hz, 1H, CH₂—N); 4.33 (dd, J 10.5, 6.4 Hz, 1H, CH₂—N); 4.61 (dd, J 9.4, 6.4 Hz, 1H, CH₂—N); 5.17-5.22 (m, 1H, CH—O); 7.23 (d, J 2.9 Hz, 1H, Ar); 7.36 (dd, J 0.7, 2.9 Hz, 1H, Ar); 7.80 (d, J 5.4 Hz, 1H, Ar); 8.28 (d, J 5.4 Hz, 1H, Ar); 8.95 (d, J 0.7 Hz, 1H, Ar); 9.43 (s, 1H, Ar); 11.77 (s, 1H, NH). M/Z (M+H)⁺=421.7. MP=230-240° C.

1-(3-hydroxyazetidin-1-yl)propan-1-one (194 mg, 33%) was prepared using procedure of (S)-1-(3-hydroxy-pyrrolidin-1-yl)-propan-1-one and starting from azetidin-3-ol hydrochloride. Purification by column chromatography on silica gel, using dichloromethane/methanol as eluent, afforded the product as an orange solid.

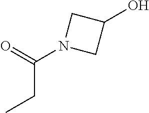

¹H-NMR (400 MHz, DMSO): 0.94 (t, J 7.5 Hz, 3H, CH₃); 2.03 (q, J 7.5 Hz, 2H, CH₂); 3.54 (dd, J 4.4, 10.0 Hz, 1H, CH₂—N); 3.80 (dd, J 4.4, 8.8 Hz, 1H, CH₂—N); 3.99 (dd, J 7.2, 9.5 Hz, 1H, CH₂—N); 4.20-4.28 (m, 1H, CH₂—N); 4.37-4.46 (m, 1H, CH—O); 5.66 (d, J 6.2 Hz, 1H, OH).

### Compound 117 (8-methyl-6-(2-morpholinoethyl)-2-[4-(trifluoromethyl)-2-pyridyl]-3H-quinazolin-4-one hydrochloride)

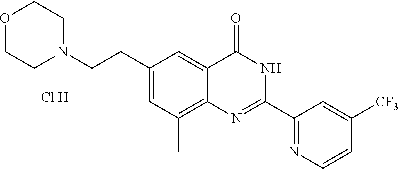

Compound 117 was prepared according to procedure of example 34, starting from 6-bromo-8-methyl-2-(4-methyl-2-pyridyl)-3-(2-trimethylsilylethoxymethyl) quinazolin-4-one (from example 35) in step 1, and using 9BBN instead of BH₃.Me₂S in step 2, to afford the product as a white solid.

¹H-NMR (400 MHz, DMSO): 2.68 (s, 3H, CH₃); 3.06-3.24 (m, 4H, CH₂); 3.39-3.48 (m, 2H, CH₂—N); 3.52 (d, J 12.0 Hz, 2H, CH₂—N); 3.78 (t, J 11.8 Hz, 2H, CH₂—O); 4.01 (d, J 12.3 Hz, 2H, CH₂—O); 7.69 (d, J 1.3 Hz, 1H, Ar); 7.98 (d, J 1.3 Hz, 1H, Ar); 8.06 (dd, J 1.1, 5.2 Hz, 1H, Ar); 8.67 (t, J 0.8 Hz, 1H, Ar); 9.04 (d, J 5.1 Hz, 1H, Ar); 10.75 (bs, 1H, HCl salt); 12.17 (s, 1H, NH). M/Z (M+H)⁺=419.8. MP>250° C.

### Compound 118 (8-Methyl-6-[(1-methyl-6-oxo-3-piperidyl)oxy]-2-pyrrolo[1,2-c]pyrimidin-3-yl-3H-quinazolin-4-one)

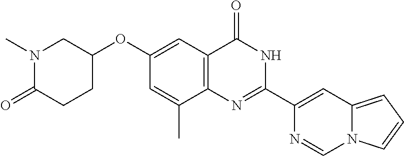 [SMILES: Cc1cc(OC2CCC(=O)N(C)C2)cc2c(=O)[nH]c(-c3cc4cccn4cn3)nc12]

Compound 118 was prepared according to procedure of example 27 step 1 and 2, starting from 6-hydroxy-8-methyl-2-pyrrolo[1,2-c]pyrimidin-3-yl-3-(2-trimethylsilanyl-ethoxymethyl)-3H-quinazolin-4-one in step 1, and from 5-hydroxy-1-methyl-piperidin-2-one in step 2, followed by procedure of example 30, step 2. Purification by preparative HPLC afforded the product as a green solid.

¹H-NMR (400 MHz, DMSO): 2.05-2.11 (m, 2H, CH₂); 2.24-2.41 (m, 2H, CH₂); 2.63 (s, 3H, CH₃); 2.82 (s, 3H, CH₃—N); 3.43 (dd, J 13.2, 3.9 Hz, 1H, CH₂—N); 3.68 (dd, J 13.1, 3.9 Hz, 1H, CH₂—N); 5.00-5.04 (m, 1H, CH—O); 6.87-6.89 (m, 1H, Ar); 7.06-7.08 (m, 1H, Ar); 7.40 (dd, J 2.9, 0.8 Hz, 1H, Ar); 7.47 (d, J 2.9 Hz, 1H, Ar); 7.89-7.90 (m, 1H, Ar); 8.53 (d, J 0.8 Hz, 1H, Ar); 9.33-9.34 (m, 1H, Ar); 11.44 (bs, 1H, NH). M/Z (M+H)⁺=404.9. MP=220-230° C.

### Compound 119 (8-Methyl-6-(morpholinomethyl)-2-(thieno[3,2-c]pyridin-6-yl)quinazolin-4(3H)-one hydrochloride)

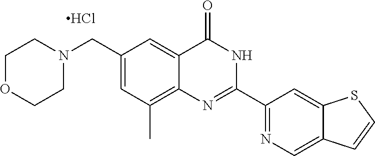 [SMILES: Cc1cc(CN2CCOCC2)cc2c(=O)[nH]c(-c3cc4sccc4cn3)nc12 ; Cl]

Compound 119 was prepared according to procedure of example 37 starting from 6-bromo-8-methyl-2-(thieno[3,2-c]pyridin-6-yl)-3-((2-(trimethylsilyl)ethoxy)methyl)quinazolin-4(3H)-one in step 1 to afford the product as a yellow solid.

¹H-NMR DMSO (400 MHz): 2.73 (s, 3H, CH₃); 3.09-3.22 (m, 2H, CH₂—N); 3.30 (d, J 12.4 Hz, 2H, CH₂—N); 3.75 (t, J 11.8 Hz, 2H, CH₂—O); 3.96 (d, J 12.4 Hz, 2H, CH₂—O); 4.44-4.52 (m, 2H, CH₂—N); 7.78 (dd, J 5.4, 0.6 Hz, 1H, Ar); 7.96 (d, J 1.3 Hz, 1H, Ar); 8.15 (d, J 5.4 Hz, 1H, Ar); 8.24 (d, J 1.6 Hz, 1H, Ar); 9.29 (t, J 0.9 Hz, 1H, Ar); 9.33 (d, J 0.9 Hz, 1H, Ar); 10.74 (bs, 1H, HCl); 12.01 (bs, 1H, NH). M/Z (M+H)⁺=393.7. MP>250° C.

6-bromo-8-methyl-2-(thieno[3,2-c]pyridin-6-yl)-3-((2-(trimethylsilyl)ethoxy)methyl)quinazolin-4(3H)-one was prepared according to procedure of example 23 step 1 to 4 starting from 2-amino-3-methylbenzoic acid in step 1, and from thieno[3,2-c]pyridine-6-carboxylic acid in step 3 and using N-methyl-pyrrolidine instead of dimethylformamide as a solvent in step 4.

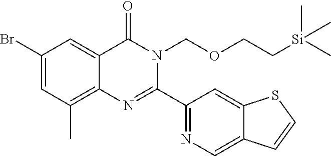 [SMILES: Cc1cc(Br)cc2c(=O)n(COCC[Si](C)(C)C)c(-c3cc4sccc4cn3)nc12]

M/Z (M[⁷⁹Br]+H)⁺=372.3.

### Compound 120 (8-Methyl-6-(3-oxa-9-azaspiro[5.5]undecan-9-yl)-2-(thieno[3,2-c]pyridin-6-yl)quinazolin-4(3H)-one)

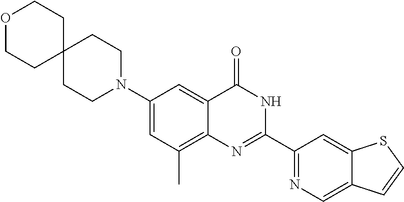 [SMILES: Cc1cc(N2CCC3(CCOCC3)CC2)cc2c(=O)[nH]c(-c3cc4sccc4cn3)nc12]

Compound 120 was prepared according to procedure of example 30 step 1, starting from 3-oxa-9-azaspiro[5.5]undecane and 6-bromo-8-methyl-2-(thieno[3,2-c]pyridin-6-yl)-3-((2-(trimethylsilyl)ethoxy)methyl)quinazolin-4(3H)-one, followed by procedure of example 27 step 3 to afford the product as a yellow solid.

¹H-NMR (400 MHz, DMSO): 1.48 (t, J 5.4 Hz, 4H, CH₂); 1.63-1.65 (m, 4H, CH₂); 2.67 (s, 3H, CH₃); 3.32-3.36 (m, 4H, CH₂—N); 3.59 (t, J 5.4 Hz, 4H, CH₂—O); 7.34 (d, J 2.8 Hz, 1H, Ar); 7.48 (d, J 2.8 Hz, 1H, Ar); 7.74 (dd, J 5.5, 0.7 Hz, 1H, Ar); 8.08 (d, J 5.5 Hz, 1H, Ar); 9.15 (bs, 1H, Ar); 9.24 (bs, 1H, Ar); 11.5 (s, 1H, NH). M/Z (M+H)⁺=447.7. MP>250° C.

### Example 38—Synthesis of compound 121 (8-Methyl-6-(2-[1,4]oxazepan-4-yl-ethyl)-2-thieno[2,3-b]pyridin-5-yl-3H-quinazolin-4-one hydrochloride)

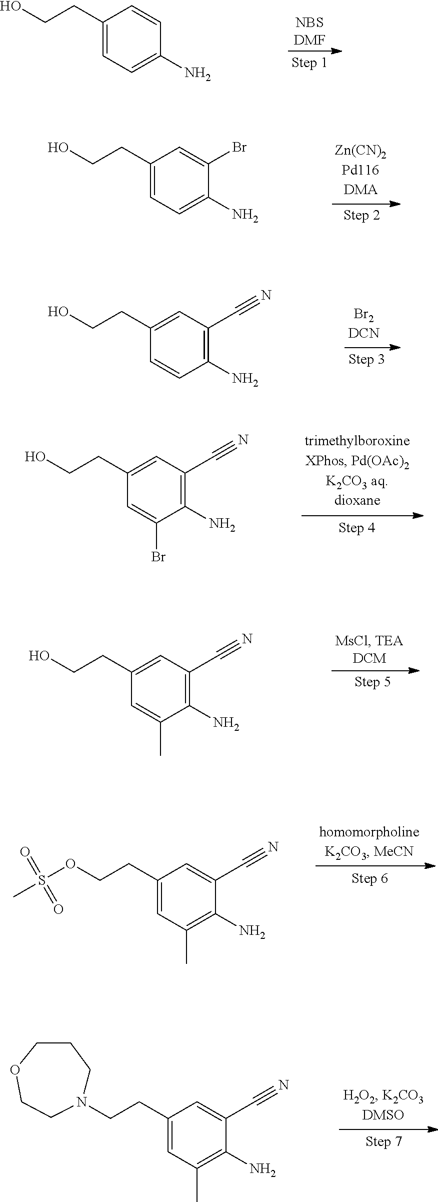

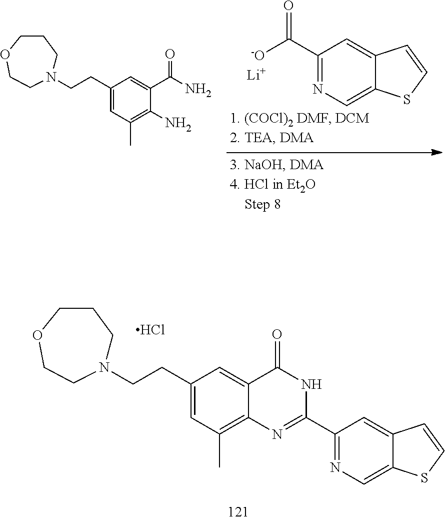

Step 1:

Under inert atmosphere, to a solution of 4-aminophenethyl alcohol (4.00 g, 29.16 mmol) in dimethylformamide (146 mL), N-bromosuccinimide (5.20 g, 29.16 mmol) was added portionwise at 0° C. The resulting mixture was stirred for 1 h at 0° C. before being diluted with an aqueous saturated solution of sodium bicarbonate (800 mL) and extracted with ethyl acetate (2×800 mL). The combined organic extracts were washed with brine and concentrated in vacuo. The crude residue was purified by flash column chromatography on silica gel using cyclohexane/ethyl acetate as eluent to afford 2-(4-amino-3-bromophenyl)ethan-1-ol (5.74 g, 91%) as a white solid.

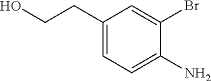 [SMILES: Nc1ccc(CCO)cc1Br]

M/Z (M[⁷⁹Br]+H)⁺=216.2.

Step 2:

Under inert atmosphere, to a degassed solution of 2-(4-amino-3-bromophenyl)ethan-1-ol (5.74 g, 26.56 mmol) and zinc cyanide (6.23 g, 53.1 mmol) in dimethylacetamide (133 mL) was added bis(tri-tert-butylphosphine)palladium(0) (672 mg, 1.31 mmol). The reaction mixture was heated for 15 min at 130° C. before being diluted with an aqueous saturated solution of sodium bicarbonate (1000 mL) and extracted with ethyl acetate (2×1000 mL). The combined organic extracts were washed with brine, dried over MgSO₄ and concentrated in vacuo. The crude residue was co-evaporated three times with toluene, and then purified by flash column chromatography on silica gel using cyclohexane/ethyl acetate as eluent to afford 2-amino-5-(2-hydroxyethyl)benzonitrile (3.47 g, 81%) as a white solid.

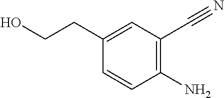 [SMILES: N#Cc1cc(CCO)ccc1N]

M/Z (M+H)⁺=163.8.

Step 3:

Under inert atmosphere, to a solution of 2-amino-5-(2-hydroxyethyl)benzonitrile (3.49 g, 21.52 mmol) in dichloromethane (107 mL) at 0° C. was added bromine (1.15 mL, 22.59 mmol) in solution in dichloromethane (55 mL) over 30 min. The reaction mixture was stirred for 2 h at room temperature before being diluted with an aqueous saturated solution of sodium bicarbonate and extracted with dichloromethane. The combined organic extracts were dried over Na₂SO₄ and concentrated in vacuo to afford 2-amino-3-bromo-5-(2-hydroxyethyl)benzonitrile (4.72 g, 94%) as a light brown solid.

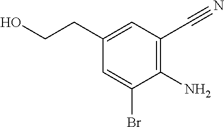 [SMILES: N#Cc1cc(CCO)cc(Br)c1N]

M/Z (M[⁷⁹Br]+H)⁺=241.7.

Step 4:

Under inert atmosphere, to a degassed suspension of 2-amino-3-bromo-5-(2-hydroxyethyl)benzonitrile (5.13 g, 21.27 mmol), aqueous potassium carbonate (1.2M, 26.6 mL, 31.9 mmol) and trimethylboroxine (5.95 mL, 42.53 mmol) in dioxane (106 mL) was added XPhos Pd G2 (837 mg, 1.06 mmol). The reaction mixture was stirred for 15 h at 100° C. before being diluted with ethyl acetate (300 mL). The combined organic extracts were washed with 1M aqueous sodium hydroxide (3×60 mL), with brine and dried over Na₂SO₄ and then concentrated in vacuo. The crude residue was purified by flash column chromatography on silica gel using cyclohexane/ethyl acetate as eluent to afford 2-amino-5-(2-hydroxyethyl)-3-methylbenzonitrile (1.945 g, 52%) as a yellow oil.

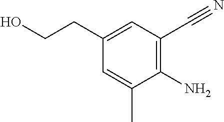

M/Z (M+H)⁺=177.7.

Step 5:

Under inert atmosphere, to a solution of 2-amino-5-(2-hydroxyethyl)-3-methylbenzonitrile (655 mg, 3.71 mmol) in dichloromethane (18.5 mL) at 0° C. were added triethylamine (622 μL, 4.46 mmol) and mesyl chloride (345 μL, 4.46 mmol). The reaction mixture was stirred for 1 h at room temperature before being diluted with an aqueous saturated solution of sodium bicarbonate (30 mL). The resulting biphasic mixture was vigorously stirred for 15 min and then extracted with dichloromethane (3×20 mL). The combined organic extracts were washed brine, dried over MgSO₄ and concentrated in vacuo. The crude residue was purified by flash column chromatography on silica gel using cyclohexane/ethyl acetate as eluent to afford 4-amino-3-cyano-5-methylphenethyl methanesulfonate (862 mg, 91%) as a pale-yellow oil.

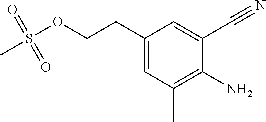 [SMILES: Cc1cc(CCOS(C)(=O)=O)cc(C#N)c1N]

M/Z (M+H)⁺=255.7.

Step 6:

To a solution of 4-amino-3-cyano-5-methylphenethylmethane sulfonate (600 mg, 2.36 mmol) in acetonitrile (24 mL) were added K₂CO₃ (979 mg, 7.08 mmol) and homomorpholine (350 mg, 3.54 mmol). The resulting mixture was heated at 100° C. for 17 h before being diluted with water (70 mL) and extracted with dichloromethane (2×70 mL). The combined organic extracts were concentrated in vacuo. The crude residue was purified by flash column chromatography on silica gel using dichloromethane/methanol as eluent to afford 5-(2-(1,4-oxazepan-4-yl)ethyl)-2-amino-3-methylbenzonitrile (340 mg, 56%).

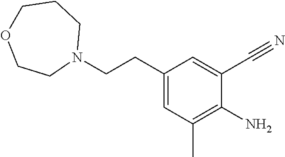 [SMILES: Cc1cc(CCN2CCCOCC2)cc(C#N)c1N]

M/Z (M+H)⁺=260.8.

Step 7:

To a solution of 5-(2-(1,4-oxazepan-4-yl)ethyl)-2-amino-3-methylbenzonitrile (340 mg, 1.31 mmol) in DMSO (7 mL) were added K₂CO₃ (217 mg, 1.57 mmol) and H₂O₂ (30% in water, 101 μL, 3.93 mmol). The resulting mixture was stirred for 17 hat room temperature before being diluted with water (30 mL) and extracted with dichloromethane (2×30 mL). The combined organic extracts were concentrated under vacuum. The crude residue was purified by flash column chromatography on silica gel using dichloromethane/methanol as eluent to afford 5-(2-(1,4-oxazepan-4-yl)ethyl)-2-amino-3-methylbenzamide (190 mg, 52%).

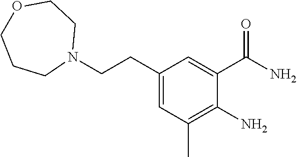 [SMILES: Cc1cc(CCN2CCCOCC2)cc(C(N)=O)c1N]

M/Z (M+H)⁺=278.8

Step 8:

Under inert atmosphere, at 0° C., to a solution of thieno[2,3-b]pyridine-5-carboxylic acid (97 mg, 0.54 mmol) in dichloromethane (1.4 mL), oxalyl chloride (69 μL, 0.81 mmol) and then DMF (2 μL, 0.03 mmol) were added dropwise. The reaction mixture was stirred at room temperature for 1 h before being concentrated to dryness, and co-evaporated twice with toluene. The crude acyl chloride was dissolved in dimethylacetamide (1.4 mL), then triethylamine (113 μL, 0.81 mmol) and 5-(2-(1,4-oxazepan-4-yl)ethyl)-2-amino-3-methylbenzamide (75 mg, 0.27 mmol) were added and the reaction mixture was stirred at room temperature for 1 h. Then an aqueous solution of NaOH (1N, 1.6 mL, 1.62 mmol) was added and the reaction mixture was heated at 100° C. for 1 h. The solution was then allowed to cool down to room temperature and water (15 mL) and ethanol (2 mL) were added. The resulting solid was collected by filtration and rinsed with a water/ethanol 1:1 mixture. It was then dried in vacuo. The HCl salt was obtained by filtration after addition of an excess of HCl (2N in Et₂O) to a solution of the free base in methanol. Compound 121 was obtained as a beige solid in 35% yield.

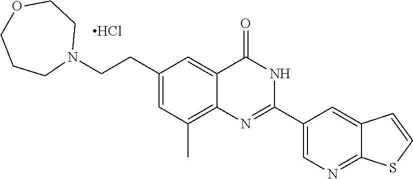 [SMILES: Cc1cc(CCN2CCCOCC2)cc2c(=O)[nH]c(-c3cnc4sccc4c3)nc12 ; Cl]

¹H-NMR (400 MHz, DMSO): 1.99-2.12 (m, 1H, CH₂); 2.20-2.37 (m, 1H, CH₂); 2.70 (s, 3H, CH₃); 3.16-3.50 (m, 6H, CH₂); 3.53-3.61 (m, 2H, CH₂); 3.71-3.95 (m, 4H, CH₂); 7.68 (s, 1H, Ar); 7.82 (d, 1H, J 5.3 Hz, Ar); 7.97 (s, 1H, Ar); 8.30 (d, 1H, J 5.3 Hz, Ar); 9.00 (s, 1H, Ar); 9.46 (s, 1H, Ar); 10.89 (bs, 1H, HCl salt); 11.83 (bs, 1H, NH). M/Z (M+H)⁺=421.8. MP>250° C.

### Compound 122 (8-Methyl-6-(2-[1,4]oxazepan-4-yl-ethyl)-2-thieno[3,2-b]pyridin-6-yl-3H-quinazolin-4-one hydrochloride)

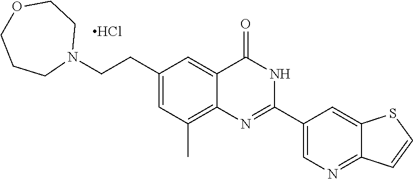 [SMILES: Cc1cc(CCN2CCCOCC2)cc2c(=O)[nH]c(-c3cnc4ccsc4c3)nc12 ; Cl]

Compound 122 was prepared according to procedure of example 38, starting from thieno[3,2-c]pyridine-6-carboxylic acid in step 8. The HCl salt was obtained by freeze-drying of a suspension of the free base in water and an excess of aqueous 1N HCl to afford the product as a yellow solid.

¹H-NMR (400 MHz, DMSO): 1.99-2.12 (m, 1H, CH₂); 2.20-2.33 (m, 1H, CH₂); 2.70 (s, 3H, CH₃); 3.15-3.49 (m, 6H, CH₂); 3.53-3.67 (m, 2H, CH₂); 3.67-3.93 (m, 4H, CH₂); 7.68 (d, J 1.4 Hz, 1H, Ar); 7.77 (dd, J 5.4, 0.6 Hz, 1H, Ar); 7.97 (d, J 1.4 Hz, 1H, Ar); 8.13 (d, J 5.4 Hz, 1H, Ar); 9.25 (bs, 1H, Ar); 9.31 (d, J 0.6 Hz, 1H, Ar); 10.99 (bs, 1H, HCl salt); 11.85 (bs, 1H, NH). M/Z (M+H)⁺=421.8. MP>250° C.

### Compound 123 (8-Methyl-6-(2-morpholin-4-yl-ethyl)-2-thieno[3,2-c]pyridin-6-yl-3H-quinazolin-4-one hydrochloride)

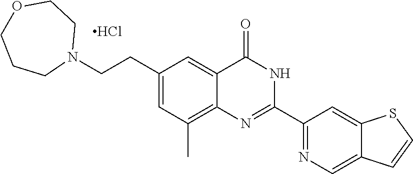 [SMILES: Cc1cc(CCN2CCCOCC2)cc2c(=O)[nH]c(-c3cc4sccc4cn3)nc12 ; Cl]

Compound 123 was prepared according to procedure of example 38, using morpholine instead of homomorpholine in step 6 and starting from thieno[3,2-c]pyridine-6-carboxylic acid in step 8. The HCl salt was obtained by freeze-drying of a suspension of the free base in water and an excess of aqueous 1N HCl to afford the product as a yellow solid.

¹H-NMR (400 MHz, DMSO): 2.72 (s, 3H, CH₃); 3.10-3.21 (m, 4H, CH₂); 3.40-3.47 (m, 2H, CH₂—N); 3.52-3.54 (m, 2H, CH₂—N); 3.74-3.80 (m, 2H, CH₂—O); 3.99-4.03 (m, 2H, CH₂—O); 7.67 (bs, 1H, Ar); 7.77 (d, J 5.2 Hz, 1H, Ar); 7.97 (bs, 1H, Ar); 8.13 (d, J 5.2 Hz, 1H, Ar); 9.25 (s, 1H, Ar); 9.31 (s, 1H, Ar); 10.59 (bs, 1H, HCl salt); 11.85 (bs, 1H, NH). M/Z (M+H)⁺=407.8. MP>250° C.

### Compound 124 (8-Methyl-6-(2-morpholin-4-yl-ethyl)-2-pyrrolo[1,2-c]pyrimidin-3-yl-3H-quinazolin-4-one hydrochloride)

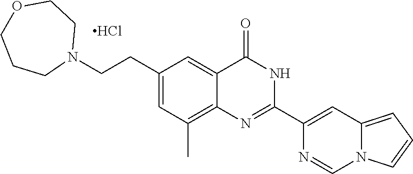 [SMILES: Cc1cc(CCN2CCCOCC2)cc2c(=O)[nH]c(-c3cc4cccn4cn3)nc12 ; Cl]

Compound 124 was prepared according to procedure of example 38, using morpholine instead of homomorpholine in step 6 and starting from pyrrolo[1,2-c]pyrimidine-3-carboxylic acid in step 8. The HCl salt was obtained by freeze-drying of a suspension of the free base in water and an excess of aqueous 1N HCl to afford the product as a yellow solid.

¹H-NMR (400 MHz, DMSO): 2.66 (s, 3H, CH₃); 3.11-3.21 (m, 4H, CH₂); 3.38-3.43 (m, 2H, CH₂—N); 3.50-3.53 (m, 2H, CH₂—N); 3.80-3.84 (m, 2H, CH₂—O); 3.99-4.02 (m, 2H, CH₂—O); 6.91 (d, J 3.7 Hz, 1H, Ar); 7.09 (dd, J 3.7, 2.7 Hz, 1H, Ar); 7.63 (s, 1H, Ar); 7.92 (bs, 2H, Ar); 8.58 (s, 1H, Ar); 9.35 (s, 1H, Ar); 11.07 (bs, 1H, HCl salt); 11.53 (bs, 1H, NH). M/Z (M+H)⁺=390.8. MP>250° C.

### Compound 125 (8-Methyl-6-(morpholinomethyl)-2-pyrrolo[1,2-c]pyrimidin-3-yl-3H-quinazolin-4-one hydrochloride)

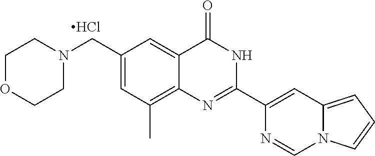 [SMILES: Cc1cc(CN2CCOCC2)cc2c(=O)[nH]c(-c3cc4cccn4cn3)nc12 ; Cl]

Compound 125 was prepared according to procedure of example 37 starting from 6-bromo-8-methyl-2-pyrrolo[1,2-c]pyrimidin-3-yl-3-(2-trimethylsilanyl-ethoxymethyl)-3H-quinazolin-4-one in step 1. The free base was purified by preparative HPLC and pure fractions were freeze-dried with water and an excess of aqueous 1N HCl to afford the product as a green solid.

¹H-NMR (400 MHz, DMSO): 2.68 (s, 3H, CH₃); 3.07-3.20 (m, 2H, CH₂—N); 3.29 (d, J 12.2 Hz, 2H, CH₂—N); 3.72 (t, J 12.2 Hz, 2H, CH₂—O); 3.96 (d, J 12.2 Hz, 2H, CH₂—O); 4.4 (d, J 4.3 Hz, 2H, CH₂—N); 6.92-6.95 (m, 1H, Ar); 7.10 (dd, J 3.9, 2.9 Hz, 1H, Ar); 7.90 (d, J 1.0 Hz, 1H, Ar); 7.92-7.95 (m, 1H, Ar); 8.20 (d, J 1.8 Hz, 1H Ar); 8.63 (d, J 1.0 Hz, 1H, Ar); 9.34-9.38 (m, 1H, Ar); 10.62 (bs, 1H, HCl salt); 11.69 (bs, 1H, NH). M/Z (M+H)⁺=376.8. MP>250° C.

### Example 39—Synthesis of compound 126 (8-Methyl-6-(2-morpholino-2-oxoethyl)-2-(thieno[3,2-c]pyridin-6-yl)quinazolin-4(3H)-one)

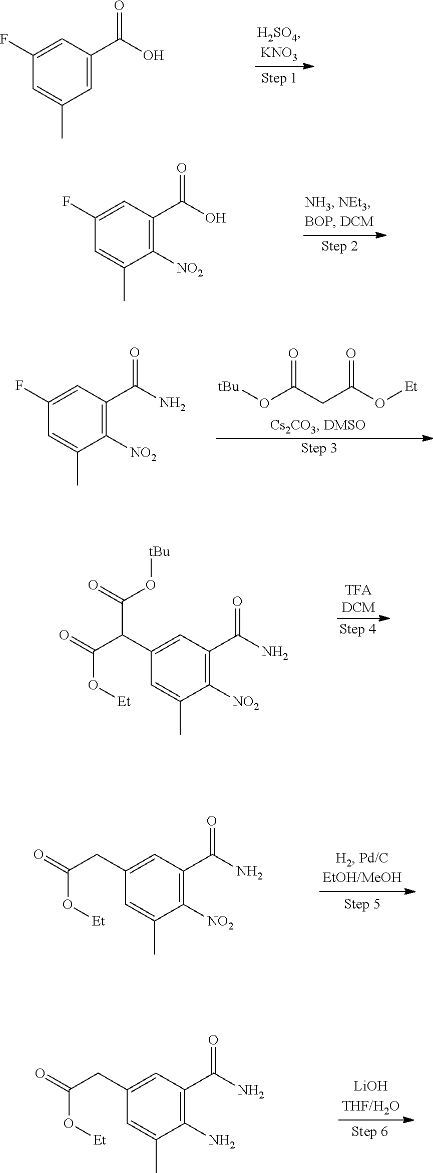

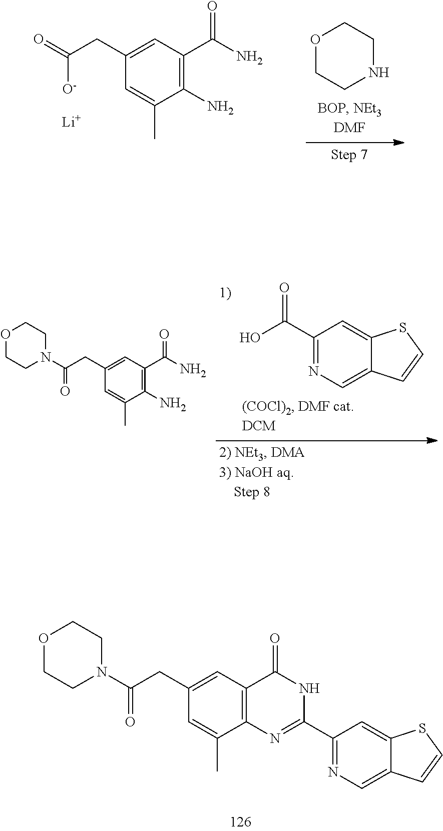 [SMILES: Cc1cc(CC(=O)[O-])cc(C(N)=O)c1N ; [Li+] ; C1COCCN1 ; Cc1cc(CC(=O)N2CCOCC2)cc(C(N)=O)c1N ; O=C(O)c1cc2sccc2cn1 ; Cc1cc(CC(=O)N2CCOCC2)cc2c(=O)[nH]c(-c3cc4sccc4cn3)nc12]

Step 1:

At 0° C. to a suspension of 3-fluoro-5-methylbenzoic acid (1.00 g, 6.88 mmol) in concentrated sulfuric acid (8 mL), KNO₃ (722 mg, 7.14 mmol) was added in one portion and the suspension was stirred at room temperature for 1.5 h. The resulting mixture was slowly poured into ice/water and the resulting precipitate was collected by filtration and rinsed with water. The solid was then dried in vacuo with P₂O₅ to afford the expected product 5-fluoro-3-methyl-2-nitrobenzoic acid (969 mg, 75%) as a white solid.

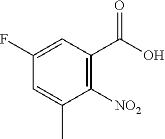

¹H-NMR (400 MHz, DMSO): 2.30 (s, 3H, CH₃); 7.60-7.67 (m, 2H, Ar); 14.19 (bs, 1H, COOH).

Step 2:

5-fluoro-3-methyl-2-nitrobenzamide was prepared from 5-fluoro-3-methyl-2-nitrobenzoic acid (640 mg, 3.214 mmol) according to procedure of example 1, step 1, and isolated as a light-yellow solid in 92% yield.

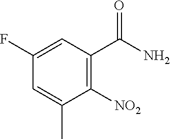

¹H-NMR (400 MHz, DMSO): 2.30 (s, 3H, CH₃); 7.46 (dd, J 8.8, 2.6 Hz, 1H; Ar); 7.51 (ddd, J 8.8, 2.6, 0.5 Hz, 1H, Ar).

Step 3:

Under inert atmosphere, to a solution of 5-fluoro-3-methyl-2-nitrobenzamide (379 mg, 1.912 mmol) in dimethyl sulfoxide (19 mL) were added tert-butyl ethyl malonate (543 μL, 2.869 mmol) and cesium carbonate (2.49 g, 7.648 mmol). The resulting mixture was stirred at 80° C. for 3 h before being diluted with water (100 mL) and extracted with dichloromethane (2×100 mL). The combined organic extracts were dried over Na₂SO₄ and concentrated in vacuo. The oily crude residue (1.39 g) containing 1-(tert-butyl) 3-ethyl 2-(3-carbamoyl-5-methyl-4-nitrophenyl)malonate in a mixture with DMSO and tert-butyl ethyl malonate was used as such for the next step.

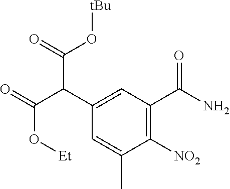 [SMILES: CCOC(=O)C(C(=O)OC(C)(C)C)c1cc(C)c([N+](=O)[O-])c(C(N)=O)c1]

Step 4:

To a solution of crude 1-(tert-butyl) 3-ethyl 2-(3-carbamoyl-5-methyl-4-nitrophenyl)malonate (1.921 mmol) in dichloromethane (8 mL), trifluoroacetic acid (8 mL) was added, and the mixture was stirred at room temperature for 2 h before being evaporated to dryness. The residue was partitioned between ethyl acetate (40 mL) and an aqueous saturated solution of sodium bicarbonate (40 mL). The aqueous phase was then adjusted to pH 11 with a sodium hydroxide solution (10 M, 12 mL) and extracted twice with ethyl acetate (2×50 mL). The combined organic extracts were dried over Na₂SO₄ and concentrated in vacuo to afford ethyl 2-(3-carbamoyl-5-methyl-4-nitrophenyl)acetate (214 mg, 42% over 2 steps) as a colorless oil that crystallized on standing.

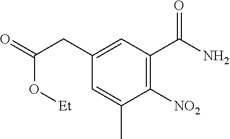 [SMILES: CCOC(=O)Cc1cc(C)c([N+](=O)[O-])c(C(N)=O)c1]

M/Z (M+H)⁺=367.6.

Step 5:

Under inert atmosphere, in a Parr high pressure reactor, to a solution of 2-(3-carbamoyl-5-methyl-4-nitrophenyl)acetate (1.12 g, 4.10 mmol) in methanol (41 mL) was added 10% palladium on charcoal (436 mg, 0.41 mmol). The suspension was placed under hydrogen gas at 5 bars and stirred overnight at room temperature. The reaction mixture was filtrated over Celite® and concentrated in vacuo. The crude residue was purified by recrystallisation from ethanol (15 mL) and methanol (2 mL) at 80° C. to afford ethyl 2-(4-amino-3-carbamoyl-5-methylphenyl)acetate (713 mg, 76%) as a yellow solid.

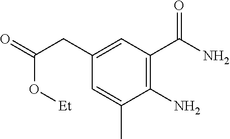 [SMILES: CCOC(=O)Cc1cc(C)c(N)c(C(N)=O)c1]

M/Z (M+H)⁺=237.7

Step 6:

To a solution of ethyl 2-(4-amino-3-carbamoyl-5-methylphenyl)acetate (350 mg, 1.48 mmol) in tetrahydrofuran (7 mL) and water (7 mL) was added lithium hydroxide (61 mg, 1.85 mmol). The reaction mixture was heated to 70° C. for 4 h before being reduced in vacuo to remove tetrahydrofuran. The resulting aqueous mixture was freeze-dried to afford crude lithium 2-(4-amino-3-carbamoyl-5-methylphenyl)acetate (323 mg) as a white solid.

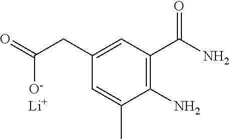

M/Z (M+H)⁺=209.7.

Step 7:

Under inert atmosphere, to a solution of crude lithium 2-(4-amino-3-carbamoyl-5-methylphenyl)acetate (1.48 mmol) and benzotriazol-1-yloxy-tris(dimethylamino)-phosphonium hexafluorophosphate (1.31 g, 2.96 mmol)) in dimethylformamide (15 mL were added triethylamine (619 μL, 4.44 mmol) and morpholine (324 μL, 3.7 mmol). The mixture was stirred at room temperature for 2 h. The mixture was then diluted with a saturated aqueous solution of ammonium chloride (20 mL) and extracted with ethyl acetate (8×100 mL). The combined organic extracts were dried over Na₂SO₄ and concentrated under vacuum. The crude residue was purified by flash column chromatography on silica gel using dichloromethane/methanol as eluent to afford 2-amino-3-methyl-5-(2-morpholino-2-oxoethyl)benzamide (274 mg, 67% over 2 steps) as a white solid.

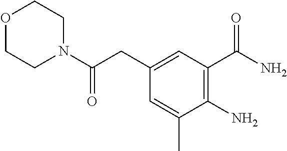 [SMILES: Cc1cc(CC(=O)N2CCOCC2)cc(C(N)=O)c1N]

M/Z (M+H)⁺=279.8.

Step 8:

Compound 126 (105 mg, 87%) was prepared according to procedure of example 38 step 8, starting from 2-amino-3-methyl-5-(2-morpholino-2-oxoethyl)benzamide (80 mg, 0.288 mmol) and thieno[3,2-c]pyridine-6-carboxylic acid (116 mg, 0.577 mmol), without HCl salt formation, to afford the product as a white solid.

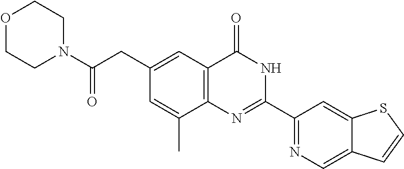 [SMILES: Cc1cc(CC(=O)N2CCOCC2)cc2c(=O)[nH]c(-c3cc4sccc4cn3)nc12]

¹H-NMR (400 MHz, DMSO): 2.69 (s, 3H, CH₃); 3.45-3.51 (m, 2H, CH₂); 3.51-3.59 (m, 6H, CH₂); 3.87 (s, 2H, CH₂—CO); 7.59 (s, 1H, Ar); 7.77 (dd, J 5.4, 0.6 Hz, 1H, Ar); 7.89 (d, J 1.5 Hz, 1H, Ar); 8.12 (d, J 5.4 Hz, 1H, Ar); 9.24 (s, 1H, Ar); 9.31 (s, 1H, Ar); 11.76 (bs, 1H, NH), M/Z (M+H)⁺=421.8. MP>250° C.

### Compound 127 (8-Methyl-6-(2-morpholino-2-oxo-ethyl)-2-thieno[2,3-c]pyridin-5-yl-3H-quinazolin-4-one)

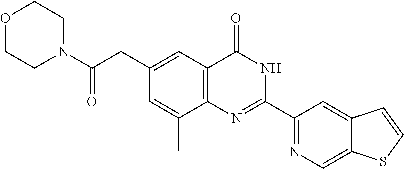 [SMILES: Cc1cc(CC(=O)N2CCOCC2)cc2c(=O)[nH]c(-c3cc4ccsc4cn3)nc12]

Compound 127 was prepared according to procedure of example 39 starting from thieno[2,3-c]pyridine-5-carboxylic acid in step 8.

¹H-NMR (400 MHz, DMSO): 2.69 (s, 3H, CH₃); 3.46-3.51 (m, 2H, CH₂); 3.52-3.58 (m, 6H, CH₂); 3.87 (s, 2H, CH₂—CO); 7.59 (d, J 1.6 Hz, 1H, Ar); 7.82 (dd, J 5.4, 0.4 Hz, 1H, Ar); 7.89 (d, J 1.6 Hz, 1H, Ar); 8.29 (d, J 5.4 Hz, 1H, Ar); 9.01 (d, J 0.8 Hz, 1H, Ar); 9.45 (bs, 1H, Ar); 11.76 (bs, 1H, NH). M/Z (M+H)⁺=421.8. MP>250° C.

### Compound 128 (8-Methyl-6-(2-piperidin-1-yl-ethyl)-2-thieno[2,3-b]pyridin-5-yl-3H-quinazolin-4-one hydrochloride)

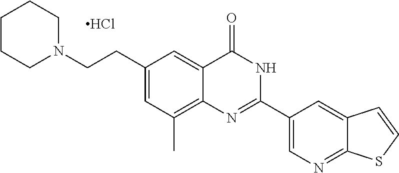 [SMILES: Cc1cc(CCN2CCCCC2)cc2c(=O)[nH]c(-c3cnc4sccc4c3)nc12 ; Cl]

Compound 128 was prepared according to procedure of example 38 using piperidine instead of homomorpholine in step 6. The HCl salt was obtained by freeze-drying of a suspension of the free base in water, acetonitrile and an excess of aqueous 1N HCl to afford the product as a yellow solid.

¹H-NMR (DMSO+D₂O, 400 MHz): 1.33-1.47 (m, 1H, CH₂); 1.61-1.75 (m, 3H, CH₂); 1.80-1.91 (m, 2H, CH₂); 2.69 (s, 3H, CH₃); 2.93 (td, J 12.3, 2.2 Hz, 2H, CH₂); 3.08-3.18 (m, 2H, CH₂); 3.28-3.35 (m, 2H, CH₂); 3.48-3.56 (m, 2H, CH₂); 7.67 (d, J 1.4 Hz, 1H, Ar); 7.78 (d, J 5.4 Hz, 1H, Ar); 7.95 (d, J 1.4 Hz, 1H, Ar); 8.24 (d, J 5.4 Hz, 1H, Hz, Ar); 8.99 (d, J 0.5 Hz, 1H, Ar); 9.41 (bs, 1H, Ar). M/Z (M+H)⁺=405.8. MP>250° C.

### Compound 129 (8-Methyl-6-(1-methyl-6-oxo-piperidin-3-yloxy)-2-thieno[3,2-c]pyridin-6-yl-3H-quinazolin-4-one)

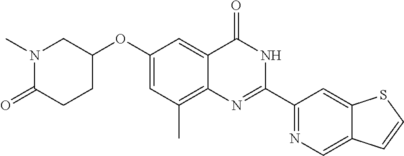 [SMILES: Cc1cc(OC2CCC(=O)N(C)C2)cc2c(=O)[nH]c(-c3cc4sccc4cn3)nc12]

Compound 129 was prepared according to procedure of example 27 starting from 6-bromo-8-methyl-2-(thieno[3,2-c]pyridin-6-yl)-3-((2-(trimethylsilyl)ethoxy)methyl)quinazolin-4(3H)-one and using diisopropyl azodicarboxylate instead of diethyl azodicarboxylate and starting from 5-hydroxy-1-methyl-piperidin-2-one in step 2 to afford the product as a beige solid.

¹H-NMR (400 MHz, DMSO): 2.06-2.12 (m, 2H, CH₂); 2.25-2.40 (m, 2H, CH₂); 2.69 (s, 3H, CH₃); 2.83 (s, 3H, CH₃—N); 3.44 (dd, J 13.2, 3.4 Hz, 1H, CH₂); 3.69 (dd, J 13.2, 3.8 Hz, 1H, CH₂); 5.02-5.07 (m, 1H, CH); 7.44 (d, J 2.6 Hz, 1H, Ar); 7.51 (d, J 2.6 Hz, 1H, Ar); 7.76 (d, J 5.3 Hz, 1H, Ar); 8.11 (d, J 5.3 Hz, 1H, Ar); 9.20 (bs, 1H, Ar); 9.29 (s, 1H, Ar); 11.74 (bs, 1H, NH). M/Z (M+H)⁺=421.8. MP=90-110° C.

### Compound 130 (8-Methyl-6-(1-methyl-2-oxo-piperidin-4-ylmethoxy)-2-thieno[2,3-c]pyridin-5-yl-3H-quinazolin-4-one)

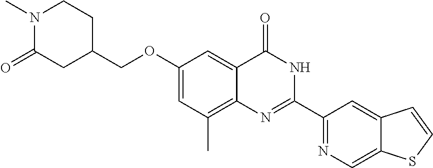 [SMILES: Cc1cc(OCC2CCN(C)C(=O)C2)cc2c(=O)[nH]c(-c3cc4ccsc4cn3)nc12]

Compound 130 was prepared according to procedure of example 27 step 1 and 2, using diisopropyl azodicarboxylate instead of diethyl azodicarboxylate and starting from 4-(hydroxymethyl)-1-methylpiperidin-2-one in step 2. Then followed by procedure of example 36 step 7. Purification by column chromatography on silica gel, using dichloromethane/methanol as eluent, afforded the product as a white solid.

¹H-NMR (400 MHz, DMSO): 1.53-1.64 (m, 1H, CH₂); 1.93-1.99 (m, 1H, CH₂); 2.04-2.11 (m, 1H, CH); 2.29-2.35 (m, 1H, CH₂—N); 2.37-2.39 (m, 1H, CH₂—N); 2.63 (s, 3H, CH₃); 2.77 (s, 3H, CH₃—N); 3.28-3.30 (m, 2H, CH₂); 3.92-3.99 (m, 2H, CH₂—O); 7.33 (dd, J 2.9, 0.8 Hz, 1H, Ar); 7.38 (d, J 2.9 Hz, 1H, Ar); 7.74 (dd, J 5.4, 0.6 Hz, 1H, Ar); 8.22 (d, J 5.4 Hz, 1H, Ar); 8.90 (d, J 0.8 Hz, 1H, Ar); 9.37 (bs, 1H, Ar); 11.68 (s, 1H, NH). M/Z (M+H)⁺=435.9. MP>250° C.

### Compound 131 (8-Methyl-6-(1-piperidylmethyl)-2-thieno[2,3-c]pyridin-5-yl-3H-quinazolin-4-one hydrochloride)

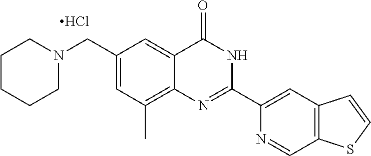 [SMILES: Cc1cc(CN2CCCCC2)cc2c(=O)[nH]c(-c3cc4ccsc4cn3)nc12 ; Cl]

Compound 131 was prepared according to procedure of example 37 using potassium (piperidin-1-yl)methyl trifluoroborate in step 1 instead of morpholinium-4-yl-methyl)trifluoroborate internal salt to afford the product as a yellow solid.

¹H-NMR (400 MHz, DMSO): 1.29-1.43 (m, 1H, CH₂); 1.64-1.86 (m, 5H, CH₂); 2.72 (s, 3H, CH₃); 2.83-2.95 (m, 2H, CH₂—N); 3.34 (d, J 11.9 Hz, 2H, CH₂—N); 4.39 (d, J 5.2 Hz, 2H, CH₂—N); 7.83 (d, J 5.2 Hz, 1H, Ar); 7.99 (d, J 1.0 Hz, 1H, Ar); 8.22 (d, J 1.8 Hz, 1H, Ar); 8.31 (d, J 5.2 Hz, 1H, Ar); 9.04 (d, J 1.0 Hz, 1H, Ar); 9.48 (s, 1H, Ar); 10.36 (bs, 1H, HCl salt); 11.99 (bs, 1H, NH). M/Z (M+H)⁺=391.9. MP=233-240° C.

### Compound 132 (8-Methyl-6-[(4-methylpiperazin-1-yl)methyl]-2-thieno[2,3-c]pyridin-5-yl-3H-quinazolin-4-one dihydrochloride)

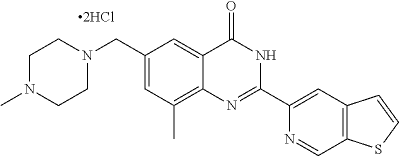 [SMILES: Cc1cc(CN2CCN(C)CC2)cc2c(=O)[nH]c(-c3cc4ccsc4cn3)nc12]

Compound 132 was prepared according to procedure of example 37, using potassium 1-methyl-4-trifluoroboratomethylpiperazine in step 1 instead of morpholinium-4-yl-methyl)trifluoroborate internal salt, to afford the product as a yellow solid.

¹H-NMR (DMSO-D₂O, 400 MHz): 2.72 (s, 3H, CH₃); 2.84 (s, 3H, CH₃—N); 3.13-3.34 (m, 4H, CH₂—N); 3.49-3.56 (m, 2H, CH₂—N); 3.57-3.68 (m, 2H, CH₂—N); 4.41 (bs, 2H, CH₂—N); 7.82 (dd, J 5.3, 0.5 Hz, 1H, Ar); 7.94 (bs, 1H, Ar); 8.22 (bs, 1H, Ar); 8.30 (d, J 5.3 Hz, 1H, Ar); 9.04 (d, J 0.9 Hz, 1H, Ar); 9.47 (bs, 1H, Ar). M/Z (M+H)⁺=406.9. MP>250° C.

### Compound 133 (8-Methyl-6-(pyrrolidin-1-ylmethyl)-2-thieno[2,3-c]pyridin-5-yl-3H-quinazolin-4-one hydrochloride)

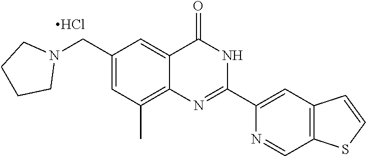 [SMILES: Cc1cc(CN2CCCC2)cc2c(=O)[nH]c(-c3cc4ccsc4cn3)nc12 ; Cl]

Compound 133 was prepared according to procedure of example 37, using potassium trifluoro[(pyrrolidin-1-yl)methyl]borate in step 1 instead of morpholinium-4-yl-methyl)trifluoroborate internal salt, to afford the product as a yellow solid.

¹H-NMR (400 MHz, DMSO): 1.82-1.96 (m, 2H, CH₂); 1.98-2.11 (m, 2H, CH₂); 2.73 (s, 3H, CH₃); 3.04-3.17 (m, 2H, CH₂—N); 3.33-3.44 (m, 2H, CH₂—N); 4.48 (d, J 5.9 Hz, 2H, CH₂—N); 7.83 (dd, J 5.4, 0.5 Hz, 1H, Ar); 7.99 (d, J 1.1 Hz, 1H, Ar); 8.24 (d, J 1.8 Hz, 1H, Ar); 8.31 (d, J 5.7 Hz, 1H, Ar); 9.04 (d, J 1.0 Hz, 1H, Ar); 9.48 (s, 1H, Ar); 10.66 (bs, 1H, HCl salt); 11.98 (bs, 1H, NH). M/Z (M+H)⁺=377.9. MP>250° C.

### Compound 134 (8-Methyl-6-(2-morpholino-2-oxo-ethyl)-2-pyrrolo[1,2-c]pyrimidin-3-yl-3H-quinazolin-4-one)

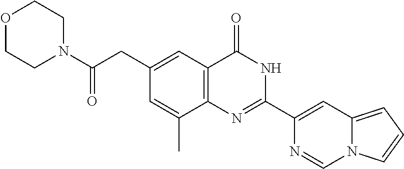 [SMILES: Cc1cc(CC(=O)N2CCOCC2)cc2c(=O)[nH]c(-c3cc4cccn4cn3)nc12]

Compound 134 was prepared according to procedure of example 39, starting from pyrrolo[1,2-c]pyrimidine-3-carboxylic acid in step 8 to afford the product as a white solid.

¹H-NMR (400 MHz, DMSO): 2.64 (s, 3H, CH₃); 3.46-3.51 (m, 2H, CH₂); 3.52-3.58 (m, 6H, CH₂); 3.85 (s, 2H, CH₂—CO); 6.90 (d, J 3.6 Hz, 1H, Ar); 7.08 (dd, J 3.6, 2.9 Hz, 1H, Ar); 7.55 (bs, 1H, Ar); 7.85 (d, J 1.0 Hz, 1H, Ar); 7.91 (d, J 2.3 Hz, 1H, Ar); 8.58 (s, 1H, Ar); 9.34 (s, 1H, Ar); 11.44 (bs, 1H, NH). M/Z (M+H)⁺=404.9. MP=254-256° C.

### Example 40—Synthesis of compound 135 (8-Methyl-6-(morpholine-4-carbonyl)-2-pyrrolo[1,2-c]pyrimidin-3-yl-3H-quinazolin-4-one)

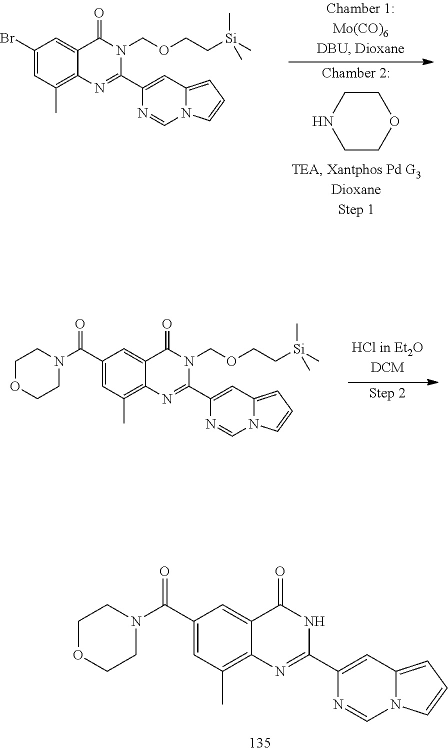 [SMILES: Cc1cc(Br)cc2c(=O)n(COCC[Si](C)(C)C)c(-c3cc4cccn4cn3)nc12 ; C1COCCN1 ; Cc1cc(C(=O)N2CCOCC2)cc2c(=O)n(COCC[Si](C)(C)C)c(-c3cc4cccn4cn3)nc12 ; Cc1cc(C(=O)N2CCOCC2)cc2c(=O)[nH]c(-c3cc4cccn4cn3)nc12]

Step 1:

Under inert atmosphere in a 2-chamber glassware system, a suspension of molybdenum hexacarbonyl (70 mg, 0.26 mmol) in dioxane (1.7 mL) was placed in the first chamber. In the second chamber, to a degassed solution of 6-bromo-8-methyl-2-(pyrrolo[1,2-c]pyrimidin-3-yl)-3-((2-(trimethylsilyl)ethoxy)methyl)quinazolin-4(3H)-one (250 mg, 0.51 mmol) in dioxane (1.7 mL) were added morpholine (89 μL, 1.02 mmol), triethylamine (142 μL, 1.02 mmol) and XantPhos Pd G3 (10 mg, 0.01 mmol). Finally, 1,8-iazabicyclo[5.4.0]undec-7-ene (114 μL, 0.77 mmol) was added to the first chamber and both chambers were stirred for 16 h at 85° C. The mixture from the second chamber was subsequently diluted with an aqueous saturated solution of sodium bicarbonate and extracted with dichloromethane. The combined organic extracts were dried over MgSO₄ and concentrated in vacuo. The crude residue was purified by flash column chromatography on silica gel using dichloromethane/methanol as eluent to afford crude 8-methyl-6-(morpholine-4-carbonyl)-2-(pyrrolo[1,2-c]pyrimidin-3-yl)-3-((2-(trimethylsilyl)ethoxy)methyl)quinazolin-4(3H)-one (181 mg) as a green oil.

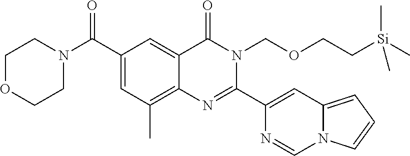 [SMILES: Cc1cc(C(=O)N2CCOCC2)cc2c(=O)n(COCC[Si](C)(C)C)c(-c3cc4cccn4cn3)nc12]

M/Z (M+H)⁺=520.9.

Step 2:

8-methyl-6-(morpholine-4-carbonyl)-2-pyrrolo[1,2-c]pyrimidin-3-yl-3H-quinazolin-4-one was prepared from 8-methyl-6-(morpholine-4-carbonyl)-2-(pyrrolo[1,2-c]pyrimidin-3-yl)-3-((2-(trimethylsilyl)ethoxy)methyl)quinazolin-4(3H)-one (0.26 mmol) according to procedure of example 27, step 3. Purification by flash column chromatography on silica gel using dichloromethane/methanol as eluent, followed by trituration in diethyl ether afforded compound 135 (24 mg, 12% over 2 steps) as a yellow solid.

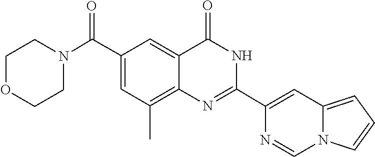 [SMILES: Cc1cc(C(=O)N2CCOCC2)cc2c(=O)[nH]c(-c3cc4cccn4cn3)nc12]

¹H-NMR (400 MHz, DMSO): 2.68 (s, 3H, CH₃); 3.54-3.68 (m, 8H, CH₂); 6.93-6.94 (m, 1H, Ar); 7.1 (dd, J 3.8, 2.8 Hz, 1H, Ar); 7.73-7.74 (m, 1H, Ar); 7.92-7.93 (m, 1H, Ar); 7.97 (dd, J 2.0, 0.6 Hz, 1H, Ar); 8.63 (d, J 1.1 Hz, 1H, Ar); 9.36 (t, J 1.1 Hz, 1H, Ar); 11.66 (bs, 1H, NH). M/Z (M+H)⁺=390.9. MP=150-170° C.

### Compound 136 (8-Methyl-6-(1-methyl-6-oxo-piperidin-3-yloxy)-2-(4-trifluoromethyl-pyridin-2-yl)-3H-quinazolin-4-one)

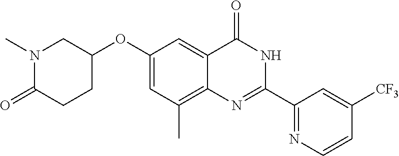 [SMILES: Cc1cc(OC2CCC(=O)N(C)C2)cc2c(=O)[nH]c(-c3cc(C(F)(F)F)ccn3)nc12]

Compound 136 was prepared according to procedure of example 27 starting from 6-bromo-8-methyl-2-(4-trifluoromethyl-pyridin-2-yl)-3-(2-trimethylsilanyl-ethoxymethyl)-3H-quinazolin-4-one in step 1 and using diisopropyl azodicarboxylate instead of diethyl azodicarboxylate and starting from 5-hydroxy-1-methyl-piperidin-2-one in step 2 to afford the product as a white solid.

¹H-NMR (400 MHz, DMSO): 2.06-2.12 (m, 2H, CH₂); 2.25-2.41 (m, 2H, CH₂); 2.65 (s, 3H, CH₃); 2.82 (s, 3H, CH₃—N); 3.41-3.46 (m, 1H, CH₂—N); 3.69 (dd, J 13.2, 3.9 Hz, 1H, CH₂—N); 5.04-5.08 (m, 1H, CH-0); 7.45 (d, J 2.8 Hz, 1H, Ar); 7.52 (d, J 2.8 Hz, 1H, Ar); 8.00-8.03 (m, 1H, Ar); 8.64 (s, 1H, Ar); 9.02 (d, J 5.1 Hz, 1H, Ar); 12.07 (s, 1H, NH). M/Z (M+H)⁺=433.9. MP>250° C.

### Compound 137 (8-Methyl-2-thieno[2,3-c]pyridin-5-yl-6-(thiomorpholinomethyl)-3H-quinazolin-4-one hydrochloride)

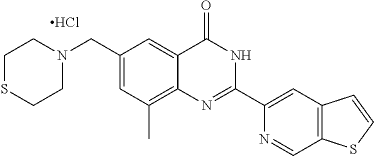 [SMILES: Cc1cc(CN2CCSCC2)cc2c(=O)[nH]c(-c3cc4ccsc4cn3)nc12 ; Cl]

Compound 137 was prepared according to procedure of example 37, using potassium 4-trifluoroboratomethylthiomorpholine in step 1 instead of morpholinium-4-yl-methyl)trifluoroborate internal salt, to afford the product as a yellow solid.

¹H-NMR (400 MHz, DMSO): 2.73 (s, 3H, CH₃); 2.79-2.88 (m, 2H, CH₂); 3.08-3.21 (m, 4H, CH₂); 3.61-3.69 (m, 2H, CH₂); 4.48 (d, J 2.4 Hz, 2H, CH₂—N); 7.83 (dd, J 5.4, 0.4 Hz, 1H, Ar); 7.98 (d, J 1.0 Hz, 1H, Ar); 8.24 (d, J 1.6 Hz, 1H, Ar); 8.31 (d, J 5.4 Hz, 1H, Ar); 9.04 (d, J 1.0 Hz, 1H, Ar); 9.48 (s, 1H, Ar); 10.71 (bs, 1H, HCl salt); 12.00 (bs, 1H, NH). M/Z (M+H)⁺=409.9. MP>250° C.

### Compound 138 (8-Methyl-6-[2-(1,4-oxazepan-4-yl)-2-oxo-ethyl]-2-thieno[2,3-c]pyridin-5-yl-3H-quinazolin-4-one)

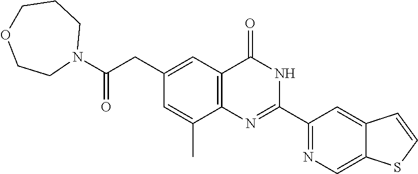 [SMILES: Cc1cc(CC(=O)N2CCCOCC2)cc2c(=O)[nH]c(-c3cc4ccsc4cn3)nc12]

Compound 138 was prepared according to procedure of example 39 using homomorpholine instead of morpholine in step 7. The product was isolated by extraction of the reaction mixture of step 8 with ethyl acetate followed by trituration in diethyl ether to afford a white solid.

¹H-NMR (400 MHz, DMSO): 1.72-1.85 (m, 2H, CH₂); 2.69 (s, 3H, CH₃); 3.56-3.68 (m, 8H, CH₂-0 & CH₂—N); 3.88 (d, J 11.2 Hz, 2H, CH₂—CO); 7.62 (bt, J 2.3 Hz, 1H, Ar); 7.82 (dd, J 5.4, 0.4 Hz, 1H, Ar); 7.92 (bt, J 2.3 Hz, 1H, Ar); 8.29 (d, J 5.4 Hz, 1H, Ar); 9.01 (d, J 0.4 Hz, 1H, Ar); 9.45 (s, 1H, Ar); 11.76 (bs, 1H, NH). M/Z (M+H)⁺=435.9. MP=215-220° C.

### Compound 139 (8-Methyl-6-(pyrrolidin-1-ylmethyl)-2-thieno[3,2-c]pyridin-6-yl-3H-quinazolin-4-one hydrochloride)

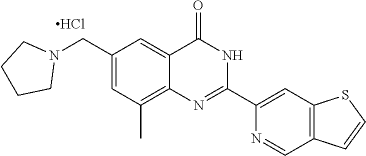 [SMILES: Cc1cc(CN2CCCC2)cc2c(=O)[nH]c(-c3cc4sccc4cn3)nc12 ; Cl]

Compound 139 was prepared according to procedure of example 37, starting from 6-bromo-8-methyl-2-(thieno[3,2-c]pyridin-6-yl)-3-((2-(trimethylsilyl)ethoxy)methyl)quinazolin-4(3H)-one and using potassium trifluoro[(pyrrolidin-1-yl)methyl]borate in step 1 instead of morpholinium-4-yl-methyl)trifluoroborate internal salt to afford the product as a yellow solid.

¹H-NMR (400 MHz, DMSO): 1.82-1.96 (m, 2H, CH₂); 1.98-2.11 (m, 2H, CH₂); 2.73 (s, 3H, CH₃); 3.04-3.17 (m, 2H, CH₂—N); 3.33-3.44 (m, 2H, CH₂—N); 4.47 (d, J 5.8 Hz, 2H, CH₂—N); 7.78 (dd, J 5.4, 0.6 Hz, 1H, Ar); 7.98 (d, J 1.0 Hz, 1H, Ar); 8.15 (d, J 5.4 Hz, 1H, Ar); 8.23 (d, J 1.6 Hz, 1H, Ar); 9.28 (bs, 1H, Ar); 9.33 (d, J 1.0 Hz, 1H, Ar); 10.66 (bs, 1H, HCl salt); 11.97 (bs, 1H, NH). M/Z (M+H)⁺=378.0. MP>250° C.

### Example 41—Synthesis of compound 140-R (8-Methyl-6-[(3R)-1-methyl-5-oxo-pyrrolidin-3-yl]oxy-2-thieno[3,2-c]pyridin-6-yl-3H-quinazolin-4-one)

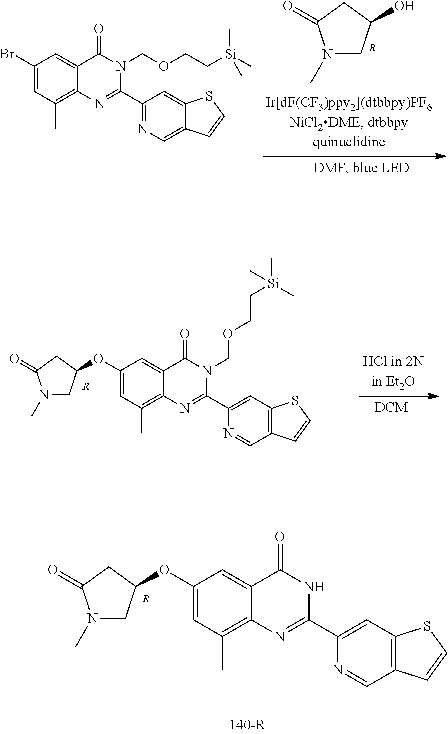

Step 1:

Under inert atmosphere, to a suspension of 6-bromo-8-methyl-2-(thieno[3,2-c]pyridin-6-yl)-3-((2-(trimethylsilyl)ethoxy)methyl)quinazolin-4(3H)-one (251 mg, 0.50 mmol) in dimethylformamide (2 mL) were added quinuclidine (60 mg, 0.60 mmol), (Ir[dF(CF₃)ppy]₂(dtbpy))P F₆ (11 mg, 0.01 mmol) and a solution of dichloro(dimethoxyethane)nickel (11 mg, 0.05 mmol) and 4,4′-di-tert-butyl-2,2′-dipyridyl (13 mg, 0.05 mmol) in dimethylformamide (2 mL) followed by (4R)-4-hydroxy-1-methylpyrrolidin-2-one (173 mg, 1.5 mmol) in solution in dimethylformamide (2 mL). After sonication and degassing with fast argon bubbling, the mixture was irradiated with blue LED light for 60 h in an EvoluChem™ PhotoRedOx Box under fan cooling before being diluted with water (20 mL) and extracted with dichloromethane (3×20 mL). The combined organic extracts were dried over hydrophobic filter and concentrated in vacuo. The crude residue was purified by flash column chromatography on silica gel using dichloromethane/methanol as eluent to afford (R)-8-methyl-6-((1-methyl-5-oxopyrrolidin-3-yl)oxy)-2-(thieno[3,2-c]pyridin-6-yl)-3-((2-(trimethylsilyl)ethoxy)methyl)quinazolin-4(3H)-one (107 mg, 40%) as yellow solid.

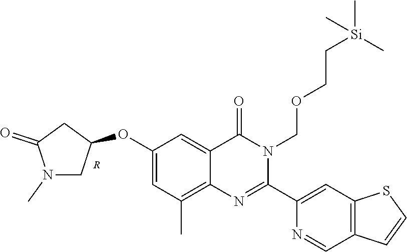

M/Z (M+H)^{T}=537.9.

Step 2:

8-methyl-6-[(3R)-1-methyl-5-oxo-pyrrolidin-3-yl]oxy-2-thieno[3,2-c]pyridin-6-yl-3H-quinazolin-4-one was prepared from (R)-8-methyl-6-((1-methyl-5-oxopyrrolidin-3-yl)oxy)-2-(thieno[3,2-c]pyridin-6-yl)-3-((2-(trimethylsilyl)ethoxy)methyl)quinazolin-4(3H)-one (106 mg, 0.197 mmol) according to procedure of example 27, step 3 to afford compound 140-R as an off-white solid in 84% yield.

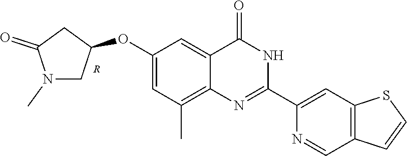

¹H-NMR (400 MHz, DMSO): 2.34 (d, J 17.3 Hz, 1H, CH₂); 2.68 (s, 3H, CH₃); 2.78 (s, 3H, CH₃—N); 2.89 (dd, J 17.3, 6.7 Hz, 1H, CH₂); 3.47 (dd, J 11.4, 0.6 Hz, 1H, CH₂); 3.88 (dd, J 11.5, 5.6 Hz, 1H, CH₂); 5.18-5.24 (m, 1H, CH—O); 7.35-7.40 (m, 2H, Ar); 7.75 (dd, J 0.6, 5.4 Hz, 1H, Ar); 8.10 (d, J 5.4 Hz, 1H, Ar); 9.19 (s, 1H, Ar); 9.29 (s, 1H, Ar); 11.75 (s, 1H, NH). M/Z (M+H)⁺=421.8. MP=190-195° C.

### Compound 140-S (8-Methyl-6-[(3S)-1-methyl-5-oxo-pyrrolidin-3-yl]oxy-2-thieno[3,2-c]pyridin-6-yl-3H-quinazolin-4-one)

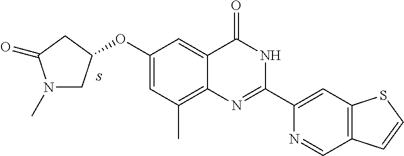

Compound 140-S was prepared according to procedure of example 41, using (45)-4-hydroxy-1-methylpyrrolidin-2-one instead of (4R)-4-hydroxy-1-methylpyrrolidin-2-one in step 1.

¹H-NMR (400 MHz, DMSO): 2.34 (d, J 17.3 Hz, 1H, CH₂); 2.68 (s, 3H, CH₃); 2.78 (s, 3H, CH₃—N); 2.89 (dd, J 17.3, 6.7 Hz, 1H, CH₂); 3.47 (d, J 11.3 Hz, 1H, CH₂); 3.88 (dd, J 11.3, 5.5 Hz, 1H, CH₂); 5.18-5.24 (m, 1H, CH-0); 7.35-7.40 (m, 2H, Ar); 7.75 (d, 5.5 Hz, 1H, Ar); 8.10 (d, J 5.5 Hz, 1H, Ar); 9.19 (s; 1H, Ar); 9.29 (s, 1H, Ar); 11.75 (s, 1H, NH). M/Z (M+H)⁺=421.8. MP=190-195° C.

### Compound 141 (Benzyl (3S)-3-[(8-methyl-4-oxo-2-thieno[3,2-c]pyridin-6-yl-3H-quinazolin-6-yl)oxy]pyrrolidine-1-carboxylate)

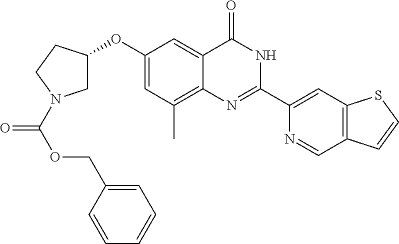 [SMILES: Cc1cc(O[C@H]2CCN(C(=O)OCc3ccccc3)C2)cc2c(=O)[nH]c(-c3cc4sccc4cn3)nc12]

Compound 141 was prepared according to procedure of example 27 step 1 and 2, starting from 6-bromo-8-methyl-2-(thieno[3,2-c]pyridin-6-yl)-3-((2-(trimethylsilyl)ethoxy)methyl)quinazolin-4(3H)-one in step 1, and from (R)-(−)-1-Cbz-3-pyrrolidinol in step 2, followed by procedure of example 30, step 2. Purification by flash column chromatography on silica gel using dichloromethane/methanol as eluent, followed by trituration in methanol and diethyl ether afforded the product as a white solid.

¹H-NMR (400 MHz, DMSO): 2.07-2.30 (m, 2H, CH₂); 2.68 (s, 3H, CH₃); 3.4-3.50 (m, 1H, CH₂); 3.51-3.62 (m, 2H, CH₂); 3.64-3.77 (m, 1H, CH₂); 5.05-5.12 (m, 2H, CH₂—N); 5.18-5.25 (m, 1H, CH—O); 7.27-7.41 (m, 6H, Ar); 7.43 (bs, 1H, Ar); 7.76 (dd, J 5.4, 0.6 Hz, 1H, Ar); 8.10 (d, J 5.4 Hz, 1H, Ar); 9.20 (s, 1H, Ar); 9.29 (s, 1H, Ar); 11.75 (bs, 1H, NH). M/Z (M+H)⁺=513.8. MP=224-227° C.

### Compound 142 (8-Methyl-6-[2-(4-methyl-3-oxo-piperazin-1-yl)ethyl]-2-thieno[2,3-c]pyridin-5-yl-3H-quinazolin-4-one hydrochloride)

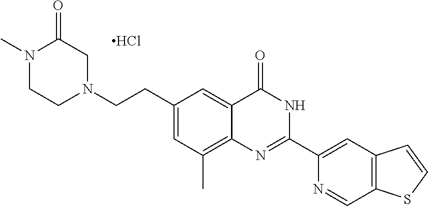 [SMILES: Cc1cc(CCN2CCN(C)C(=O)C2)cc2c(=O)[nH]c(-c3cc4ccsc4cn3)nc12 ; Cl]

Compound 142 was prepared according to procedure of example 38 using 1-methylpiperazin-2-one instead of homomorpholine in step 6. The free base was isolated by extraction of the reaction mixture of step 8 with ethyl acetate. The hydrochloride salt was obtained by filtration after addition of an excess of HCl (2N in Et₂O) to a solution of the free base in dichloromethane, and then freeze-drying of a suspension of the resulting solid in water and an excess of aqueous 1N HCl to afford the product as a yellow solid.

¹H-NMR (400 MHz, DMSO): 2.71 (s, 3H, CH₃); 2.91 (s, 3H, CH₃—N); 3.18-3.27 (m, 2H, CH₂); 3.35-3.59 (m, 4H, CH₂—N); 3.68-3.85 (m, 2H, CH₂—N); 3.86-4.04 (m, 2H, CH₂—N); 7.67 (d, J 1.6 Hz, 1H, Ar); 7.82 (d, J 5.4 Hz, 1H, Ar); 7.97 (d, J 1.6 Hz, 1H, Ar); 8.31 (d, J 5.4 Hz, 1H, Ar); 9.01 (d, J 0.8 Hz, 1H, Ar); 9.47 (bs, 1H, Ar); 11.77 (bs, 2H, NH+HCl salt). M/Z (M+H)⁺=434.8. MP>250° C.

### Compound 143 (8-Methyl-6-[2-(4-methyl-3-oxo-piperazin-1-yl)ethyl]-2-thieno[3,2-c]pyridin-6-yl-3H-quinazolin-4-one hydrochloride)

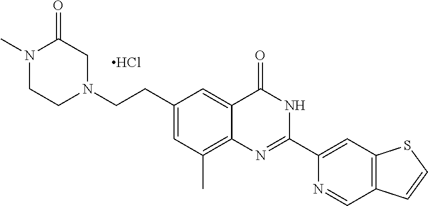 [SMILES: Cc1cc(CCN2CCN(C)C(=O)C2)cc2c(=O)[nH]c(-c3cc4sccc4cn3)nc12 ; Cl]

Compound 143 was prepared according to procedure of example 38 using 1-methylpiperazin-2-one instead of homomorpholine in step 6 and starting from thieno[3,2-c]pyridine-6-carboxylic acid in step 8. The HCl salt was obtained by freeze-drying of a suspension of the free base in water and an excess of aqueous 1N HCl to afford the product as a yellow solid.

¹H-NMR (400 MHz, DMSO): 2.71 (s, 3H, CH₃); 2.91 (s, 3H, CH₃—N); 3.16-3.25 (m, 2H, CH₂); 3.36-3.55 (m, 4H, CH₂—N); 3.62-3.74 (m, 2H, CH₂—N); 3.88-4.02 (m, 2H, CH₂—N); 7.68 (d, J 1.6 Hz, 1H, Ar); 7.78 (dd, J 5.4, 0.8 Hz, 1H, Ar); 7.99 (d, J 1.6 Hz, 1H, Ar); 8.13 (d, J 5.4 Hz, 1H, Ar); 9.25 (bt, J 0.8 Hz, 1H, Ar); 9.32 (d, J 0.8 Hz, 1H, Ar); 11.26 (bs, 1H, HCl salt); 11.84 (bs, 1H, NH). M/Z (M+H)⁺=434.8. MP>250° C.

### Compound 144 (8-Methyl-6-[2-(4-methyl-3-oxo-piperazin-1-yl)ethyl]-2-[4-(trifluoromethyl)-2-pyridyl]-3H-quinazolin-4-one hydrochloride)

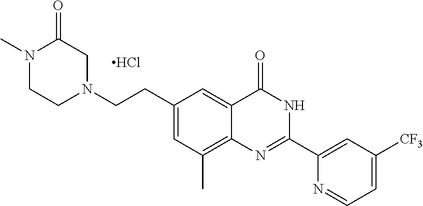 [SMILES: Cc1cc(CCN2CCN(C)C(=O)C2)cc2c(=O)[nH]c(-c3cc(C(F)(F)F)ccn3)nc12 ; Cl]

Compound 144 was prepared according to procedure of example 38 using 1-methylpiperazin-2-one instead of homomorpholine in step 6 and 4-(trifluoromethyl)picolinic acid in step 8. The free base was isolated by extraction of the reaction mixture of step 8 with ethyl acetate. The hydrochloride salt was obtained by filtration after addition of an excess of HCl (2N in Et₂O) to a solution of the free base in dichloromethane, and then freeze-drying of a suspension of the resulting solid in water and an excess of aqueous 1N HCl to afford the product as a yellow solid.

¹H-NMR (400 MHz, DMSO): 2.68 (s, 3H, CH₃); 2.94 (s, 3H, CH₃—N); 3.18-3.26 (m, 2H, CH₂); 3.35-3.59 (m, 4H, CH₂—N); 3.68-3.83 (m, 2H, CH₂—N); 3.84-4.07 (m, 2H, CH₂—N); 7.70 (d, J 1.6 Hz, 1H, Ar); 8.00 (d, J 1.6 Hz, 1H, Ar); 8.06 (dd, J 5.1, 1.2 Hz, 1H, Ar); 8.67 (bs, 1H, Ar); 9.05 (d, J 5.1 Hz, 1H, Ar); 11.36 (bs, 1H, HCl salt); 12.17 (bs, 1H, NH). M/Z (M+H)⁺=446.8. MP=148-150° C.

### Compound 145 (8-Methyl-6-[2-(2-oxa-6-azaspiro[3.3]heptan-6-yl)ethyl]-2-thieno[2,3-c]pyridin-5-yl-3H-quinazolin-4-one)

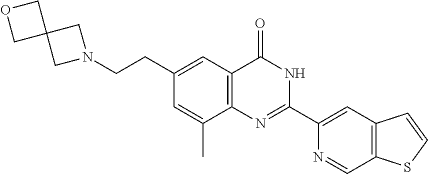 [SMILES: Cc1cc(CCN2CC3(COC3)C2)cc2c(=O)[nH]c(-c3cc4ccsc4cn3)nc12]

Compound 145 was prepared according to procedure of example 38 using 2-oxa-6-aza-spiro[3.3]heptane instead of homomorpholine, and triethylamine instead of potassium carbonate in step 6, and without salt formation in step 8. Purification by flash column chromatography on silica gel using dichloromethane/methanol as eluent, followed by trituration diethyl ether afforded the product as a yellow solid.

¹H-NMR (400 MHz, DMSO): 2.67-2.69 (m, 7H, CH₃+CH₂+CH₂—N); 3.38 (bs, 4H, 2 CH₂—N); 4.59 (s, 4H, 2 CH₂—O); 7.57 (bs, 1H, Ar); 7.80-7.83 (m, 2H, Ar); 8.27 (d, J 5.4 Hz, 1H, Ar); 8.98 (s, 1H, Ar); 9.44 (s, 1H, Ar); 11.71 (bs, 1H, NH), M/Z (M+H)⁺=419.8. MP=220-225° C.

### Compound 146 (8-Methyl-6-[2-(2-oxa-6-azaspiro[3.3]heptan-6-yl)ethyl]-2-thieno[3,2-c]pyridin-6-yl-3H-quinazolin-4-one)

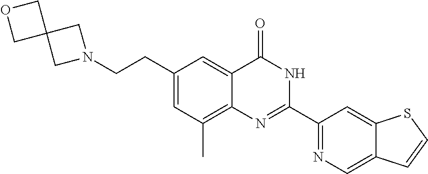 [SMILES: Cc1cc(CCN2CC3(COC3)C2)cc2c(=O)[nH]c(-c3cc4sccc4cn3)nc12]

Compound 146 was prepared according to procedure of example 38 step 1 to 7 using 2-oxa-6-aza-spiro[3.3]heptane instead of homomorpholine, and triethylamine instead of potassium carbonate in step 6, followed by procedure of example 1 step 4 and starting from thieno[3,2-c]pyridine-6-carboxylic acid. Purification by flash column chromatography on silica gel using dichloromethane/methanol as eluent, followed by trituration diethyl ether afforded the product as a white solid.

¹H-NMR (400 MHz, DMSO): 2.68-2.76 (m, 5H, CH₃+CH₂); 2.95 (bs, 2H, CH₂—N); 3.68 (bs, 4H, 2 CH₂—N); 4.61 (s, 4H, CH₂—O); 7.60 (bs, 1H, Ar); 7.76 (dd, J 5.4, 0.7 Hz, 1H, Ar); 7.87 (bs, 1H, Ar); 8.11 (d, J 5.4 Hz, 1H, Ar); 9.23 (t, J 0.9 Hz, 1H, Ar); 9.30 (d, J 0.9 Hz, 1H, Ar); 11.76 (bs, 1H, NH). M/Z (M+H)⁺=419.8. MP=160-200° C.

### Compound 147 (8-Methyl-6-[(4-methyl-3-oxo-piperazin-1-yl)methyl]-2-thieno[3,2-c]pyridin-6-yl-3H-quinazolin-4-one hydrochloride)

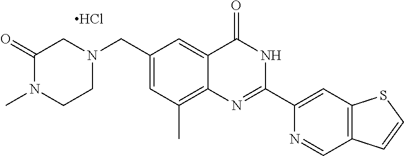 [SMILES: Cc1cc(CN2CCN(C)C(=O)C2)cc2c(=O)[nH]c(-c3cc4sccc4cn3)nc12 ; Cl]

Compound 147 was prepared according to procedure of example 37, starting from 6-bromo-8-methyl-2-(thieno[3,2-c]pyridin-6-yl)-3-((2-(trimethylsilyl)ethoxy)methyl)quinazolin-4(3H)-one and using trifluoro((4-methyl-3-oxopiperazin-1-ium-1-yl)methyl)borate internal salt in step 1 instead of morpholinium-4-yl-methyl)trifluoroborate internal salt to afford the product as a yellow solid.

¹H-NMR (400 MHz, DMSO): 2.73 (s, 3H, CH₃); 2.87 (s, 3H, CH₃—N); 4.45 (bs, 6H, CH₂—N); 4.23-4.66 (m, 2H, CH₂—N); 7.78 (dd, J 5.4, 0.8 Hz, 1H, Ar); 7.91 (bs, 1H, Ar); 8.15 (d, J 5.4 Hz, 1H, Ar); 8.21 (bs, 1H, Ar); 9.27-9.30 (m, 1H, Ar); 9.33 (d, J 0.8 Hz, 1H, Ar); 11.20 (bs, 1H, HCl salt); 11.97 (bs, 1H, NH). M/Z (M+H)⁺=434.8. MP>250° C.

Trifluoro((4-methyl-3-oxopiperazin-1-ium-1-yl)methyl)borate internal salt was prepared as follows:

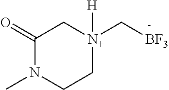

Under inert atmosphere a suspension of potassium chloromethyltrifluoroborate (205 mg, 1.31 mmol) and 1-methylpiperazin-2-one (300 mg, 2.62 mmol) in tetrahydrofuran (3 mL) and tert-butanol (1.5 mL) was heated for 3 h at 80° C. The reaction mixture was evaporated to dryness. The resulting solid residue was taken up and triturated in HPLC grade acetone (15 mL) and then diethyl ether (1 mL) was slowly added to fully precipitate the product which was collected by filtration and dried in vacuo to afford the product (205 mg, 55 wt % purity) as a white solid in a mixture with potassium chloride.

¹H-NMR (400 MHz, DMSO): 2.05 (q, J 5.0 Hz, 2H, CH₂—B); 2.84 (s, 3H, CH₃—N); 2.43-2.56 (m, 2H, CH₂—N); 2.63-2.74 (m, 2H, CH₂—N); 2.28-2.36 (m, 2H, CH₂—N); 9.23 (bs, 1H, internal salt).

### Compound 148 (6-(2-((2-Methoxyethyl)(methyl)amino)ethyl)-8-methyl-2-(thieno[3,2-c]pyridin-6-yl)quinazolin-4(3H)-one hydrochloride)

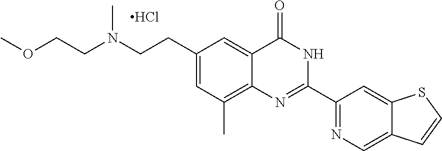 [SMILES: COCCN(C)CCc1cc(C)c2nc(-c3cc4sccc4cn3)[nH]c(=O)c2c1 ; Cl]

Compound 148 was prepared according to procedure of example 38 using 2-methoxy-N-methylethan-1-amine instead of homomorpholine and triethylamine instead of potassium carbonate in step 6 and starting from thieno[3,2-c]pyridine-6-carboxylic acid in step 8. The HCl salt was obtained by freeze-drying of a suspension of the free base in water, acetonitrile and an excess of aqueous 1N HCl to afford the product as a yellow solid.

¹H-NMR (400 MHz, DMSO): 2.71 (s, 3H, CH₃); 2.88 (d, J 5.0 Hz, 3H, CH₃—N); 3.12-3.17 (m, 2H, CH₂—N); 3.26-3.39 (m, 4H, 2 CH₂—N); 3.34 (s, 3H, CH₃—O); 3.72 (t, J 5.0 Hz, 2H, CH₂—O); 7.67 (bs, 1H, Ar); 7.78 (d, J 5.5 Hz, 1H, Ar); 7.98 (bs, 1H, Ar); 8.14 (d, J 5.5 Hz, 1H, Ar); 9.25 (s, 1H, Ar); 9.31 (s, 1H, Ar); 9.84 (bs, 1H, HCl); 11.83 (bs, 1H, NH). M/Z (M+H)⁺=409.2. MP=149-162° C.

### Compound 149 (6-(2-(1,1-Dioxidothiomorpholino)ethyl)-8-methyl-2-(thieno[3,2-c]pyridin-6-yl)quinazolin-4(3H)-one)

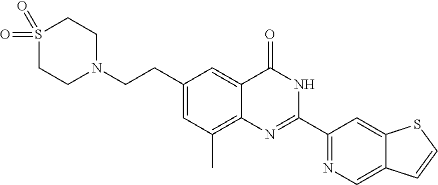 [SMILES: Cc1cc(CCN2CCS(=O)(=O)CC2)cc2c(=O)[nH]c(-c3cc4sccc4cn3)nc12]

Compound 149 was prepared according to procedure of example 38 using thiomorpholine 1,1-dioxide instead of homomorpholine and triethylamine instead of potassium carbonate in step 6 and starting from thieno[3,2-c]pyridine-6-carboxylic acid in step 8. The crude product was isolated by extraction of the reaction mixture of step 8 with ethyl acetate. Filtration after addition of an excess of HCl (2N in Et₂O) to a solution of the free base in dichloromethane, followed by freeze-drying of a suspension of the resulting solid in water and an excess of aqueous 1N HCl failed to form the HCl salt and afforded the free base as a yellow solid.

¹H-NMR (DMSO+D₂O, 400 MHz): 2.68 (s, 3H, CH₃); 2.91 (bs, 4H, CH₂—N+CH₂); 3.16 (bs, 8H, 2 CH₂—N+2 CH₂—SO₂); 7.64 (d, J 1.4 Hz, 1H, Ar); 7.76 (d, J 5.5 Hz, 1H, Ar); 7.88 (d, J 1.4 Hz, 1H, Ar); 8.09 (d, J 5.5 Hz, 1H, Ar); 9.21 (bs, 1H, Ar); 9.28-9.31 (m, 1H, Ar). M/Z (M+H)⁺=455.0. MP=200-212° C.

### Compound 150 (6-[(1,1-Dioxo-1,4-thiazinan-4-yl)methyl]-8-methyl-2-thieno[3,2-c]pyridin-6-yl-3H-quinazolin-4-one)

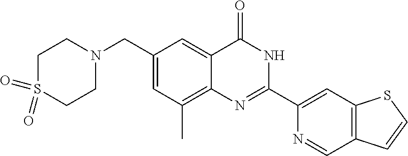 [SMILES: Cc1cc(CN2CCS(=O)(=O)CC2)cc2c(=O)[nH]c(-c3cc4sccc4cn3)nc12]

Compound 150 was prepared according to procedure of example 37, starting from 6-bromo-8-methyl-2-(thieno[3,2-c]pyridin-6-yl)-3-((2-(trimethylsilyl)ethoxy)methyl)quinazolin-4(3H)-one and using ((1,1-dioxidothiomorpholino-4-ium)methyl)trifluoroborate internal salt in step 1 instead of morpholinium-4-yl-methyl)trifluoroborate internal salt. The procedure failed to form the HCl salt and afforded the free base as a yellow solid.

¹H-NMR (400 MHz, DMSO): 2.72 (s, 3H, CH₃); 3.64 (bs, 8H, 2 CH₂—S+2 CH₂—N); 44.61 (m, 2H, CH₂—N); 7.78 (dd, J 5.4, 0.8 Hz, 1H, Ar); 8.01 (bs, 1H, Ar); 8.15 (d, J 5.4 Hz, 1H, Ar); 8.22 (bs, 1H, Ar); 9.28 (bt, J 0.8 Hz, 1H, Ar); 9.33 (d, J 0.8 Hz, 1H, Ar); 11.98 (bs, 1H, NH). M/Z (M+H)⁺=441.0. MP=245-250° C.

((1,1-Dioxidothiomorpholino-4-ium)methyl)trifluoroborate internal salt was prepared using procedure of trifluoro((4-methyl-3-oxopiperazin-1-ium-1-yl)methyl)borate internal salt and starting from thiomorpholine 1,1-dioxide.

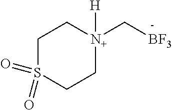

¹H-NMR (400 MHz, DMSO): 2.05 (q, J 5.0 Hz, 2H, CH₂—B); 3.44-3.78 (m, 8H, 2 CH₂—S+2 CH₂—N); 9.36 (bs, 1H, internal salt).

### Compound 151 (6-(((2-Methoxyethyl)(methyl)amino)methyl)-8-methyl-2-(thieno[3,2-c]pyridin-6-yl)quinazolin-4(3H)-one hydrochloride)

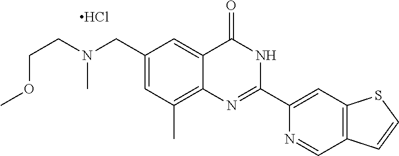 [SMILES: COCCN(C)Cc1cc(C)c2nc(-c3cc4sccc4cn3)[nH]c(=O)c2c1 ; Cl]

Compound 151 was prepared according to procedure of example 37, starting from 6-bromo-8-methyl-2-(thieno[3,2-c]pyridin-6-yl)-3-((2-(trimethylsilyl)ethoxy)methyl)quinazolin-4(3H)-one and using trifluoro(((2-methoxyethyl)(methyl)ammonio)methyl)borate internal salt in step 1 instead of morpholinium-4-yl-methyl)trifluoroborate internal salt to afford the product as a yellow solid.

¹H-NMR (400 MHz, DMSO): 2.74 (m, 6H, 2 CH₃); 3.20-3.29 (m, 1H, CH₂—N); 3.31 (s, 3H, CH₃); 3.32-3.38 (m, 1H, CH₂—N); 3.74 (t, J 4.9 Hz, 2H, CH₂—O); 4.40 (dd, J 13.0, 5.8 Hz, 1H, CH₂—N); 4.52 (dd, J 13.0, 4.9 Hz, 1H, CH₂—N); 7.79 (dd, J 5.4, 0.8 Hz, 1H, Ar); 7.95 (d, J 1.6 Hz, 1H, Ar); 8.15 (d, J 5.4 Hz, 1H, Ar); 8.29 (d, 1H, J 1.6 Hz, Ar); 9.29 (bt, J 0.8 Hz, 1H, Ar); 9.33 (d, J 0.8 Hz, 1H, Ar); 10.36 (bs, 1H, HCl salt); 12.0 (bs, 1H, NH). M/Z (M+H)⁺=395.1. MP=205-215° C.

Trifluoro(((2-methoxyethyl)(methyl)ammonio)methyl)borate internal salt was prepared using procedure of trifluoro((4-methyl-3-oxopiperazin-1-ium-1-yl)methyl)borate internal salt and starting from thiomorpholine 1,1-dioxide. The product was isolated by removing the insolubles by filtration after trituration of the crude in acetone/Et₂O 1:1, followed by evaporation to dryness of the filtrate to afford the product as a brown oil.

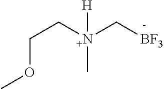

¹H-NMR (400 MHz, DMSO): 1.88-2.10 (m, 2H, CH₂—B); 2.68 (s, 3H, CH₃—N); 3.02-3.12 (m, 1H, CH₂—N); 3.18-3.26 (m, 1H, CH₂—N); 3.28 (s, 3H, CH₃—O); 3.58-3.63 (m, 2H, CH₂—O); 8.34 (bs, 1H, internal salt).

### Compound 152 (6-[(4-Methoxy-1-piperidyl)methyl]-8-methyl-2-thieno[3,2-c]pyridin-6-yl-3H-quinazolin-4-one hydrochloride)

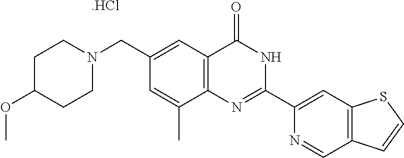 [SMILES: ".HCl" ; COC1CCN(Cc2cc(C)c3nc(-c4cc5sccc5cn4)[nH]c(=O)c3c2)CC1]

Compound 152 was prepared according to procedure of example 37, starting from 6-bromo-8-methyl-2-(thieno[3,2-c]pyridin-6-yl)-3-((2-(trimethylsilyl)ethoxy)methyl)quinazolin-4(3H)-one and using trifluoro((4-methoxypiperidin-1-ium-1-yl)methyl)borate internal salt in step 1 instead of morpholinium-4-yl-methyl)trifluoroborate internal salt to afford the product as a yellow solid.

¹H-NMR (400 MHz, DMSO): 1.60-1.73 (m, 1H, CH₂); 1.87-2.04 (m, 2H, CH₂); 2.14 (d, J 12.0 Hz, 1H, CH₂); 2.72 (s, 3H, CH₃); 2.91-3.11 (m, 2H, CH₂); 3.20 (d, J 12.0 Hz, 1H, CH₂); 3.2-3.28 (m, 3H, CH₃—O); 3.31-3.43 (m, 1H, CH₂); 3.52-3.57 (m, 1H, CH—O); 4.39-4.45 (m, 2H, CH₂—N); 7.79 (dd, J 5.4, 0.5 Hz, 1H, Ar); 8.00 (dd, J 10.6, 1.5 Hz, 1H, Ar); 8.15 (d, J 5.4 Hz, 1H, Ar); 8.21 (dd, J 8.0, 1.5 Hz, 1H, Ar); 9.27-9.30 (m, 1H, Ar); 9.33 (d, J 0.5 Hz, 1H, Ar); 10.56 (bs, 1H, HCl salt); 11.98 (bs, 1H, NH). M/Z (M+H)⁺=420.1. MP=205-215° C.

Trifluoro(((2-methoxyethyl)(methyl)ammonio)methyl)borate internal salt was prepared using procedure of trifluoro(((2-methoxyethyl)(methyl)ammonio)methyl)borate internal salt, starting from 4-methoxypiperidine hydrochloride and adding one equivalent of potassium carbonate in the reaction mixture.

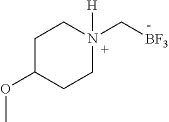

¹H-NMR (400 MHz, DMSO): 1.60-1.74 (m, 2H, CH₂—B); 1.77-1.98 (m, 4H, 2 CH₂); 2.76-2.92 (m, 2H, CH₂—N); 3.00-3.16 (m, 2H, CH₂—N); 3.22 (s, 3H, CH₃—O); 3.37-3.43 (m, 1H, CH—O); no internal salt signal observed.

### Compound 153 (6-[(2,2-Dimethylmorpholin-4-yl)methyl]-8-methyl-2-thieno[3,2-c]pyridin-6-yl-3H-quinazolin-4-one hydrochloride)

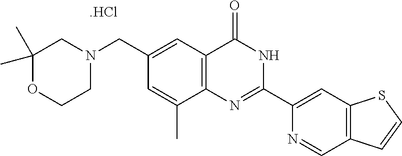 [SMILES: Cc1cc(CN2CCOC(C)(C)C2)cc2c(=O)[nH]c(-c3cc4sccc4cn3)nc12 ; ".HCl"]

Compound 153 was prepared according to procedure of example 37, starting from 6-bromo-8-methyl-2-(thieno[3,2-c]pyridin-6-yl)-3-((2-(trimethylsilyl)ethoxy)methyl)quinazolin-4(3H)-one and using ((2,2-dimethylmorpholino-4-ium)methyl)trifluoroborate internal salt in step 1 instead of morpholinium-4-yl-methyl)trifluoroborate internal salt to afford the product as a yellow solid.

¹H-NMR (400 MHz, DMSO): 1.17 (s, 3H, CH₃); 1.39 (s, 3H, CH₃); 2.73 (s, 3H, CH₃); 2.89 (t, J 11.5 Hz, 1H, CH₂—N); 3.02 (q, J 10.0 Hz, 1H, CH₂—N); 3.16 (d, J 12.0 Hz, 1H, CH₂—N); 3.29 (d, J 12.0 Hz, 1H, CH₂—N); 3.77-3.96 (m, 2H, CH₂—N); 4.35-4.55 (m, 2H, CH₂—N); 7.78 (d, J 5.4 Hz, 1H, Ar); 8.08 (bs, 1H, Ar); 8.15 (d, J 5.4 Hz, 1H, Ar); 8.21 (bs, 1H, Ar); 9.29 (s, 1H, Ar); 9.33 (s, 1H, Ar); 10.63 (bs, 1H, HCl salt); 12.00 (bs, 1H, NH). M/Z (M+H)⁺=421.2 MP=220-240° C.

((2,2-Dimethylmorpholino-4-ium)methyl)trifluoroborate internal was prepared using procedure of trifluoro((4-methyl-3-oxopiperazin-1-ium-1-yl)methyl)borate internal salt and starting from 2,2-dimethylmorpholine.

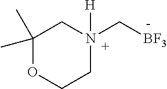

¹H-NMR (400 MHz, DMSO): 1.16 (s, 3H, CH₃); 1.31 (s, 3H, CH₃); 1.90-2.08 (m, 2H, CH₂—B); 2.58-2.73 (m, 1H, CH₂—N); 2.74-2.91 (m, 1H, CH₂—N); 3.05-3.18 (m, 1H, CH₂—N); 2.21-2.30 (m, 1H, CH₂—N); 3.63-3.90 (m, 2H, CH₂—O); 8.40 (bs, 1H, internal salt).

### Compound 154 (8-Chloro-6-(2-morpholinoethyl)-2-(4-(trifluoromethyl)pyridin-2-yl)quinazolin-4(3H)-one hydrochloride)

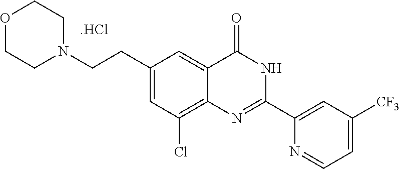 [SMILES: O=c1[nH]c(-c2cc(C(F)(F)F)ccn2)nc2c(Cl)cc(CCN3CCOCC3)cc12 ; ".HCl"]

Compound 154 was prepared from 2-amino-3-chloro-5-(2-hydroxyethyl)benzonitrile according to procedure of example 38 step 5 to 8 using morpholine instead of homomorpholine and triethylamine instead of potassium carbonate in step 6 and starting from 4-(trifluoromethyl)picolinic acid in step 8. The free base was isolated by extraction of the reaction mixture of step 8 with ethyl acetate followed by purification by flash column chromatography on silica gel using dichloromethane/methanol followed by trituration in diethyl ether. The HCl salt was obtained by filtration after addition of an excess of HCl (2N in Et₂O) to a solution of the free base in dichloromethane to afford the product as an off-white solid.

¹H-NMR (400 MHz, DMSO): 3.09-3.17 (m, 2H, CH₂—N); 3.23-3.27 (m, 2H, CH₂—N); 3.42-3.53 (m, 4H, 2 CH₂—N); 3.78 (t, J 11.5 Hz, 2H, CH₂—O); 4.01 (d, J 12.3 Hz, 2H, CH₂—O); 8.04 (bs, 1H, Ar); 8.09 (d, J 4.8 Hz, 1H, Ar); 8.11 (bs, 1H, Ar); 8.64 (s, 1H, Ar); 9.07 (d, J 4.8 Hz, 1H, Ar); 10.75 (bs, 1H, HCl salt); 12.52 (s, 1H, NH). M/Z (M+H)⁺=439.0. MP>250° C.

2-amino-3-chloro-5-(2-hydroxyethyl)benzonitrile was prepared as follows:

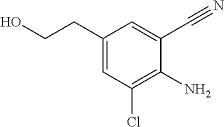 [SMILES: N#Cc1cc(CCO)cc(Cl)c1N]

Under inert atmosphere to a solution of 2-amino-5-(2-hydroxyethyl)benzonitrile (351 mg, 2.16 mmol) in dimethylformamide (11 mL) was added N-Chlorosuccinimide (578 mg, 4.33 mmol). The reaction mixture was stirred for 1 h at 50° C. before being diluted with water and extracted with ethyl acetate. The combined organic extracts were washed with brine, dried over Na₂SO₄ and concentrated in vacuo. The crude residue was purified by flash column chromatography on silica gel using cyclohexane/ethyl acetate as eluent to afford the product (213 mg, 50%) as a yellow solid.

M/Z (M+H)⁺=197.0.

### Compound 155 (8-Methyl-6-(2-oxa-7-azaspiro[3.5]nonan-7-ylmethyl)-2-thieno[3,2-c]pyridin-6-yl-3H-quinazolin-4-one)

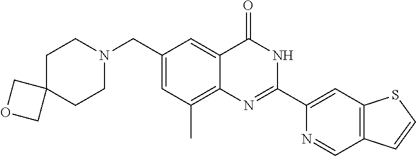 [SMILES: Cc1cc(CN2CCC3(CC2)COC3)cc2c(=O)[nH]c(-c3cc4sccc4cn3)nc12]

Compound 155 was prepared according to procedure of example 37 step 1 starting from 6-bromo-8-methyl-2-(thieno[3,2-c]pyridin-6-yl)-3-((2-(trimethylsilyl)ethoxy)methyl)quinazolin-4(3H)-one and using ((2-oxa-7-azaspiro[3.5]nonan-7-ium-7-yl)methyl)trifluoroborate internal salt instead of morpholinium-4-yl-methyl)trifluoroborate internal salt, followed by procedure of example 30, step 2. Trituration in ethanol and diethyl ether afforded the product as a beige solid.

¹H-NMR (400 MHz, DMSO): 1.70-1.88 (m, 4H, 2 CH₂); 2.18-2.35 (m, 2H, CH₂—N); 2.49-2.57 (m, 2H, CH₂—N); 2.71 (s, 3H, CH₃); 3.45-3.60 (m, 2H, CH₂—N); 4.28 (bs, 4H, 2 CH₂—O); 7.66 (bs, 1H, Ar); 7.77 (d, J 5.4 Hz, 1H, Ar); 7.92 (bs, 1H, Ar); 8.13 (d, J 5.4 Hz, 1H, Ar); 9.25 (s, 1H, Ar); 9.31 (s, 1H, Ar); 11.76 (bs, 1H, NH). M/Z (M+H)⁺=433.2 MP>250° C.

((2-oxa-7-azaspiro[3.5]nonan-7-ium-7-yl)methyl)trifluoroborate internal salt was prepared using procedure of trifluoro((4-methyl-3-oxopiperazin-1-ium-1-yl)methyl)borate internal salt and starting from 2-oxa-7-azaspiro[3.5]nonane.

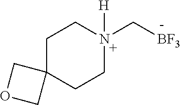

¹H-NMR (400 MHz, DMSO): 1.76-1.88 (m, 2H, CH₂); 1.91 (q, J 5.1 Hz, 2H, CH₂—B); 2.00-2.15 (m, 2H, CH₂); 2.64-2.81 (m, 2H, CH₂—N); 3.17-3.30 (m, 2H, CH₂—N); 4.17-4.30 (m, 2H, CH₂—O); 4.31-4.42 (m, 2H, CH₂—O); 8.28 (bs, 1H, internal salt).

### Compound 156 (N,N-Dimethyl-14(8-methyl-4-oxo-2-(thieno[3,2-c]pyridin-6-yl)-3,4-dihydroquinazolin-6-yl)methyl)piperidine-4-carboxamide hydrochloride)

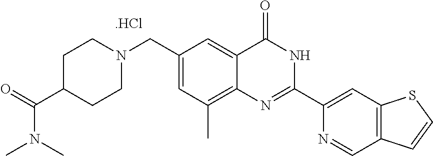 [SMILES: Cc1cc(CN2CCC(C(=O)N(C)C)CC2)cc2c(=O)[nH]c(-c3cc4sccc4cn3)nc12 ; ".HCl"]

Compound 156 was prepared according to procedure of example 37, starting from 6-bromo-8-methyl-2-(thieno[3,2-c]pyridin-6-yl)-3-((2-(trimethylsilyl)ethoxy)methyl)quinazolin-4(3H)-one and using ((4-(dimethylcarbamoyl)piperidin-1-ium-1-yl)methyl)trifluoroborate in step 1 instead of morpholinium-4-yl-methyl)trifluoroborate internal salt to afford the product as a yellow solid.

¹H-NMR (400 MHz, DMSO, 80° C.): 1.80-2.07 (m, 4H, 2 CH₂); 2.76 (s, 3H, CH₃); 2.93 (bs, 6H, 2 CH₃—N); 3.04 (t, J 11.6 Hz, 2H, CH₂—N); 3.22-3.37 (m, 1H, CH); 3.45 (d, J 12.9 Hz, 2H, CH₂—N); 4.40 (s, 2H, CH₂—N); 7.77 (dd, J 5.4, 0.8 Hz, 1H, Ar); 7.97 (bs, 1H, Ar); 8.11 (d, J 5.4 Hz, 1H, Ar); 8.26 (bs, 1H, Ar); 9.24 (t, J 0.8 Hz, 1H, Ar); 9.32 (d, J 0.8 Hz, 1H, Ar); 10.40 (bs, 1H, HCl salt). No NH signal observed. M/Z (M+H)⁺=462.1. MP=215-230° C.

((4-(dimethylcarbamoyl)piperidin-1-ium-1-yl)methyl)trifluoroborate was prepared using procedure of trifluoro((4-methyl-3-oxopiperazin-1-ium-1-yl)methyl)borate internal salt and starting from N,N-dimethylpiperidine-4-carboxamide

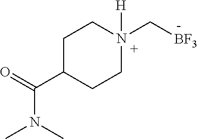

¹H-NMR (400 MHz, DMSO): 1.66-1.85 (m, 5H, CH+CH₂); 1.91 (q, J 5.0 Hz, 2H, CH₂—B); 2.80 (s, 3H, CH₃—N); 3.01 (s, 3H, CH₃—N); 2.81-2.86 (m, 2H, CH₂—N); 3.33-3.40 (m, 2H, CH₂—N); 8.34 (bs, 1H, internal salt).

### Compound 157 (6-((4-(Methoxymethyl)piperidin-1-yl)methyl)-8-methyl-2-(thieno[3,2-c]pyridin-6-yl)quinazolin-4(3H)-one hydrochloride)

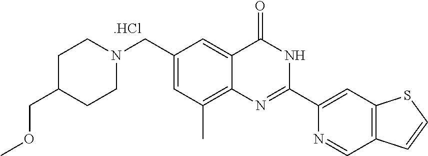 [SMILES: COCC1CCN(Cc2cc(C)c3nc(-c4cc5sccc5cn4)[nH]c(=O)c3c2)CC1 ; ".HCl"]

Compound 157 was prepared according to procedure of example 37, starting from 6-bromo-8-methyl-2-(thieno[3,2-c]pyridin-6-yl)-3-((2-(trimethylsilyl)ethoxy)methyl)quinazolin-4(3H)-one and using trifluoro((4-(methoxymethyl)piperidin-1-ium-1-yl)methyl)borate in step 1 instead of morpholinium-4-yl-methyl)trifluoroborate internal salt to afford the product as a yellow solid.

¹H-NMR (400 MHz, DMSO, 80° C.): 1.53-1.71 (m, 2H, CH₂); 1.75-1.92 (m, 3H, CH+CH₂); 2.76 (s, 3H, CH₃); 2.92-3.04 (m, 2H, CH₂—N); 3.22 (d, J 5.2 Hz, 2H, CH₂—N); 3.26 (s, 3H, CH₃—O); 3.42 (d, J 12.3 Hz, 2H, CH₂—N); 4.39 (s, 2H, CH₂—O); 7.77 (dd, J 5.4, 0.8 Hz, 1H, Ar); 7.99 (bs, 1H, Ar); 8.11 (d, J 5.4 Hz, 1H, Ar); 8.25 (bs, 1H, Ar); 9.24 (t, J 0.8 Hz, 1H, Ar); 9.32 (d, J 0.8 Hz, 1H, Ar); 10.37 (bs, 1H, HCl salt). No NH signal observed. M/Z (M+H)⁺=435.2. MP=200-210° C.

Trifluoro((4-(methoxymethyl)piperidin-1-ium-1-yl)methyl)borate internal was prepared using procedure of trifluoro(((2-methoxyethyl)(methyl)ammonio)methyl)borate internal salt, starting from 4-methoxypiperidine hydrochloride and adding one equivalent of potassium carbonate in the reaction mixture.

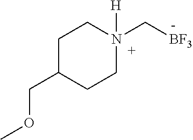

### Example 42—Synthesis of compound 158 (8-Methoxy-6-(2-morpholinoethyl)-2-(4-(trifluoromethyl)pyridin-2-yl)quinazolin-4(3H)-one hydrochloride)

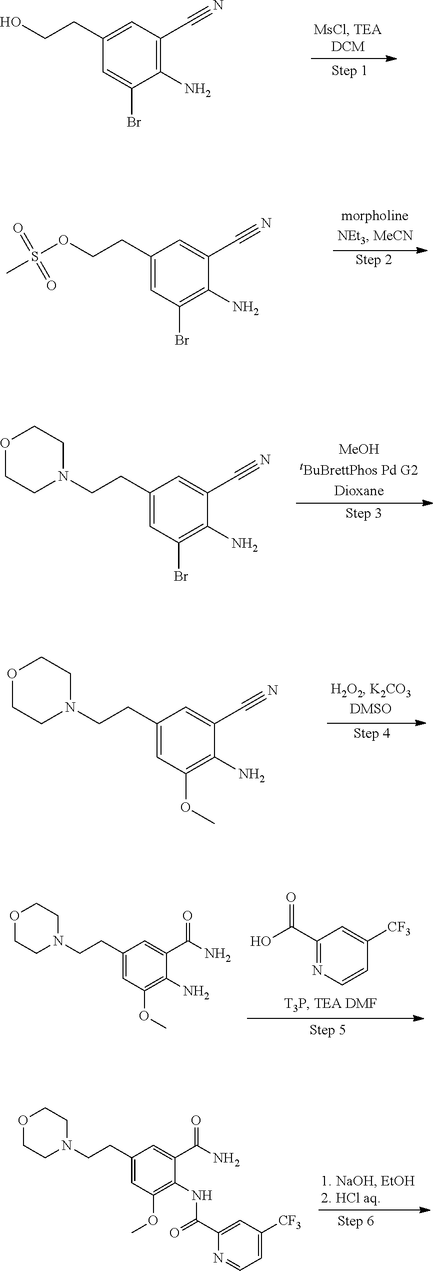

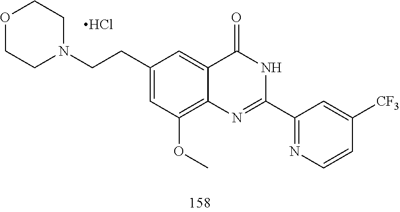 [SMILES: COc1cc(CCN2CCOCC2)cc2c(=O)[nH]c(-c3cc(C(F)(F)F)ccn3)nc12 ; Cl]

Step 1:

4-Amino-3-bromo-5-cyanophenethyl methanesulfonate was prepared from 2-amino-3-bromo-5-(2-hydroxyethyl)benzonitrile according to procedure of example 38, step 5, and isolated as a red oil that was used as such in the next step.

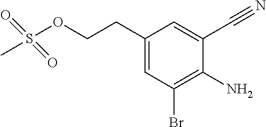 [SMILES: CS(=O)(=O)OCCc1cc(Br)c(N)c(C#N)c1]

M/Z (M[⁷⁹Br]+H)⁺: 318.9

Step 2:

2-amino-3-bromo-5-(2-morpholinoethyl)benzonitrile was prepared from 4-amino-3-bromo-5-cyanophenethyl methanesulfonate according to procedure of example 38, step 6 using morpholine instead of homomorpholine and triethylamine instead of potassium carbonate. The crude residue was purified by flash column chromatography on silica gel using cyclohexane/ethyl acetate to afford the product as a beige solid in 61% yield over 2 steps.

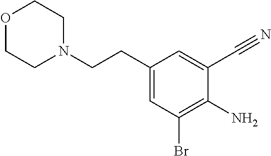 [SMILES: N#Cc1cc(CCN2CCOCC2)cc(Br)c1N]

M/Z (M[⁷⁹Br]+H)⁺: 312.0.

Step 3:

Under inert atmosphere, to a degassed suspension of 2-amino-3-bromo-5-(2-morpholinoethyl)benzonitrile (285 mg, 0.92 mmol), methanol (186 μL, 4.59 mmol) and sodium tert-butylate (177 mg, 0.33 mmol) in dioxane (1 mL) was added ^{t}BuBrettPhos Pd G2 (79 mg, 0.09 mmol). The reaction mixture was stirred at 50° C. for 1 h before being filtered over Celite® and evaporated to dryness. The crude residue was purified by flash column chromatography on silica gel using dichloromethane/methanol to afford 2-amino-3-methoxy-5-(2-morpholinoethyl)benzonitrile (153 mg, 64%) as an orange solid.

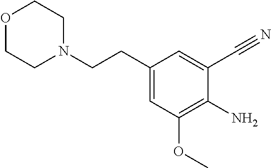 [SMILES: COc1cc(CCN2CCOCC2)cc(C#N)c1N]

M/Z (M+H)⁺=262.1.

Step 4:

2-amino-3-methoxy-5-(2-morpholinoethyl)benzamide was prepared from 2-amino-3-methoxy-5-(2-morpholinoethyl)benzonitrile according to procedure of example 38, step 7 to afford the product as a white solid in 84% yield.

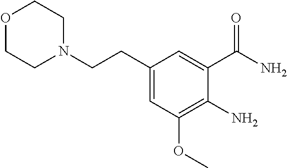 [SMILES: COc1cc(CCN2CCOCC2)cc(C(N)=O)c1N]

¹H-NMR (400 MHz, DMSO): 2.38-2.48 (m, 4H, CH₂+CH₂—N); 2.45-2.49 (m, 2H, CH₂—N); 2.56-2.63 (m, 2H, CH₂—N); 3.58 (t, J 4.4 Hz, 4H, CH₂—O); 3.78 (s, 3H, CH₃—O); 6.07 (s, 2H, NH₂); 6.79 (d, J 1.5 Hz, 1H, Ar); 7.02 (bs, 1H, CO—NH₂); 7.05 (d, J 1.5 Hz, 1H, Ar); 7.65 (bs, 1H, CO—NH₂).

Step 5:

Under inert atmosphere to a solution of 2-amino-3-methoxy-5-(2-morpholinoethyl)benzamide (154 mmol, 0.55 mmol), 4-(trifluoromethyl)picolinic acid (115 mg, 0.61 mmol) and triethylamine (156 μL, 1.10 mmol) in dimethylformamide (4 mL) was added T3P in solution in dimethylformamide (50% w/w, 198 μL, 0.66 mmol). The reaction mixture was stirred at room temperature for 4 h before being concentrated in vacuo. The residue was taken up in an aqueous saturated solution of sodium bicarbonate and extracted several times with dichloromethane and ethyl acetate. The combined organic extracts were concentrated in vacuo to afford crude N-(2-carbamoyl-6-methoxy-4-(2-morpholinoethyl)phenyl)-4-(trifluoromethyl)picolinamide that was used as such in the next step.

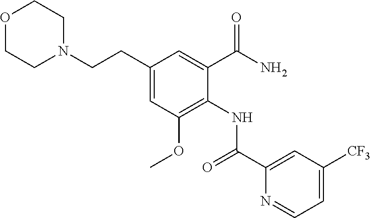 [SMILES: COc1cc(CCN2CCOCC2)cc(C(N)=O)c1NC(=O)c1cc(C(F)(F)F)ccn1]

M/Z (M+H)⁺=453.1

Step 6:

To a solution of crude N-(2-carbamoyl-6-methoxy-4-(2-morpholinoethyl)phenyl)-4-(trifluoromethyl)picolinamide (0.55 mmol) in ethanol (3 mL) was added dropwise aqueous NaOH (1N, 3.30 mL, 3.30 mmol). The reaction mixture was refluxed for 1 h before being extracted 6 times with methanol/dichloromethane 9:1. The combined organic extracts were concentrated in vacuo and the crude residue was purified by preparative HPLC. Pure fractions were freeze-dried with water and an excess of aqueous 1N HCl to afford compound 158 (23 mg, 9% over 2 steps) as a white solid.

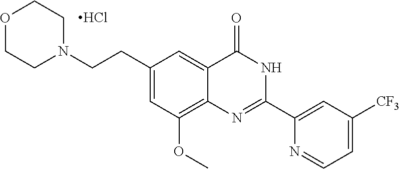 [SMILES: COc1cc(CCN2CCOCC2)cc2c(=O)[nH]c(-c3cc(C(F)(F)F)ccn3)nc12 ; Cl]

¹H-NMR (400 MHz, DMSO): 3.07-3.13 (m, 2H, CH₂); 3.13-3.26 (m, 2H, CH₂—N); 3.45-3.57 (m, 4H, 2 CH₂—N); 3.71-3.83 (t, J 12.4 Hz, 2H, CH₂—O); 3.97-4.05 (m, 5H, CH₂—O, CH₃—O); 7.38 (d, J 0.8 Hz, 1H, Ar); 7.69 (s, 1H, Ar); 8.07 (dd, J 5.0, 0.8 Hz, 1H, Ar); 8.58 (bs, 1H, Ar); 9.04 (d, J 5.0 Hz, 1H, Ar); 10.64 (bs, 1H, HCl salt); 12.21 (bs, 1H, NH). M/Z (M+H)⁺=435.0. MP>250° C.

### Compound 159 (8-Bromo-6-(2-morpholinoethyl)-2-(4-(trifluoromethyl)pyridin-2-yl)quinazolin-4(3H)-one hydrochloride)

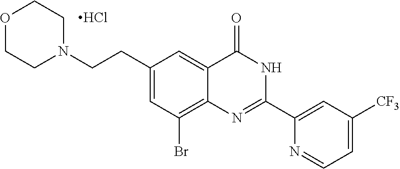 [SMILES: O=c1[nH]c(-c2cc(C(F)(F)F)ccn2)nc2c(Br)cc(CCN3CCOCC3)cc12 ; Cl]

Compound 159 was prepared according to procedure of example 42 steps 1 to 3 followed by steps 5 and 6. The free base was purified by flash column chromatography on silica gel using dichloromethane/methanol followed by trituration in ethanol. The HCl salt was obtained by filtration after addition of an excess of HCl (2N in Et₂O) to a solution of the free base in dichloromethane to afford the product as a beige solid.

¹H-NMR (400 MHz, DMSO): 3.12 (m, 2H, CH₂); 3.23 (m, 2H, CH₂—N); 3.45-3.55 (m, 4H, CH₂—N); 3.73 (t, J 12.4 Hz, 2H, CH₂—O); 3.99-4.01 (m, 2H, CH₂—O); 8.09 (dd, J 5.0, 1.8 Hz, 1H, Ar); 8.15 (bs, 1H, Ar); 8.19-8.21 (m, 1H, Ar); 8.67 (bs, 1H, Ar); 9.07 (d, J 5.0 Hz, 1H, Ar); 10.37 (bs, 1H, HCl salt); 12.53 (bs, 1H, NH). M/Z (M+H)⁺=485.0. MP>250° C.

### Compound 160 (6-(2-(2,2-dimethylmorpholino)ethyl)-8-methyl-2-(4-(trifluoromethyl)pyridin-2-yl)quinazolin-4(3H)-one hydrochloride)

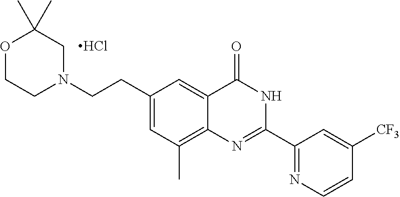 [SMILES: Cc1cc(CCN2CCOC(C)(C)C2)cc2c(=O)[nH]c(-c3cc(C(F)(F)F)ccn3)nc12 ; Cl]

Compound 160 was prepared according to procedure of example 38 using 2,2-dimethylmorpholine instead of homomorpholine and triethylamine instead of potassium carbonate in step 6, followed by procedure of example 42 steps 5 and 6 without isolation of the product at step 5. The free base was purified by flash column chromatography on silica gel using dichloromethane/methanol and by preparative HPLC. Pure fractions were freeze-dried with water and an excess of aqueous 1N HCl to afford the product as a white solid.

¹H-NMR (400 MHz, DMSO): 1.22 (s, 3H, CH₃); 1.44 (s, 3H, CH₃); 2.68 (s, 3H, CH₃); 2.86-3.02 (m, 2H, CH₂); 3.18-3.26 (m, 2H, CH₂—N); 3.36-3.54 (m, 4H, 2 CH₂—N); 3.82-3.94 (m, 2H, CH₂—O); 7.67 (d, J 1.2 Hz, 1H, Ar); 7.97 (bs, 1H, Ar); 8.05 (dd, J 1.2, 5.0 Hz, 1H, Ar); 8.67 (s, 1H, Ar); 9.04 (d, J 5.0 Hz, 1H, Ar); 10.22 (bs, 1H, HCl salt); 12.17 (s, 1H, NH). M/Z (M+H)⁺=447.8. MP=106-116° C.

### Compound 161 (8-Methyl-6-((4-methyl-3-oxopiperazin-1-yl)methyl)-2-(4-(trifluoromethyl)pyridin-2-yl)quinazolin-4(3H)-one hydrochloride)

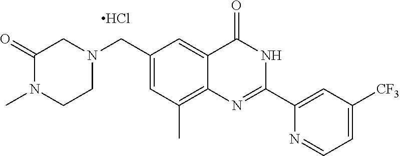 [SMILES: Cc1cc(CN2CCN(C)C(=O)C2)cc2c(=O)[nH]c(-c3cc(C(F)(F)F)ccn3)nc12 ; Cl]

Compound 161 was prepared according to procedure of example 37, starting from 6-bromo-8-methyl-2-(4-methyl-2-pyridyl)-3-(2-trimethylsilylethoxymethyl) quinazolin-4-one and using trifluoro((4-methyl-3-oxopiperazin-1-ium-1-yl)methyl)borate internal salt in step 1 instead of morpholinium-4-yl-methyl)trifluoroborate internal salt to afford the product as a white solid.

¹H-NMR (400 MHz, DMSO): 2.70 (s, 3H, CH₃); 2.86 (s, 3H, CH₃—N); 3.36-3.90 (m, 6H, CH₂—N); 4.22-4.62 (m, 2H, CH₂—N); 7.99 (bs, 1H, Ar); 8.11 (dd, J 5.1, 0.9 Hz, 1H, Ar); 8.22 (bs, 1H, Ar); 8.68 (s, 1H, Ar); 9.06 (d, J 5.1 Hz, 1H, Ar); 11.91 (bs, 1H, HCl salt); 12.31 (s, 1H, NH). M/Z (M+H)⁺=432.1. MP>250° C.

### Example 43—Synthesis of compound 162 (6-(2-(8-Oxa-3-azabicyclo[3.2.1]octan-3-yl)ethyl)-8-methyl-2-(4-(trifluoromethyl)pyridin-2-yl)quinazolin-4(3H)-one hydrochloride)

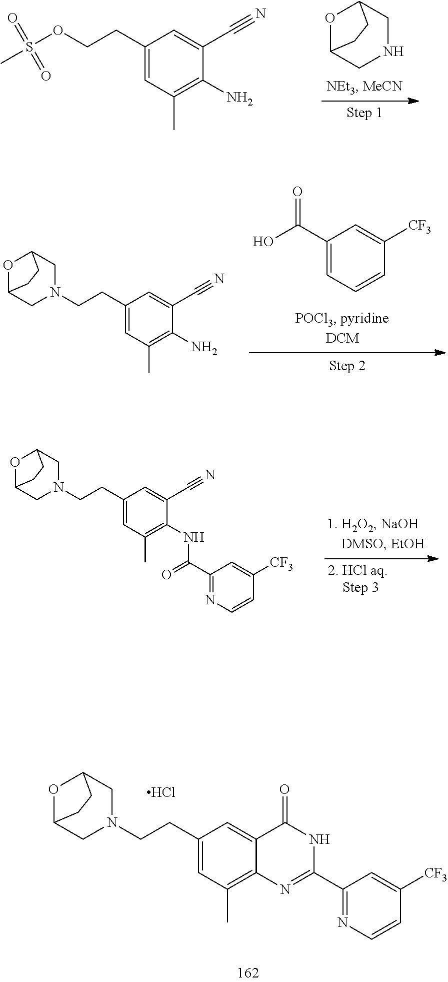

Step 1:

5-(2-(8-oxa-3-azabicyclo[3.2.1]octan-3-yl)ethyl)-2-amino-3-methylbenzonitrile was prepared from 4-amino-3-cyano-5-methylphenethyl methanesulfonate according to procedure of example 42, step 2 using 8-oxa-3-azabicyclo[3.2.1]octane instead of morpholine. The crude residue was purified by flash column chromatography on silica gel using dichloromethane/methanol to afford the product as a yellow oil in 41% yield.

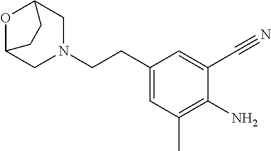 [SMILES: Cc1cc(CCN2CC3CCC(C2)O3)cc(C#N)c1N]

M/Z (M+H)⁺: 272.2.

Step 2:

To a solution of 5-(2-(8-oxa-3-azabicyclo[3.2.1]octan-3-yl)ethyl)-2-amino-3-methylbenzonitrile (87 mg, 0.32 mmol) in dichloromethane (4.3 mL) at 0° C. were added 4-(trifluoromethyl)picolinic acid (79 mg, 0.41 mmol), phosphorus(V) oxychloride (58 μL, 0.64 mmol) and pyridine (52 μL, 0.64 mmol). The reaction mixture was stirred for 30 min then was washed with aqueous 1N NaOH (2*4 mL). The aqueous phases were extracted with dichloromethane (2*30 mL). The combined organic extracts were washed with brine, dried over MgSO₄ and evaporated to dryness to afford crude N-(4-(2-(8-oxa-3-azabicyclo[3.2.1]octan-3-yl)ethyl)-2-cyano-6-methylphenyl)-4-(trifluoromethyl)picolinamide that was used as such.

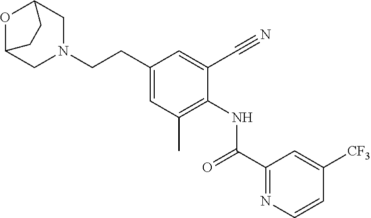 [SMILES: Cc1cc(CCN2CC3CCC(C2)O3)cc(C#N)c1NC(=O)c1cc(C(F)(F)F)ccn1]

M/Z (M+H)⁺: 445.2.

Step 3:

To a solution of N-(4-(2-(8-oxa-3-azabicyclo[3.2.1]octan-3-yl)ethyl)-2-cyano-6-methylphenyl)-4-(trifluoromethyl)picolinamide (0.292 mmol) in ethanol (3 mL) was added hydrogen peroxide (30% in water, 687 μL, 6.727 mmol), aqueous 1N NaOH solution (3.5 mL, 3.509 mmol) and dimethylsulfoxide (477 μL, 6.727 mmol). The reaction mixture was stirred at room temperature for 30 min. Then it was quenched with a solution of saturated sodium thiosulfate (30 mL), extracted twice with ethyl acetate (2*30 mL), washed with brine, dried over MgSO₄ and evaporated to dryness. The crude residue was purified by preparative HPLC. Pure fractions were freeze-dried with water and an excess of aqueous 1N HCl to afford compound 162 as a white solid (21 mg, 15% over 2 steps.

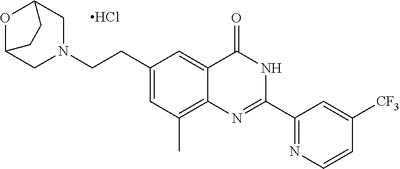 [SMILES: Cc1cc(CCN2CC3CCC(C2)O3)cc2c(=O)[nH]c(-c3cc(C(F)(F)F)ccn3)nc12 ; Cl]

¹H-NMR (400 MHz, DMSO, 80° C.): 1.95 (bs, 2H, CH₂); 2.14 (bs, 2H, CH₂); 2.69 (s, 3H, CH₃); 3.15-3.26 (m, 8H, CH₂); 4.46 (bs, 2H, CH—O); 7.66 (d, J 1.3 Hz, 1H, Ar); 7.97 (d, J 1.3 Hz, 1H, Ar); 7.99 (bs, 1H, Ar); 8.67 (s, 1H, Ar); 9.03 (d, J 5.2 Hz, 1H, Ar); 11.57 (bs, 1H, HCl salt); NH signal not observed. M/Z (M+H)⁺=445.1. MP=160-170° C.

### Compound 163 (6-(2-(3-oxa-8-azabicyclo[3.2.1]octan-8-yl)ethyl)-8-methyl-2-(4-(trifluoromethyl)pyridin-2-yl)quinazolin-4(3H)-one hydrochloride)

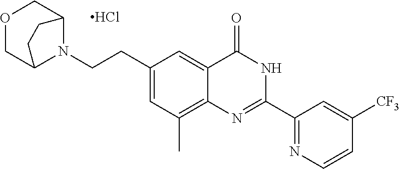 [SMILES: Cc1cc(CCN2C3CCC2COC3)cc2c(=O)[nH]c(-c3cc(C(F)(F)F)ccn3)nc12 ; Cl]

Compound 163 was prepared according to procedure of example 43, using 3-oxa-8-azabicyclo[3.2.1]octane in step 1 instead of 8-oxa-3-azabicyclo[3.2.1]octane. The free base was purified by flash column chromatography on silica gel using dichloromethane/methanol. The HCl salt was obtained by freeze-drying of a suspension of the free base in water and an excess of aqueous 1N HCl to afford the product as a white solid.

¹H-NMR (400 MHz, DMSO, 80° C.): 2.07 (bs, 2H, CH₂); 2.22 (bs, 2H, CH₂); 2.70 (s, 3H, CH₃); 3.28 (bs, 4H, 2 CH₂); 3.69 (d, J 13.1 Hz, 2H, CH₂—O); 4.02 (bs, 2H, 2 CH—N); 4.15 (d, J 13.1 Hz, 2H, CH₂—O); 7.71 (bs, 1H, Ar); 7.98 (d, J 5.0 Hz, 1H, Ar); 8.02 (bs, 1H, Ar); 8.67 (bs, 1H, Ar); 9.03 (d, J 5.0 Hz, 1H, Ar); 10.77 (bs, 1H, HCl salt); 11.59 (bs, 1H, NH); M/Z (M+H)⁺=445.2. MP=200-250° C.

### Compound 164 (6-(2-(4-hydroxypiperidin-1-yl)ethyl)-8-methyl-2-(4-(trifluoromethyl)pyridin-2-yl)quinazolin-4(3H)-one hydrochloride)

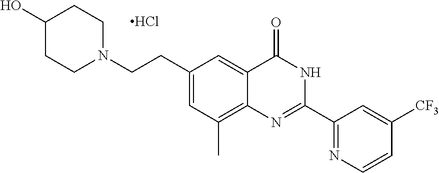 [SMILES: Cc1cc(CCN2CCC(O)CC2)cc2c(=O)[nH]c(-c3cc(C(F)(F)F)ccn3)nc12 ; Cl]

Compound 164 was prepared according to procedure of example 43, using piperidin-4-ol in step 1 instead of 8-oxa-3-azabicyclo[3.2.1]octane. The free base was purified by flash column chromatography on silica gel using dichloromethane/methanol. The HCl salt was obtained by freeze-drying of a suspension of the free base in water and an excess of aqueous 1N HCl to afford the product as a pale yellow solid.

¹H-NMR (400 MHz, CD₃OD): 1.88 (bs, 2H, CH₂); 2.10 (bs, 2H, CH₂); 2.75 (s, 3H, CH₃); 3.19-3.24 (m, 2H, CH₂); 3.31-3.36 (m, 2H, CH₂—N); 3.40-3.44 (m, 2H, CH₂—N); 3.52 (bs, 2H, CH₂—N); 4.00 (bs, 1H, CH—O); 7.77 (bs, 1H, Ar); 7.89 (bd, J 5.3 Hz, 1H, Ar); 8.04 (bs, 1H, Ar); 8.78 (bs, 1H, Ar); 8.99 (d, J 5.3 Hz, 1H, Ar); M/Z (M+H)⁺=433.2. MP=115-125° C.

### Compound 165 (6-(2-(4,4-difluoropiperidin-1-yl)ethyl)-8-methyl-2-(4-(trifluoromethyl)pyridin-2-yl)quinazolin-4(3H)-one hydrochloride)

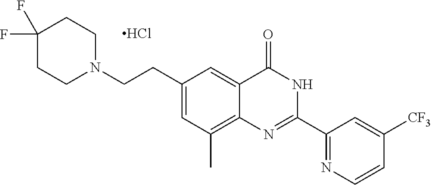 [SMILES: Cc1cc(CCN2CCC(F)(F)CC2)cc2c(=O)[nH]c(-c3cc(C(F)(F)F)ccn3)nc12 ; Cl]

Compound 165 was prepared according to procedure of example 43, using 4,4-difluoropiperidine in step 1 instead of 8-oxa-3-azabicyclo[3.2.1]octane. The free base was purified by flash column chromatography on silica gel using dichloromethane/methanol. The HCl salt was obtained by freeze-drying of a suspension of the free base in water and an excess of aqueous 1N HCl to afford the product as a white solid.

¹H-NMR (400 MHz, DMSO): 2.39 (bs, 2H, CH₂—CF₂); 2.67 (s, 3H, CH₃); 3.21 (bs, 4H, CH₂—CF₂+CH₂); 3.47 (bs, 4H, CH₂—N); 3.71 (bs, 2H, CH₂—N); 7.68 (bs, 1H, Ar); 7.98 (bs, 1H, Ar); 8.04 (bd, J 4.6 Hz, 1H, Ar); 8.66 (bs, 1H, Ar); 9.03 (bd, J 4.6 Hz, 1H, Ar); 10.96 (bs, 1H, HCl salt); 12.16 (s, 1H, NH); M/Z (M+H)⁺=453.2. MP=240-250° C.

### Compound 166 (6-(2-(4-methoxypiperidin-1-yl)ethyl)-8-methyl-2-(4-(trifluoromethyl)pyridin-2-yl)quinazolin-4(3H)-one hydrochloride)

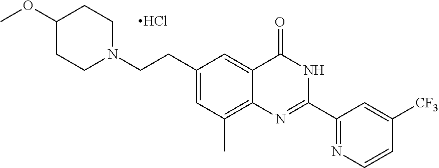 [SMILES: COC1CCN(CCc2cc(C)c3nc(-c4cc(C(F)(F)F)ccn4)[nH]c(=O)c3c2)CC1 ; Cl]

Compound 166 was prepared according to procedure of example 43, using 4-methoxypiperidine in step 1 instead of 8-oxa-3-azabicyclo[3.2.1]octane. The free base was purified by flash column chromatography on silica gel using dichloromethane/methanol. The HCl salt was obtained by freeze-drying of a suspension of the free base in water and an excess of aqueous 1N HCl to afford the product as a white solid.

¹H-NMR (400 MHz, DMSO): 1.59-1.72 (m, 1H, CH₂); 1.87-2.07 (m, 2H, CH₂); 2.11-2.23 (m, 1H, CH₂); 2.67 (s, 3H, CH₃); 2.94-3.13 (m, 2H, CH₂); 3.14-3.23 (m, 2H, CH₂—N); 3.28 (s, 3H, CH₃—O); 3.34-3.47 (m, 4H, CH₂—N); 3.53-3.63 (m, 1H, CH—O); 7.68 (bs, 1H, Ar); 7.97 (bs, 1H, Ar); 8.04 (bd, J 5.2 Hz, 1H, Ar); 8.66 (bs, 1H, Ar); 9.03 (d, J 5.2 Hz, 1H, Ar); 10.26 (bs, 1H, HCl salt); 12.15 (s, 1H, NH); M/Z (M+H)⁺=447.2. MP>250° C.

### Example 44—Synthesis of compound 167 (8-methyl-6-(2-morpholinoethyl)-2-(4-(trifluoromethyl)pyridin-2-yl)pyrido[3,2-d]pyrimidin-4(3H)-one hydrochloride)

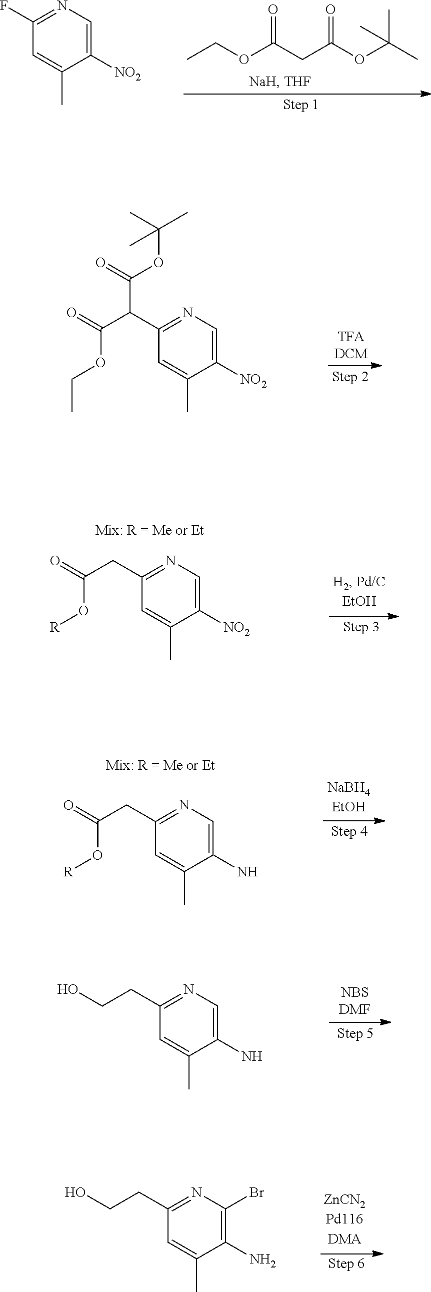

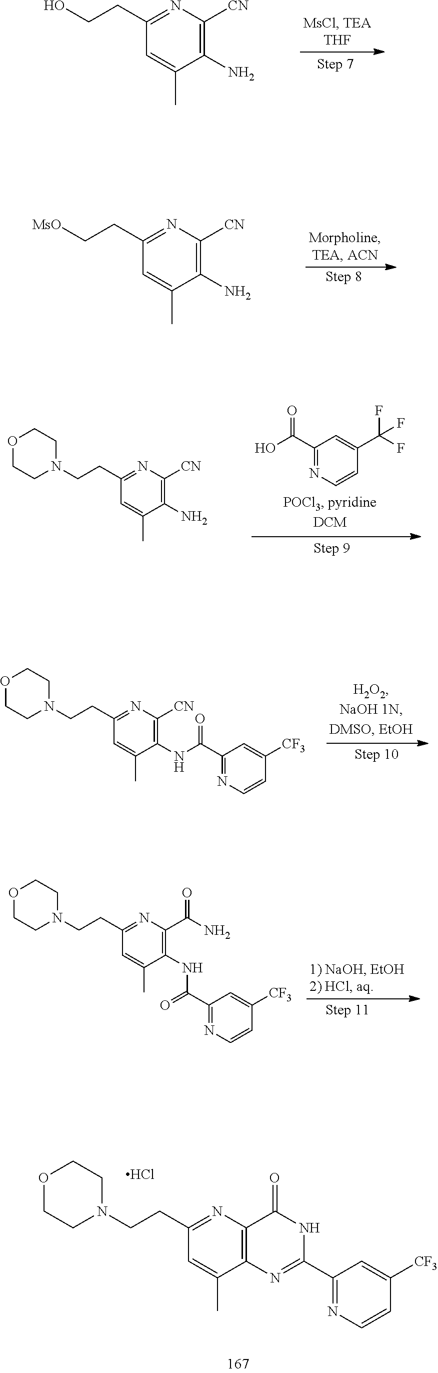

Step 1:

Under inert atmosphere, to a solution of tert-butyl ethyl malonate (2.43 mL, 12.82 mmol) in tetrahydrofuran (42 mL) at 0° C. sodium hydride (60% in mineral oil, 512 mg, 12.82 mmol) was added portionwise. The mixture was stirred at 0° C. for 30 min, then 2-fluoro-4-methyl-5-nitropyridine (1 g, 6.41 mmol) was added and the reaction was stirred for 2 h at room temperature. Then it was poured in an aqueous saturated solution of ammonium chloride and extracted twice with ethyl acetate. The combined organic extracts were dried over Na₂SO₄ and concentrated in vacuo. The oily crude residue (3.4 g) containing 1-(tert-butyl) 3-ethyl 2-(4-methyl-5-nitropyridin-2-yl)malonate in a mixture with tert-butyl ethyl malonate was used as such for the next step.

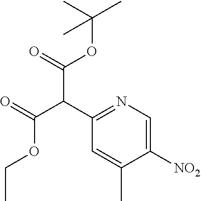 [SMILES: CCOC(=O)C(C(=O)OC(C)(C)C)c1cc(C)c([N+](=O)[O-])cn1]

Step 2:

To a solution of crude 1-(tert-butyl) 3-ethyl 2-(4-methyl-5-nitropyridin-2-yl)malonate (6.41 mmol) in dichloromethane (20 mL) at 0° C. was added trifluoroacetic acid (12 mL) and the mixture was stirred at room temperature for 1 h before being slowly hydrolyzed by an aqueous saturated solution of sodium carbonate. The product was then extracted once with dichloromethane, twice with ethyl acetate. The combined organic extracts were dried over Na₂SO₄ and concentrated in vacuo. The crude residue was purified by flash column chromatography on silica gel preparing a solid loading in methanol, resulting in partial transesterification of the product, and using cyclohexane/ethyl acetate as eluent to afford a mixture of methyl 2-(4-methyl-5-nitropyridin-2-yl)acetate and ethyl 2-(4-methyl-5-nitropyridin-2-yl)acetate (1.33 g, 93%) as an orange oil.

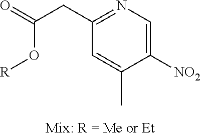

M/Z (M+H)⁺=211.1 & 225.1

Step 3:

Under inert atmosphere, to a solution of a mixture of methyl 2-(4-methyl-5-nitropyridin-2-yl)acetate and ethyl 2-(4-methyl-5-nitropyridin-2-yl)acetate (100 mg, 0.45 mmol) in ethanol was added palladium on charcoal (10 wt %, 4 mg, 0.045 mmol). The mixture was sparged dihydrogen gas and stirred overnight at room temperature under dihydrogen atmosphere. Then the reaction mixture was filtrated over Celite® and concentrated in vacuo. The crude residue was purified by flash column chromatography on silica gel using dichloromethane/methanol to afford a mixture of methyl 2-(5-amino-4-methylpyridin-2-yl)acetate and ethyl 2-(5-amino-4-methylpyridin-2-yl)acetate (70 mg, 80%) as a brown solid.

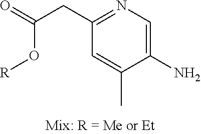

M/Z (M+H)⁺=195.1

Step 4:

Under inert atmosphere, to a solution of a mixture of a mixture of methyl 2-(5-amino-4-methylpyridin-2-yl)acetate and ethyl 2-(5-amino-4-methylpyridin-2-yl)acetate (627 mg, 3.23 mmol) in ethanol (16 mL) was added sodium borohydride (611 mg, 16.14 mmol). The mixture was stirred overnight at 50° C., then aqueous NaOH solution (3N, 16 mL) was added and the mixture was stirred at room temperature for 20 h. The pH was adjusted to 8 with 1 N HCl and the mixture was freeze-dried. The crude residue was triturated in dichloromethane/methanol and filtrated. The filtrate was purified by flash column chromatography on silica gel using dichloromethane/methanol to afford 2-(5-amino-4-methylpyridin-2-yl)ethan-1-ol (325 mg, 66%) as an orange solid.

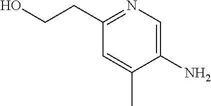

¹H-NMR (400 MHz, DMSO): 2.05 (m, 3H, CH₃); 2.67 (t, J 7.0 Hz, 2H, CH₂); 3.62 (t, J 7.0 Hz, 2H, CH₂—O); 4.94 (bs, 2H, NH₂); 6.85 (s, 1H, Ar); 7.80 (s, 1H, Ar), OH signal not observed.

Step 5:

2-(5-amino-6-bromo-4-methylpyridin-2-yl)ethan-1-ol was prepared according the procedure of example, 38 step 1 starting from 2-(5-amino-4-methylpyridin-2-yl)ethan-1-ol to afford the product as a brown oil. The crude was not purified and used as such in the next step.

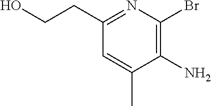

M/Z (M[⁷⁹Br]+H)⁺=233.0

Step 6:

3-amino-6-(2-hydroxyethyl)-4-methylpicolinonitrile was prepared according to the procedure of example 38, step 2, starting from crude 2-(5-amino-6-bromo-4-methylpyridin-2-yl)ethan-1-ol to afford the product as a beige solid in 55% yield over 2 steps.

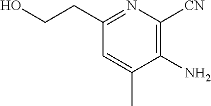

M/Z (M+H)⁺=178.1

Step 7:

2-(5-amino-6-cyano-4-methylpyridin-2-yl)ethyl methanesulfonate was prepared according to procedure of example 38, step 3, starting from 3-amino-6-(2-hydroxyethyl)-4-methylpicolinonitrile and using tetrahydrofurane instead of dichloromethane as a solvent to afford the product as a beige solid. The crude was directly used as such in the next step.

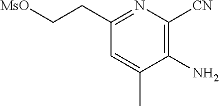 [SMILES: Cc1cc(CCOS(C)(=O)=O)nc(C#N)c1N]

M/Z (M+H)⁺=256.1

Step 8:

3-amino-4-methyl-6-(2-morpholinoethyl)picolinonitrile was prepared according to procedure of example 43, step 1, starting from 2-(5-amino-6-cyano-4-methylpyridin-2-yl)ethyl methanesulfonate and using morpholine instead of 8-oxa-3-azabicyclo[3.2.1]octane to afford the product as a white solid in 75% yield over 2 steps.

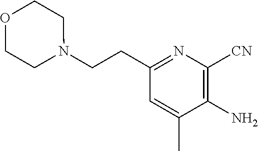 [SMILES: Cc1cc(CCN2CCOCC2)nc(C#N)c1N]

M/Z (M+H)⁺=247.2

Step 9:

N-(2-cyano-4-methyl-6-(2-morpholinoethyl)pyridin-3-yl)-4-(trifluoromethyl)picolinamide (80 mg, 67%) was prepared according to procedure of example 43, step 2, starting from 3-amino-4-methyl-6-(2-morpholinoethyl)picolinonitrile (70 mg, 0.284 mmol) to afford the product as a white solid.

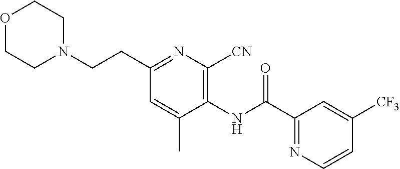 [SMILES: Cc1cc(CCN2CCOCC2)nc(C#N)c1NC(=O)c1cc(C(F)(F)F)ccn1]

M/Z (M+H)⁺=247.2

Step 10:

To a solution of N-(2-cyano-4-methyl-6-(2-morpholinoethyl)pyridin-3-yl)-4-(trifluoromethyl)picolinamide (78 mg, 0.186 mmol) in ethanol (2 mL) was added hydrogen peroxide (30% in water, 128 μL, 4.28 mmol), aqueous NaOH solution (1N, 2.2 mL, 2.23 mmol) and dimethylsulfoxide (301 μL, 4.28 mmol). The reaction mixture was stirred at room temperature for 1.5 h. Then it was quenched with a solution of saturated sodium thiosulfate (20 mL), extracted with ethyl acetate (20 mL), washed with brine, dried over MgSO₄ and evaporated to dryness. The crude residue was purified by flash column chromatography on silica gel using dichloromethane/methanol to afford 4-methyl-6-(2-morpholinoethyl)-3-(4-(trifluoromethyl)picolinamido)picolinamide (47 mg, 58%) as a white solid.

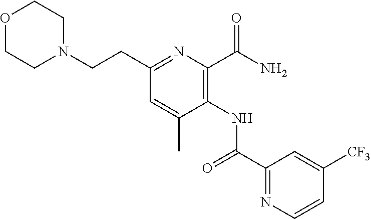 [SMILES: Cc1cc(CCN2CCOCC2)nc(C(N)=O)c1NC(=O)c1cc(C(F)(F)F)ccn1]

M/Z (M+H)⁺=438.3

Step 11:

To a solution of 4-methyl-6-(2-morpholinoethyl)-3-(4-(trifluoromethyl)picolinamido)picolinamide (46 mg, 0.105 mmol) in ethanol (1 mL) was added aqueous NaOH solution (1 N, 1.05 mL, 1.05 mmol) and the solution was stirred for 3 h at room temperature. The mixture was diluted with water (7 mL) and the product was extracted with dichloromethane (5*15 mL). The combined organic extracts were filtrated over hydrophobic filter and evaporated to dryness. The crude residue was purified by flash column chromatography on silica gel using dichloromethane/methanol. The HCl salt was obtained by freeze-drying of a suspension of the free base in water and an excess of aqueous 1N HCl to afford compound 167 (45 mg, 94%) as a white solid.

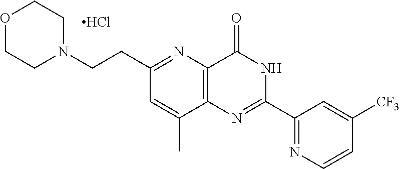 [SMILES: Cc1cc(CCN2CCOCC2)nc2c(=O)[nH]c(-c3cc(C(F)(F)F)ccn3)nc12 ; Cl]

¹H-NMR (400 MHz, DMSO): 2.70 (s, 3H, CH₃); 3.06-3.26 (m, 2H, CH₂); 3.40 (t, J 7.5 Hz, 2H, CH₂—N); 3.48-3.69 (m, 4H, 2 CH₂—N); 3.71-3.91 (m, 2H, CH₂—O); 3.91-4.11 (m, 2H, CH₂—O); 7.75 (s, 1H, Ar); 8.08 (dd, J 5.1, 1.1 Hz, 1H, Ar); 8.68 (bs, 1H, Ar); 9.06 (d, J 5.1 Hz, 1H, Ar); 10.80 (bs, 1H, HCl salt); 12.55 (s, 1H, NH). M/Z (M+H)⁺=420.2. MP=168-173° C.

### II. Biological Assays

Compounds are tested successively for their agonist and positive allosteric modulator activities on human mGluR4 (hmGluR4) transiently over-expressed in HEK-293 cells. Compounds exert agonist activity if, by themselves in absence of glutamate, they are able to activate hmGluR4; and they exert positive allosteric modulator activity if they increase the action of glutamate.

Cell Culture and Transfection

HEK-293 cells are maintained in Modified Eagle's Medium supplemented with 10% Foetal Calf Serum, 1% Penicillin/Streptomycin and 1% non-essential amino acids at 37° C./5% CO₂.

Cells are co-transfected by electroporation with four DNA plasmids encoding hmGluR4, a chimeric G protein allowing redirection of the activation signal to intracellular calcium pathway, and glutamate transporters to decrease extracellular glutamate concentration so as to limit receptor desensitization. After transfection, cells are cultured for 24 h at 37° C./5% CO₂.

Calcium Assay EC50 determination

Receptor activity is detected by changes in intracellular calcium measured using the fluorescent Ca²⁺ sensitive dye, Fluo4AM (Molecular Probes).

On the day of the assay, culture medium is aspirated and replaced during 3 hours by medium without serum supplemented with 1% Glutamax, 1% Penicillin/Streptomycin and 1% non-essential amino acids. Then, cells are washed with freshly prepared buffer B (HBSS 1× (PAA), Hepes 20 mM, MgSO₄-7H₂O 1 mM, Na₂CO₃ 3.3 mM, CaCl₂-2H₂O 1.3 mM, 0.1% BSA, Probenecid 2.5 mM) and loaded at 37° C. in 5% CO₂ for 1.5 hours with buffer B containing 1 μM Fluo4AM, 0.1 mg/mL Pluronic Acid, 7 μg/mL Glutamate Pyruvate Transaminase and 2 mM sodium pyruvate. Afterwards cells are washed twice with buffer B. Then cells are detached using StemPro Accutase (Fisher Scientific), resuspended in buffer B and seeded in 384 well plate at a density of 30,000 cells per well. Addition of compounds and intracellular Ca²⁺ measurements (excitation 485 nm, emission 525 nm) are performed by the fluorescence microplate reader FLIP^{Tetra} (Molecular Devices).

Agonist and positive allosteric modulator activities of compounds are consecutively evaluated on the same cell plate. Agonist activity is first tested during 10 minutes with the addition of compound alone on the cells. Then, cells are stimulated by an EC20 glutamate concentration and fluorescence is recorded for additional 3 minutes. EC20 glutamate concentration is the concentration giving 20% of the maximal glutamate response. Agonist or positive allosteric modulator activity(ies) are evaluated in comparison to basal signals evoked by buffer B or EC20 glutamate alone, respectively.

For EC50 determination, a dose-response test is performed using 20 concentrations (ranging over 6 logs) of each compound. Dose-response curves are fitted using the sigmoidal dose-response (variable slope) analysis in GraphPad Prism program (Graph Pad Inc) and EC50 of agonist/positive allosteric modulator activity is calculated. Dose-response experiments are performed in duplicate, two to three times independently.
The following list represents selected compounds of the present invention showing mGluR4 positive allosteric modulator activity with a measured half maximal effective concentration (EC50)>10 μM:
   Compounds: 24, 27, 33, 34, 36, 41, 68, 71.
The following list represents selected compounds of the present invention showing mGluR4 positive allosteric modulator activity with 1 μM<EC50≤10 μM:
   Compounds: 2, 7, 8, 13, 14, 16, 22, 35, 39, 54, 70, 128, 132, 133, 139, 145, 148, 158.
The following list represents selected compounds of the present invention showing mGluR4 positive allosteric modulator activity with 0.1 μM<EC50≤1 μM:
   Compounds: 1, 3, 4, 5, 9, 10, 11, 12, 15, 18, 19, 23, 30, 32, 37, 44, 47, 48, 51, 57, 58, 59, 60, 61, 62, 63, 64, 66, 67, 69, 72, 73, 74, 75, 77, 79, 82-R, 83-R, 84, 87, 94, 97, 99, 101, 103, 104, 111-R, 111-S, 113, 116, 121, 127, 131, 135, 141, 142, 144, 146, 151, 154, 157, 161, 164, 165, 166.
The following list represents selected compounds of the present invention showing mGluR4 positive allosteric modulator activity with an EC50≤0.1 μM:
   Compounds: 17, 65, 76, 78, 80, 80-R, 80-S, 81, 81-E1, 81-E2, 82-S, 83-S, 86, 88, 89, 90, 91, 92, 93, 96, 100, 102, 105, 106, 107, 108, 109, 110-R, 110-S, 112, 114, 115, 117, 118, 119, 120, 122, 123, 124, 125, 126, 129, 130, 134, 136, 137, 138, 140-R, 140-S, 143, 147, 149, 150, 152, 153, 155, 156, 159, 160, 162, 163.

### III. In Vivo Evaluation on a Haloperidol-Induced Catalepsy Model in the Mouse

This method, which detects anti-cataleptic activity, follows those well-known by one skilled in the art and described in the literature (e.g. Pires et al., Braz J Med and Biol Res 38, 1867-1872, 2005; Shiozaki et al., Psychopharmacology 147, 90-95, 1999). Catalepsy is a symptom of Parkinson's disease, and is characterized by muscular rigidity and fixity of posture.

The procedure applied to the compounds of the invention is as follows:

Catalepsy is assessed using the bar test in mice submitted to acute administration of haloperidol (1 mg/kg, intra-peritoneal or i.p.). Mice (male RjOrl:SWISS mice, weighing 30-35 g at the beginning of the experiment) were placed (6 to 9 in each group) in Plexiglas cages, and injected with haloperidol (1 mg/kg i.p.). The catalepsy response of one mouse was measured when the animal maintained an imposed posture with both forelimbs placed on a horizontal 0.9 cm diameter wire bar suspended 4 cm above a platform. The end point of catalepsy was considered to occur when both forelimbs were removed from the bar, the mouse climbed onto the bar or if the animal moved its head in an exploratory manner. A cut-off time of 180 seconds was applied. The degree of catalepsy was scored 45 minutes after haloperidol administration and continued at 45 minutes intervals for a total of 270 minutes. Between determinations, the animals were returned to their home cages.

Compounds 81, 100, 114, 119, 143 and 144 were evaluated at 1 mg/kg, administered per os, 60 minutes after haloperidol, and compared with a vehicle control group.

FIG. 1 shows the mean time of latency spent on the bar in each group of animals and measured between 135 and 270 min after haloperidol injection. The anti-cataleptic effect of the compound was compared to vehicle-treated group using ANOVA test followed by the Dunnett's test.

In FIG. 1, compounds 81, 100, 114, 119, 143, 144 administered at 1 mg/kg per os 60 minutes after haloperidol injection showed a significant anti-cataleptic effect (with adjusted p values of <0.0001, 0.0065, 0.0066, 0.0307, 0.0176, 0.0115, respectively).

These results demonstrate that the compounds of the invention exhibit anti-cataleptic activity in the haloperidol-induced catalepsy mouse model, which confirms that these compounds are suitable for the treatment of Parkinson's disease.

1. A compound of formula (I) 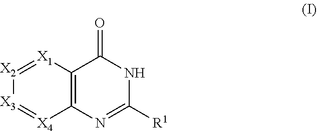
wherein:
R¹ is either selected from any one of the following groups: 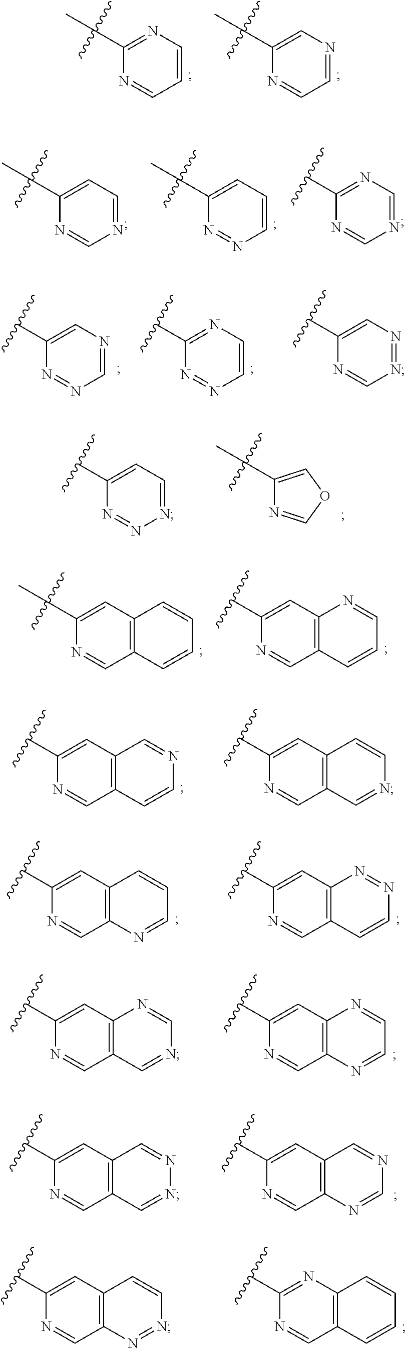 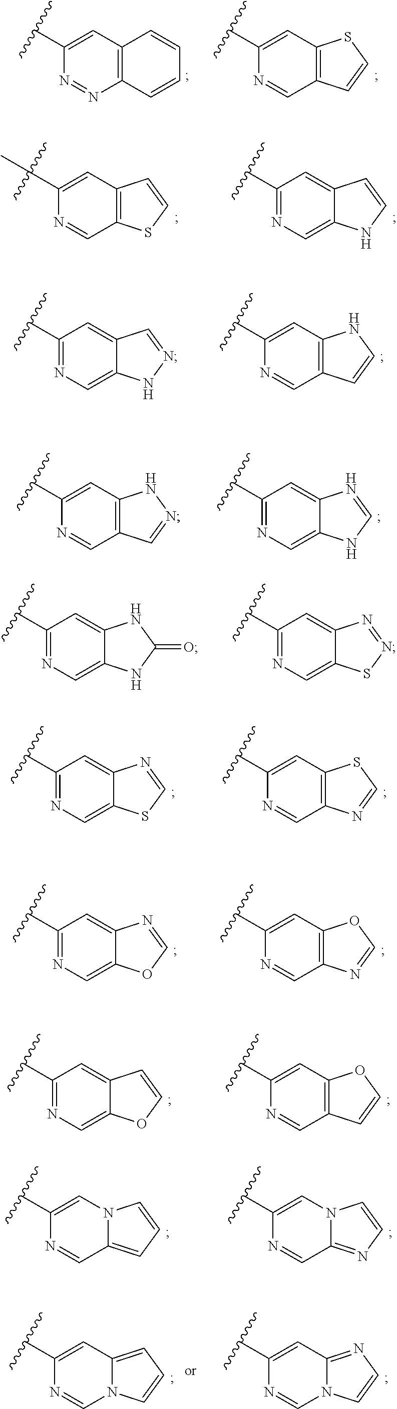
wherein each one of the above-depicted groups is optionally substituted with one or more groups R¹¹;
or R¹ is a group 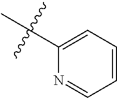 [SMILES: *c1ccccn1]
which is optionally substituted with one or more groups R^{11A};
each R¹¹ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R¹²;
each R^{11A} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-heteroaryl, —(C₀₋₃ alkylene)-cycloalkyl, and —(C₀₋₃ alkylene)-heterocycloalkyl, wherein the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R¹²;
each R¹² is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl;
X₁ is C(R^{X1}) or N;
X₂ is C(-L-R^{X2}) or N;
X₃ is C(R^{X3}) or N;
X₄ is C(R^{X4}) or N;
wherein at least one of the ring atoms X₂, X₃ and X₄ is not N;
R^{X1} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R^{X11};
each R^{X11} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl;
L is selected from a covalent bond, C₁₋₁₀ alkylene, C₂₋₁₀ alkenylene, and C₂₋₁₀ alkynylene, wherein one or more —CH₂— units comprised in said C₁₋₁₀ alkylene, said C₂₋₁₀ alkenylene, or said C₂₋₁₀ alkynylene are each optionally replaced by a group independently selected from —O—, —CO—, —C(═O)O—, —O—C(═O)—, —NH—, —N(C₁₋₅ alkyl)-, —NH—CO—, —N(C₁₋₅ alkyl)-CO—, —CO—NH—, —CO—N(C₁₋₅ alkyl)-, —S—, —SO—, —SO₂—, —SO₂—NH—, —SO₂—N(C₁₋₅ alkyl)-, —NH—SO₂—, —N(C₁₋₅ alkyl)-SO₂—, carbocyclylene, and heterocyclylene, wherein said carbocyclylene and said heterocyclylene are each optionally substituted with one or more groups independently selected from C₁₋₄ alkyl, —OH, —O(C₁₋₄ alkyl), —SH, —S(C₁₋₅ alkyl), —NH₂, —NH(C₁₋₄ alkyl), —N(C₁₋₄ alkyl)(C₁₋₄ alkyl), halogen, C₁₋₅ haloalkyl, —O—(C₁₋₅ haloalkyl), and —CN, and further wherein said C₁₋₁₀ alkylene, said C₂₋₁₀ alkenylene, and said C₂₋₁₀ alkynylene are each optionally substituted with one or more groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
R^{X2} is selected from C₂₋₁₀ alkyl, carbocyclyl, heterocyclyl, and -L¹-R^{X21}, wherein said C₂₋₁₀ alkyl, said carbocyclyl and said heterocyclyl are each optionally substituted with one or more groups R^{X22};
L¹ is selected from a covalent bond, C₁₋₁₀ alkylene, C₂₋₁₀ alkenylene, and C₂₋₁₀ alkynylene, wherein one or more —CH₂— units comprised in said C₁₋₁₀ alkylene, said C₂₋₁₀ alkenylene, or said C₂₋₁₀ alkynylene are each optionally replaced by a group independently selected from —O—, —CO—, —C(═O)O—, —O—C(═O)—, —NH—, —N(C₁₋₅ alkyl)-, —NH—CO—, —N(C₁₋₅ alkyl)-CO—, —CO—NH—, —CO—N(C₁₋₅ alkyl)-, —S—, —SO—, —SO₂—, —SO₂—NH—, —SO₂—N(C₁₋₅ alkyl)-, —NH—SO₂—, and —N(C₁₋₅ alkyl)-SO₂—, and further wherein said C₁₋₁₀ alkylene, said C₂₋₁₀ alkenylene, and said C₂₋₁₀ alkynylene are each optionally substituted with one or more groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
R^{X21} is selected from C₂₋₅ alkyl, carbocyclyl, and heterocyclyl, wherein said carbocyclyl and said heterocyclyl are each optionally substituted with one or more groups R^{X22};
each R^{X22} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R^{X23};
each R^{X23} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), cycloalkyl, and heterocycloalkyl;
R^{X3} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R^{X31};
each R^{X31} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl;
R^{X4} is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-heteroaryl, —(C₀₋₃ alkylene)-cycloalkyl, and —(C₀₋₃ alkylene)-heterocycloalkyl, wherein the aryl moiety in said —(C₀₋₃ alkylene)-aryl, the heteroaryl moiety in said —(C₀₋₃ alkylene)-heteroaryl, the cycloalkyl moiety in said —(C₀₋₃ alkylene)-cycloalkyl, and the heterocycloalkyl moiety in said —(C₀₋₃ alkylene)-heterocycloalkyl are each optionally substituted with one or more groups R^{X41};
each R^{X41} is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, and heterocycloalkyl;
or a pharmaceutically acceptable salt thereof;
with the proviso that the following compounds are excluded from formula (I): 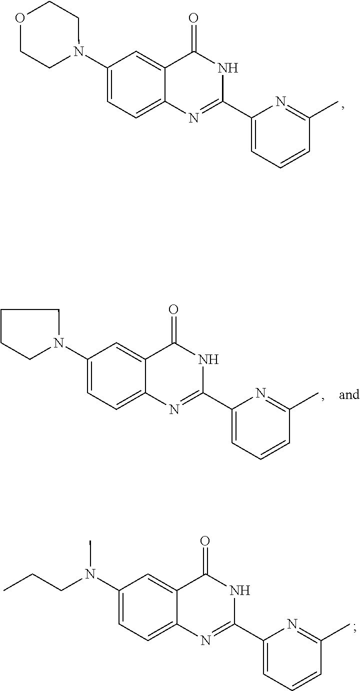
and with the further proviso that the following compound is excluded:

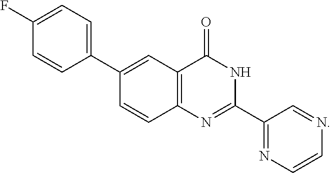 [SMILES: O=c1[nH]c(-c2cnccn2)nc2ccc(-c3ccc(F)cc3)cc12]

2. The compound of claim 1, wherein R¹ is either selected from one of the following groups: 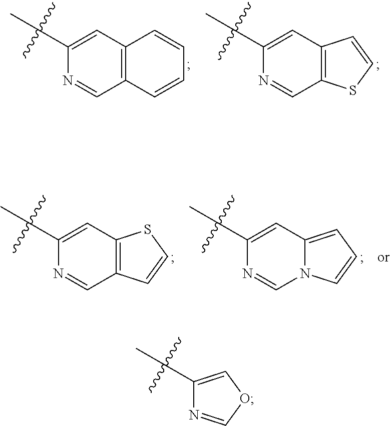 [SMILES: *c1cc2ccccc2cn1 ; *c1cc2ccsc2cn1 ; *c1cc2sccc2cn1 ; *c1cc2cccn2cn1 ; *c1cocn1]
wherein each one of the above-depicted groups is optionally substituted with one or more groups R¹¹;
or R¹ is a group 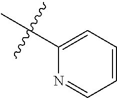 [SMILES: *c1ccccn1]
which is optionally substituted with one or more groups R^{11A}.

3. The compound of claim 1, wherein each R¹¹ and each R^{11A} is independently selected from C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl).

4. The compound of claim 1, wherein X₂ is C(-L-R^{X2}).

5. The compound of claim 1, wherein X₁ is C(R^{X1}), X₂ is C(-L-R^{X2}), X₃ is C(R^{X3}), and X₄ is C(R^{X4}).

6. The compound of claim 1, wherein R^{X2} is selected from cycloalkyl, aryl, heterocycloalkyl, and heteroaryl, wherein said cycloalkyl, said aryl, said heterocycloalkyl, and said heteroaryl are each optionally substituted with one or more groups R^{X22}.

7. The compound of claim 1, wherein the group -L-R^{X2} is selected from —R^{X2}, —(C₁₋₅ alkylene)-R^{X2}, —O—R^{X2}, and —O—(C₁₋₅ alkylene)-R^{X2}, wherein R^{X2} is selected from cycloalkyl, aryl, heterocycloalkyl, and heteroaryl, wherein said cycloalkyl, said aryl, said heterocycloalkyl, and said heteroaryl are each optionally substituted with one or more groups R^{X22}.

8. The compound of claim 1, wherein R^{X2} is selected from azetidinyl, oxetanyl, pyrrolidinyl, oxopyrrolidinyl, tetrahydrofuranyl, piperidinyl, oxopiperidinyl, piperazinyl, morpholinyl, tetrahydropyranyl, 2-oxa-7-aza-spiro[3.5]nonyl, 6-oxa-2-aza-spiro[3.4]octyl, 3-oxa-9-aza-spiro[5.5]undecyl, 7-oxa-2-aza-spiro[4.5]decyl, 8-oxa-2-aza-spiro[4.5]decyl, phenyl, oxazolyl, pyridinyl, pyrazinyl, and pyrimidinyl, wherein each one of the aforementioned cyclic groups is optionally substituted with one or more groups R^{X22}.

9. The compound of claim 1, wherein R^{X4} is selected from hydrogen, methyl, —OCH₃, halogen, and cyclopropyl.

10. The compound of claim 1, wherein R^{X4} is selected from methyl, —OCH₃, halogen, and cyclopropyl.

11. The compound of claim 1, wherein said compound is selected from:
6-(3-Pyridin-4-yl-propoxy)-2-pyrrolo[1,2-c]pyrimidin-3-yl-3H-quinazolin-4-one;
2-Isoquinolin-3-yl-6-(3-pyridin-4-yl-propoxy)-3H-quinazolin-4-one;
6-(3-Pyridin-4-yl-propoxy)-2-thieno[3,2-c]pyridin-6-yl-3H-quinazolin-4-one;
6-(3-Pyridin-4-yl-propoxy)-2-thieno[2,3-c]pyridin-5-yl-3H-quinazolin-4-one;
2-Pyridin-2-yl-6-(3-pyridin-4-yl-propoxy)-3H-quinazolin-4-one;
2-(4-Methoxy-pyridin-2-yl)-6-(3-pyridin-4-yl-propoxy)-3H-quinazolin-4-one;
2-(5-Fluoro-pyridin-2-yl)-6-(3-pyridin-4-yl-propoxy)-3H-quinazolin-4-one;
6-(3-Pyridin-4-yl-propoxy)-2-(5-trifluoromethyl-pyridin-3-yl)-3H-quinazolin-4-one;
6-[3-(4-Pyridyl)propoxy]-2-[5-(trifluoromethyl)-2-pyridyl]-3H-quinazolin-4-one;
2-(4-Methyl-2-pyridyl)-6-[3-(4-pyridyl)propoxy]-3H-quinazolin-4-one;
2-(6-Methyl-2-pyridyl)-6-[3-(4-pyridyl)propoxy]-3H-quinazolin-4-one;
2-(5-Methylpyrazin-2-yl)-6-[3-(4-pyridyl)propoxy]-3H-quinazolin-4-one;
2-[5-Chloro-4-(trifluoromethyl)-2-pyridyl]-6-[3-(4-pyridyl)propoxy]3H-quinazolin-4-one;
2-(4-Chloro-2-pyridyl)-6-[3-(4-pyridyl)propoxy]-3H-quinazolin-4-one;
2-(4-Ethyl-2-pyridyl)-6-[3-(4-pyridyl)propoxy]-3H-quinazolin-4-one;
6-[3-(4-Pyridyl)propoxy]-2-[6-(trifluoromethyl)-2-pyridyl]-3H-quinazolin-4-one;
2-(4-Bromo-2-pyridyl)-6-[3-(4-pyridyl)propoxy]-3H-quinazolin-4-one;
2-(4-Cyclopropyl-2-pyridyl)-6-[3-(4-pyridyl)propoxy]-3H-quinazolin-4-one;
2-(2-Methyl-oxazol-4-yl)-6-(3-pyridin-4-yl-propoxy)-3H-quinazolin-4-one;
6-(2-Pyridin-3-yl-ethoxy)-2-thieno[3,2-c]pyridin-6-yl-3H-quinazolin-4-one;
6-(4-Bromo-benzyloxy)-2-thieno[3,2-c]pyridin-6-yl-3H-quinazolin-4-one;
Tert-butyl 3-(4-hydroxy-2-pyrrolo[1,2-c]pyrimidin-3-yl-quinazolin-6-yl)oxyazetidine-1-carboxylate;
6-(Azetidin-3-yloxy)-2-pyrrolo[1,2-c]pyrimidin-3-yl-3H-quinazolin-4-one;
6-(1-Pyrimidin-4-yl-azetidin-3-yloxy)-2-pyrrolo[1,2-c]pyrimidin-3-yl-3H-quinazolin-4-one;
3-(4-Hydroxy-2-thieno[2,3-c]pyridin-5-yl-quinazolin-6-yloxy)-azetidine-1-carboxylic acid tert-butyl ester;
6-(Azetidin-3-yloxy)-2-thieno[2,3-c]pyridin-5-yl-3H-quinazolin-4-one;
6-(1-Propionyl-azetidin-3-yloxy)-2-thieno[2,3-c]pyridin-5-yl-3H-quinazolin-4-one;
6-(Piperidin-4-yloxy)-2-thieno[3,2-c]pyridin-6-yl-3H-quinazolin-4-one;
6-(1-Propionyl-piperidin-4-yloxy)-2-thieno[3,2-c]pyridin-6-yl-3H-quinazolin-4-one;
6-(2-Morpholin-4-yl-ethoxy)-2-pyrrolo[1,2-c]pyrimidin-3-yl-3H-quinazolin-4-one;
6-(2-Methoxy-ethoxy)-2-pyrrolo[1,2-c]pyrimidin-3-yl-3H-quinazolin-4-one;
6-(2-Morpholin-4-yl-ethoxy)-2-thieno[3,2-c]pyridin-6-yl-3H-quinazolin-4-one;
6-(2-Methoxy-ethoxy)-2-thieno[3,2-c]pyridin-6-yl-3H-quinazolin-4-one;
6-(3-Pyridin-3-yl-propoxy)-2-thieno[3,2-c]pyridin-6-yl-3H-quinazolin-4-one;
4-(4-Oxo-2-pyridin-2-yl-3,4-dihydro-quinazolin-6-yloxy)-piperidine-1-carboxylic acid tert-butyl ester;
6-(Piperidin-4-yloxy)-2-pyridin-2-yl-3H-quinazolin-4-one;
6-(1-Acetyl-piperidin-4-yloxy)-2-pyridin-2-yl-3H-quinazolin-4-one;
4-[4-Oxo-2-(4-trifluoromethyl-pyridin-2-yl)-3,4-dihydro-quinazolin-6-yloxymethyl]-piperidine-1-carboxylic acid tert-butyl ester;
6-(Piperidin-4-ylmethoxy)-2-(4-trifluoromethyl-pyridin-2-yl)-3H-quinazolin-4-one;
6-(1-Acetyl-piperidin-4-ylmethoxy)-2-(4-trifluoromethyl-pyridin-2-yl)-3H-quinazolin-4-one;
tert-butyl 4-[(4-oxo-2-thieno[2,3-c]pyridin-5-yl-3H-quinazolin-6-yl)oxymethyl]piperidine-1-carboxylate;
6-(4-piperidylmethoxy)-2-thieno[2,3-c]pyridin-5-yl-3H-quinazolin-4-one;
6-(1-Acetyl-piperidin-4-ylmethoxy)-2-thieno[2,3-c]pyridin-5-yl-3H-quinazolin-4-one;
6-(1-Propionyl-piperidin-4-ylmethoxy)-2-thieno[2,3-c]pyridin-5-yl-3H-quinazolin-4-one;
3-(4-Oxo-2-thieno[2,3-c]pyridin-5-yl-3,4-dihydro-quinazolin-6-yloxy)-pyrrolidine-1-carboxylic acid tert-butyl ester;
6-(Pyrrolidin-3-yloxy)-2-thieno[2,3-c]pyridin-5-yl-3H-quinazolin-4-one;
6-(1-Acetyl-pyrrolidin-3-yloxy)-2-thieno[2,3-c]pyridin-5-yl-3H-quinazolin-4-one;
4-[4-Oxo-2-(4-trifluoromethyl-pyridin-2-yl)-3,4-dihydro-quinazolin-6-yl]-piperazine-1-carboxylic acid tert-butyl ester;
6-Piperazin-1-yl-2-(4-trifluoromethyl-pyridin-2-yl)-3H-quinazolin-4-one;
6-(4-Propionyl-piperazin-1-yl)-2-(4-trifluoromethyl-pyridin-2-yl)-3H-quinazolin-4-one;
4-(4-Oxo-2-thieno[2,3-c]pyridin-5-yl-3,4-dihydro-quinazolin-6-yl)-piperidine-1-carboxylic acid tert-butyl ester;
6-Piperidin-4-yl-2-thieno[2,3-c]pyridin-5-yl-3H-quinazolin-4-one;
6-(1-Acetyl-piperidin-4-yl)-2-thieno[2,3-c]pyridin-5-yl-3H-quinazolin-4-one;
6-[2-(Tetrahydro-pyran-4-yl)-ethoxy]-2-(4-trifluoromethyl-pyridin-2-yl)-3H-quinazolin-4-one;
6-[3-(3-Fluoro-pyridin-4-yl)-propoxy]-2-thieno[2,3-c]pyridin-5-yl-3H-quinazolin-4-one;
6-[3-(4-Methanesulfonyl-phenyl)-propoxy]-2-thieno[2,3-c]pyridin-5-yl-3H-quinazolin-4-one;
6-(3-Pyrazin-2-yl-propoxy)-2-thieno[2,3-c]pyridin-5-yl-3H-quinazolin-4-one;
6-[3-(3-Methoxy-pyridin-4-yl)-propoxy]-2-thieno[2,3-c]pyridin-5-yl-3H-quinazolin-4-one;
6-[3-(2-Methyl-pyridin-4-yl)-propoxy]-2-thieno[2,3-c]pyridin-5-yl-3H-quinazolin-4-one;
6-(3-Oxazol-4-yl-propoxy)-2-thieno[2,3-c]pyridin-5-yl-3H-quinazolin-4-one;
8-Methyl-6-(3-pyridin-4-yl-propoxy)-2-thieno[2,3-c]pyridin-5-yl-3H-quinazolin-4-one;
6-(3-Pyridin-4-yl-propoxy)-2-thieno[2,3-c]pyridin-5-yl-3H-pyrido[3,2-d]pyrimidin-4-one;
6-(3-Pyridin-4-yl-propoxy)-2-(4-trifluoromethyl-pyridin-2-yl)-3H-pyrido[3,2-d]pyrimidin-4-one;
6-(3-Pyridin-4-yl-propoxy)-2-(4-trifluoromethyl-pyridin-2-yl)-3H-pyrido[2,3-d]pyrimidin-4-one;
6-(3-Pyridin-4-yl-propoxy)-2-(4-trifluoromethyl-pyridin-2-yl)-3H-pyrido[3,4-d]pyrimidin-4-one;
6-(3-Pyridin-4-yl-propoxy)-2-thieno[2,3-c]pyridin-5-yl-7-trifluoromethyl-3H-quinazolin-4-one;
5-Chloro-6-(3-pyridin-4-yl-propoxy)-2-thieno[2,3-c]pyridin-5-yl-3H-quinazolin-4-one;
8-Chloro-6-(3-pyridin-4-yl-propoxy)-2-thieno[2,3-c]pyridin-5-yl-3H-quinazolin-4-one;
8-Cyclopropyl-6-(3-pyridin-4-yl-propoxy)-2-thieno[2,3-c]pyridin-5-yl-3H-quinazolin-4-one;
8-Ethyl-6-(3-pyridin-4-yl-propoxy)-2-thieno[2,3-c]pyridin-5-yl-3H-quinazolin-4-one;
8-Fluoro-6-(3-pyridin-4-yl-propoxy)-2-thieno[2,3-c]pyridin-5-yl-3H-quinazolin-4-one;
8-Methyl-6-(tetrahydro-pyran-4-ylmethoxy)-2-thieno[2,3-c]pyridin-5-yl-3H-quinazolin-4-one;
8-Methyl-6-(2-oxetan-3-yl-ethoxy)-2-thieno[2,3-c]pyridin-5-yl-3H-quinazolin-4-one;
8-Methyl-6-[2-(tetrahydro-furan-3-yl)-ethoxy]-2-thieno[2,3-c]pyridin-5-yl-3H-quinazolin-4-one;
8-Methyl-6-[2-(tetrahydro-pyran-4-yl)-ethoxy]-2-thieno[2,3-c]pyridin-5-yl-3H-quinazolin-4-one;
8-Methyl-6-(tetrahydro-furan-3-ylmethoxy)-2-thieno[2,3-c]pyridin-5-yl-3H-quinazolin-4-one;
R-8-Methyl-6-(tetrahydro-furan-3-ylmethoxy)-2-thieno[2,3-c]pyridin-5-yl-3H-quinazolin-4-one;
S-8-Methyl-6-(tetrahydro-furan-3-ylmethoxy)-2-thieno[2,3-c]pyridin-5-yl-3H-quinazolin-4-one;
8-Methyl-6-(1-methyl-6-oxo-piperidin-3-yloxy)-2-thieno[2,3-c]pyridin-5-yl-3H-quinazolin-4-one;
R-8-Methyl-6-((1-methyl-6-oxopiperidin-3-yl)oxy)-2-(thieno[2,3-c]pyridin-5-yl)quinazolin-4(3H)-one;
S-8-Methyl-6-((1-methyl-6-oxopiperidin-3-yl)oxy)-2-(thieno[2,3-c]pyridin-5-yl)quinazolin-4(3H)-one;
8-Methyl-6-(1-propionyl-pyrrolidin-3-yloxy)-2-thieno[2,3-c]pyridin-5-yl-3H-quinazolin-4-one;
R-8-Methyl-6-(1-propionyl-pyrrolidin-3-yloxy)-2-thieno[2,3-c]pyridin-5-yl-3H-quinazolin-4-one;
S-8-Methyl-6-(1-propionyl-pyrrolidin-3-yloxy)-2-thieno[2,3-c]pyridin-5-yl-3H-quinazolin-4-one;
8-Methyl-6-(1-oxetan-3-yl-pyrrolidin-3-yloxy)-2-thieno[2,3-c]pyridin-5-yl-3H-quinazolin-4-one;
R-8-Methyl-6-(1-oxetan-3-yl-pyrrolidin-3-yloxy)-2-thieno[2,3-c]pyridin-5-yl-3H-quinazolin-4-one;
S-8-Methyl-6-(1-oxetan-3-yl-pyrrolidin-3-yloxy)-2-thieno[2,3-c]pyridin-5-yl-3H-quinazolin-4-one;
8-Methyl-6-[2-(2-oxa-7-aza-spiro[3.5]non-7-yl)-ethoxy]-2-thieno[2,3-c]pyridin-5-yl-3H-quinazolin-4-one;
8-methyl-6-(piperidin-4-ylmethoxy)-2-thieno[2,3-c]pyridin-5-yl-3H-quinazolin-4-one;
8-Methyl-6-(1-oxetan-3-yl-piperidin-4-ylmethoxy)-2-thieno[2,3-c]pyridin-5-yl-3H-quinazolin-4-one;
8-Methyl-6-(1-propionyl-piperidin-4-ylmethoxy)-2-thieno[2,3-c]pyridin-5-yl-3H-quinazolin-4-one;
6-(1-Methanesulfonyl-piperidin-4-ylmethoxy)-8-methyl-2-thieno[2,3-c]pyridin-5-yl-3H-quinazolin-4-one;
8-Methyl-6-(2-oxa-7-aza-spiro[3.5]non-7-yl)-2-thieno[2,3-c]pyridin-5-yl-3H-quinazolin-4-one;
8-Methyl-6-(6-oxa-2-aza-spiro[3.4]oct-2-yl)-2-thieno[2,3-c]pyridin-5-yl-3H-quinazolin-4-one;
8-Methyl-6-(3-oxa-9-aza-spiro[5.5]undec-9-yl)-2-thieno[2,3-c]pyridin-5-yl-3H-quinazolin-4-one;
8-Methyl-6-(7-oxa-2-aza-spiro[4.5]dec-2-yl)-2-thieno[2,3-c]pyridin-5-yl-3H-quinazolin-4-one;
8-Methyl-6-(8-oxa-2-aza-spiro[4.5]dec-2-yl)-2-thieno[2,3-c]pyridin-5-yl-3H-quinazolin-4-one;
6-(2-Hydroxy-2-methyl-propylamino)-8-methyl-2-thieno[2,3-c]pyridin-5-yl-3H-quinazolin-4-one;
8-Methyl-6-(2-piperidin-3-yl-ethoxy)-2-thieno[2,3-c]pyridin-5-yl-3H-quinazolin-4-one;
6-[2-(1-Acetyl-piperidin-3-yl)-ethoxy]-8-methyl-2-thieno[2,3-c]pyridin-5-yl-3H-quinazolin-4-one;
6-[2-(4-Acetyl-piperazin-1-yl)-ethoxy]-8-methyl-2-thieno[2,3-c]pyridin-5-yl-3H-quinazolin-4-one;
3-(8-Methyl-4-oxo-2-thieno[2,3-c]pyridin-5-yl-3,4-dihydro-quinazolin-6-yl)-propionaldehyde;
8-Methyl-6-(3-morpholin-4-yl-propyl)-2-thieno[2,3-c]pyridin-5-yl-3H-quinazolin-4-one;
8-Methyl-6-(2-morpholin-4-yl-ethyl)-2-thieno[2,3-c]pyridin-5-yl-3H-quinazolin-4-one;
8-Methyl-6-(3-pyridin-4-yl-propoxy)-2-(4-trifluoromethyl-pyridin-2-yl)-3H-quinazolin-4-one;
8-Methyl-6-(tetrahydro-furan-3-ylmethoxy)-2-(4-trifluoromethyl-pyridin-2-yl)-3H-quinazolin-4-one;
8-Methyl-6-(1-propionyl-azetidin-3-yloxy)-2-(4-trifluoromethyl-pyridin-2-yl)-3H-quinazolin-4-one;
8-Methyl-6-(1-oxetan-3-yl-piperidin-4-yloxy)-2-(4-trifluoromethyl-pyridin-2-yl)-3H-quinazolin-4-one;
8-Methyl-6-(3-oxa-9-aza-spiro[5.5]undec-9-yl)-2-(4-trifluoromethyl-pyridin-2-yl)-3H-quinazolin-4-one;
8-Methyl-6-(3-pyridin-4-yl-propoxy)-2-pyrrolo[1,2-c]pyrimidin-3-yl-3H-quinazolin-4-one;
8-Methyl-2-pyrrolo[1,2-c]pyrimidin-3-yl-6-(tetrahydro-furan-3-ylmethoxy)-3H-quinazolin-4-one;
8-Methyl-6-(3-oxa-9-aza-spiro[5.5]undec-9-yl)-2-pyrrolo[1,2-c]pyrimidin-3-yl-3H-quinazolin-4-one;
8-Methyl-6-(1-oxetan-3-yl-piperidin-4-yloxy)-2-pyrrolo[1,2-c]pyrimidin-3-yl-3H-quinazolin-4-one;
8-Methyl-6-[1-(tetrahydro-pyran-4-yl)-ethoxy]-2-thieno[2,3-c]pyridin-5-yl-3H-quinazolin-4-one;
R-8-Methyl-6-[1-(tetrahydro-pyran-4-yl)-ethoxy]-2-thieno[2,3-c]pyridin-5-yl-3H-quinazolin-4-one;
S-8-Methyl-6-[1-(tetrahydro-pyran-4-yl)-ethoxy]-2-thieno[2,3-c]pyridin-5-yl-3H-quinazolin-4-one;
8-Methyl-6-[1-(tetrahydro-pyran-4-yl)-ethoxy]-2-(4-trifluoromethyl-pyridin-2-yl)-3H-quinazolin-4-one;
R-8-Methyl-6-[1-(tetrahydro-pyran-4-yl)-ethoxy]-2-(4-trifluoromethyl-pyridin-2-yl)-3H-quinazolin-4-one;
S-8-Methyl-6-[1-(tetrahydro-pyran-4-yl)-ethoxy]-2-(4-trifluoromethyl-pyridin-2-yl)-3H-quinazolin-4-one;
6-[(3-fluorotetrahydrofuran-3-yl)methoxy]-8-methyl-2-[4-(trifluoromethyl)-2-pyridyl]-3H-quinazolin-4-one;
8-methyl-6-(3-oxa-9-azaspiro[5.5]undecan-9-yl)-2-thieno[2,3-c]pyridin-5-yl-3-(2-trimethylsilylethoxymethyl)pyrido[3,2-d]pyrimidin-4-one;
8-methyl-6-(morpholinomethyl)-2-thieno[2,3-c]pyridin-5-yl-3H-quinazolin-4-one;
8-methyl-6-(morpholinomethyl)-2-[4-(trifluoromethyl)-2-pyridyl]-3H-quinazolin-4-one;
8-methyl-6-(1-propanoylazetidin-3-yl)oxy-2-thieno[2,3-c]pyridin-5-yl-3H-quinazolin-4-one;
8-methyl-6-(2-morpholinoethyl)-2-[4-(trifluoromethyl)-2-pyridyl]-3H-quinazolin-4-one;
8-Methyl-6-[(1-methyl-6-oxo-3-piperidyl)oxy]-2-pyrrolo[1,2-c]pyrimidin-3-yl-3H-quinazolin-4-one;
8-Methyl-6-(morpholinomethyl)-2-(thieno[3,2-c]pyridin-6-yl)quinazolin-4(3H)-one;
8-Methyl-6-(3-oxa-9-azaspiro[5.5]undecan-9-yl)-2-(thieno[3,2-c]pyridin-6-yl)quinazolin-4(3H)-one;
8-Methyl-6-(2-[1,4]oxazepan-4-yl-ethyl)-2-thieno[2,3-b]pyridin-5-yl-3H-quinazolin-4-one;
8-Methyl-6-(2-[1,4]oxazepan-4-yl-ethyl)-2-thieno[3,2-b]pyridin-6-yl-3H-quinazolin-4-one;
8-Methyl-6-(2-morpholin-4-yl-ethyl)-2-thieno[3,2-c]pyridin-6-yl-3H-quinazolin-4-one;
8-Methyl-6-(2-morpholin-4-yl-ethyl)-2-pyrrolo[1,2-c]pyrimidin-3-yl-3H-quinazolin-4-one;
8-Methyl-6-(morpholinomethyl)-2-pyrrolo[1,2-c]pyrimidin-3-yl-3H-quinazolin-4-one;
8-Methyl-6-(2-morpholino-2-oxoethyl)-2-(thieno[3,2-c]pyridin-6-yl)quinazolin-4(3H)-one;
8-Methyl-6-(2-morpholino-2-oxo-ethyl)-2-thieno[2,3-c]pyridin-5-yl-3H-quinazolin-4-one;
8-Methyl-6-(2-piperidin-1-yl-ethyl)-2-thieno[2,3-b]pyridin-5-yl-3H-quinazolin-4-one;
8-Methyl-6-(1-methyl-6-oxo-piperidin-3-yloxy)-2-thieno[3,2-c]pyridin-6-yl-3H-quinazolin-4-one;
8-Methyl-6-(1-methyl-2-oxo-piperidin-4-ylmethoxy)-2-thieno[2,3-c]pyridin-5-yl-3H-quinazolin-4-one
8-Methyl-6-(1-piperidylmethyl)-2-thieno[2,3-c]pyridin-5-yl-3H-quinazolin-4-one;
8-Methyl-6-(2-morpholino-2-oxo-ethyl)-2-thieno[3,2-c]pyridin-6-yl-3H-quinazolin-4-one;
8-Methyl-6-[(4-methylpiperazin-1-yl)methyl]-2-thieno[2,3-c]pyridin-5-yl-3H-quinazolin-4-one;
8-Methyl-6-(pyrrolidin-1-ylmethyl)-2-thieno[2,3-c]pyridin-5-yl-3H-quinazolin-4-one;
8-Methyl-6-(2-morpholino-2-oxo-ethyl)-2-pyrrolo[1,2-c]pyrimidin-3-yl-3H-quinazolin-4-one;
8-Methyl-6-(morpholine-4-carbonyl)-2-pyrrolo[1,2-c]pyrimidin-3-yl-3H-quinazolin-4-one;
8-Methyl-6-(1-methyl-6-oxo-piperidin-3-yloxy)-2-(4-trifluoromethyl-pyridin-2-yl)-3H-quinazolin-4-one;
8-Methyl-2-thieno[2,3-c]pyridin-5-yl-6-(thiomorpholinomethyl)-3H-quinazolin-4-one;
8-Methyl-6-[2-(1,4-oxazepan-4-yl)-2-oxo-ethyl]-2-thieno[2,3-c]pyridin-5-yl-3H-quinazolin-4-one;
8-Methyl-6-(pyrrolidin-1-ylmethyl)-2-thieno[3,2-c]pyridin-6-yl-3H-quinazolin-4-one;
8-Methyl-6-(1-methyl-5-oxo-pyrrolidin-3-yl)oxy-2-thieno[3,2-c]pyridin-6-yl-3H-quinazolin-4-one;
8-Methyl-6-[(3R)-1-methyl-5-oxo-pyrrolidin-3-yl]oxy-2-thieno[3,2-c]pyridin-6-yl-3H-quinazolin-4-one;
8-Methyl-6-[(3S)-1-methyl-5-oxo-pyrrolidin-3-yl]oxy-2-thieno[3,2-c]pyridin-6-yl-3H-quinazolin-4-one;
Benzyl 3-[(8-methyl-4-oxo-2-thieno[3,2-c]pyridin-6-yl-3H-quinazolin-6-yl)oxy]pyrrolidine-1-carboxylate;
Benzyl (3S)-3-[(8-methyl-4-oxo-2-thieno[3,2-c]pyridin-6-yl-3H-quinazolin-6-yl)oxy]pyrrolidine-1-carboxylate;
Benzyl (3R)-3-[(8-methyl-4-oxo-2-thieno[3,2-c]pyridin-6-yl-3H-quinazolin-6-yl)oxy]pyrrolidine-1-carboxylate;
8-Methyl-6-[2-(4-methyl-3-oxo-piperazin-1-yl)ethyl]-2-thieno[2,3-c]pyridin-5-yl-3H-quinazolin-4-one;
8-Methyl-6-[2-(4-methyl-3-oxo-piperazin-1-yl)ethyl]-2-[4-(trifluoromethyl)-2-pyridyl]-3H-quinazolin-4-one;
8-Methyl-6-[2-(2-oxa-6-azaspiro[3.3]heptan-6-yl)ethyl]-2-thieno[2,3-c]pyridin-5-yl-3H-quinazolin-4-one;
8-Methyl-6-[2-(2-oxa-6-azaspiro[3.3]heptan-6-yl)ethyl]-2-thieno[3,2-c]pyridin-6-yl-3H-quinazolin-4-one;
8-Methyl-6-[(4-methyl-3-oxo-piperazin-1-yl)methyl]-2-thieno[3,2-c]pyridin-6-yl-3H-quinazolin-4-one;
6-(2-((2-Methoxyethyl)(methyl)amino)ethyl)-8-methyl-2-(thieno[3,2-c]pyridin-6-yl)quinazolin-4(3H)-one;
6-(2-(1,1-Dioxidothiomorpholino)ethyl)-8-methyl-2-(thieno[3,2-c]pyridin-6-yl)quinazolin-4(3H)-one;
6-[(1,1-Dioxo-1,4-thiazinan-4-yl)methyl]-8-methyl-2-thieno[3,2-c]pyridin-6-yl-3H-quinazolin-4-one;
6-(((2-Methoxyethyl)(methyl)amino)methyl)-8-methyl-2-(thieno[3,2-c]pyridin-6-yl)quinazolin-4(3H)-one;
6-[(4-Methoxy-1-piperidyl)methyl]-8-methyl-2-thieno[3,2-c]pyridin-6-yl-3H-quinazolin-4-one;
6-[(2,2-Dimethylmorpholin-4-yl)methyl]-8-methyl-2-thieno[3,2-c]pyridin-6-yl-3H-quinazolin-4-one;
8-Chloro-6-(2-morpholinoethyl)-2-(4-(trifluoromethyl)pyridin-2-yl)quinazolin-4(3H)-one;
8-Methyl-6-(2-oxa-7-azaspiro[3.5]nonan-7-ylmethyl)-2-thieno[3,2-c]pyridin-6-yl-3H-quinazolin-4-one;
N,N-Dimethyl-1-((8-methyl-4-oxo-2-(thieno[3,2-c]pyridin-6-yl)-3,4-dihydroquinazolin-6-yl)methyl)piperidine-4-carboxamide;
6-((4-(Methoxymethyl)piperidin-1-yl)methyl)-8-methyl-2-(thieno[3,2-c]pyridin-6-yl)quinazolin-4(3H)-one;
8-Methoxy-6-(2-morpholinoethyl)-2-(4-(trifluoromethyl)pyridin-2-yl)quinazolin-4(3H)-one;
8-Bromo-6-(2-morpholinoethyl)-2-(4-(trifluoromethyl)pyridin-2-yl)quinazolin-4(3H)-one;
6-(2-(2,2-Dimethylmorpholino)ethyl)-8-methyl-2-(4-(trifluoromethyl)pyridin-2-yl)quinazolin-4(3H)-one;
8-Methyl-6-((4-methyl-3-oxopiperazin-1-yl)methyl)-2-(4-(trifluoromethyl)pyridin-2-yl)quinazolin-4(3H)-one;
6-(2-(8-Oxa-3-azabicyclo[3.2.1]octan-3-yl)ethyl)-8-methyl-2-(4-(trifluoromethyl)pyridin-2-yl)quinazolin-4(3H)-one;
6-(2-(3-Oxa-8-azabicyclo[3.2.1]octan-8-yl)ethyl)-8-methyl-2-(4-(trifluoromethyl)pyridin-2-yl)quinazolin-4(3H)-one;
6-(2-(4-Hydroxypiperidin-1-yl)ethyl)-8-methyl-2-(4-(trifluoromethyl)pyridin-2-yl)quinazolin-4(3H)-one;
6-(2-(4,4-Difluoropiperidin-1-yl)ethyl)-8-methyl-2-(4-(trifluoromethyl)pyridin-2-yl)quinazolin-4(3H)-one;
6-(2-(4-Methoxypiperidin-1-yl)ethyl)-8-methyl-2-(4-(trifluoromethyl)pyridin-2-yl)quinazolin-4(3H)-one;
8-Methyl-6-(2-morpholinoethyl)-2-(4-(trifluoromethyl)pyridin-2-yl)pyrido[3,2-d]pyrimidin-4(3H)-one;
and pharmaceutically acceptable salts of any one of the aforementioned compounds.

12. A pharmaceutical composition comprising the compound of claim 1 and a pharmaceutically acceptable excipient.

13.-14. (canceled)

15. A method of treating or preventing a condition associated with altered glutamatergic signalling and/or functions or a condition which can be affected by alteration of glutamate level or signalling, the method comprising administering the compound of claim 1 to a subject in need thereof.

16. The method of claim 15, wherein the condition to be treated or prevented is selected from any one of: dementias and related diseases, including dementias of the Alzheimer's type, Alzheimer's disease, Pick's disease, vascular dementias, Lewy-body disease, dementias due to metabolic, toxic and deficiency diseases, AIDS-dementia complex, Creutzfeld-Jacob disease and atypical subacute spongiform encephalopathy; parkinsonism and movement disorders, including Parkinson's disease, multiple system atrophy, progressive supranuclear palsy, corticobasal degeneration, hepatolenticular degeneration, chorea, Huntington's disease, hemiballismus, athetosis, dystonias, spasmodic torticollis, occupational movement disorder, Gilles de la Tourette syndrome, tardive or drug induced dyskinesias, levodopa-induced dyskinesia, tremor and myoclonus; social skill disorders including autism or autism spectrum disorders, or fragile X syndrome; acute and chronic pain; anxiety disorders, including panic disorders, phobias, obsessive-compulsive disorders, stress disorders and generalized anxiety disorders; schizophrenia and other psychotic disorders; mood disorders, including depressive disorders and bipolar disorders; endocrine and metabolic diseases including diabetes, disorders of the endocrine glands and hypoglycaemia; and cancers.

17.-18. (canceled)

19. A method of treating or preventing Parkinson's disease, the method comprising administering the compound of claim 1 to a subject in need thereof.

20. The method of claim 15, wherein said subject is a human.

21. A method of identifying a test agent that binds to metabotropic glutamate receptor 4 (mGluR4), comprising the following steps:
(a) contacting mGluR4 with the compound of claim 1, wherein said compound is radio-labeled or fluorescence-labeled, under conditions that permit binding of the compound to mGluR4, thereby generating bound, labeled compound;
(b) detecting a signal that corresponds to the amount of bound, labeled compound in the absence of test agent;
(c) contacting the bound, labeled compound with a test agent;
(d) detecting a signal that corresponds to the amount of bound labeled compound in the presence of test agent; and
(e) comparing the signal detected in step (d) to the signal detected in step (b) to determine whether the test agent binds to mGluR4.

22. (canceled)

23. The method of claim 19, wherein said subject is a human.
